(19) United States
(12) Patent Application Publication
(10) Pub. No.: US 2024/0262822 A1
(43) Pub. Date: Aug. 8, 2024

(54) WDR5 INHIBITORS AND MODULATORS

(71) Applicant: Vanderbilt University, Nashville, TN (US)

(72) Inventors: Taekyu Lee, Brentwood, TN (US); Kevin B. Teuscher, Nashville, TN (US); Somenath Chowdhury, Nashville, TN (US); Jianhua Tian, Montgomery Village, MD (US); Kenneth M. Meyers, Nashville, TN (US); Stephen W. Fesik, Nashville, TN (US)

(21) Appl. No.: 18/559,184

(22) PCT Filed: May 6, 2022
(86) PCT No.: PCT/US2022/028141

§ 371 Date: Nov. 6, 2023

Related U.S. Application Data
(60) Provisional application No. 63/184,904, filed on May 6, 2021.

(51) Int. Cl. C07D 471/04; A61K 31/4725; A61K 31/496; A61K 31/502; A61K 31/517; A61K 31/5377; C07D 401/14; C07D 405/14; C07D 491/052

(52) U.S. Cl. CPC C07D 471/04; A61K 31/4725; A61K 31/496; A61K 31/502; A61K 31/517; A61K 31/5377; C07D 401/14; C07D 405/14; C07D 491/052

(57) ABSTRACT

Isoquinolinone compounds and derivatives inhibit WDR5 and associated protein-protein interactions, and the compounds and their pharmaceutical compositions are useful for treating disorders and conditions in a subject, such as cancer cell proliferation.

### RELATED APPLICATIONS

This application claims priority to U.S. Provisional Application No. 63/184,904, filed May 6, 2021, which is hereby incorporated by reference in its entirety.

### STATEMENT OF GOVERNMENT INTEREST

This invention was made with government support under Contract No. HHSN261200800001E, awarded by the National Institutes of Health. The government has certain rights in the invention.

### TECHNICAL FIELD

The present invention relates generally to compounds that inhibit the binding of transcription factors, regulatory regulators, and chromatin to WDR5 and methods of use thereof. In particular embodiments, the present invention provides compositions comprising imino-azacycle-benzamide compounds and methods of use thereof to inhibit or modulate the interaction of WDR5 with chromatin, cognate transcription and other regulatory factors, including for example the histone methyltransferase MLL1, for the treatment of leukemia, solid cancers and other diseases dependent on activity of WDR5.

### BACKGROUND

Mixed lineage leukemia (MLL) presents a heterogeneous group of acute myeloid leukemia and acute lymphoblastic leukemia bearing features of more than one hematopoietic cell lineage. MLL accounts for about 80% of infant acute leukemia cases (Tomizawa, D.; et. al. Leukemia, 2007, 21, 2258-63) and 10% of all acute leukemia cases (Marschalek, R. Br. J. Haematol. 2011, 152, 141-54). MLL leukemia patients have a poor prognosis with overall 5-year survival ratio around 35% (Dimartino, J. F.; Cleary, M. L., Br. J. Haematol. 1999, 106, 614-626; Pui, C., et al. Leukemia, 2003, 4, 700-706; Tomizawa, D.; et. al. Leukemia, 2007, 21, 2258-63).

MLL is composed of heterogeneous cell lineages with different molecular biology, cell biology and immunology features. However, MLL does share a common feature, which involves the chromosomal rearrangement of Mixed Lineage Leukemia (MLL) gene. MLL gene locates on chromosome 11q23 and the encoded MLL protein is a homolog of Drosophila trithorax (Trx) (Thachuk, D. C.; et al. Cell, 1992, 71, 691-700). Wild type MLL binds to regulatory regions of homeox (HOX) genes (Milne, T. A.; et al. Cancer Res., 2005, 65, 11367-74) through the amino terminal fragment while the catalytic C-terminal domain catalyzes the Histone 3 lysine 4 (H3K4) methylation via interaction with WDR5 and up regulates target gene transcription (Nakamura, T.; et al. Mol. Cell, 2002, 10, 1119-28; Yokoyama, A. et al. Mol. Cell Biol., 2004, 24, 5639-49; Milne, T. A.; et al. Mol. Cell, 2002, 10, 1107-17). Wild type MLL in conjunction with WDR5 is required for maintenance HOX genes expression and is widely expressed not only during embryo development but also in adult tissues including myeloid and lymphoid cells (Yu, B. D.; et al. Proc. Natl. Acad. Sci., 1998, 95, 10632-10636). Reciprocal translocations of MLL gene result in-frame fusion of the 5′-end MLL with the 3′-end of another partner gene. A common feature of MLL1 abnormality in leukemia is the preservation of one wild-type MLL1 allele. Currently, more than 80 partner genes have been identified, with MLL-AF4, MLL-AF9 and MLL-ENL being the three most frequently found fusion genes (Pui, C., et al. Leukemia, 2003, 4, 700-706; herein incorporated by reference in its entirety). Expression of MLL fusion proteins promotes over expression of target genes such as HOXA9 and MEIS1, which blocks differentiation, enhances blast expansion and ultimately leads to leukemic transformation (Caslini, C.; et al. Cancer Res., 2007, 67, 7275-83; Yokoyama, A.; et al. Cell, 2005, 123, 207-18). The numerous chromosomal translocations of MLL gene and partner genes add to the complexity of MLL leukemia treatment. Although HOX9 and MEIS1 overexpression are commonly observed among MLL leukemia patients, each rearrangement leads to distinct dysregulated target gene expression patterns and downstream events (Slany, R. K., Haematologica, 2009, 94, 984-993). Clinical studies reveal that MLL of different chromosomal translocations are associated with different prognosis and are treated differently under current protocols (Tamai, H., et al. J. Clin. Exp. Hematop., 2010, 50, 91-98; Balgobind, B. V., et al. Leukemia, 2011, 8, 1239-1248; Pigazzi, M.; et al. Leukemia, 2011, 25, 560-563).

Intrinsic histone methyltransferase (HMT) activity of MLL1 is extremely low and requires a complex assembly of WDR5, RbBP5, ASH2L, and DPY30 protein partners for effective H3K4 trimethylation, the so-called WRAD complex (Patel, A.; et al. J. Biol. Chem., 2009, 284, 24242-56). The binding of MLL1 to WDR5 (WD40 repeat protein 5) is particularly critical for HMT activity and occurs through a conserved arginine containing motif on MLL1 called the “Win” or WDR5 interaction motif. Thus, targeting inhibitors of the MLL1-WDR5 interaction at the WIN site in order to block MLL1 methyltransferase activity could represent a promising therapeutic strategy for treating MLL leukemia patients. Peptidomimetics have been discovered that bind tightly to WDR5 at the MLL site, inhibit MLL1 methyltransferase activity, and block proliferation of MLL1 cells by inducing cell-cycle arrest, apoptosis, and myeloid differentiation (Cao, F.; et al. Molecular Cell, 2014, 53, 247-61, Karatas, H.; et al. J. Med. Chem., 2017, 60, 4818-4839). In addition, altered gene expression patterns similar to MLL1 deletion are observed, supporting a role for MLL1 activity in regulating MLL1-dependent leukemia transcription. Thus, interruption of the WDR5-MLL1 interaction may be a useful strategy for treating patients with MLL leukemias. In addition to the highly characterized WDR5-MLL1 interaction, disruption of WDR5 with other transcription factors/epigenetic writers or displacement from chromatin itself could have a desirable benefit as a cancer treatment strategy. For example, WDR5 acts as a scaffold protein with the following chromatin complexes/structures, including histone H3 (via R2 residues, e.g. see Song, J.-J., et al. J. Biol. Chem. 2008, 283, 35258-64), NSL/MOF (Li, X., et al. Molecular and Cellular Biology, 2010, 30, 5335-47, Dias, J., et al. Genes & Development, 2014, 28, 929-942), C/EBP□ p 30 (Senisterra, G., et al. Biochem. J., 2013, 449, 151-159), c-MYC (Thomas, L. R.; et al. Molecular Cell, 2015, 58, 440-52, herein incorporated by reference in its entirety), and the NuRD complex (Ee, L.-S., et al. Stem Cell Reports, 2017, 8, 1488-96). In addition, WDR5 expression levels have been reported to be correlative and connected to patient prognosis in several other cancer types, including neuroblastoma (Sun, Y. et al. Cancer Research, 2015, 75, 5143-54), breast cancer (Dai, X. et al. PLoSOne, 2015, 10, PMC4565643), bladder cancer (Chen, X. et al. Scientific Reports, 2015, 5, 8293), and colorectal cancer (Tan, X. et al. Cell Death & Disease, 2017, 8, PMC5386518). In addition, in an unbiased shRNA screen in human xenografts, WDR5 was identified as an important target in pancreatic cancer (Carugo, A. et al. Cell Reports, 2016, 16, 133-147). Based on the growing number of complexes identified, which utilize WDR5 to maintain tumor fitness and growth, the emerging importance of WDR5 in several cancer types is not unexpected. In the case of the c-MYC-WDR5 interaction, the MYC oncoprotein utilizes a molecularly defined interaction with WDR5 to bind to its target genes on chromatin. MYC is overexpressed in a majority of malignancies and contributes to an estimated 70,000-100,000 cancer deaths per year in the United States. Thus, disruption of WDR5 from chromatin as a strategy to displace MYC from its target genes may provide a beneficial strategy to treat MYC-driven tumors.

### SUMMARY

The molecules described herein can inhibit or modulate the interaction of WDR5 with chromatin, cognate transcription and other regulatory factors, including for example the histone methyltransferase MLL1, and can provide a therapeutic approach to treat cancers associated with such interactions (e.g., the MLL1-WDR5 interaction).

In one aspect, the invention provides compounds of formula (I),

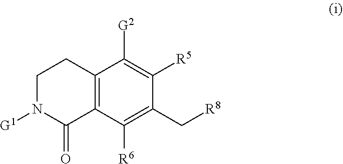

or a tautomer thereof or a pharmaceutically acceptable salt of the compound or tautomer, wherein:
G¹ is

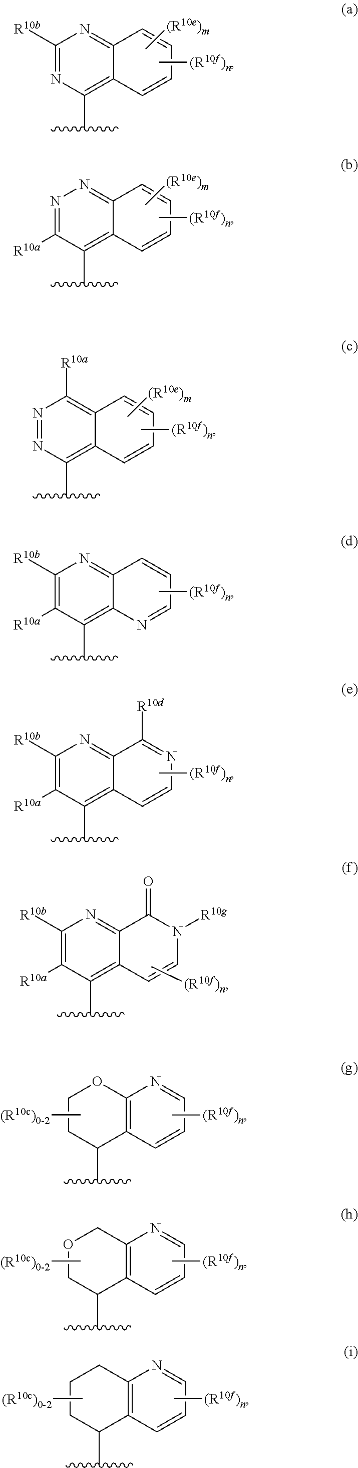

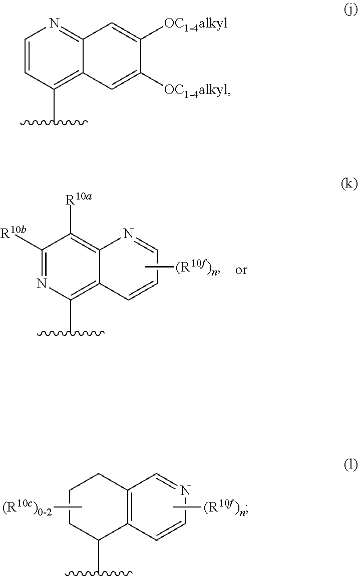

R^{10a} is hydrogen, fluoro, chloro, C₁₋₃alkyl, or C₁₋₃fluoroalkyl;
R^{10b} is hydrogen, fluoro, chloro, C₁₋₃alkyl, C₁₋₃fluoroalkyl, C₃₋₆cycloalkyl, NH₂, —NHC₁₋₄alkyl, —N(C₁₋₄alkyl)₂, or a 4- to 8-membered monocyclic heterocyclyl containing 1-2 heteroatoms independently selected from the group consisting of N, O, and S, wherein the heterocyclyl is optionally substituted with 1-4 substituents independently selected from the group consisting of halogen, cyano, oxo, C₁₋₄alkyl, C₁₋₄fluoroalkyl, OH, —OC₁₋₄alkyl, —OC₁₋₄fluoroalkyl, NH₂, —NHC₁₋₄alkyl, and —N(C₁₋₄alkyl)₂;
R^{10c}, at each occurrence, is independently fluoro, chloro, C₁₋₃alkyl, or C₁₋₃fluoroalkyl;
R^{10d} is X¹, -L¹-X¹, hydrogen, halogen, C₁₋₄alkyl, C₁₋₄fluoroalkyl, G^{1a}, or -L¹-G^{1a};
R^{10e} is halogen, cyano, C₁₋₄alkyl, C₁₋₄fluoroalkyl, OH, —OC₁₋₄alkyl, —OC₁₋₄fluoroalkyl, —OG^{1c}, —OC₁₋₃alkylene-G^{1c}, —O—C₂₋₃alkylene-Y, NH₂, —NHC₁₋₄alkyl, —N(C₁₋₄alkyl)₂, or a 4- to 8-membered monocyclic heterocyclyl containing 1-2 heteroatoms independently selected from the group consisting of N, O, and S, wherein the heterocyclyl is optionally substituted with 1-4 substituents independently selected from the group consisting of halogen, cyano, oxo, C₁₋₄alkyl, C₁₋₄fluoroalkyl, OH, —OC₁₋₄alkyl, —OC₁₋₄fluoroalkyl, NH₂, —NHC₁₋₄alkyl, and —N(C₁₋₄alkyl)₂;
R^{10f}, at each occurrence, is independently halogen, cyano, C₁₋₄alkyl, C₁₋₄fluoroalkyl, OH, —OC₁₋₄alkyl, —OC₁₋₄fluoroalkyl, —OC₃₋₄cycloalkyl, —OC₁₋₃alkylene-C₃₋₄cycloalkyl, —OPG, or —OSO₂CF₃;
R^{10g} is hydrogen, C₁₋₄alkyl, C₁₋₄fluoroalkyl, C₃₋₄cycloalkyl, or —C₁₋₃alkylene-C₃₋₄cycloalkyl;
m is 0 or 1;
n is 0, 1, or 2;
PG is a hydroxy protecting group;
L¹ is C₁₋₃alkylene;
X¹ is —C(O)N(R^{1a})₂, —OR^{1a}, —N(R^{1a})₂, cyano, —C(O)OR^{1a}, —C(O)R^{1b}, —SO₂R^{1b}, —SO₂N(R^{1a})₂, —NR^{1a}C(O)H, or —NR^{1a}C(O)R^{1b};
R^{1a}, at each occurrence, is independently hydrogen, C₁₋₄alkyl, C₁₋₄fluoroalkyl, —C₂₋₃alkylene-OR^{1e}, —C₂₋₃alkylene-N(R^{1e})₂, —C₂₋₃alkylene-N(R^{1e})C(O)R^{1e}, G^{1a}, or —C₁₋₃alkylene-G^{1a};
R^{1b}, at each occurrence, is independently C₁₋₄alkyl, C₁₋₄fluoroalkyl, —C₁₋₃alkylene-OR^{1e}, —C₁₋₃alkylene-N(R^{1e})₂, —C₁₋₃alkylene-N(R^{1e})C(O)R^{1e}, G^{1a}, or —C₁₋₃alkylene-G^{1a};
G^{1a}, at each occurrence, is independently C₃₋₈cycloalkyl, 5- to 6-membered heteroaryl, or 4- to 8-membered heterocyclyl, wherein G^{1a} is optionally substituted with 1-4 substituents independently selected from the group consisting of halogen, C₁₋₄alkyl, C₁₋₄fluoroalkyl, oxo, -L²-X², and -L²-G^{1b};
L², at each occurrence, is independently a bond or C₁₋₃alkylene;
X², at each occurrence, is independently —OR^{1c}, —N(R^{1c})₂, —SR^{1c}, cyano, —C(O)OR^{1c}, —C(O)N(R^{1c})₂, —C(O)R^{1c}, —SOR^{1d}, —SO₂R^{1d}, —SO₂N(R^{1c})₂, —NR^{1c}C(O)R^{1c}, —NR^{1c}C(O)OR^{1c}, —NR^{1c}C(O)N(R^{1c})₂, —NR^{1c}S(O)₂R^{1d}, or —NR^{1c}S(O)₂N(R^{1c})₂;
R^{1c}, at each occurrence, is independently hydrogen, C₁₋₄alkyl, C₁₋₄fluoroalkyl, G^{1b}, or —C₁₋₃alkylene-G^{1b}, wherein alternatively two R^{1c}, together with a common nitrogen atom to which the R^{1c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{1d}, at each occurrence, is independently C₁₋₄alkyl, C₁₋₄fluoroalkyl, G^{1b}, or —C₁₋₃alkylene-G^{1b};
R^{1e}, at each occurrence, is independently hydrogen, C₁₋₄alkyl, C₁₋₄fluoroalkyl, G^{1b}, or —C₁₋₃alkylene-G^{1b}, wherein alternatively two R^{1c}, together with a common nitrogen atom to which the R^{1c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
G^{1b}, at each occurrence, is independently a C₃₋₆cycloalkyl, a 4- to 6-membered monocyclic heterocyclyl containing 1-2 heteroatoms independently selected from O, N, and S, a 5- to 6-membered heteroaryl containing 1-4 heteroatoms independently selected from O, N, and S, or a phenyl, wherein G^{1b} is optionally substituted with 1-4 substituents independently selected from the group consisting of halogen, cyano, C₁₋₄alkyl, C₁₋₄fluoroalkyl, oxo, —OH, and —OC₁₋₄alkyl;
Y is OH, —OC₁₋₄alkyl, —OC₁₋₄fluoroalkyl, —OC₃₋₄cycloalkyl, or —OC₁₋₃alkylene-C₃₋₄cycloalkyl;
G^{1c} is C₃₋₆cycloalkyl, a 4- to 7-membered monocyclic heterocyclyl containing 1-2 heteroatoms independently selected from O, N, and S, or a 5- to 6-membered heteroaryl containing 1-3 heteroatoms independently selected from O, N, and S, wherein G^{1c} is optionally substituted with 1-4 substituents independently selected from the group consisting of halogen, cyano, C₁₋₄alkyl, C₁₋₄fluoroalkyl, oxo, —OH, —OC₁₋₄alkyl, C₃₋₄cycloalkyl, and —C₁₋₃alkylene-C₃₋₄cycloalkyl;
G² is a 5- to 6-membered heteroaryl or phenyl, wherein G² is optionally substituted with 1-4 substituents independently selected from the group consisting of C₁₋₆alkyl, C₁₋₆haloalkyl, halogen,
   —OR^{4c}, —N(R^{4c})₂, cyano, —C(O)OR^{4c}, —C(O)N(R^{4c})₂, —C(O)R^{4c}, —SO₂R^{4d}, —SO₂N(R^{4c})₂, —NR^{4c}C(O)R⁴, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of C₁₋₄alkyl and halogen;
R⁴, at each occurrence, is independently hydrogen, C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen, wherein alternatively two R⁴, together with a common nitrogen atom to which the R^{4c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{4d}, at each occurrence, is independently C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen.
R⁵ and R⁶ are each independently hydrogen, halogen, C₁₋₄alkyl, C₁₋₄fluoroalkyl, or —OC₁₋₄alkyl; and
R⁸ is an imidazolyl unsubstituted or substituted with 1-3 substituents independently selected from the group consisting of halogen, C₁₋₄alkyl, C₁₋₄fluoroalkyl, NO₂, NH₂, —NH(C₁₋₄alkyl), —N(C₁₋₄alkyl)₂, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of halogen, C₁₋₄alkyl, C₁₋₄fluoroalkyl, OH, and —OC₁₋₄alkyl; provided the compound is not:
7-((1H-imidazol-1-yl)methyl)-2-(6-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-methoxy-1,7-naphthyridin-4-yl)-5-(1-methyl-3-(trifluoromethyl)-1H-pyrazol-4-yl)-3,4-dihydroisoquinolin-1(2H)-one;
((S)-7-((1H-imidazol-1-yl)methyl)-2-(6-chloro-3,4-dihydro-2H-pyrano[2,3-b]pyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxycinnolin-4-yl)-5-(1-methyl-3-(trifluoromethyl)-1H-pyrazol-4-yl)-3,4-dihydroisoquinolin-1(2H)-one;
7-((2-methyl-1H-imidazol-1-yl)methyl)-5-(1-methyl-3-(trifluoromethyl)-1H-pyrazol-4-yl)-2-(5,6,7,8-tetrahydroquinolin-5-yl)-3,4-dihydroisoquinolin-1(2H)-one;
2-(3-methoxy-5,6,7,8-tetrahydroquinolin-5-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one;
(S)-2-(3-methoxy-5,6,7,8-tetrahydroquinolin-5-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one;
(S)-7-((1H-imidazol-1-yl)methyl)-2-(3-methoxy-5,6,7,8-tetrahydroquinolin-5-yl)-5-(1-methyl-3-(trifluoromethyl)-1H-pyrazol-4-yl)-3,4-dihydroisoquinolin-1(2H)-one;
((S)-7-((1H-imidazol-1-yl)methyl)-2-(6-chloro-3,4-dihydro-2H-pyrano[2,3-b]pyridin-4-yl)-5-(1-methyl-3-(trifluoromethyl)-1H-pyrazol-4-yl)-3,4-dihydroisoquinolin-1(2H)-one;
   or a pharmaceutically acceptable salt thereof.

In another aspect, the invention provides compounds of formula (I),

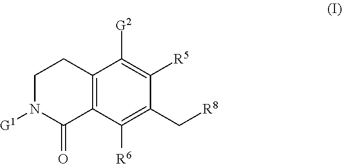

or a pharmaceutically acceptable salt thereof, wherein:
G¹ is

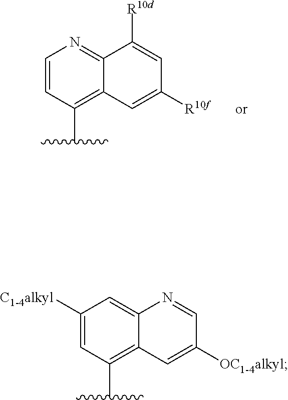

R^{10d} is X¹, hydrogen, halogen, C₁₋₄alkyl, C₁₋₄fluoroalkyl, or G^{1a};
R^{10f} is halogen, cyano, C₁₋₄alkyl, C₁₋₄fluoroalkyl, OH, —OC₁₋₄alkyl, —OC₁₋₄fluoroalkyl, —OC₃₋₄cycloalkyl, —OC₁₋₃alkylene-C₃₋₄cycloalkyl, —OPG, or —OSO₂CF₃;
PG is a hydroxy protecting group;
X¹ is —C(O)N(R^{1a})₂, —OR^{1a}, —N(R^{1a})₂, cyano, —C(O)OR^{1a}, —C(O)R^{1b}, —SO₂R^{1b}, —SO₂N(R^{1a})₂, —NR^{1a}C(O)H, or —NR^{1a}C(O)R^{1b};
R^{1a}, at each occurrence, is independently hydrogen, C₁₋₄alkyl, C₁₋₄fluoroalkyl, —C₂₋₃alkylene-OR^{1e}, —C₂₋₃alkylene-N(R^{1e})₂, —C₂₋₃alkylene-N(R^{1e})C(O)R^{1e}, G^{1a}, or —C₁₋₃alkylene-G^{1a};
R^{1b}, at each occurrence, is independently C₁₋₄alkyl, C₁₋₄fluoroalkyl, —C₁₋₃alkylene-OR^{1e}, —C₁₋₃alkylene-N(R^{1e})₂, —C₁₋₃alkylene-N(R^{1e})C(O)R, G^{1a}, or —C₁₋₃alkylene-G^{1a};
G^{1a}, at each occurrence, is independently C₃₋₈cycloalkyl, 5- to 6-membered heteroaryl, or 4- to 8-membered heterocyclyl, wherein G^{1a} is optionally substituted with 1-4 substituents independently selected from the group consisting of halogen, C₁₋₄alkyl, C₁₋₄fluoroalkyl, oxo, -L²-X², and -L²-G^{1b};
L², at each occurrence, is independently a bond or C₁₋₃alkylene;
X², at each occurrence, is independently —OR^{1c}, —N(R^{1c})₂, —SR^{1c}, cyano, —C(O)OR^{1c}, —C(O)N(R^{1c})₂, —C(O)R^{1c}, —SOR^{1d}, —SO₂R^{1d}, —SO₂N(R^{1c})₂, —NR^{1c}C(O)R^{1c}, —NR^{1c}C(O)OR^{1c}, —NR^{1c}C(O)N(R^{1c})₂, —NR^{1c}S(O)₂R^{1d}, or —NR^{1c}S(O)₂N(R^{1c})₂;
R^{1c}, at each occurrence, is independently hydrogen, C₁₋₄alkyl, C₁₋₄fluoroalkyl, G^{1b}, or —C₁₋₃alkylene-G^{1b}, wherein alternatively two R^{1c}, together with a common nitrogen atom to which the R^{1c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{1d}, at each occurrence, is independently C₁₋₄alkyl, C₁₋₄fluoroalkyl, G^{1b}, or —C₁₋₃alkylene-G^{1b};
R^{1e}, at each occurrence, is independently hydrogen, C₁₋₄alkyl, C₁₋₄fluoroalkyl, G^{1b}, or —C₁₋₃alkylene-G^{1b}, wherein alternatively two R^{1e}, together with a common nitrogen atom to which the R^{1e} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
G^{1b}, at each occurrence, is independently a C₃₋₆cycloalkyl, a 4- to 6-membered monocyclic heterocyclyl containing 1-2 heteroatoms independently selected from O, N, and S, a 5- to 6-membered heteroaryl containing 1-4 heteroatoms independently selected from O, N, and S, or a phenyl, wherein G^{1b} is optionally substituted with 1-4 substituents independently selected from the group consisting of halogen, cyano, C₁₋₄alkyl, C₁₋₄fluoroalkyl, oxo, —OH, and —OC₁₋₄alkyl;
G² is a 5- to 6-membered heteroaryl, wherein G² is optionally substituted with 1-4 substituents independently selected from the group consisting of C₁₋₄alkyl, C₁₋₄fluoroalkyl, halogen, —OR^{4c}, —N(R^{4c})₂, cyano, —C(O)OR^{4c}, —C(O)N(R^{4c})₂, —C(O)R^{4c}, —SO₂R^{4d}, —SO₂N(R^{4c})₂, —NR^{4c}C(O)R^{4c}, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of C₁₋₄alkyl and halogen;
R^{4c}, at each occurrence, is independently hydrogen, C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen, wherein alternatively two R^{4c}, together with a common nitrogen atom to which the R^{4c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{4d}, at each occurrence, is independently C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen.
R⁵ and R⁶ are each independently hydrogen, halogen, C₁₋₄alkyl, C₁₋₄fluoroalkyl, or —OC₁₋₄alkyl; and
R⁸ is

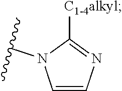

provided the compound is not:
2-(6-methoxy-8-methylquinolin-4-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one, or a pharmaceutically acceptable salt thereof.

In another aspect, the invention provides a pharmaceutical composition comprising a compound of formula (I), or a pharmaceutically acceptable salt thereof, and a pharmaceutically acceptable carrier.

In another aspect, the invention provides a method for the treatment of cancer, comprising administering to a subject in need thereof a therapeutically effective amount of a compound of formula (I), or a pharmaceutically acceptable salt or composition thereof.

In another aspect, the invention provides a method for inhibiting the binding of MLL1 to WDR5, comprising administering to a subject in need thereof a therapeutically effective amount of a compound of formula (I), or a pharmaceutically acceptable salt or composition thereof.

In another aspect, the invention provides a compound of formula (I), or a pharmaceutically acceptable salt or composition thereof, for use in the treatment of cancer.

In another aspect, the invention provides a compound of formula (I), or a pharmaceutically acceptable salt or composition thereof, for use in the inhibition of binding of MLL1 to WDR5.

In another aspect, the invention provides the use of a compound of formula (I), or a pharmaceutically acceptable salt or composition thereof, in the manufacture of a medicament for the treatment of cancer.

In another aspect, the invention provides the use of a compound of formula (I), or a pharmaceutically acceptable salt or composition thereof, in the manufacture of a medicament for the inhibition of binding of MLL1 to WDR5.

In another aspect, the invention provides a kit comprising a compound of formula (I), or a pharmaceutically acceptable salt or composition thereof, and instructions for use.

### DETAILED DESCRIPTION

Disclosed herein are inhibitors of WDR5, which bind at the WDR5 interaction or WIN-site. The inhibitors can be compounds of formula (I). Compounds of formula (I) can be used to treat cancers associated with the MLL1-WDR5 interaction. In one aspect, disclosed are compounds of formula (I) as WDR5-WIN-site inhibitors.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10)%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, iso-pentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkylene,” as used herein, refers to a divalent group derived from a straight or branched chain hydrocarbon, for example, of 1 to 6 carbon atoms. Representative examples of alkylene include, but are not limited to, —CH₂—, —CD₂-, —CH₂CH₂—, —C(CH₃)(H)—, —C(CH₃)(D)-, —CH₂CH₂CH₂—, —CH(CH₃)CH₂CH₂—, —C(CH₃)₂CH₂CH₂—, —CH₂C(CH₃)₂CH₂—, —CH₂CH₂CH₂CH₂—, and —CH₂CH₂CH₂CH₂CH₂—.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “fluoroalkyl,” as used herein, means an alkyl group, as defined herein, in which one, two, three, four, five, six, seven or eight hydrogen atoms are replaced by a fluoro group. Representative examples of fluoroalkyl include CH₂F, CHF₂, CF₃, and CH₂CHF₂.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8 to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, and tetrahydroisoquinolinyl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocycles are connected to the parent molecular moiety at a non-aromatic ring atom.

The term “imino” refers to the group “═NH.”

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups may include, for example, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### 2. Compounds

In one aspect, disclosed are compounds of formula (I), wherein G¹, G², R⁵, R⁶, and R⁸ are as defined herein. Embodiments of formula (I) include the following descriptions of G¹, G², R⁵, R⁶, and R⁸, and any combinations thereof.

Throughout the embodiments and description of the compounds of the invention, all instances of haloalkyl may be fluoroalkyl (e.g., any C₁₋₄haloalkyl may be C₁₋₄fluoroalkyl).

Hydroxy protecting groups PG are well known in the art, as described in P G M Wuts and T W Greene, in Greene's book titled Protective Groups in Organic Synthesis (4^{th} ed.), John Wiley & Sons, NY (2006), which is incorporated herein by reference in its entirety. Suitable hydroxy protecting groups include, for example, trityl groups (e.g., trityl, dimethoxytrityl, methoxytrityl), acetyl, benzoyl, benzyl, p-methoxybenzyl, β-methoxyethoxymethyl (MEM), methoxymethyl (MOM), methylthiomethyl, pivaloyl, tetrahydropyranyl (THP), tetrahydrofuranyl (THF), silyl (e.g., trimethylsilyl (TMS), tert-butyldimethylsilyl (TBDMS), tri-isopropylsilyloxymethyl (TOM), triisopropylsilyl (TIPS), methyl, and ethoxyethyl.

Unsubstituted or substituted rings such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

In the following, numbered embodiments of the invention are disclosed. In the numbered embodiments, the reference to a range of preceding embodiments in multiple dependent format is a reference, in the alternative, to each embodiment sequentially listed herein in the recited range.
E1. A compound of formula (I)

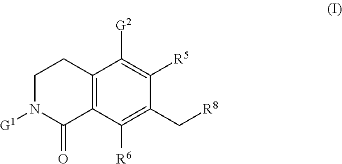

or a tautomer thereof or a pharmaceutically acceptable salt of the compound or tautomer, wherein:
G¹ is

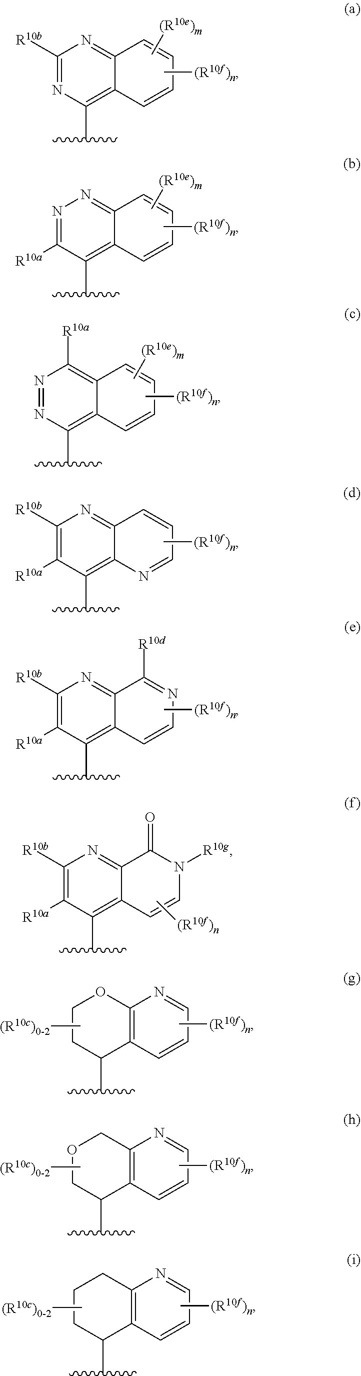

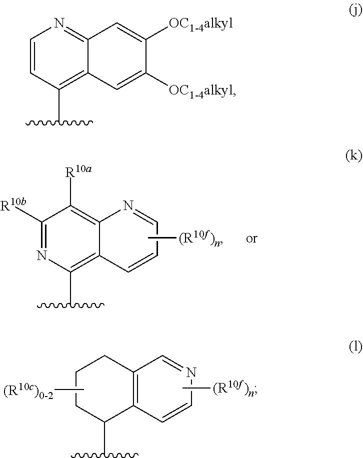

R^{10a} is hydrogen, fluoro, chloro, C₁₋₃alkyl, or C₁₋₃fluoroalkyl;
R^{10b} is hydrogen, fluoro, chloro, C₁₋₃alkyl, C₁₋₃fluoroalkyl, C₃₋₆cycloalkyl, NH₂, —NHC₁₋₄alkyl, —N(C₁₋₄alkyl)₂, or a 4- to 8-membered monocyclic heterocyclyl containing 1-2 heteroatoms independently selected from the group consisting of N, O, and S, wherein the heterocyclyl is optionally substituted with 1-4 substituents independently selected from the group consisting of halogen, cyano, oxo, C₁₋₄alkyl, C₁₋₄fluoroalkyl, OH, —OC₁₋₄alkyl, —OC₁₋₄fluoroalkyl, NH₂, —NHC₁₋₄alkyl, and —N(C₁₋₄alkyl)₂;
R^{10c}, at each occurrence, is independently fluoro, chloro, C₁₋₃alkyl, or C₁₋₃fluoroalkyl;
R^{10d} is X¹, -L¹-X¹, hydrogen, halogen, C₁₋₄alkyl, C₁₋₄fluoroalkyl, G^{1a}, or -L¹-G^{1a};
R^{10e} is halogen, cyano, C₁₋₄alkyl, C₁₋₄fluoroalkyl, OH, —OC₁₋₄alkyl, —OC₁₋₄fluoroalkyl, —OG^{1c}, —OC₁₋₃alkylene-G^{1c}, —O—C₂₋₃alkylene-Y, NH₂, —NHC₁₋₄alkyl, —N(C₁₋₄alkyl)₂, or a 4- to 8-membered monocyclic heterocyclyl containing 1-2 heteroatoms independently selected from the group consisting of N, O, and S, wherein the heterocyclyl is optionally substituted with 1-4 substituents independently selected from the group consisting of halogen, cyano, oxo, C₁₋₄alkyl, C₁₋₄fluoroalkyl, OH, —OC₁₋₄alkyl, —OC₁₋₄fluoroalkyl, NH₂, —NHC₁₋₄alkyl, and —N(C₁₋₄alkyl)₂;
R^{10f}, at each occurrence, is independently halogen, cyano, C₁₋₄alkyl, C₁₋₄fluoroalkyl, OH, —OC₁₋₄alkyl, —OC₁₋₄fluoroalkyl, —OC₃₋₄cycloalkyl, —C₁₋₃alkylene-C₃₋₄cycloalkyl, —OPG, or —OSO₂CF₃;
R^{10g} is hydrogen, C₁₋₄alkyl, C₁₋₄fluoroalkyl, C₃₋₄cycloalkyl, or —C₁₋₃alkylene-C₃₋₄cycloalkyl;
m is 0 or 1;
n is 0, 1, or 2;
PG is a hydroxy protecting group;
L¹ is C₁₋₃alkylene;
X¹ is —C(O)N(R^{1a})₂, —OR^{1a}, —N(R^{1a})₂, cyano, —C(O)OR^{1a}, —C(O)R^{1b}, —SO₂R^{1b}, —SO₂N(R^{1a})₂, —NR^{1a}C(O)H, or —NR^{1a}C(O)R^{1b};
R^{1a}, at each occurrence, is independently hydrogen, C₁₋₄alkyl, C₁₋₄fluoroalkyl, —C₂₋₃alkylene-OR^{1c}, —C₂₋₃alkylene-N(R^{1c})₂, —C₂₋₃alkylene-N(R^{1c})C(O)R^{1c}, G^{1a}, or —C₁₋₃alkylene-G^{1a};
R^{1b}, at each occurrence, is independently C₁₋₄alkyl, C₁₋₄fluoroalkyl, —C₁₋₃alkylene-OR^{1e}, —C₁₋₃alkylene-N(R^{1e})₂, —C₁₋₃alkylene-N(R^{1e})C(O)R^{1e}, G^{1a}, or —C₁₋₃alkylene-G^{1a};
G^{1a}, at each occurrence, is independently C₃₋₈cycloalkyl, 5- to 6-membered heteroaryl, or 4- to 8-membered heterocyclyl, wherein G^{1a} is optionally substituted with 1-4 substituents independently selected from the group consisting of halogen, C₁₋₄alkyl, C₁₋₄fluoroalkyl, oxo, -L²-X², and -L²-G^{1b};
L², at each occurrence, is independently a bond or C₁₋₃alkylene;
X², at each occurrence, is independently —OR^{1c}, —N(R^{1c})₂, —SR^{1c}, cyano, —C(O)OR^{1c}, —C(O)N(R^{1c})₂, —C(O)R^{1c}, —SOR^{1d}, —SO₂R^{1d}, —SO₂N(R^{1c})₂, —NR^{1c}C(O)R^{1c}, —NR^{1c}C(O)OR^{1c}, —NR^{1c}C(O)N(R^{1c})₂, —NR^{1c}S(O)₂R^{1d}, or —NR^{1c}S(O)₂N(R^{1c})₂;
R^{1c}, at each occurrence, is independently hydrogen, C₁₋₄alkyl, C₁₋₄fluoroalkyl, G^{1b}, or —C₁₋₃alkylene-G^{1b}, wherein alternatively two R^{1c}, together with a common nitrogen atom to which the R^{1c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{1d}, at each occurrence, is independently C₁₋₄alkyl, C₁₋₄fluoroalkyl, G^{1b}, or —C₁₋₃alkylene-G^{1b};
R^{1e}, at each occurrence, is independently hydrogen, C₁₋₄alkyl, C₁₋₄fluoroalkyl, G^{1b}, or —C₁₋₃alkylene-G^{1b}, wherein alternatively two R^{1c}, together with a common nitrogen atom to which the R^{1c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
G^{1b}, at each occurrence, is independently a C₃₋₆cycloalkyl, a 4- to 6-membered monocyclic heterocyclyl containing 1-2 heteroatoms independently selected from O, N, and S, a 5- to 6-membered heteroaryl containing 1-4 heteroatoms independently selected from O, N, and S, or a phenyl, wherein G^{1b} is optionally substituted with 1-4 substituents independently selected from the group consisting of halogen, cyano, C₁₋₄alkyl, C₁₋₄fluoroalkyl, oxo, —OH, and —OC₁₋₄alkyl;
Y is OH, —OC₁₋₄alkyl, —OC₁₋₄fluoroalkyl, —OC₃₋₄cycloalkyl, or —OC₁₋₃alkylene-C₃₋₄cycloalkyl;
G^{1c} is C₃₋₆cycloalkyl, a 4- to 7-membered monocyclic heterocyclyl containing 1-2 heteroatoms independently selected from O, N, and S, or a 5- to 6-membered heteroaryl containing 1-3 heteroatoms independently selected from O, N, and S, wherein G^{1c} is optionally substituted with 1-4 substituents independently selected from the group consisting of halogen, cyano, C₁₋₄alkyl, C₁₋₄fluoroalkyl, oxo, —OH, —OC₁₋₄alkyl, C₃₋₄cycloalkyl, and —C₁₋₃alkylene-C₃₋₄cycloalkyl;
G² is a 5- to 6-membered heteroaryl or phenyl, wherein G² is optionally substituted with 1-4 substituents independently selected from the group consisting of C₁₋₄alkyl, C₁₋₄fluoroalkyl, halogen, —OR^{4c}, —N(R^{4c})₂, cyano, —C(O)OR^{4c}, —C(O)N(R^{4c})₂, —C(O)R^{4c}, —SO₂R^{4d}, —SO₂N(R^{4c})₂, —NR^{4c}C(O)R^{4c}, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of C₁₋₄alkyl and halogen;
R^{4c}, at each occurrence, is independently hydrogen, C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen, wherein alternatively two R^{4c}, together with a common nitrogen atom to which the R^{4c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{4d}, at each occurrence, is independently C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen.
R⁵ and R⁶ are each independently hydrogen, halogen, C₁₋₄alkyl, C₁₋₄fluoroalkyl, or —OC₁₋₄alkyl; and
R⁸ is an imidazolyl unsubstituted or substituted with 1-3 substituents independently selected from the group consisting of halogen, C₁₋₄alkyl, C₁₋₄fluoroalkyl, NO₂, NH₂, —NH(C₁₋₄alkyl), —N(C₁₋₄alkyl)₂, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of halogen, C₁₋₄alkyl, C₁₋₄fluoroalkyl, OH, and —OC₁₋₄alkyl;
provided the compound is not:
7-((1H-imidazol-1-yl)methyl)-2-(6-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-methoxy-1,7-naphthyridin-4-yl)-5-(1-methyl-3-(trifluoromethyl)-1H-pyrazol-4-yl)-3,4-dihydroisoquinolin-1(2H)-one;
((S)-7-((1H-imidazol-1-yl)methyl)-2-(6-chloro-3,4-dihydro-2H-pyrano[2,3-b]pyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxycinnolin-4-yl)-5-(1-methyl-3-(trifluoromethyl)-1H-pyrazol-4-yl)-3,4-dihydroisoquinolin-1(2H)-one;
7-((2-methyl-1H-imidazol-1-yl)methyl)-5-(1-methyl-3-(trifluoromethyl)-1H-pyrazol-4-yl)-2-(5,6,7,8-tetrahydroquinolin-5-yl)-3,4-dihydroisoquinolin-1(2H)-one;
2-(3-methoxy-5,6,7,8-tetrahydroquinolin-5-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one;
(S)-2-(3-methoxy-5,6,7,8-tetrahydroquinolin-5-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one;
(S)-7-((1H-imidazol-1-yl)methyl)-2-(3-methoxy-5,6,7,8-tetrahydroquinolin-5-yl)-5-(1-methyl-3-(trifluoromethyl)-1H-pyrazol-4-yl)-3,4-dihydroisoquinolin-1(2H)-one;
((S)-7-((1H-imidazol-1-yl)methyl)-2-(6-chloro-3,4-dihydro-2H-pyrano[2,3-b]pyridin-4-yl)-5-(1-methyl-3-(trifluoromethyl)-1H-pyrazol-4-yl)-3,4-dihydroisoquinolin-1(2H)-one;
   or a pharmaceutically acceptable salt thereof.

E1.1. The compound of E1, or a tautomer thereof or a pharmaceutically acceptable salt of the compound or tautomer, wherein:
G¹ is

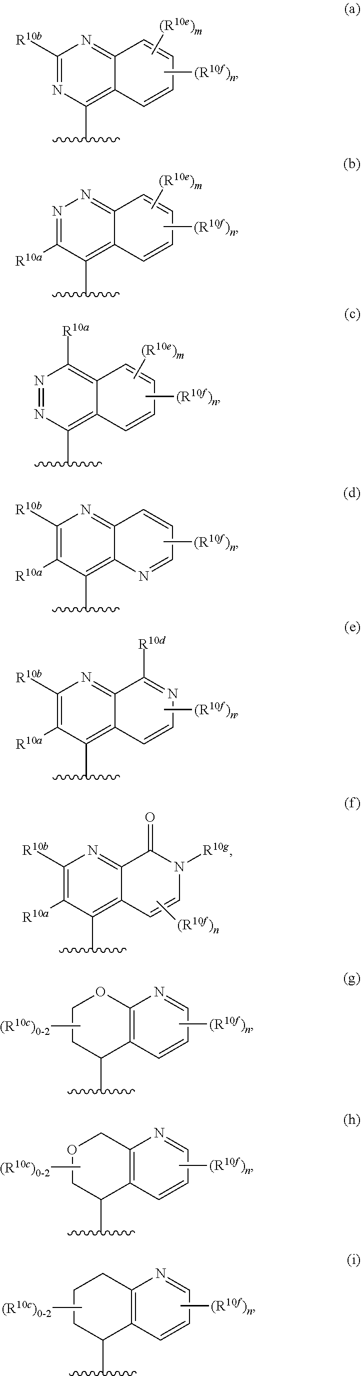

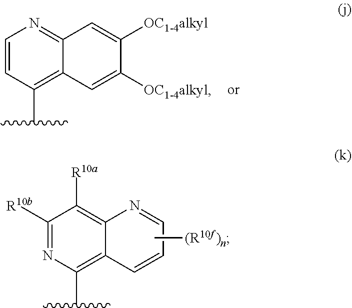

R^{10b} is hydrogen, fluoro, chloro, C₁₋₃alkyl, or C₁₋₃fluoroalkyl;
R^{10d} is X¹, hydrogen, halogen, C₁₋₄alkyl, C₁₋₄fluoroalkyl, or G^{1a};
R^{10e} is halogen, cyano, C₁₋₄alkyl, C₁₋₄fluoroalkyl, OH, —OC₁₋₄alkyl, —OC₁₋₄fluoroalkyl, —OG^{1c}, —OC₁₋₃alkylene-G^{1c}, or —O—C₂₋₃alkylene-Y; and
G² is a 5- to 6-membered heteroaryl, wherein G² is optionally substituted with 1-4 substituents independently selected from the group consisting of C₁₋₄alkyl, C₁₋₄fluoroalkyl, halogen, —OR^{4c}, —N(R^{4c})₂, cyano, —C(O)OR^{4c}, —C(O)N(R^{4c})₂, —C(O)R^{4c}, —SO₂R^{4d}, —SO₂N(R^{4c})₂, —NR^{4c}C(O)R^{4c}, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of C₁₋₄alkyl and halogen.
E1.2. The compound of E1 or E1.1, or a tautomer thereof or a pharmaceutically acceptable salt of the compound or tautomer, wherein m is 0.
E1.3. The compound of E1 or E1.1, or a tautomer thereof or a pharmaceutically acceptable salt of the compound or tautomer, wherein m is 1.
E1.4. The compound of any of E1-E1.3, or a tautomer thereof or a pharmaceutically acceptable salt of the compound or tautomer, wherein n is 0.
E1.5. The compound of any of E1-E1.3, or a tautomer thereof or a pharmaceutically acceptable salt of the compound or tautomer, wherein n is 1.
E1.6. The compound of any of E1-E1.3, or a tautomer thereof or a pharmaceutically acceptable salt of the compound or tautomer, wherein n is 2.
E2. The compound of any of E1-E1.6, or a tautomer thereof or a pharmaceutically acceptable salt of the compound or tautomer, wherein

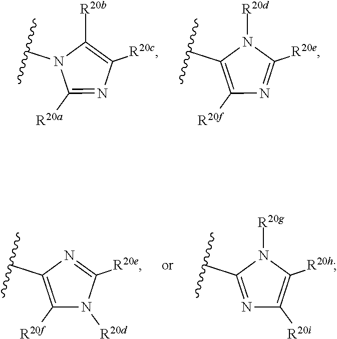

R^{20a} is hydrogen, C₁₋₄alkyl, NH₂, —NH(C₁₋₄alkyl), —N(C₁₋₄alkyl)₂, or C₃₋₈cycloalkyl; and
R^{20b}, R^{20c}, R^{20d}, R^{20e}, R^{20f}, R^{20g}, R^{20h}, and R²⁰ⁱ are each independently hydrogen, C₁₋₄alkyl, or C₃₋₈cycloalkyl.
E3. The compound of E2, or a tautomer thereof or a pharmaceutically acceptable salt of the compound or tautomer, wherein R⁸ is

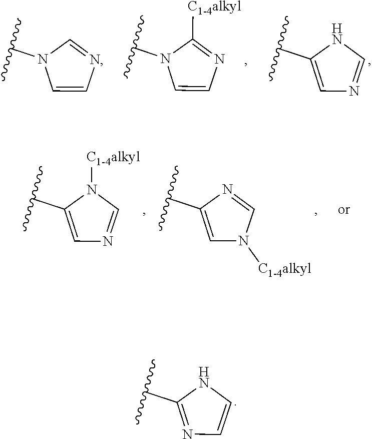

E3.1. The compound of E3, or a tautomer thereof or a pharmaceutically acceptable salt of the compound or tautomer, wherein R⁸ is

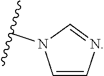 [SMILES: *n1ccnc1]

E3.2. The compound of E3, or a tautomer thereof or a pharmaceutically acceptable salt of the compound or tautomer, wherein R⁸ is

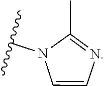 [SMILES: *n1ccnc1C]

E4. The compound of any of E1-E3.2, or a tautomer thereof or a pharmaceutically acceptable salt of the compound or tautomer, wherein G¹ is

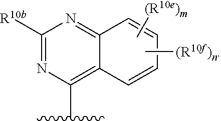

E4.1. The compound of E4, or a tautomer thereof or a pharmaceutically acceptable salt of the compound or tautomer, wherein G¹ is

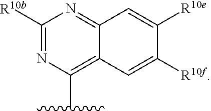

E4.2. The compound of E4, or a tautomer thereof or a pharmaceutically acceptable salt of the compound or tautomer, wherein G¹ is

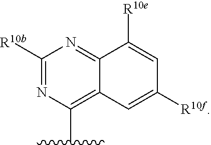

E4.3. The compound of any of E4-E4.2, or a tautomer thereof or a pharmaceutically acceptable salt of the compound or tautomer, wherein R^{10b} is hydrogen.
E4.4. The compound of any of E4-E4.2, or a tautomer thereof or a pharmaceutically acceptable salt of the compound or tautomer, wherein R^{10b} is C₁₋₃alkyl.
E4.5a. The compound of E4.4, or a tautomer thereof or a pharmaceutically acceptable salt of the compound or tautomer, wherein R^{10b} is methyl.
E4.5b. The compound of E4.4, or a tautomer thereof or a pharmaceutically acceptable salt of the compound or tautomer, wherein R^{10b} is ethyl.
E4.6. The compound of any of E4-E4.2, or a tautomer thereof or a pharmaceutically acceptable salt of the compound or tautomer, wherein R^{10b} is C₃₋₆cycloalkyl.
E4.7. The compound of E4.6, or a tautomer thereof or a pharmaceutically acceptable salt of the compound or tautomer, wherein R^{10b} is cyclopropyl.
E4.8. The compound of any of E4-E4.2, or a tautomer thereof or a pharmaceutically acceptable salt of the compound or tautomer, wherein R^{10b} is —NHC₁₋₄alkyl (e.g., —NHCH₃) or —N(C₁₋₄alkyl)₂ (e.g., —N(CH₃)CH₂CH₃).
E4.9. The compound of any of E4-E4.2, or a tautomer thereof or a pharmaceutically acceptable salt of the compound or tautomer, wherein R^{10b} is the optionally substituted 4- to 8-membered monocyclic heterocyclyl.
E4.10. The compound of any of E4-E4.2 or E4.9, or a tautomer thereof or a pharmaceutically acceptable salt of the compound or tautomer, wherein the ring system of the optionally substituted 4- to 8-membered monocyclic heterocyclyl contains at least one ring nitrogen atom and is bonded to the parent molecular moiety at the at least one ring nitrogen atom (e.g., azetidin-1-yl, pyrrolidin-1-yl, piperidin-1-yl, morpholino, 1,4-oxazepan4-yl, thiomorpholino, piperazin-1-yl, 1,4-diazepan-1-yl).
E4.11. The compound of E4.10, or a tautomer thereof or a pharmaceutically acceptable salt of the compound or tautomer, wherein R^{10b} is

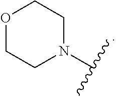 [SMILES: *N1CCOCC1]

E4.12. The compound of any of E4-E4.2 or E4.9, or a tautomer thereof or a pharmaceutically acceptable salt of the compound or tautomer, wherein the optionally substituted 4- to 8-membered monocyclic heterocyclyl is bonded to the parent molecular moiety at a ring carbon atom.
E4.13. The compound of E4.12, or a tautomer thereof or a pharmaceutically acceptable salt of the compound or tautomer, wherein the ring system of the optionally substituted 4- to 8-membered monocyclic heterocyclyl contains one ring heteroatom that is oxygen (e.g., oxetanyl, tetrahydrofuranyl, tetrahydropyranyl, oxepanyl).
E4.14. The compound of E4.13, or a tautomer thereof or a pharmaceutically acceptable salt of the compound or tautomer, wherein R^{10b} is

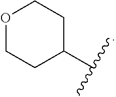 [SMILES: *C1CCOCC1]

E4.15. The compound of E4 or E4.1, or a tautomer thereof or a pharmaceutically acceptable salt of the compound or tautomer, wherein G¹ is

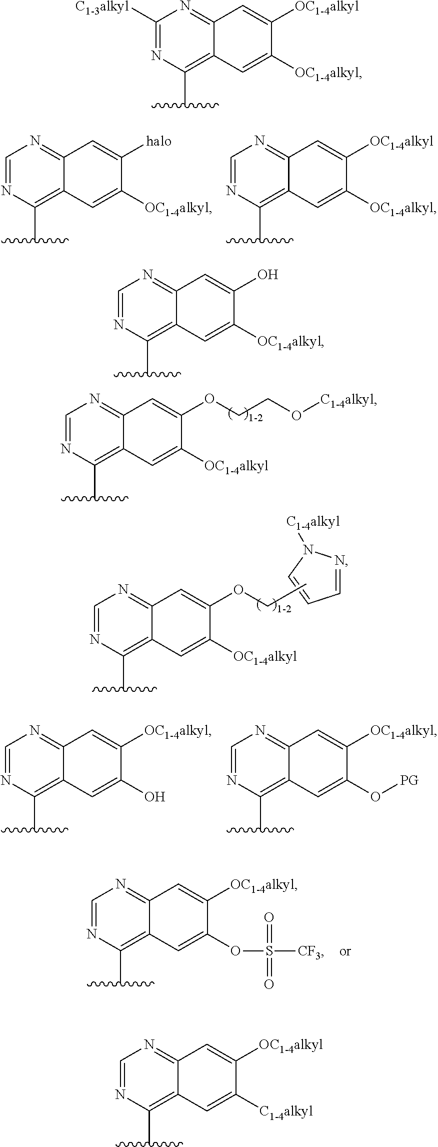

E4.16. The compound of E4.15, or a tautomer thereof or a pharmaceutically acceptable salt of the compound or tautomer, wherein G¹ is

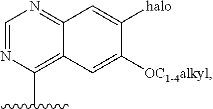

E4.17. The compound of any of E4, E4.1, or E4.15, or a tautomer thereof or a pharmaceutically acceptable salt of the compound or tautomer, wherein G¹ is

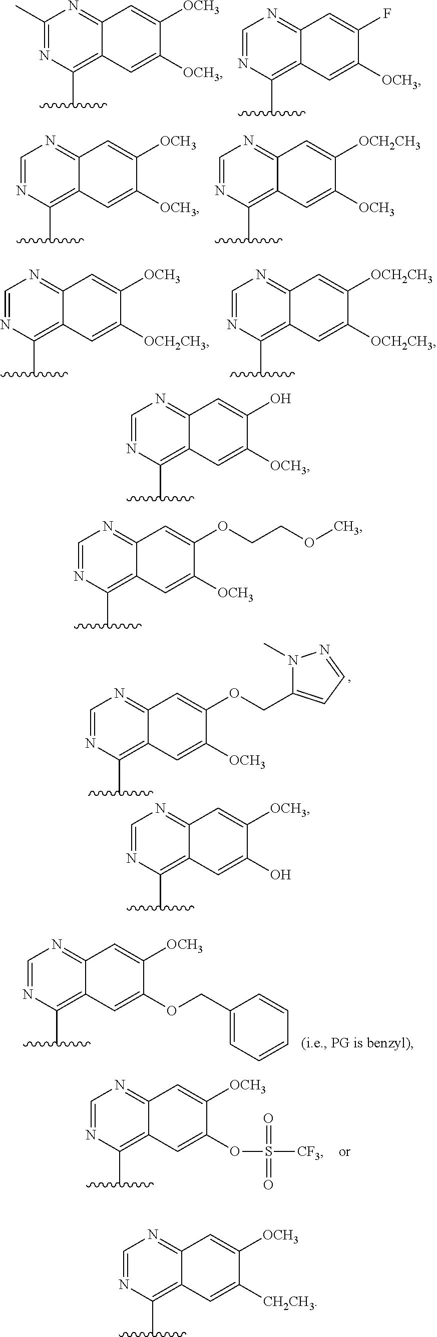

E4.18. The compound of E4 or E4.1, or a tautomer thereof or a pharmaceutically acceptable salt of the compound or tautomer, wherein G¹ is

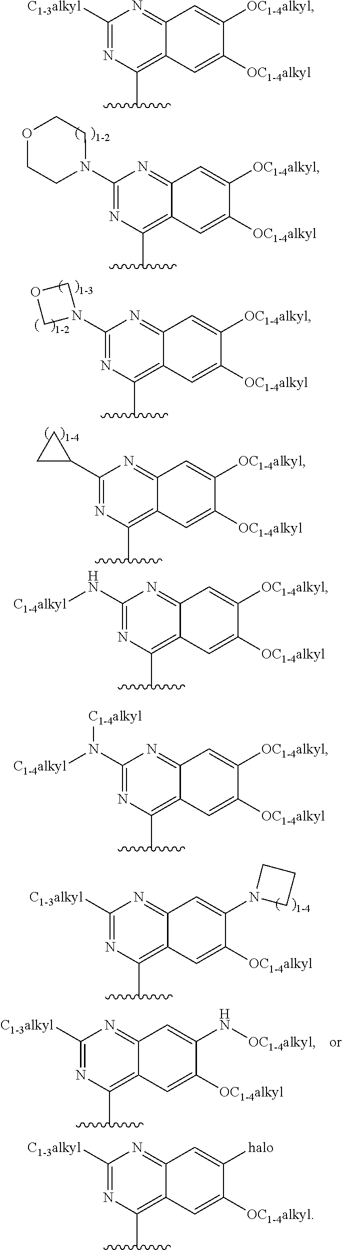

E4.19. The compound of E4.15 or E4.18, or a tautomer thereof or a pharmaceutically acceptable salt of the compound or tautomer, wherein G¹ is

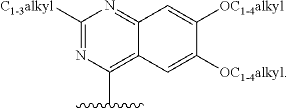

E4.20. The compound of E4.19, or a tautomer thereof or a pharmaceutically acceptable salt of the compound or tautomer, wherein G¹ is

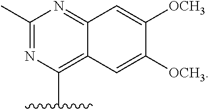 [SMILES: *c1nc(C)nc2cc(OC)c(OC)cc12]

E4.21. The compound of E4.19, or a tautomer thereof or a pharmaceutically acceptable salt of the compound or tautomer, wherein G¹ is

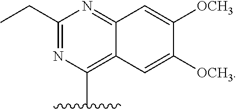 [SMILES: *c1nc(CC)nc2cc(OC)c(OC)cc12]

E4.22. The compound of E4.18, or a tautomer thereof or a pharmaceutically acceptable salt of the compound or tautomer, wherein G¹ is

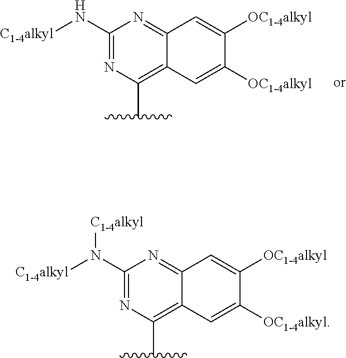

E4.23. The compound of E4.22, or a tautomer thereof or a pharmaceutically acceptable salt of the compound or tautomer, wherein G¹ is

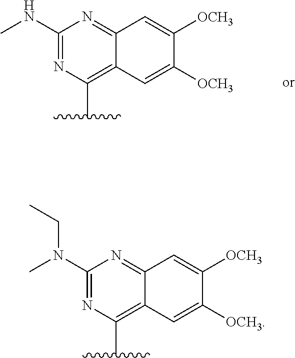 [SMILES: *c1nc(NC)nc2cc(OC)c(OC)cc12 ; "or" ; *c1nc(N(C)CC)nc2cc(OC)c(OC)cc12]

E4.24. The compound of E4.18, or a tautomer thereof or a pharmaceutically acceptable salt of the compound or tautomer, wherein G¹ is

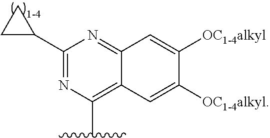

E4.25. The compound of E4.24, or a tautomer thereof or a pharmaceutically acceptable salt of the compound or tautomer, wherein G¹ is

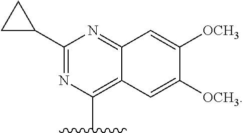 [SMILES: *c1nc(C2CC2)nc2cc(OC)c(OC)cc12]

E4.26. The compound of E4.18, or a tautomer thereof or a pharmaceutically acceptable salt of the compound or tautomer, wherein G¹ is

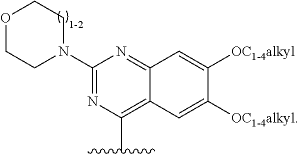

E4.27. The compound of E4.26, or a tautomer thereof or a pharmaceutically acceptable salt of the compound or tautomer, wherein G¹ is

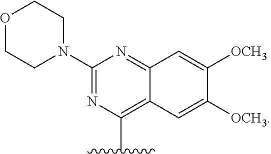 [SMILES: *c1nc(N2CCOCC2)nc2cc(OC)c(OC)cc12]

E4.28. The compound of E4.18, or a tautomer thereof or a pharmaceutically acceptable salt of the compound or tautomer, wherein G¹ is

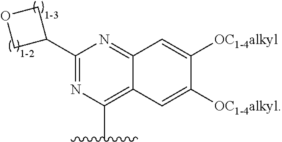

E4.29. The compound of E4.28, or a tautomer thereof or a pharmaceutically acceptable salt of the compound or tautomer, wherein G¹ is

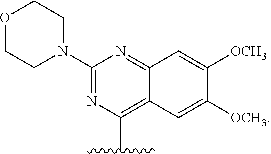 [SMILES: *c1nc(N2CCOCC2)nc2cc(OC)c(OC)cc12]

E4.30. The compound of E4.18, or a tautomer thereof or a pharmaceutically acceptable salt of the compound or tautomer, wherein G¹ is

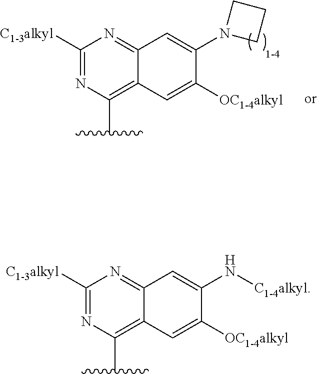

E4.31. The compound of E4.30, or a tautomer thereof or a pharmaceutically acceptable salt of the compound or tautomer, wherein G¹ is

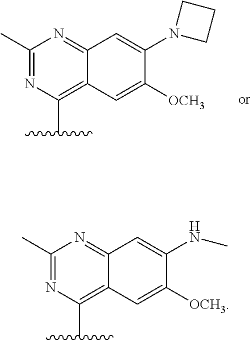

E4.32. The compound of E4.18, or a tautomer thereof or a pharmaceutically acceptable salt of the compound or tautomer, wherein G¹ is

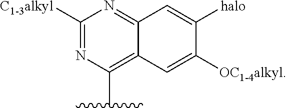

E4.33. The compound of E4.32, or a tautomer thereof or a pharmaceutically acceptable salt of the compound or tautomer, wherein G¹ is

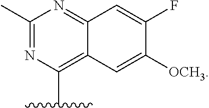 [SMILES: *c1nc(C)nc2cc(F)c(OC)cc12]

E4.34. The compound of E4 or E4.2, or a tautomer thereof or a pharmaceutically acceptable salt of the compound or tautomer, wherein G¹ is

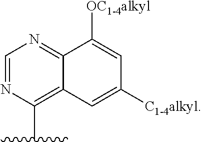

E4.35. The compound of any of E4, E4.2, or E4.34, or a tautomer thereof or a pharmaceutically acceptable salt of the compound or tautomer, wherein G¹ is

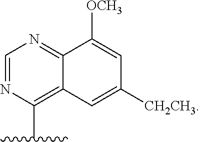 [SMILES: *c1ncnc2c(OC)cc(CC)cc12]

E4.36. The compound of E4 or E4.2, or a tautomer thereof or a pharmaceutically acceptable salt of the compound or tautomer, wherein G¹ is

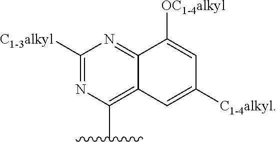

E4.37. The compound of any of E4, E4.2, or E4.36, or a tautomer thereof or a pharmaceutically acceptable salt of the compound or tautomer, wherein G¹ is

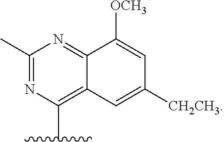 [SMILES: *c1nc(C)nc2c(OC)cc(CC)cc12]

E4.38. The compound of E4 or E4.2, or a tautomer thereof or a pharmaceutically acceptable salt of the compound or tautomer, wherein G¹ is

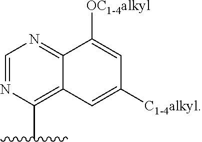

E4.39. The compound of any of E4, E4.2, or E4.38, or a tautomer thereof or a pharmaceutically acceptable salt of the compound or tautomer, wherein G¹ is

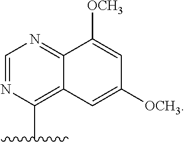 [SMILES: *c1ncnc2c(OC)cc(OC)cc12]

E5. The compound of any of E1-E3.2, or a tautomer thereof or a pharmaceutically acceptable salt of the compound or tautomer, wherein G¹ is

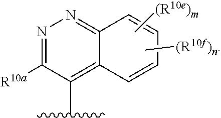

E5.1. The compound of E5, or a tautomer thereof or a pharmaceutically acceptable salt of the compound or tautomer, wherein G¹ is

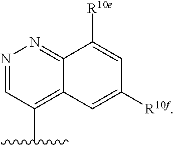

E5.2. The compound of E5 or E5.1, or a tautomer thereof or a pharmaceutically acceptable salt of the compound or tautomer, wherein G¹ is

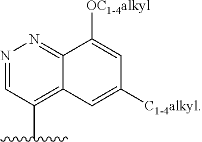

E5.3. The compound of any of E5-E5.2, or a tautomer thereof or a pharmaceutically acceptable salt of the compound or tautomer, wherein G¹ is

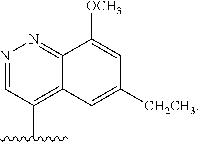 [SMILES: *c1cnnc2c(OC)cc(CC)cc12]

E5.4. The compound of E5, or a tautomer thereof or a pharmaceutically acceptable salt of the compound or tautomer, wherein G¹ is

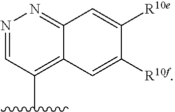

E5.5. The compound of E5.4, or a tautomer thereof or a pharmaceutically acceptable salt of the compound or tautomer, wherein G¹ is

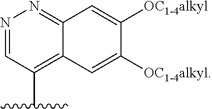

E5.6. The compound of E5.5, or a tautomer thereof or a pharmaceutically acceptable salt of the compound or tautomer, wherein G¹ is

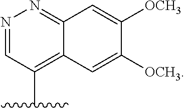 [SMILES: *c1cnnc2cc(OC)c(OC)cc12]

E6. The compound of any of E1-E3.2, or a tautomer thereof or a pharmaceutically acceptable salt of the compound or tautomer, wherein G¹ is

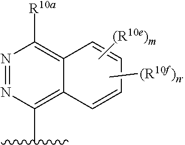

E6.1. The compound of E6, or a tautomer thereof or a pharmaceutically acceptable salt of the compound or tautomer, wherein G¹ is

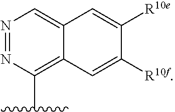

E6.2. The compound of E6 or E6.1, or a tautomer thereof or a pharmaceutically acceptable salt of the compound or tautomer, wherein G¹ is

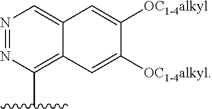

E6.3. The compound of any of E6-E6.2, or a tautomer thereof or a pharmaceutically acceptable salt of the compound or tautomer, wherein G¹ is

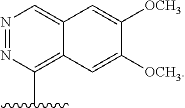 [SMILES: *c1nncc2cc(OC)c(OC)cc12]

E7. The compound of any of E1-E3.2, or a tautomer thereof or a pharmaceutically acceptable salt of the compound or tautomer, wherein G¹ is

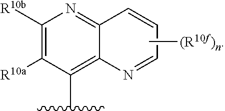

E7.1. The compound of E7, or a tautomer thereof or a pharmaceutically acceptable salt of the compound or tautomer, wherein G¹ is

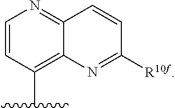

E7.2. The compound of E7 or E7.1, or a tautomer thereof or a pharmaceutically acceptable salt of the compound or tautomer, wherein G¹ is

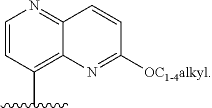

E7.3. The compound of any of E7-E7.2, or a tautomer thereof or a pharmaceutically acceptable salt of the compound or tautomer, wherein G¹ is

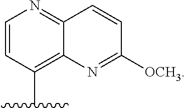 [SMILES: *c1ccnc2ccc(OC)nc12]

E8. The compound of any of E1-E3.2, or a tautomer thereof or a pharmaceutically acceptable salt of the compound or tautomer, wherein G¹ is

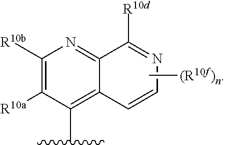

E8.1. The compound of E8, or a tautomer thereof or a pharmaceutically acceptable salt of the compound or tautomer, wherein G¹ is

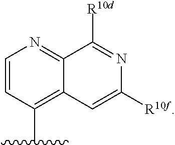

E8.2. The compound of E8 or E8.1, or a tautomer thereof or a pharmaceutically acceptable salt of the compound or tautomer, wherein G¹ is

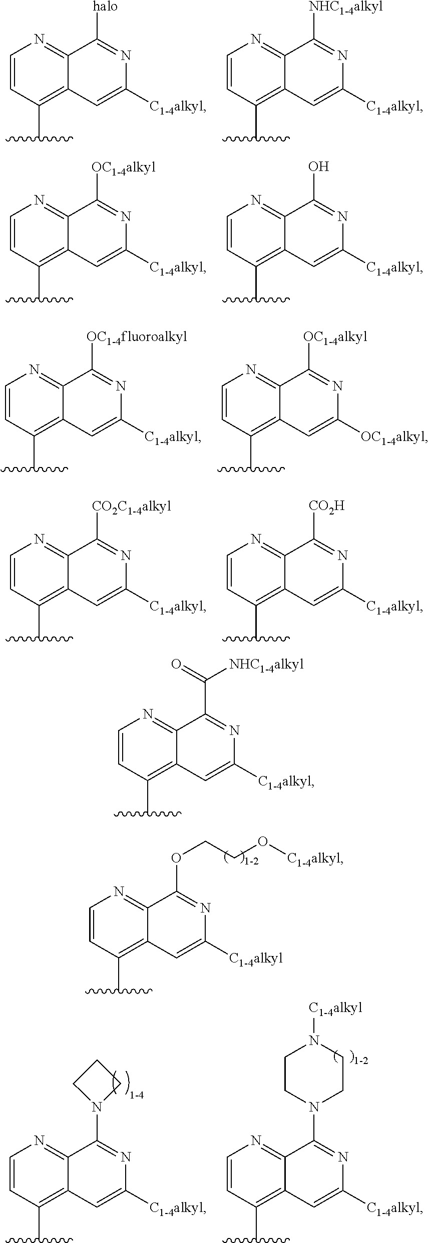

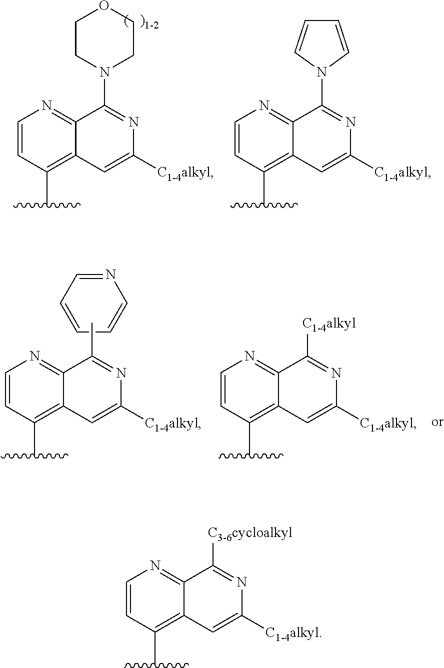

E8.3. The compound of any of E8-E8.2, or a tautomer thereof or a pharmaceutically acceptable salt of the compound or tautomer, wherein G¹ is

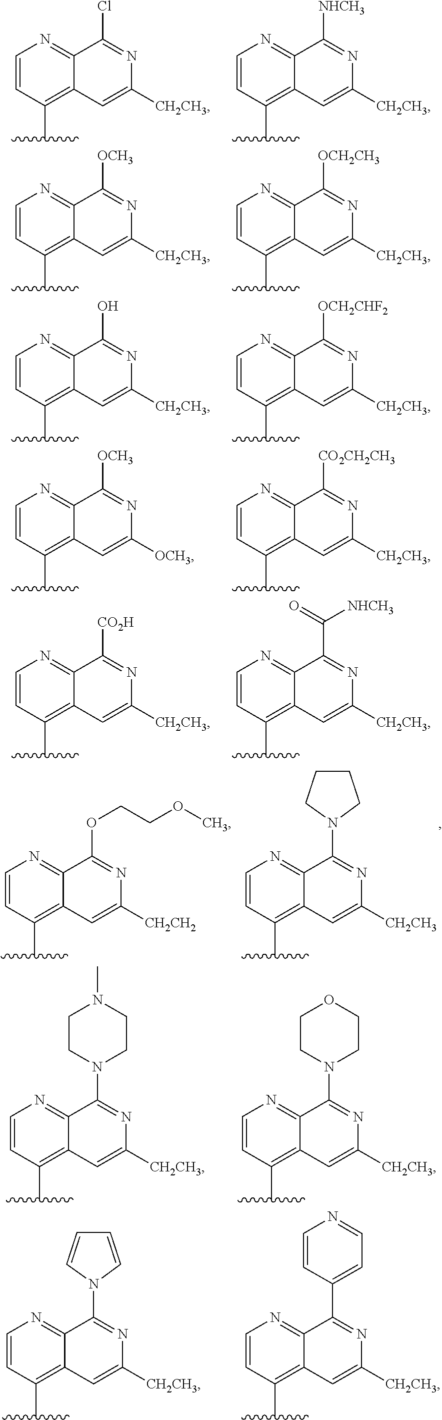

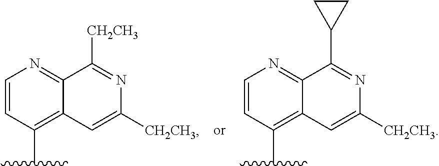 [SMILES: *c1ccnc2c(CC)nc(CC)cc12 ; *c1ccnc2c(C3CC3)nc(CC)cc12]

E8.4. The compound of E8 or E8.1, or a tautomer thereof or a pharmaceutically acceptable salt of the compound or tautomer, wherein G¹ is

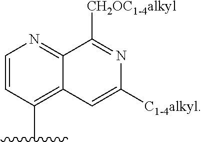

E8.5. The compound of E8.4, or a tautomer thereof or a pharmaceutically acceptable salt of the compound or tautomer, wherein G¹ is

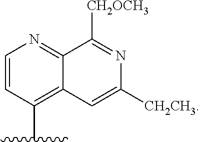

E8.6. The compound of E8, or a tautomer thereof or a pharmaceutically acceptable salt of the compound or tautomer, wherein G¹ is

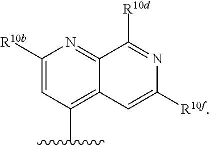

E8.7. The compound of E8.6, or a tautomer thereof or a pharmaceutically acceptable salt of the compound or tautomer, wherein G¹ is

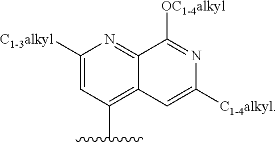

E8.8. The compound of E8.7, or a tautomer thereof or a pharmaceutically acceptable salt of the compound or tautomer, wherein G¹ is

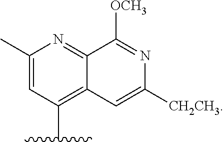 [SMILES: *c1cc(C)nc2c(OC)nc(CC)cc12]

E9. The compound of any of E1-E3.2, or a tautomer thereof or a pharmaceutically acceptable salt of the compound or tautomer, wherein G¹ is

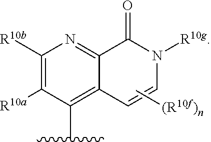

E9.1. The compound of E9, or a tautomer thereof or a pharmaceutically acceptable salt of the compound or tautomer, wherein G¹ is

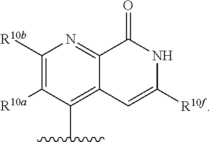

E9.2. The compound of E9 or E9.1, or a tautomer thereof or a pharmaceutically acceptable salt of the compound or tautomer, wherein G¹ is

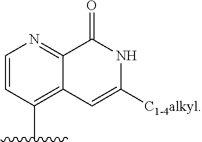

E9.3. The compound of any of E9-E9.2, or a tautomer thereof or a pharmaceutically acceptable salt of the compound or tautomer, wherein G¹ is

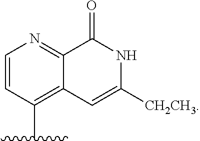 [SMILES: *c1ccnc2c(=O)[nH]c(CC)cc12]

E10. The compound of any of E1-E3.2, or a tautomer thereof or a pharmaceutically acceptable salt of the compound or tautomer, wherein G¹ is

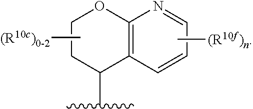

E10.1. The compound of E10, or a tautomer thereof or a pharmaceutically acceptable salt of the compound or tautomer, wherein G¹ is

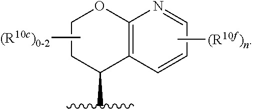

E10.2. The compound of E10, or a tautomer thereof or a pharmaceutically acceptable salt of the compound or tautomer, wherein G¹ is

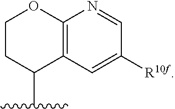

E10.3. The compound of any of E10-E10.2, or a tautomer thereof or a pharmaceutically acceptable salt of the compound or tautomer, wherein G¹ is

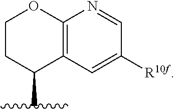

E10.4. The compound of E10 or E10.2, or a tautomer thereof or a pharmaceutically acceptable salt of the compound or tautomer, wherein G¹ is

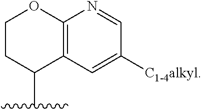

E10.5. The compound of any of E10-E10.4, or a tautomer thereof or a pharmaceutically acceptable salt of the compound or tautomer, wherein G¹ is

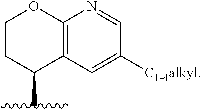

E10.6. The compound of any of E10, E10.2, or E10.4, or a tautomer thereof or a pharmaceutically acceptable salt of the compound or tautomer, wherein G¹ is

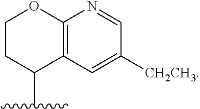 [SMILES: *C1CCOc2ncc(CC)cc21]

E10.7. The compound of any of E10-E10.6, or a tautomer thereof or a pharmaceutically acceptable salt of the compound or tautomer, wherein G¹ is

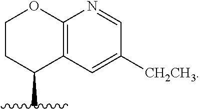 [SMILES: *[C@H]1CCOc2ncc(CC)cc21]

E11. The compound of any of E1-E3.2, or a tautomer thereof or a pharmaceutically acceptable salt of the compound or tautomer, wherein G¹ is

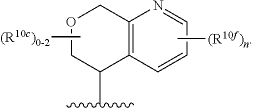

E11.1. The compound of E11, or a tautomer thereof or a pharmaceutically acceptable salt of the compound or tautomer, wherein G¹ is

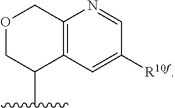

E11.2. The compound of E11 or E11.1, or a tautomer thereof or a pharmaceutically acceptable salt of the compound or tautomer, wherein G¹ is

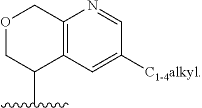

E11.3. The compound of any of E11-E11.2, or a tautomer thereof or a pharmaceutically acceptable salt of the compound or tautomer, wherein G¹ is

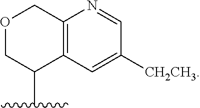 [SMILES: *C1COCc2ncc(CC)cc21]

E12. The compound of any of E1-E3.2, or a tautomer thereof or a pharmaceutically acceptable salt of the compound or tautomer, wherein G¹ is

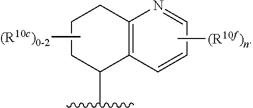

E12.1. The compound of E12, or a tautomer thereof or a pharmaceutically acceptable salt of the compound or tautomer, wherein G¹ is

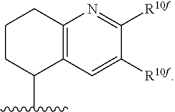

E12.2. The compound of E12 or E12.1, or a tautomer thereof or a pharmaceutically acceptable salt of the compound or tautomer, wherein G¹ is

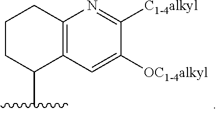

E12.3. The compound of any of E12-E12.2, or a tautomer thereof or a pharmaceutically acceptable salt of the compound or tautomer, wherein G¹ is

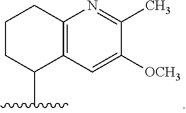 [SMILES: *C1CCCc2nc(C)c(OC)cc21]

E13. The compound of any of E1-E3.2, or a tautomer thereof or a pharmaceutically acceptable salt of the compound or tautomer, wherein G¹ is

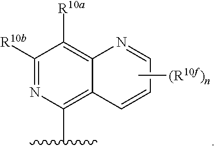

E13.1. The compound of E13, or a tautomer thereof or a pharmaceutically acceptable salt of the compound or tautomer, wherein G¹ is

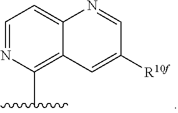

E13.2. The compound of E13 or E13.1, or a tautomer thereof or a pharmaceutically acceptable salt of the compound or tautomer, wherein G¹ is

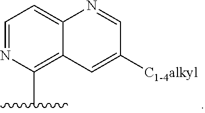

E13.3. The compound of any of E13-E13.2, or a tautomer thereof or a pharmaceutically acceptable salt of the compound or tautomer, wherein G¹ is

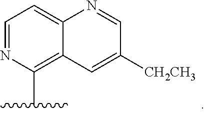 [SMILES: *c1nccc2ncc(CC)cc12]

E14. The compound of any of E1 or E1.2-E3.2, or a tautomer thereof or a pharmaceutically acceptable salt of the compound or tautomer, wherein G¹ is

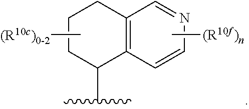

E14.1. The compound of E14, or a tautomer thereof or a pharmaceutically acceptable salt of the compound or tautomer, wherein G¹ is

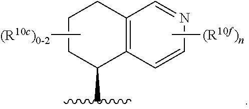

E14.2. The compound of E14, or a tautomer thereof or a pharmaceutically acceptable salt of the compound or tautomer, wherein G¹ is

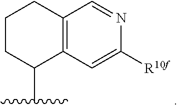

E14.3. The compound of any of E14-E14.2, or a tautomer thereof or a pharmaceutically acceptable salt of the compound or tautomer, wherein G¹ is

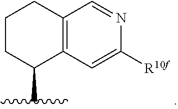

E14.4. The compound of E14 or E14.2, or a tautomer thereof or a pharmaceutically acceptable salt of the compound or tautomer, wherein G¹ is

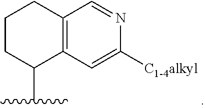

E14.5. The compound of E14.4, or a tautomer thereof or a pharmaceutically acceptable salt of the compound or tautomer, wherein G¹ is

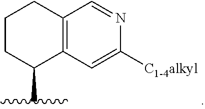

E14.6. The compound of E14.4, or a tautomer thereof or a pharmaceutically acceptable salt of the compound or tautomer, wherein G¹ is

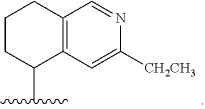 [SMILES: *C1CCCc2cnc(CC)cc21]

E14.7. The compound of E14.6, or a tautomer thereof or a pharmaceutically acceptable salt of the compound or tautomer, wherein G¹ is

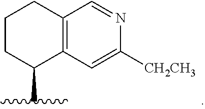 [SMILES: *[C@H]1CCCc2cnc(CC)cc21]

E15. The compound of any of E1-E3.2, or a tautomer thereof or a pharmaceutically acceptable salt of the compound or tautomer, wherein G¹ is

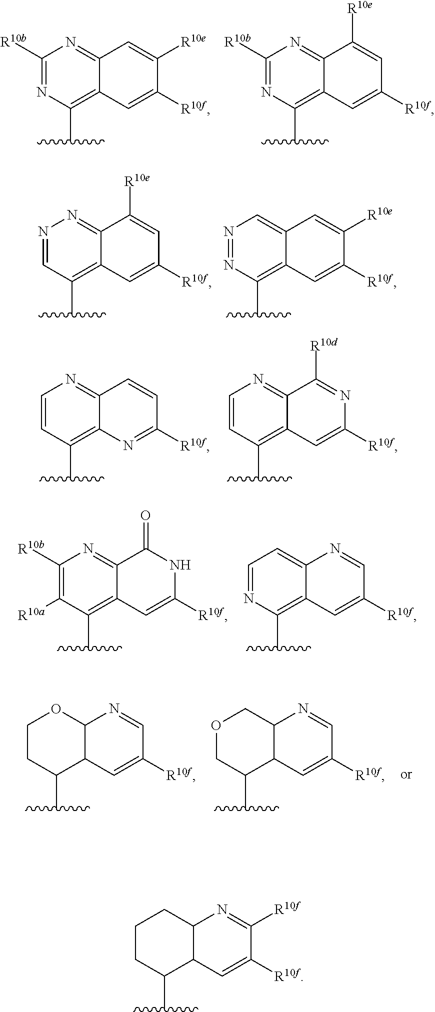

E16. The compound of any of E1-E3.2, E8, E8.1, E8.6, or E15, or a tautomer thereof or a pharmaceutically acceptable salt of the compound or tautomer, wherein R^{1d} is X¹, -L¹-X¹, halogen, C₁₋₄alkyl, or G^{1a}.
E16.1. The compound of E16, or a tautomer thereof or a pharmaceutically acceptable salt of the compound or tautomer, wherein R^{10d} is X¹, halogen, C₁₋₄alkyl, or G^{1a}.
E16.2. The compound of E16 or E16.1, or a tautomer thereof or a pharmaceutically acceptable salt of the compound or tautomer, wherein R^{10d} is X¹.
E16.3. The compound of E16, or a tautomer thereof or a pharmaceutically acceptable salt of the compound or tautomer, wherein R^{10d} is -L¹-X¹.
E16.4. The compound of any of E1-E3.2, E8, E8.1, E8.6, E15, or E16-E16.3, or a tautomer thereof or a pharmaceutically acceptable salt of the compound or tautomer, wherein X¹ is —C(O)N(R^{1a})₂.
E16.5. The compound of E16.4, or a tautomer thereof or a pharmaceutically acceptable salt of the compound or tautomer, wherein X¹ is —C(O)NHR^{1a}.
E16.6. The compound of E16.5, or a tautomer thereof or a pharmaceutically acceptable salt of the compound or tautomer, wherein X¹ is —C(O)NHCH₃.
E16.7. The compound of E16.4 or E16.5, or a tautomer thereof or a pharmaceutically acceptable salt of the compound or tautomer, wherein X¹ is

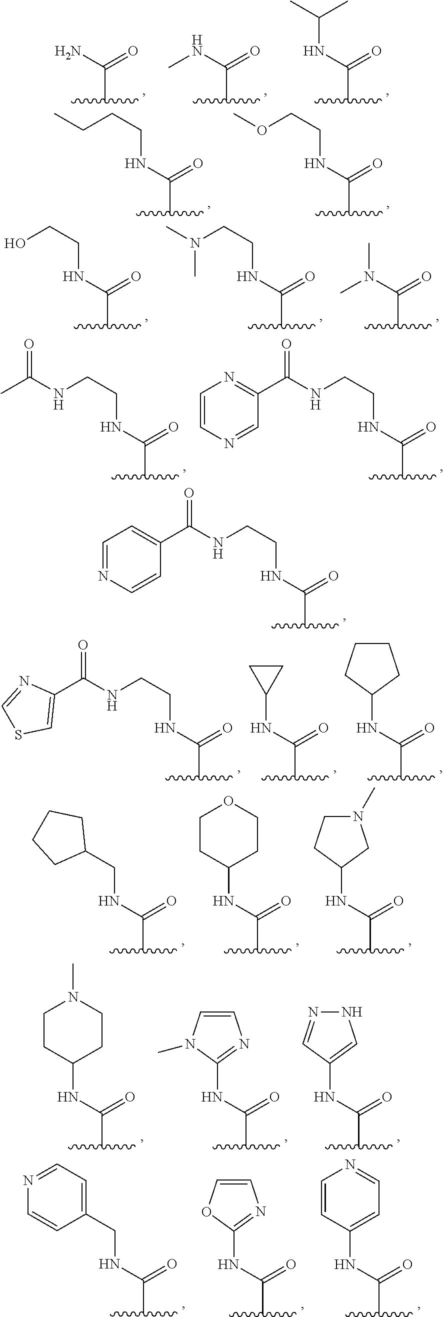

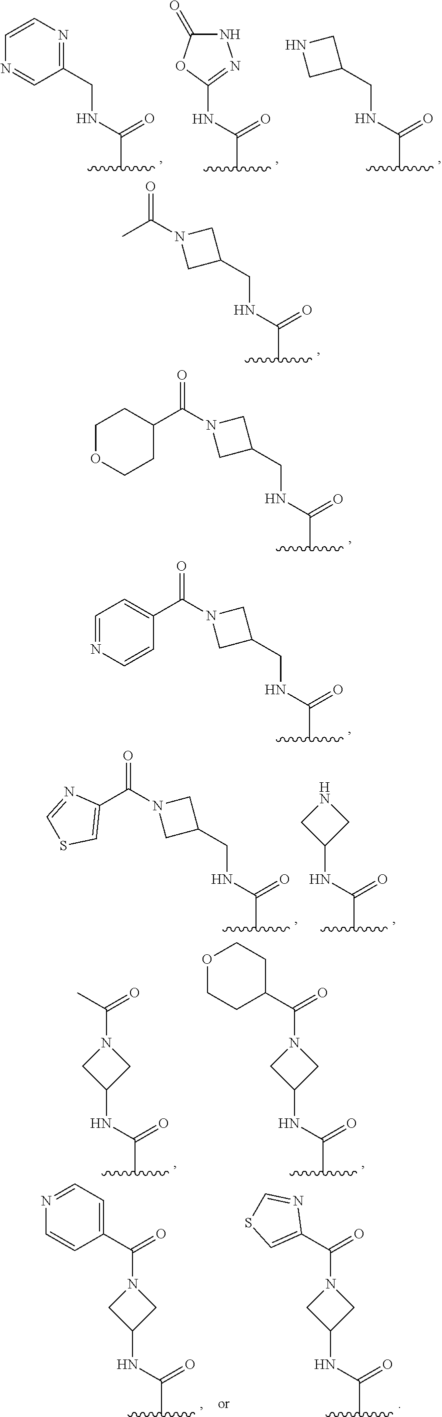

E16.8. The compound of any of E1-E3.2, E8, E8.1, E8.6, E15, or E16-E16.3, or a tautomer thereof or a pharmaceutically acceptable salt of the compound or tautomer, wherein X¹ is —C(O)OR^{1a}.
E16.9. The compound of E16.8, or a tautomer thereof or a pharmaceutically acceptable salt of the compound or tautomer, wherein X¹ is —C(O)OCH₂CH₃.
E16.10. The compound of any of E1-E3.2, E8, E8.1, E8.6, E15, or E16-E16.3, or a tautomer thereof or a pharmaceutically acceptable salt of the compound or tautomer, wherein X¹ is —OR^{1a}.
E16.11. The compound of any of E1-E3.2, E8, E8.1, E8.6, E15, or E16-E16.3, or a tautomer thereof or a pharmaceutically acceptable salt of the compound or tautomer, wherein X¹ is —N(R^{1a})₂.
E16.12. The compound of E16.11, or a tautomer thereof or a pharmaceutically acceptable salt of the compound or tautomer, wherein X¹ is —NHR^{1a}.
E16.13. The compound of any of E1-E3.2, E8, E8.1, E8.6, E15, E16-E16.5, E16.8, or E16.10-E16.12, or a tautomer thereof or a pharmaceutically acceptable salt of the compound or tautomer, wherein R^{1a}, at each occurrence is independently hydrogen, C₁₋₄alkyl, C₁₋₄fluoroalkyl, or —C₂₋₃alkylene-OC₁₋₄alkyl.
E16.14. The compound of E16.13, or a tautomer thereof or a pharmaceutically acceptable salt of the compound or tautomer, wherein R^{1a}, at each occurrence is independently hydrogen.
E16.15. The compound of E16.13, or a tautomer thereof or a pharmaceutically acceptable salt of the compound or tautomer, wherein R^{1a}, at each occurrence is independently C₁₋₄alkyl, e.g., methyl or ethyl.
E16.16. The compound of E16.13, or a tautomer thereof or a pharmaceutically acceptable salt of the compound or tautomer, wherein R^{1a}, at each occurrence is independently C₁₋₄fluoroalkyl, e.g., CH₂CHF₂.
E16.17. The compound of E16.13, or a tautomer thereof or a pharmaceutically acceptable salt of the compound or tautomer, wherein R^{1a}, at each occurrence is independently —C₂₋₃alkylene-OC₁₋₄alkyl, e.g., —CH₂CH₂—OC₁₋₄alkyl, such as —CH₂CH₂—OCH₃.
E16.18. The compound of E16.1, or a tautomer thereof or a pharmaceutically acceptable salt of the compound or tautomer, wherein R^{10d} is halogen.
E16.19. The compound of any of E1-E3.2, E8, E8.1, E8.6, E15, E16, E16.1, or E16.3-E16.18, or a tautomer thereof or a pharmaceutically acceptable salt of the compound or tautomer, wherein the halogen at R^{10d} is chloro.
E16.20. The compound of E16.1, or a tautomer thereof or a pharmaceutically acceptable salt of the compound or tautomer, wherein R^{10d} is C₁₋₄alkyl, e.g., ethyl.
E16.21. The compound of E16.1, or a tautomer thereof or a pharmaceutically acceptable salt of the compound or tautomer, wherein R^{10d} is G^{1a}.
E16.22. The compound of any of E1-E3.2, E8, E8.1, E8.6, E15, or E16-E16.21, or a tautomer thereof or a pharmaceutically acceptable salt of the compound or tautomer, wherein G^{1a} is the 4- to 8-membered heterocyclyl.
E16.23. The compound of any of E1-E3.2, E8, E8.1, E8.6, E15, or E16-E16.22, or a tautomer thereof or a pharmaceutically acceptable salt of the compound or tautomer, wherein the ring system of the 4- to 8-membered heterocyclyl at G^{1a} contains 1-2 heteroatoms independently selected from the group consisting of nitrogen, oxygen, and sulfur.
E16.24. The compound of E16.23, or a tautomer thereof or a pharmaceutically acceptable salt of the compound or tautomer, wherein the ring system of the 4- to 8-membered heterocyclyl at G^{1a} is pyrrolidin-1-yl, piperazin-1-yl, or morpholino.
E16.25. The compound of any of E1-E3.2, E8, E8.1, E8.6, E15, or E16-E16.21, or a tautomer thereof or a pharmaceutically acceptable salt of the compound or tautomer, wherein G^{1a} is the 5- to 6-membered heteroaryl.
E16.26. The compound of E16.25, or a tautomer thereof or a pharmaceutically acceptable salt of the compound or tautomer, wherein the ring system of the 5- to 6-membered heteroaryl at G^{1a} contains 1-2 nitrogen atoms.
E16.27. The compound of E16.26, or a tautomer thereof or a pharmaceutically acceptable salt of the compound or tautomer, wherein the ring system of the 5- to 6-membered heteroaryl at G^{1a} is pyridinyl or pyrrolyl.
E16.28. The compound of E16.27, or a tautomer thereof or a pharmaceutically acceptable salt of the compound or tautomer, wherein the ring system of the 5- to 6-membered heteroaryl at G^{1a} is pyridin-4-yl or pyrrol-1-yl.
E16.29. The compound of any of E1-E3.2, E8, E8.1, E8.6, E15, or E16-E16.28, wherein G^{1a} is optionally substituted with 1-2 substituents independently selected from the group consisting of oxo, C₁₋₄alkyl, OC₁₋₄alkyl, C(O)C₁₋₄alkyl, —NHC(O)C₁₋₄alkyl, —C₂₋₃alkylene-OC₁₋₄alkyl, G^{1b}, and —C₁₋₃alkylene-G^{1b}; and G^{1b} is C₃₋₆cycloalkyl or a 4- to 6-membered monocyclic heterocyclyl containing 1-2 heteroatoms independently selected from O, N, and S.
E16.30. The compound of any of E1-E3.2, E8, E8.1, E8.6, E15, or E16-E16.29, or a tautomer thereof or a pharmaceutically acceptable salt of the compound or tautomer, wherein G¹ is

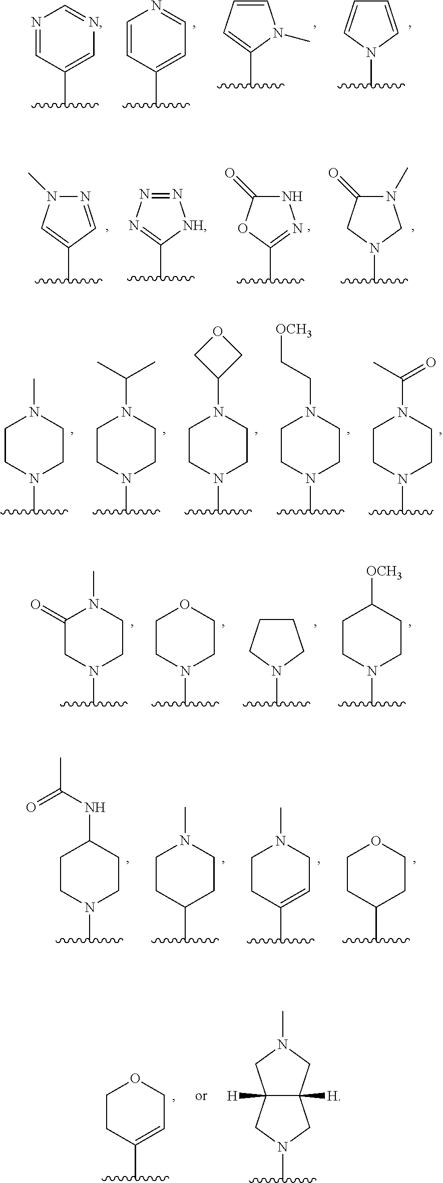

E16.31. The compound of E16.30, or a tautomer thereof or a pharmaceutically acceptable salt of the compound or tautomer, wherein G^{1a} is

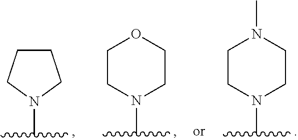

E16.32. The compound of E16.30, or a tautomer thereof or a pharmaceutically acceptable salt of the compound or tautomer, wherein G¹ is

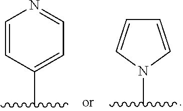 [SMILES: *c1ccncc1 ; *n1cccc1]

E16.33. The compound of any of E1-E3.2, E8, E8.1, E8.6, E15, E16-E16.21, or E16.29, or a tautomer thereof or a pharmaceutically acceptable salt of the compound or tautomer, wherein G^{1a} is the C₃₋₈cycloalkyl.
E16.34. The compound of any of E1-E3.2, E8, E8.1, E8.6, E15, E16-E16.21, or E16.33, or a tautomer thereof or a pharmaceutically acceptable salt of the compound or tautomer, wherein the C₃₋₈cycloalkyl at G^{1a} is unsubstituted.
E16.35. The compound of any of E1-E3.2, E8, E8.1, E8.6, E15, E16-E16.21, E16.33, or E16.34, or a tautomer thereof or a pharmaceutically acceptable salt of the compound or tautomer, wherein the ring system of the C₃₋₈cycloalkyl at G^{1a} is cyclopropyl.
E16.36. The compound of any of E1-E3.2, E8, E8.1, E8.6, or E15-E16.35, or a tautomer thereof or a pharmaceutically acceptable salt of the compound or tautomer, wherein L¹ is CH₂.
E17. The compound of any of E1-E4.14, E5-E-5.1, E5.4, E6-E6.1, or E15-E16.36, or a tautomer thereof or a pharmaceutically acceptable salt of the compound or tautomer, wherein R^{10c} is halogen, OH, —OC₁₋₄alkyl, —OC₁₋₃alkylene-G^{1c}, or —O—C₂₋₃alkylene-Y.
E17.1. The compound of E17, or a tautomer thereof or a pharmaceutically acceptable salt of the compound or tautomer, wherein R^{10c} is halogen.
E17.2. The compound of any of E1-E4.14, E5-E-5.1, E5.4, E6-E6.1, or E15-E17.1, or a tautomer thereof or a pharmaceutically acceptable salt of the compound or tautomer, wherein the halogen at R^{10c} is fluoro.
E17.3. The compound of E17, or a tautomer thereof or a pharmaceutically acceptable salt of the compound or tautomer, wherein R^{10c} is OH.
E17.4. The compound of E17, or a tautomer thereof or a pharmaceutically acceptable salt of the compound or tautomer, wherein R¹⁰ is —OC₁₋₄alkyl.
E17.5. The compound of any of E1-E4.14, E5-E-5.1, E5.4, E6-E6.1, E15-E17, E17.2, or E17.4.4, or a tautomer thereof or a pharmaceutically acceptable salt of the compound or tautomer, wherein the —OC₁₋₄alkyl at R^{10c} is —OCH₃ or —OCH₂CH₃.
E17.6. The compound of E17.5, or a tautomer thereof or a pharmaceutically acceptable salt of the compound or tautomer, wherein the —OC₁₋₄alkyl at R^{10c} is —OCH₃.
E17.7. The compound of E17, or a tautomer thereof or a pharmaceutically acceptable salt of the compound or tautomer, wherein R^{10c} is —OC₁₋-3alkylene-G^{1c}.
E17.8. The compound of any of E1-E4.5, E5-E-5.1, E5.4, E6-E6.1, E15-E17, E17.2, or E17.5-E17.7, or a tautomer thereof or a pharmaceutically acceptable salt of the compound or tautomer, wherein the —OC₁₋₃alkylene-G^{1c} at R^{10c} is —OCH₂-G^{1c}.
E17.9. The compound of any of E1-E4.5, E5-E-5.1, E5.4, E6-E6.1, E15-E17, E17.2, or E17.5-E17.8, or a tautomer thereof or a pharmaceutically acceptable salt of the compound or tautomer, wherein G^{1c} is the 5- to 6-membered heteroaryl containing 1-3 heteroatoms independently selected from O, N, and S.
E17.10. The compound of of any of E1-E4.5, E5-E-5.1, E5.4, E6-E6.1, E15-E17, E17.2, or E17.5-E17.9, or a tautomer thereof or a pharmaceutically acceptable salt of the compound or tautomer, wherein the ring system of the 5- to 6-membered heteroaryl at G¹C is pyrazolyl.
E17.11. The compound of E17.10, or a tautomer thereof or a pharmaceutically acceptable salt of the compound or tautomer, wherein the ring system of the 5- to 6-membered heteroaryl at G^{1c} is pyrazol-5-yl.
E17.12. The compound of any of E1-E4.5, E5-E-5.1, E5.4, E6-E6.1, or E15-E17, E17.2, or E17.5-E17.11, or a tautomer thereof or a pharmaceutically acceptable salt of the compound or tautomer, wherein G^{1c} is optionally independently substituted with 1-3 C₁₋₄alkyl, such as methyl or ethyl.
E17.13. The compound of E17.12, or a tautomer thereof or a pharmaceutically acceptable salt of the compound or tautomer, wherein G^{1c} is

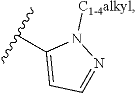

e.g.

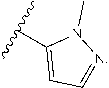 [SMILES: *c1ccnn1C]

E17.14. The compound of E17, or a tautomer thereof or a pharmaceutically acceptable salt of the compound or tautomer, wherein R^{10c} is —O—C₂₋₃alkylene-Y.
E17.15. The compound of any of E1-E4.5, E5-E-5.1, E5.4, E6-E6.1, or E15-E17, E17.2, or E17.5-E17.14, or a tautomer thereof or a pharmaceutically acceptable salt of the compound or tautomer, wherein the —O—C₂₋₃alkylene-Y at R^{10c} is —O—CH₂CH₂—Y.
E17.16. The compound of any of E1-E4.5, E5-E-5.1, E5.4, E6-E6.1, or E15-E17, E17.2, or E17.5-E17.15, or a tautomer thereof or a pharmaceutically acceptable salt of the compound or tautomer, wherein Y is —OC₁₋₄alkyl, e.g., —OCH₃.
E17.17. The compound of any of E1-E4.14, E5-E-5.1, E5.4, E6-E6.1, E15, or E16-E16.36, or a tautomer thereof or a pharmaceutically acceptable salt of the compound or tautomer, wherein R^{10e} is NH₂, —NHC₁₋₄alkyl, —N(C₁₋₄alkyl)₂, or a 4- to 8-membered monocyclic heterocyclyl containing 1-2 heteroatoms selected from N, O, and S, wherein the heterocyclyl is optionally substituted with 1-4 substituents independently selected from the group consisting of halogen, cyano, oxo, C₁₋₄alkyl, C₁₋₄fluoroalkyl, OH, OC₁₋₄alkyl, OC₁₋₄fluoroalkyl, NH₂, —NHC₁₋₄alkyl, and —N(C₁₋₄alkyl)₂.
E17.18. The compound of E17.17, or a tautomer thereof or a pharmaceutically acceptable salt of the compound or tautomer, wherein R^{10e} is NH₂, —NHC₁₋₄alkyl, or —N(C₁₋₄alkyl)₂.
E17.19. The compound of E17.18, or a tautomer thereof or a pharmaceutically acceptable salt of the compound or tautomer, wherein R^{10e} is —NHCH₃.
E17.20. The compound of E17.17, or a tautomer thereof or a pharmaceutically acceptable salt of the compound or tautomer, wherein R^{10e} is the optionally substituted 4- to 8-membered monocyclic heterocyclyl.
E17.21. The compound of any of E1-E4.14, E5-E-5.1, E5.4, E6-E6.1, E15, E16-E16.35, E17.17, or E17.20, or a tautomer thereof or a pharmaceutically acceptable salt of the compound or tautomer, wherein the optionally substituted 4- to 8-membered monocyclic heterocyclyl at R^{10e} contains at least one ring nitrogen atom and is bonded to the parent molecular moiety at the at least one ring nitrogen atom (e.g., azetidin-1-yl, pyrrolidin-1-yl, piperidin-1-yl, morpholino, 1,4-oxazepan4-yl, thiomorpholino, piperazin-1-yl, 1,4-diazepan-1-yl).
E17.22. The compound of E17.21, or a tautomer thereof or a pharmaceutically acceptable salt of the compound or tautomer, wherein R^{10e} is

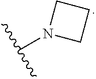 [SMILES: *N1CCC1]

E18. The compound of any of E1-E4.14, E5-E5.1, E5.4, E6-E6.1, E7-E7.1, E8-E8.1, E8.6, E9-E9.1, E10-E10.3, E11-E11.1, E12-E12.1, E13-E13.1, E14-E14.3, or E15-E17.22, or a tautomer thereof or a pharmaceutically acceptable salt of the compound or tautomer, wherein R^{10f}, at each occurrence, is independently C₁₋₄alkyl, OH, —OC₁₋₄alkyl, —OPG, or —OSO₂CF₃.
E18.1. The compound of E18, or a tautomer thereof or a pharmaceutically acceptable salt of the compound or tautomer, wherein R^{10f}, at each occurrence, is independently C₁₋₄alkyl.
E18.2. The compound of any of E1-E4.14, E5-E5.1, E5.4, E6-E6.1, E7-E7.1, E8-E8.1, E8.6, E9-E9.1, E10-E10.3, E11-E11.1, E12-E12.1, E13-E13.1, E14-E14.3, or E15-E18.1, or a tautomer thereof or a pharmaceutically acceptable salt of the compound or tautomer, wherein the C₁₋₄alkyl at R^{10f}, at each occurrence, is independently ethyl.
E18.3. The compound of E18, or a tautomer thereof or a pharmaceutically acceptable salt of the compound or tautomer, wherein R^{10f}, at each occurrence, is independently OH.
E18.4. The compound of E18, or a tautomer thereof or a pharmaceutically acceptable salt of the compound or tautomer, wherein R^{10f}, at each occurrence, is independently —OC₁₋₄alkyl.
E18.5. The compound of any of E1-E4.14, E5-E5.1, E5.4, E6-E6.1, E7-E7.1, E8-E8.1, E8.6, E9-E9.1, E10-E10.3, E11-E11.1, E12-E12.1, E13-E13.1, E14-E14.3, E15-E18, E18.2, or E18.4, or a tautomer thereof or a pharmaceutically acceptable salt of the compound or tautomer, wherein the —OC₁₋₄alkyl at R^{10f}, at each occurrence, is independently —OCH₃ or —OCH₂CH₃.
E18.6. The compound of any of E1-E4.14, E5-E5.1, E5.4, E6-E6.1, E7-E7.1, E8-E8.1, E8.6, E9-E9.1, E10-E10.3, E11-E11.1, E12-E12.1, E13-E13.1, E14-E14.3, E15-E18, E18.2, or E18.4-E18.5, or a tautomer thereof or a pharmaceutically acceptable salt of the compound or tautomer, wherein the —C₁₋₄alkyl at R^{10f}, at each occurrence, is independently —OCH₃.
E18.7. The compound of E18, or a tautomer thereof or a pharmaceutically acceptable salt of the compound or tautomer, wherein R^{10f}, at each occurrence, is independently —OPG.
E18.8. The compound of any of E1-E4.14, E5-E5.1, E5.4, E6-E6.1, E7-E7.1, E8-E8.1, E8.6, E9-E9.1, E10-E10.3, E11-E11.1, E12-E12.1, E13-E13.1, E14-E14.3, E15-E18, E18.2, or E18.5-E18.7, or a tautomer thereof or a pharmaceutically acceptable salt of the compound or tautomer, wherein PG is benzyl.
E18.9. The compound of E18, or a tautomer thereof or a pharmaceutically acceptable salt of the compound or tautomer, wherein R^{10f}, at each occurrence, is independently —OSO₂CF₃.
E19. The compound of any of E1-E3.2, E15, or E18.8, or a tautomer thereof or a pharmaceutically acceptable salt of the compound or tautomer, wherein G¹ is

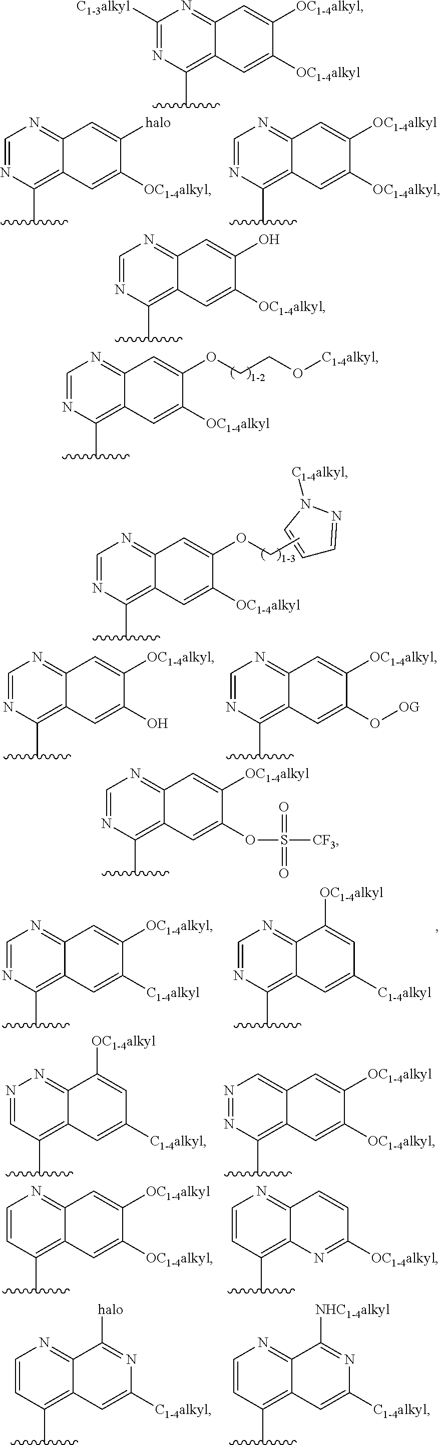

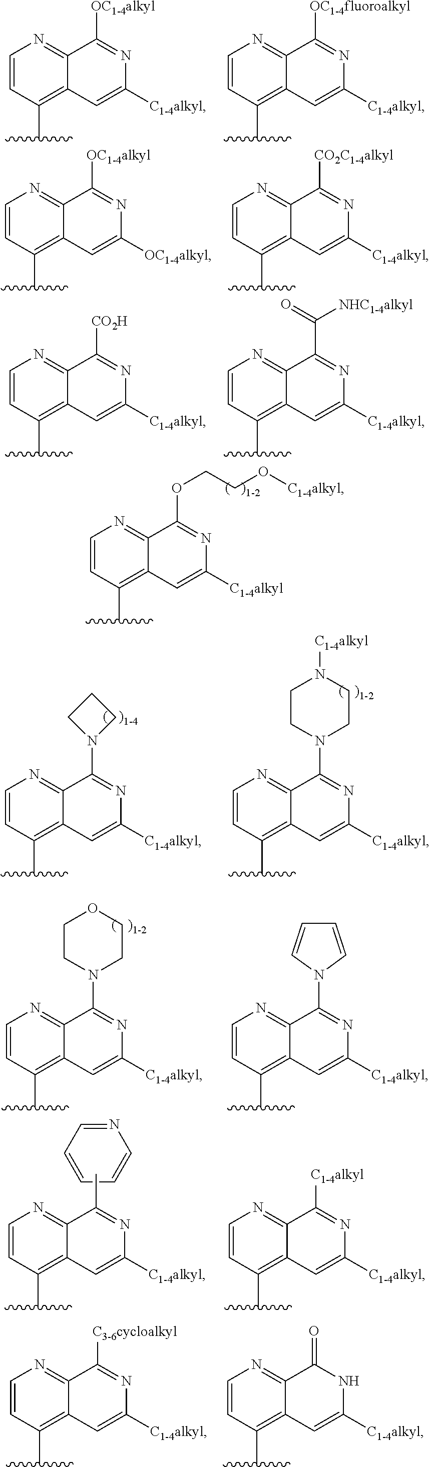

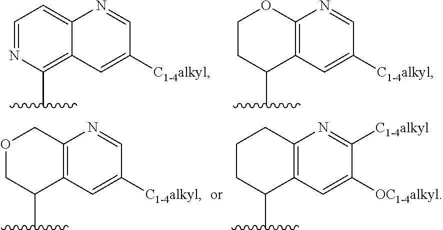

E19.1. The compound of E19, or a pharmaceutically acceptable salt thereof, wherein G¹ is

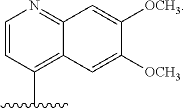 [SMILES: *c1ccnc2cc(OC)c(OC)cc12]

E20. A compound of formula (I)

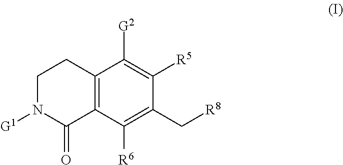

or a pharmaceutically acceptable salt thereof, wherein;
G¹ is

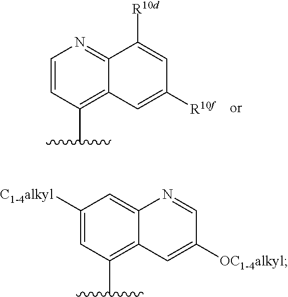

R^{10d} is X¹, hydrogen, halogen, C₁₋₄alkyl, C₁₋₄fluoroalkyl, or G^{1a};
R^{10f} is halogen, cyano, C₁₋₄alkyl, C₁₋₄fluoroalkyl, OH, —OC₁₋₄alkyl, —OC₁₋₄fluoroalkyl, —OC₃₋₄cycloalkyl, —OC₁₋₃alkylene-C₃₋₄cycloalkyl, —OPG, or —OSO₂CF₃;
PG is a hydroxy protecting group;
X¹ is —C(O)N(R^{1a})₂, —OR^{1a}, —N(R^{1a})₂, cyano, —C(O)OR^{1a}, —C(O)R^{1b}, —SO₂R^{1b}, —SO₂N(R^{1a})₂, —NR^{1a}C(O)H, or —NR^{1a}C(O)R^{1b};
R^{1a}, at each occurrence, is independently hydrogen, C₁₋₄alkyl, C₁₋₄fluoroalkyl, —C₂₋₃alkylene-OR^{1e}, —C₂₋₃alkylene-N(R^{1e})₂, —C₂₋₃alkylene-N(R^{1e})C(O)R^{1e}, G^{1a}, or —C₁₋₃alkylene-G^{1a};
R^{1b}, at each occurrence, is independently C₁₋₄alkyl, C₁₋₄fluoroalkyl, —C₁₋₃alkylene-OR^{1e}, —C₁₋₃alkylene-N(R^{1e})₂, —C₁₋₃alkylene-N(R^{1e})C(O)R^{1e}, G^{1a}, or —C₁₋₃alkylene-G^{1a};
G^{1a}, at each occurrence, is independently C₃₋₈cycloalkyl, 5- to 6-membered heteroaryl, or 4- to 8-membered heterocyclyl, wherein G^{1a} is optionally substituted with 1-4 substituents independently selected from the group consisting of halogen, C₁₋₄alkyl, C₁₋₄fluoroalkyl, oxo, -L²-X², and -L²-G^{1b};
L², at each occurrence, is independently a bond or C₁₋₃alkylene;
X², at each occurrence, is independently —OR^{1c}, —N(R^{1c})₂, —SR^{1c}, cyano, —C(O)OR^{1c}, —C(O)N(R^{1c})₂, —C(O)R^{1c}, —SOR^{1d}, —SO₂R^{1d}, —SO₂N(R^{1c})₂, —NR^{1c}C(O)R^{1c}, —NR^{1c}C(O)OR^{1c}, —NR^{1c}C(O)N(R^{1c})₂, —NR^{1c}S(O)₂R^{1d}, or —NR^{1c}S(O)₂N(R^{1c})₂,
R^{1c}, at each occurrence, is independently hydrogen, C₁₋₄alkyl, C₁₋₄fluoroalkyl, G^{1b}, or —C₁₋₃alkylene-G^{1b}, wherein alternatively two R^{1c}, together with a common nitrogen atom to which the R^{1c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{1d}, at each occurrence, is independently C₁₋₄alkyl, C₁₋₄fluoroalkyl, G^{1b}, or —C₁₋₃alkylene-G^{1b};
R^{1e}, at each occurrence, is independently hydrogen, C₁₋₄alkyl, C₁₋₄fluoroalkyl, G^{1b}, or —C₁₋₃alkylene-Gb, wherein alternatively two R^{1c}, together with a common nitrogen atom to which the R^{1c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
G^{1b}, at each occurrence, is independently a C₃₋₆cycloalkyl, a 4- to 6-membered monocyclic heterocyclyl containing 1-2 heteroatoms independently selected from O, N, and S, a 5- to 6-membered heteroaryl containing 1-4 heteroatoms independently selected from O, N, and S, or a phenyl, wherein G^{1b} is optionally substituted with 1-4 substituents independently selected from the group consisting of halogen, cyano, C₁₋₄alkyl, C₁₋₄fluoroalkyl, oxo, —OH, and —OC₁₋₄alkyl;
G² is a 5- to 6-membered heteroaryl, wherein G² is optionally substituted with 1-4 substituents independently selected from the group consisting of C₁₋₆alkyl, C₁₋₆haloalkyl, halogen, —OR^{4c}, —N(R^{4c})₂, cyano, —C(O)OR^{4c}, —C(O)N(R^{4c})₂, —C(O)R^{4c}, —SO₂R^{4d}, —SO₂N(R^{4c})₂, —NR^{4c}C(O)R^{4c}, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of C₁₋₄alkyl and halogen;
R^{4c}, at each occurrence, is independently hydrogen, C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen, wherein alternatively two R⁴, together with a common nitrogen atom to which the R^{4c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{4d}, at each occurrence, is independently C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen.
R⁵ and R⁶ are each independently hydrogen, halogen, C₁₋₄alkyl, C₁₋₄fluoroalkyl, or —OC₁₋₄alkyl; and
R⁸ is

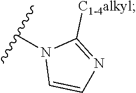

provided the compound is not:
2-(6-methoxy-8-methylquinolin-4-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one, or a pharmaceutically acceptable salt thereof.
   E21. The compound of claim 19, or a pharmaceutically acceptable salt thereof, wherein G¹ is

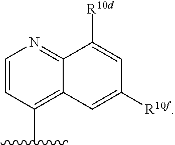

E22. The compound of E20 or E21, or a pharmaceutically acceptable salt thereof, wherein R^{10d} is X¹.
E22.1. The compound of any of E20-E22, or a pharmaceutically acceptable salt thereof, wherein is X¹ is —C(O)N(R^{1a})₂.
E22.2. The compound of E22.1, or a pharmaceutically acceptable salt thereof, wherein X¹ is —C(O)NHR^{1a}.
E22.3. The compound of E22.1, or a pharmaceutically acceptable salt thereof, wherein X¹ is

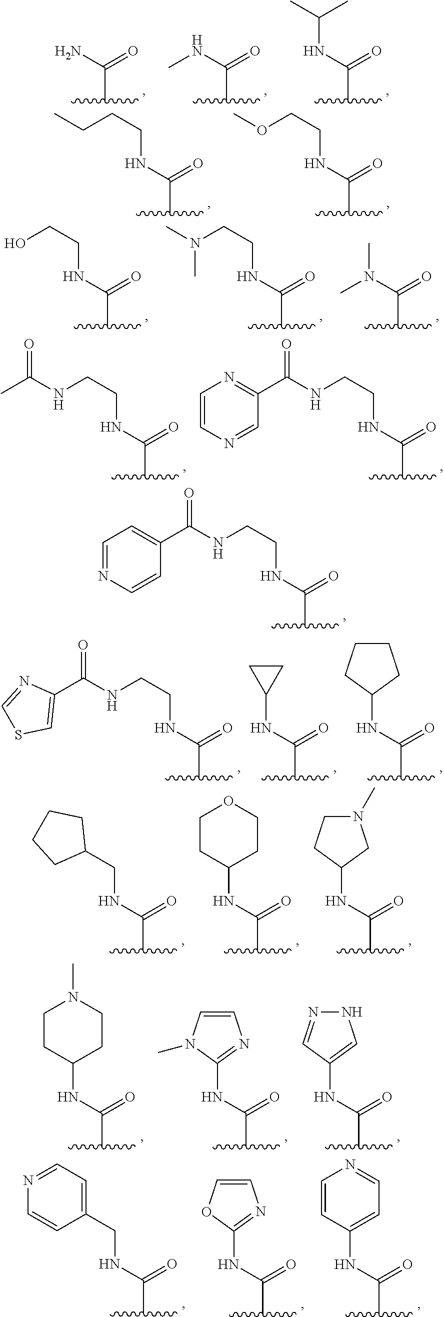

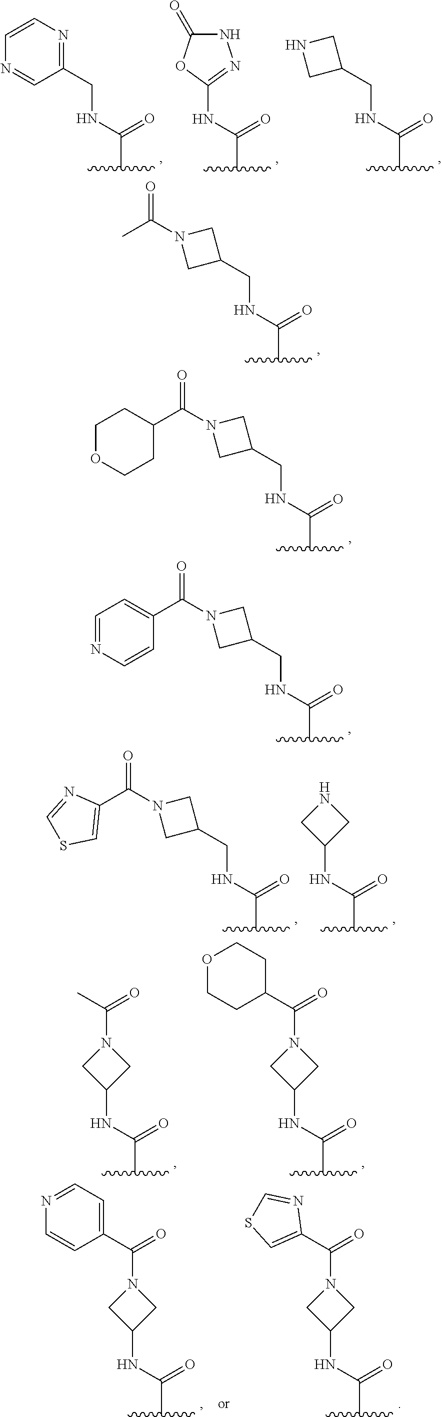

E22.4. The compound of any of E20-E22, or a pharmaceutically acceptable salt thereof, wherein X¹ is —C(O)OR^{1a}.
E22.5. The compound of any of E20-E22.4, or a pharmaceutically acceptable salt thereof, wherein R^{1a}, at each occurrence is independently hydrogen, C₁₋₄alkyl, C₁₋₄fluoroalkyl, or —C₂₋₃alkylene-OC₁₋₄alkyl.
E22.6. The compound of E22.5, or a pharmaceutically acceptable salt thereof, wherein R^{1a}, at each occurrence is independently hydrogen.
E22.7. The compound of E22.5, or a pharmaceutically acceptable salt thereof, wherein R^{1a}, at each occurrence is independently C₁₋₄alkyl, e.g., methyl or ethyl.
E23. The compound of any of E20-E22.7, or a pharmaceutically acceptable salt thereof, wherein R¹⁰ is C₁₋₄alkyl.
E23.1. The compound of any of E20-E23, or a pharmaceutically acceptable salt thereof, wherein the C₁₋₄alkyl at R¹⁰ is ethyl.
E24. The compound of E20, or a pharmaceutically acceptable salt thereof, wherein G¹ is

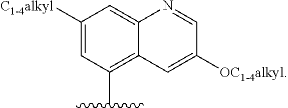

E24.1. The compound of any of E20-E24, or a pharmaceutically acceptable salt thereof, wherein the

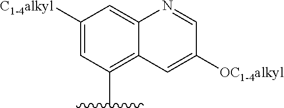

at G¹ is

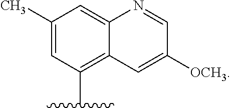 [SMILES: *c1cc(C)cc2ncc(OC)cc12]

E24.2. The compound of any of E20-E24.1, or a pharmaceutically acceptable salt thereof, wherein R⁸ is

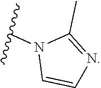 [SMILES: *n1ccnc1C]

E25. The compound of any of E1-E24.2, or a tautomer thereof or a pharmaceutically acceptable salt of the compound or tautomer, wherein G² is the optionally substituted 5- to 6-membered heteroaryl.
E25.1. The compound of any of E1-E25, or a tautomer thereof or a pharmaceutically acceptable salt of the compound or tautomer, wherein the ring system of the optionally substituted 5- to 6-membered heteroaryl at G² is pyrazolyl.
E25.2. The compound of any of E1-E24.2, or a tautomer thereof or a pharmaceutically acceptable salt of the compound or tautomer, wherein G² is the optionally substituted phenyl.
E25.3. The compound of any of E1-E25.2, or a tautomer thereof or a pharmaceutically acceptable salt of the compound or tautomer, wherein G² is optionally substituted with 1-4 substituents independently selected from the group consisting of halogen, C₁₋₄alkyl, and C₁₋₄fluoroalkyl.
E25.4. The compound of E25.3, or a tautomer thereof or a pharmaceutically acceptable salt of the compound or tautomer, wherein G² is optionally substituted with 1-2 substituents independently selected from the group consisting of C₁₋₂alkyl and C₁₋₂fluoroalkyl.
E26. The compound of E25-E25.1 or E25.3, or a tautomer thereof or a pharmaceutically acceptable salt of the compound or tautomer, wherein, G² is

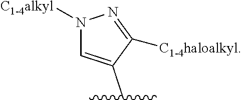

E26.1. The compound of E25.4 or E26, or a tautomer thereof or a pharmaceutically acceptable salt of the compound or tautomer, wherein, G² is

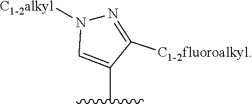

E26.2. The compound of E26 or E26.1, or a tautomer thereof or a pharmaceutically acceptable salt of the compound or tautomer, wherein, G² is

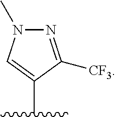 [SMILES: *c1cn(C)nc1C(F)(F)F]

E26.3. The compound of E26 or E26.1, or a tautomer thereof or a pharmaceutically acceptable salt of the compound or tautomer, wherein, G² is

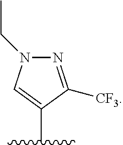 [SMILES: *c1cn(CC)nc1C(F)(F)F]

E26.4. The compound of any of E25-E25.1 or E25.3, or a tautomer thereof or a pharmaceutically acceptable salt of the compound or tautomer, wherein, G² is

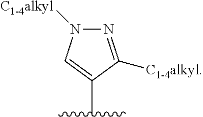

E26.5. The compound of E25.4 or E26.4, or a tautomer thereof or a pharmaceutically acceptable salt of the compound or tautomer, wherein, G² is

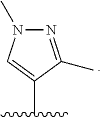

E26.6. The compound of any of E25-E25.1 or E25.3, or a tautomer thereof or a pharmaceutically acceptable salt of the compound or tautomer, wherein, G² is

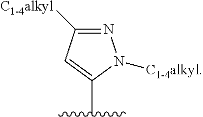

E26.7. The compound of E25.4 or E26.6, or a tautomer thereof or a pharmaceutically acceptable salt of the compound or tautomer, wherein, G² is

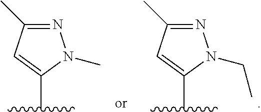

E26.8. The compound of any of E25-E25.1 or E25.3, or a tautomer thereof or a pharmaceutically acceptable salt of the compound or tautomer, wherein, G² is

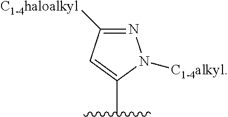

E26.9. The compound of E25.4 or E26.8, or a tautomer thereof or a pharmaceutically acceptable salt of the compound or tautomer, wherein, G³ is

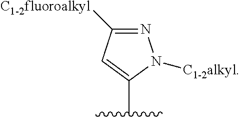

E26.10. The compound of E26.9, or a tautomer thereof or a pharmaceutically acceptable salt of the compound or tautomer, wherein, G² is

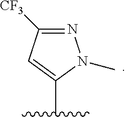 [SMILES: *c1cc(C(F)(F)F)nn1C]

E26.11. The compound of any of E25-E25.1 or E25.3, or a tautomer thereof or a pharmaceutically acceptable salt of the compound or tautomer, wherein, G² is

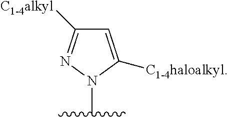

E26.12. The compound of E25.4 or E26.11, or a tautomer thereof or a pharmaceutically acceptable salt of the compound or tautomer, wherein, G² is

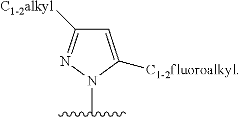

E26.13. The compound of E26.12, or a tautomer thereof or a pharmaceutically acceptable salt of the compound or tautomer, wherein, G² is

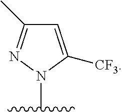 [SMILES: *n1nc(C)cc1C(F)(F)F]

E26.14. The compound of E25.2, or a tautomer thereof or a pharmaceutically acceptable salt of the compound or tautomer, wherein, G² is

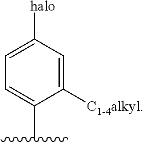

E26.15. The compound of E26.14, or a tautomer thereof or a pharmaceutically acceptable salt of the compound or tautomer, wherein, G² is

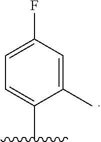 [SMILES: *c1ccc(F)cc1C]

E27. The compound of any of E1-E26.15, or a tautomer thereof or a pharmaceutically acceptable salt of the compound or tautomer, wherein R⁵ is hydrogen.
E28. The compound of any of E1-E27, or a tautomer thereof or a pharmaceutically acceptable salt of the compound or tautomer, wherein R⁶ is hydrogen.
E29.1. A compound selected from the group consisting of:
7-((1H-imidazol-1-yl)methyl)-2-(3-ethyl-1,6-naphthyridin-5-yl)-5-(1-methyl-3-(trifluoromethyl)-1H-pyrazol-4-yl)-3,4-dihydroisoquinolin-1(2H)-one;
2-(3-methoxy-7-methylquinolin-5-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one;
ethyl 6-ethyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxylate;
6-ethyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxylic acid;
6-ethyl-N-methyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxamide;
6-ethyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxamide;
4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridin-8(7H)-one;
7-((1H-imidazol-1-yl)methyl)-2-(6-ethyl-8-hydroxy-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-(methylamino)-1,7-naphthyridin-4-yl)-5-(1-methyl-3-(trifluoromethyl)-1H-pyrazol-4-yl)-3,4-dihydroisoquinolin-1(2H)-one;
2-(6-ethyl-8-methoxy-1,7-naphthyridin-4-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one;
(S)-7-((1H-imidazol-1-yl)methyl)-2-(6-ethyl-3,4-dihydro-2H-pyrano[2,3-b]pyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,8-dimethoxy-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-methoxycinnolin-4-yl)-5-(1-methyl-3-(trifluoromethyl)-1H-pyrazol-4-yl)-3,4-dihydroisoquinolin-1(2H)-one;
2-(6-ethyl-8-methoxycinnolin-4-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxyquinolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(6-methoxy-1,5-naphthyridin-4-yl)-5-(1-methyl-3-(trifluoromethyl)-1H-pyrazol-4-yl)-3,4-dihydroisoquinolin-1(2H)-one;
ethyl 4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridine-8-carboxylate;
4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridine-8-carboxylic acid;
4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-N-methyl-1,7-naphthyridine-8-carboxamide;
7-((1H-imidazol-1-yl)methyl)-2-(3-ethyl-5,8-dihydro-6H-pyrano[3,4-b]pyridin-5-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(3-methoxy-2-methyl-5,6,7,8-tetrahydroquinolin-5-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(7-hydroxy-6-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxy-7-(2-methoxyethoxy)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxy-7-((1-methyl-1H-pyrazol-5-yl)methoxy)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethoxy-7-methoxyquinazolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(7-ethoxy-6-methoxyquinazolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(6,7-diethoxyquinazolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(6,7-dimethoxyphthalazin-1-yl)-5-(1-methyl-3-(trifluoromethyl)-1H-pyrazol-4-yl)-3,4-dihydroisoquinolin-(2H)-one;
7-((1H-imidazol-1-yl)methyl)-2-(7-fluoro-6-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-(2-methoxyethoxy)-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-(pyrrolidin-1-yl)-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-(1H-pyrrol-1-yl)-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(8-ethoxy-6-ethyl-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,8-diethyl-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(8-cyclopropyl-6-ethyl-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(8-(2,2-difluoroethoxy)-6-ethyl-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-(benzyloxy)-7-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-hydroxy-7-methoxyquinazolin-4-yl)-5-(1-methyl-3-(trifluoromethyl)-1H-pyrazol-4-yl)-3,4-dihydroisoquinolin-1(2H)-one;
4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-7-methoxyquinazolin-6-yl trifluoromethanesulfonate;
7-((1H-imidazol-1-yl)methyl)-2-(6-ethyl-7-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-morpholino-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-(pyridin-4-yl)-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-(4-methylpiperazin-1-yl)-1,7-naphthyridin-4-yl)-5-(1-methyl-3-(trifluoromethyl)-1H-pyrazol-4-yl)-3,4-dihydroisoquinolin-1(2H)-one;
   or a pharmaceutically acceptable salt thereof.
   E29.2. A compound selected from the group consisting of:
7-((1H-imidazol-1-yl)methyl)-2-(6-ethyl-8-methoxy-1,7-naphthyridin-4-yl)-5-(3-methyl-5-(trifluoromethyl)-1H-pyrazol-1-yl)-3,4-dihydroisoquinolin-1(2H)-one;
7-((1H-imidazol-1-yl)methyl)-2-(6-ethyl-8-methoxy-2-methyl-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxycinnolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,8-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-methoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-(methoxymethyl)-1,7-naphthyridin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(7-fluoro-6-methoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(7-(azetidin-1-yl)-6-methoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxy-2-methyl-7-(methylamino)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-morpholinoquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-(methylamino)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-methylquinazolin-4-yl)-5-(1-ethyl-3-methyl-1H-pyrazol-5-yl)-3,4-dihydroisoquinolin-1(2H)-one;
7-((1H-imidazol-1-yl)methyl)-2-(6,7-dimethoxy-2-methylquinazolin-4-yl)-5-(1,3-dimethyl-1H-pyrazol-5-yl)-3,4-dihydroisoquinolin-1(2H)-one;
(S)-7-((1H-imidazol-1-yl)methyl)-3′-ethyl-5-(1-methyl-3-(trifluoromethyl)-1H-pyrazol-4-yl)-3,4,5′,6′,7′,8′-hexahydro-1H-[2,5′-biisoquinolin]-1-one;
7-((1H-imidazol-1-yl)methyl)-2-(6,7-dimethoxy-2-methylquinazolin-4-yl)-5-(1-methyl-3-(trifluoromethyl)-1H-pyrazol-5-yl)-3,4-dihydroisoquinolin-1(2H)-one;
7-((1H-imidazol-1-yl)methyl)-2-(2-(ethyl(methyl)amino)-6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(2-ethyl-6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(2-cyclopropyl-6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-methylquinazolin-4-yl)-5-(1,3-dimethyl-1H-pyrazol-4-yl)-3,4-dihydroisoquinolin-1(2H)-one;
7-((1H-imidazol-1-yl)methyl)-2-(6,7-dimethoxy-2-methylquinazolin-4-yl)-5-(4-fluoro-2-methylphenyl)-3,4-dihydroisoquinolin-1(2H)-one;
7-((1H-imidazol-1-yl)methyl)-2-(6,7-dimethoxy-2-(tetrahydro-2H-pyran-4-yl)quinazolin-4-yl)-5-(1-methyl-3-(trifluoromethyl)-1H-pyrazol-4-yl)-3,4-dihydroisoquinolin-1(2H)-one;
   or a pharmaceutically acceptable salt thereof.
   E30. A pharmaceutical composition comprising the compound of any of E1-E29.2, or a tautomer thereof or a pharmaceutically acceptable salt of the compound or tautomer, and a pharmaceutically acceptable carrier.
   E31. The compound of any of E1-E29.2, or a tautomer thereof or a pharmaceutically acceptable salt of the compound or tautomer, or the pharmaceutical composition of E30, for use in the treatment of cancer.
   E32. The compound of any of E1-E29.2, or a tautomer thereof or a pharmaceutically acceptable salt of the compound or tautomer, or the pharmaceutical composition of E30, for use in the inhibition of cancer cell proliferation.
   E33. A method of treating cancer comprising administering to a subject in need thereof, a therapeutically effective amount of the compound of any of E1-E29.2, or a tautomer thereof or a pharmaceutically acceptable salt of the compound or tautomer, or the pharmaceutical composition of E30.
   E34. A method of inhibiting cancer cell proliferation, comprising administering to a subject in need thereof, the compound of any of E1-E29.2, or a tautomer thereof or a pharmaceutically acceptable salt of the compound or tautomer, or the pharmaceutical composition of E30, in an amount effective to inhibit the cancer cell proliferation.
   E35. Use of the compound of any of E1-E29.2, or a tautomer thereof or a pharmaceutically acceptable salt of the compound or tautomer, or the pharmaceutical composition of E30, in the manufacture of a medicament for the treatment of cancer.
   E36. Use of the compound of any of E1-E29.2, or a tautomer thereof or a pharmaceutically acceptable salt of the compound or tautomer, or the pharmaceutical composition of E30, in the manufacture of a medicament for the inhibition of cancer cell proliferation.

In certain embodiments, the compound of formula (I) is selected from the group consisting of the compounds in Table 1, or a pharmaceutically acceptable salt thereof.

TABLE 1
Exemplary compounds.

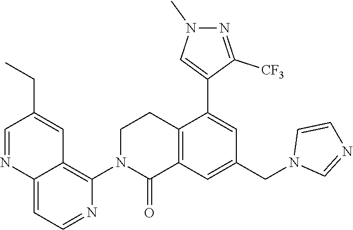 [SMILES: CCc1cnc2ccnc(N3CCc4c(cc(Cn5ccnc5)cc4-c4cn(C)nc4C(F)(F)F)C3=O)c2c1]

I-1

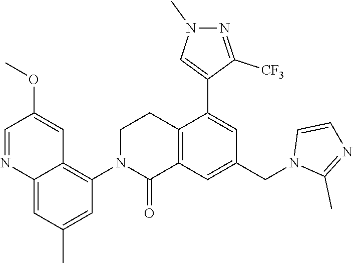 [SMILES: COc1cnc2cc(C)cc(N3CCc4c(cc(Cn5ccnc5C)cc4-c4cn(C)nc4C(F)(F)F)C3=O)c2c1]

I-2

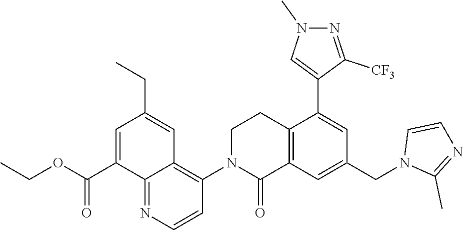 [SMILES: CCOC(=O)c1cc(CC)cc2c(N3CCc4c(cc(Cn5ccnc5C)cc4-c4cn(C)nc4C(F)(F)F)C3=O)ccnc12]

I-3

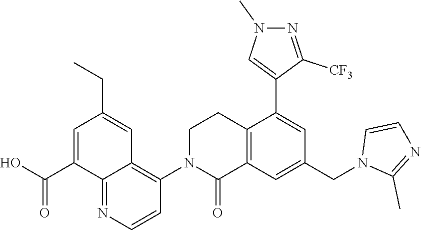 [SMILES: CCc1cc(C(=O)O)c2nccc(N3CCc4c(cc(Cn5ccnc5C)cc4-c4cn(C)nc4C(F)(F)F)C3=O)c2c1]

I-4

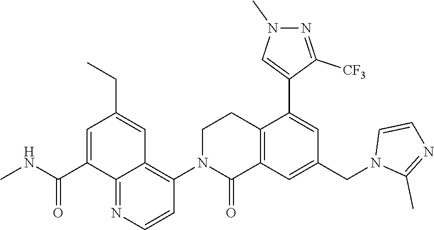 [SMILES: CCc1cc(C(=O)NC)c2nccc(N3CCc4c(cc(Cn5ccnc5C)cc4-c4cn(C)nc4C(F)(F)F)C3=O)c2c1]

I-5

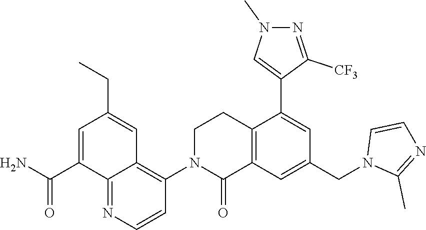 [SMILES: CCc1cc(C(N)=O)c2nccc(N3CCc4c(cc(Cn5ccnc5C)cc4-c4cn(C)nc4C(F)(F)F)C3=O)c2c1]

I-6

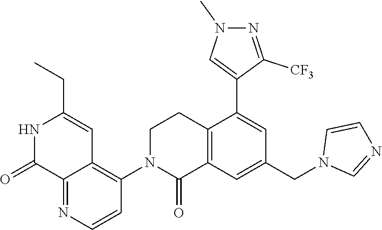 [SMILES: CCc1cc2c(N3CCc4c(cc(Cn5ccnc5)cc4-c4cn(C)nc4C(F)(F)F)C3=O)ccnc2c(=O)[nH]1]

and

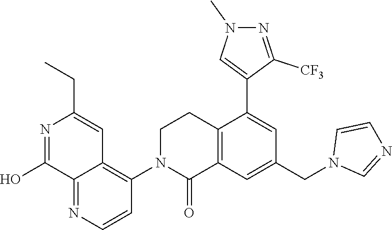 [SMILES: CCc1cc2c(N3CCc4c(cc(Cn5ccnc5)cc4-c4cn(C)nc4C(F)(F)F)C3=O)ccnc2c(O)n1]

I-7

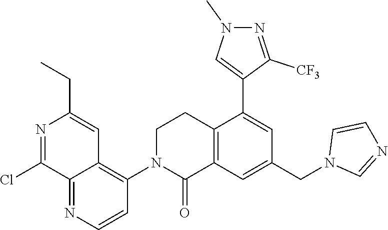 [SMILES: CCc1cc2c(N3CCc4c(cc(Cn5ccnc5)cc4-c4cn(C)nc4C(F)(F)F)C3=O)ccnc2c(Cl)n1]

I-8

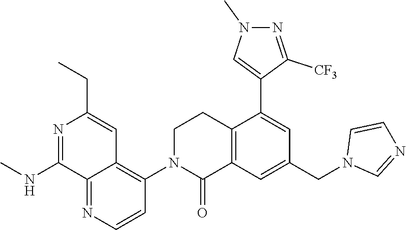 [SMILES: CCc1cc2c(N3CCc4c(cc(Cn5ccnc5)cc4-c4cn(C)nc4C(F)(F)F)C3=O)ccnc2c(NC)n1]

I-9

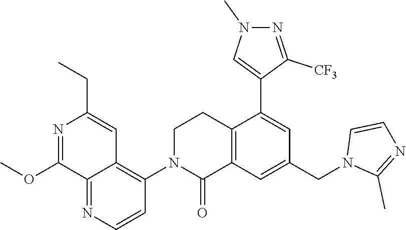 [SMILES: CCc1cc2c(N3CCc4c(cc(Cn5ccnc5C)cc4-c4cn(C)nc4C(F)(F)F)C3=O)ccnc2c(OC)n1]

I-10

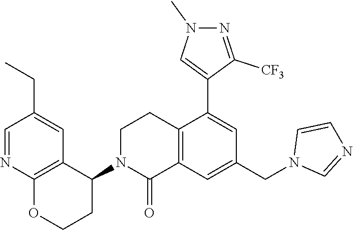 [SMILES: CCc1cnc2c(c1)[C@@H](N1CCc3c(cc(Cn4ccnc4)cc3-c3cn(C)nc3C(F)(F)F)C1=O)CCO2]

I-11

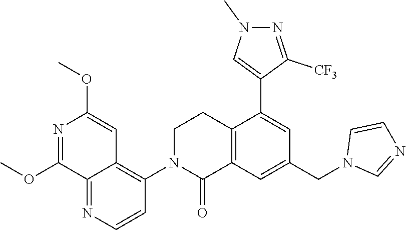 [SMILES: COc1cc2c(N3CCc4c(cc(Cn5ccnc5)cc4-c4cn(C)nc4C(F)(F)F)C3=O)ccnc2c(OC)n1]

I-12

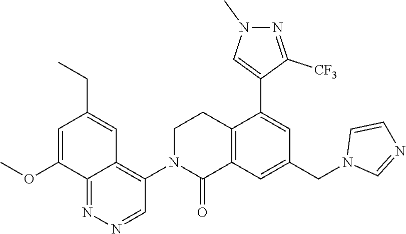 [SMILES: CCc1cc(OC)c2nncc(N3CCc4c(cc(Cn5ccnc5)cc4-c4cn(C)nc4C(F)(F)F)C3=O)c2c1]

I-13

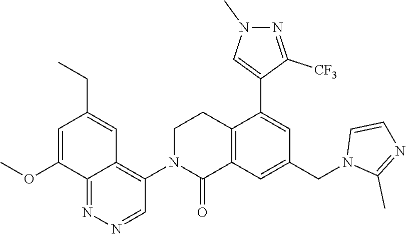 [SMILES: CCc1cc(OC)c2nncc(N3CCc4c(cc(Cn5ccnc5C)cc4-c4cn(C)nc4C(F)(F)F)C3=O)c2c1]

I-14

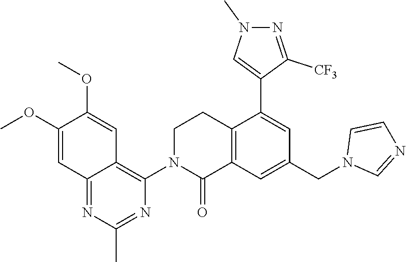 [SMILES: COc1cc2nc(C)nc(N3CCc4c(cc(Cn5ccnc5)cc4-c4cn(C)nc4C(F)(F)F)C3=O)c2cc1OC]

I-15

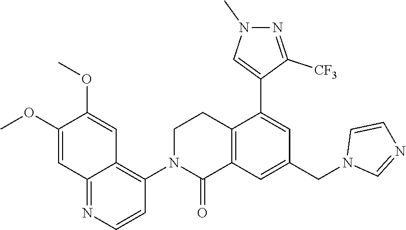 [SMILES: COc1cc2nccc(N3CCc4c(cc(Cn5ccnc5)cc4-c4cn(C)nc4C(F)(F)F)C3=O)c2cc1OC]

I-16

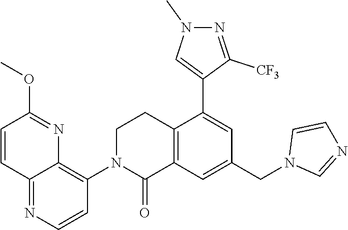 [SMILES: COc1ccc2nccc(N3CCc4c(cc(Cn5ccnc5)cc4-c4cn(C)nc4C(F)(F)F)C3=O)c2n1]

I-17

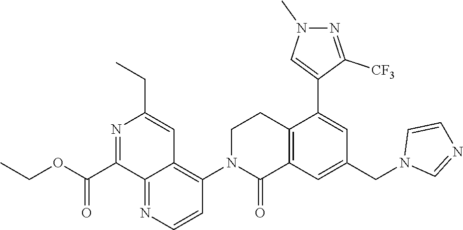 [SMILES: CCOC(=O)c1nc(CC)cc2c(N3CCc4c(cc(Cn5ccnc5)cc4-c4cn(C)nc4C(F)(F)F)C3=O)ccnc12]

I-18

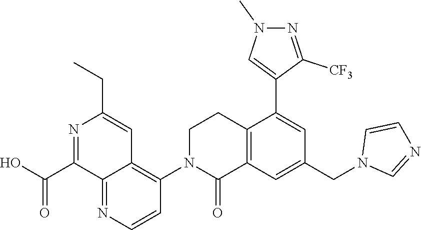 [SMILES: CCc1cc2c(N3CCc4c(cc(Cn5ccnc5)cc4-c4cn(C)nc4C(F)(F)F)C3=O)ccnc2c(C(=O)O)n1]

I-19

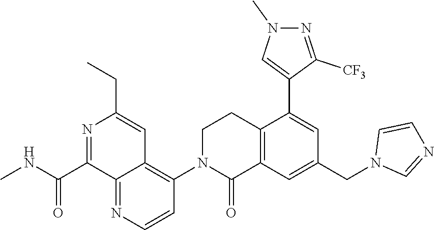 [SMILES: CCc1cc2c(N3CCc4c(cc(Cn5ccnc5)cc4-c4cn(C)nc4C(F)(F)F)C3=O)ccnc2c(C(=O)NC)n1]

I-20

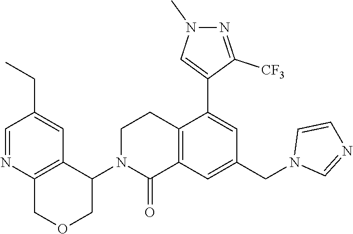 [SMILES: CCc1cnc2c(c1)C(N1CCc3c(cc(Cn4ccnc4)cc3-c3cn(C)nc3C(F)(F)F)C1=O)COC2]

I-21

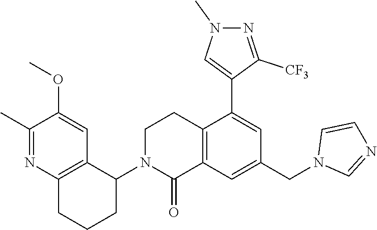 [SMILES: COc1cc2c(nc1C)CCCC2N1CCc2c(cc(Cn3ccnc3)cc2-c2cn(C)nc2C(F)(F)F)C1=O]

I-22

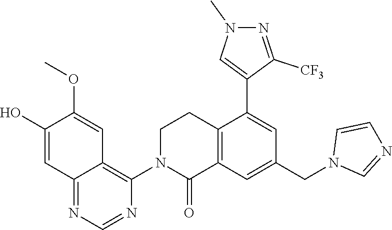 [SMILES: COc1cc2c(N3CCc4c(cc(Cn5ccnc5)cc4-c4cn(C)nc4C(F)(F)F)C3=O)ncnc2cc1O]

I-23

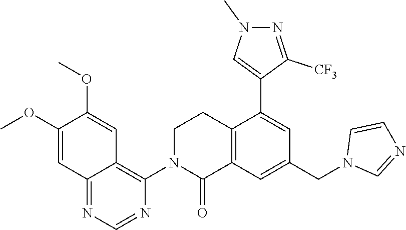 [SMILES: COc1cc2ncnc(N3CCc4c(cc(Cn5ccnc5)cc4-c4cn(C)nc4C(F)(F)F)C3=O)c2cc1OC]

I-24

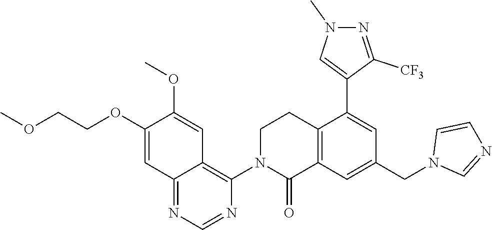 [SMILES: COCCOc1cc2ncnc(N3CCc4c(cc(Cn5ccnc5)cc4-c4cn(C)nc4C(F)(F)F)C3=O)c2cc1OC]

I-25

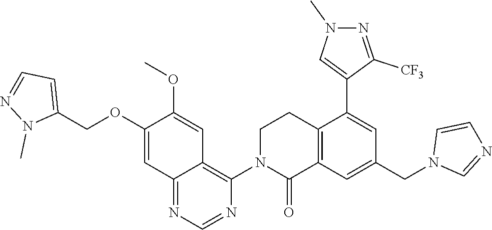 [SMILES: COc1cc2c(N3CCc4c(cc(Cn5ccnc5)cc4-c4cn(C)nc4C(F)(F)F)C3=O)ncnc2cc1OCc1ccnn1C]

I-26

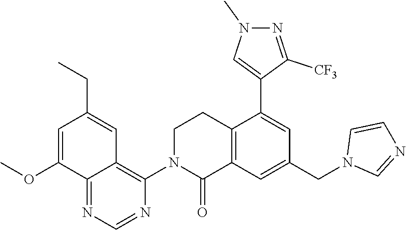 [SMILES: CCc1cc(OC)c2ncnc(N3CCc4c(cc(Cn5ccnc5)cc4-c4cn(C)nc4C(F)(F)F)C3=O)c2c1]

I-27

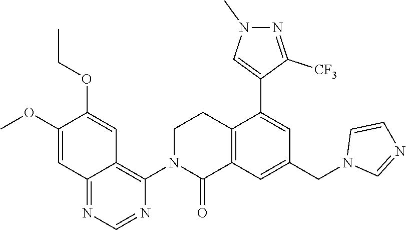 [SMILES: CCOc1cc2c(N3CCc4c(cc(Cn5ccnc5)cc4-c4cn(C)nc4C(F)(F)F)C3=O)ncnc2cc1OC]

I-28

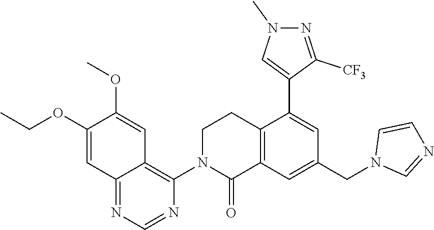 [SMILES: CCOc1cc2ncnc(N3CCc4c(cc(Cn5ccnc5)cc4-c4cn(C)nc4C(F)(F)F)C3=O)c2cc1OC]

I-29

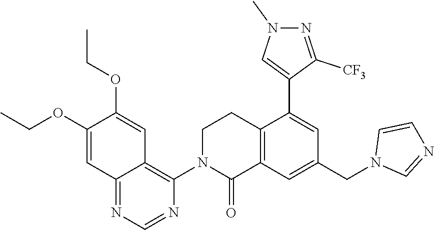 [SMILES: CCOc1cc2ncnc(N3CCc4c(cc(Cn5ccnc5)cc4-c4cn(C)nc4C(F)(F)F)C3=O)c2cc1OCC]

I-30

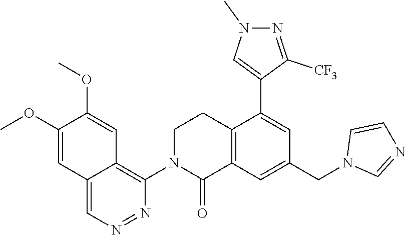 [SMILES: COc1cc2cnnc(N3CCc4c(cc(Cn5ccnc5)cc4-c4cn(C)nc4C(F)(F)F)C3=O)c2cc1OC]

I-31

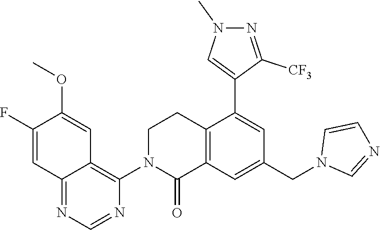 [SMILES: COc1cc2c(N3CCc4c(cc(Cn5ccnc5)cc4-c4cn(C)nc4C(F)(F)F)C3=O)ncnc2cc1F]

I-32

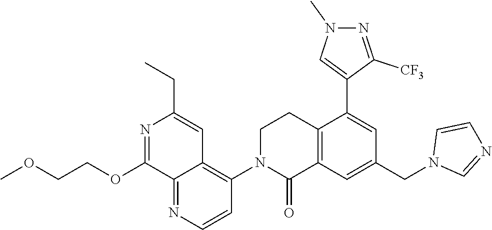 [SMILES: CCc1cc2c(N3CCc4c(cc(Cn5ccnc5)cc4-c4cn(C)nc4C(F)(F)F)C3=O)ccnc2c(OCCOC)n1]

I-33

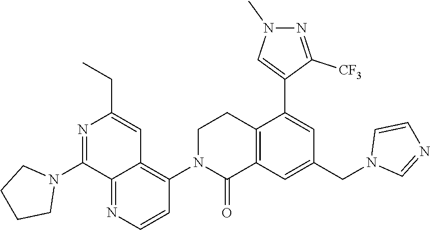 [SMILES: CCc1cc2c(N3CCc4c(cc(Cn5ccnc5)cc4-c4cn(C)nc4C(F)(F)F)C3=O)ccnc2c(N2CCCC2)n1]

I-34

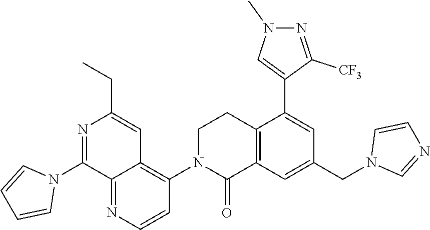 [SMILES: CCc1cc2c(N3CCc4c(cc(Cn5ccnc5)cc4-c4cn(C)nc4C(F)(F)F)C3=O)ccnc2c(-n2cccc2)n1]

I-35

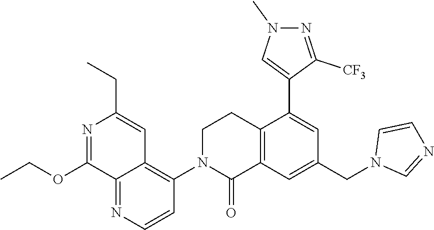 [SMILES: CCOc1nc(CC)cc2c(N3CCc4c(cc(Cn5ccnc5)cc4-c4cn(C)nc4C(F)(F)F)C3=O)ccnc12]

I-36

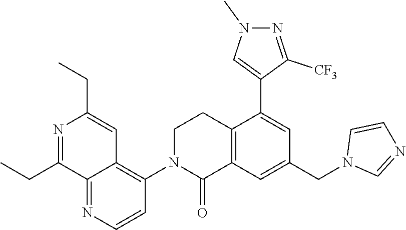 [SMILES: CCc1cc2c(N3CCc4c(cc(Cn5ccnc5)cc4-c4cn(C)nc4C(F)(F)F)C3=O)ccnc2c(CC)n1]

I-37

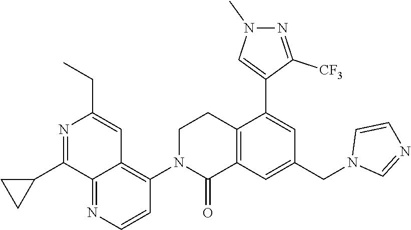 [SMILES: CCc1cc2c(N3CCc4c(cc(Cn5ccnc5)cc4-c4cn(C)nc4C(F)(F)F)C3=O)ccnc2c(C2CC2)n1]

I-38

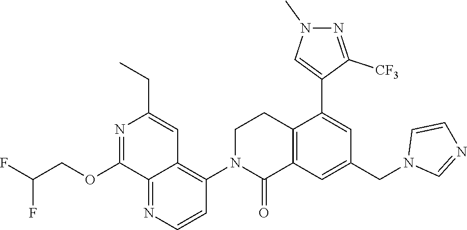 [SMILES: CCc1cc2c(N3CCc4c(cc(Cn5ccnc5)cc4-c4cn(C)nc4C(F)(F)F)C3=O)ccnc2c(OCC(F)F)n1]

I-39

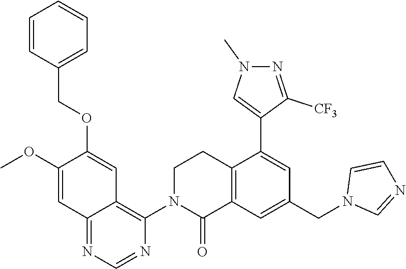 [SMILES: COc1cc2ncnc(N3CCc4c(cc(Cn5ccnc5)cc4-c4cn(C)nc4C(F)(F)F)C3=O)c2cc1OCc1ccccc1]

I-40

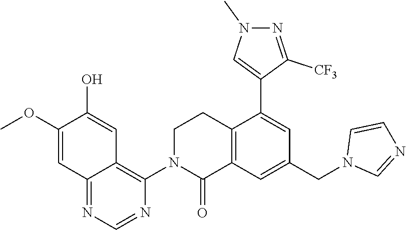 [SMILES: COc1cc2ncnc(N3CCc4c(cc(Cn5ccnc5)cc4-c4cn(C)nc4C(F)(F)F)C3=O)c2cc1O]

I-41

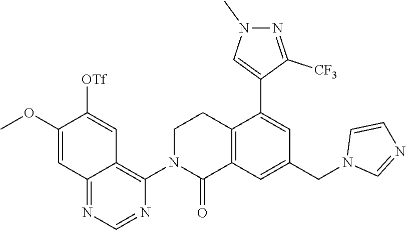 [SMILES: COc1cc2ncnc(N3CCc4c(cc(Cn5ccnc5)cc4-c4cn(C)nc4C(F)(F)F)C3=O)c2cc1OS(=O)(=O)C(F)(F)F]

I-42

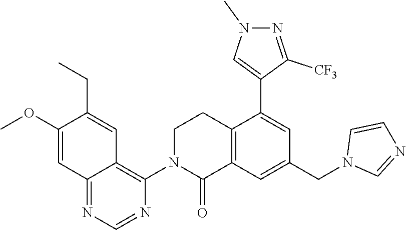 [SMILES: CCc1cc2c(N3CCc4c(cc(Cn5ccnc5)cc4-c4cn(C)nc4C(F)(F)F)C3=O)ncnc2cc1OC]

I-43

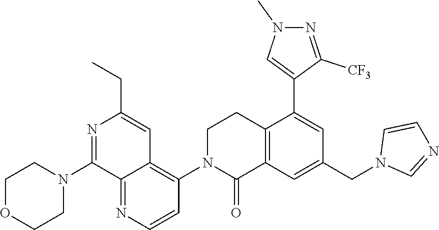 [SMILES: CCc1cc2c(N3CCc4c(cc(Cn5ccnc5)cc4-c4cn(C)nc4C(F)(F)F)C3=O)ccnc2c(N2CCOCC2)n1]

I-44

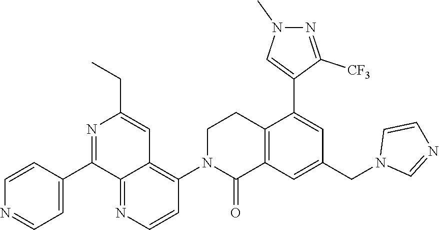 [SMILES: CCc1cc2c(N3CCc4c(cc(Cn5ccnc5)cc4-c4cn(C)nc4C(F)(F)F)C3=O)ccnc2c(-c2ccncc2)n1]

I-45

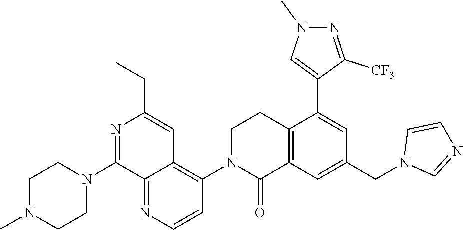 [SMILES: CCc1cc2c(N3CCc4c(cc(Cn5ccnc5)cc4-c4cn(C)nc4C(F)(F)F)C3=O)ccnc2c(N2CCN(C)CC2)n1]

I-46

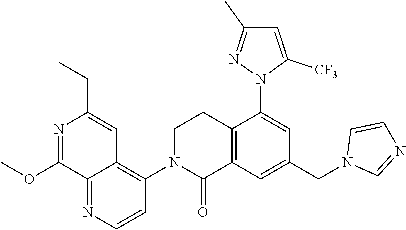 [SMILES: CCc1cc2c(N3CCc4c(cc(Cn5ccnc5)cc4-n4nc(C)cc4C(F)(F)F)C3=O)ccnc2c(OC)n1]

I-47

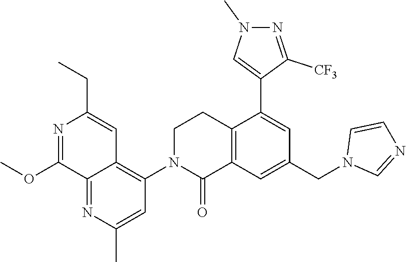 [SMILES: CCc1cc2c(N3CCc4c(cc(Cn5ccnc5)cc4-c4cn(C)nc4C(F)(F)F)C3=O)cc(C)nc2c(OC)n1]

I-48

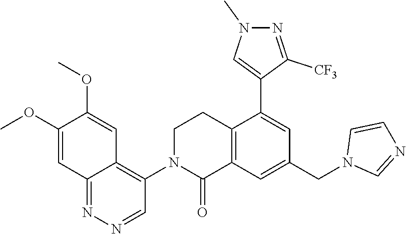 [SMILES: COc1cc2nncc(N3CCc4c(cc(Cn5ccnc5)cc4-c4cn(C)nc4C(F)(F)F)C3=O)c2cc1OC]

I-49

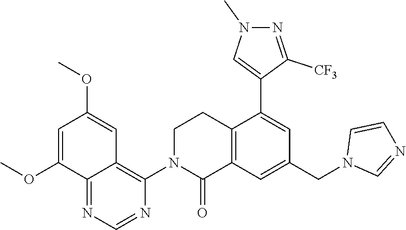 [SMILES: COc1cc(OC)c2ncnc(N3CCc4c(cc(Cn5ccnc5)cc4-c4cn(C)nc4C(F)(F)F)C3=O)c2c1]

I-50

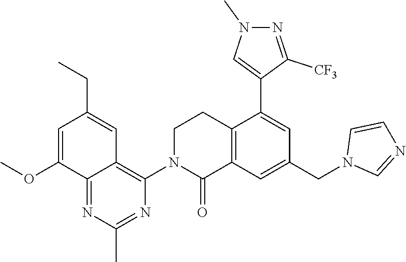 [SMILES: CCc1cc(OC)c2nc(C)nc(N3CCc4c(cc(Cn5ccnc5)cc4-c4cn(C)nc4C(F)(F)F)C3=O)c2c1]

I-51

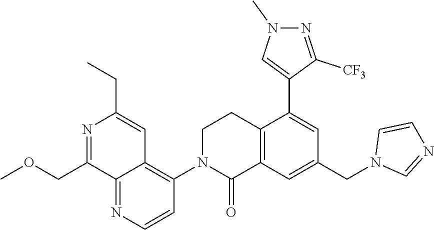 [SMILES: CCc1cc2c(N3CCc4c(cc(Cn5ccnc5)cc4-c4cn(C)nc4C(F)(F)F)C3=O)ccnc2c(COC)n1]

I-52

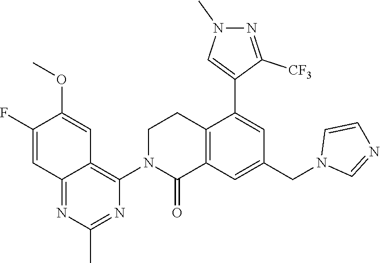 [SMILES: COc1cc2c(N3CCc4c(cc(Cn5ccnc5)cc4-c4cn(C)nc4C(F)(F)F)C3=O)nc(C)nc2cc1F]

I-53

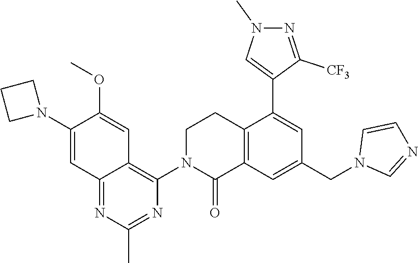 [SMILES: COc1cc2c(N3CCc4c(cc(Cn5ccnc5)cc4-c4cn(C)nc4C(F)(F)F)C3=O)nc(C)nc2cc1N1CCC1]

I-54

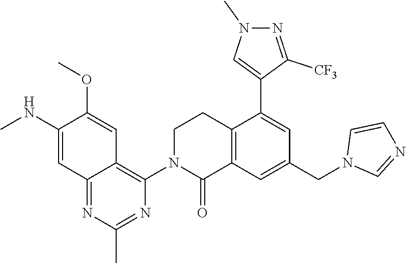 [SMILES: CNc1cc2nc(C)nc(N3CCc4c(cc(Cn5ccnc5)cc4-c4cn(C)nc4C(F)(F)F)C3=O)c2cc1OC]

I-55

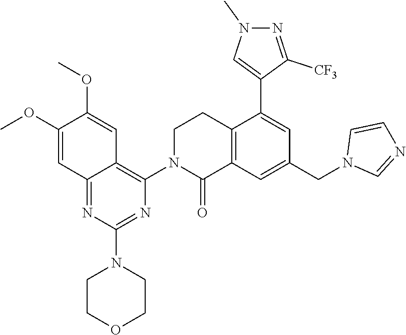 [SMILES: COc1cc2nc(N3CCOCC3)nc(N3CCc4c(cc(Cn5ccnc5)cc4-c4cn(C)nc4C(F)(F)F)C3=O)c2cc1OC]

I-56

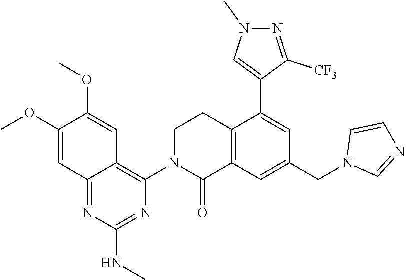 [SMILES: CNc1nc(N2CCc3c(cc(Cn4ccnc4)cc3-c3cn(C)nc3C(F)(F)F)C2=O)c2cc(OC)c(OC)cc2n1]

I-57

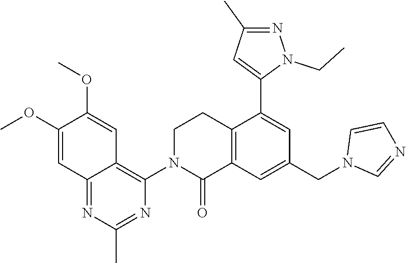 [SMILES: CCn1nc(C)cc1-c1cc(Cn2ccnc2)cc2c1CCN(c1nc(C)nc3cc(OC)c(OC)cc13)C2=O]

I-58

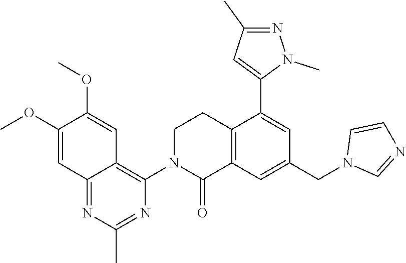 [SMILES: COc1cc2nc(C)nc(N3CCc4c(cc(Cn5ccnc5)cc4-c4cc(C)nn4C)C3=O)c2cc1OC]

I-59

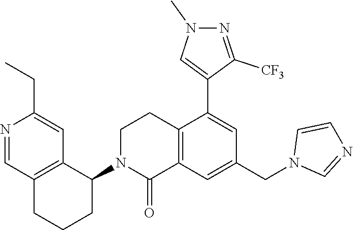 [SMILES: CCc1cc2c(cn1)CCC[C@@H]2N1CCc2c(cc(Cn3ccnc3)cc2-c2cn(C)nc2C(F)(F)F)C1=O]

I-60

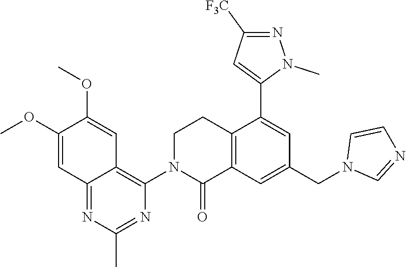 [SMILES: COc1cc2nc(C)nc(N3CCc4c(cc(Cn5ccnc5)cc4-c4cc(C(F)(F)F)nn4C)C3=O)c2cc1OC]

I-61

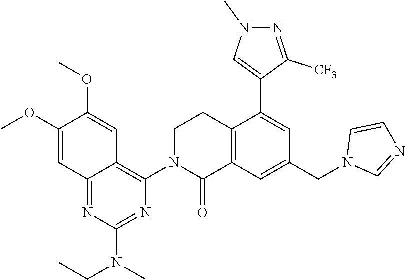 [SMILES: CCN(C)c1nc(N2CCc3c(cc(Cn4ccnc4)cc3-c3cn(C)nc3C(F)(F)F)C2=O)c2cc(OC)c(OC)cc2n1]

I-62

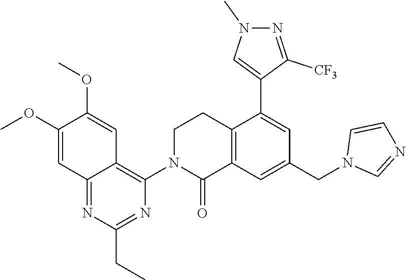 [SMILES: CCc1nc(N2CCc3c(cc(Cn4ccnc4)cc3-c3cn(C)nc3C(F)(F)F)C2=O)c2cc(OC)c(OC)cc2n1]

I-63

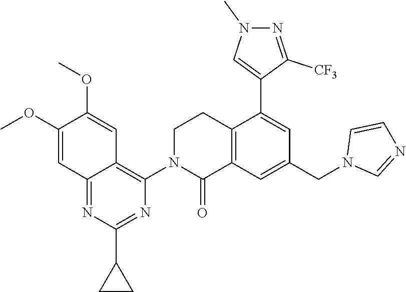 [SMILES: COc1cc2nc(C3CC3)nc(N3CCc4c(cc(Cn5ccnc5)cc4-c4cn(C)nc4C(F)(F)F)C3=O)c2cc1OC]

I-64

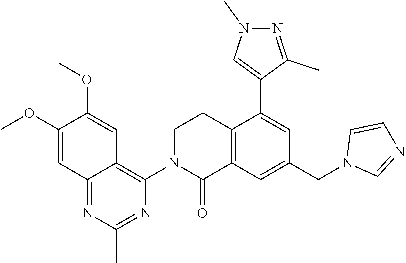 [SMILES: COc1cc2nc(C)nc(N3CCc4c(cc(Cn5ccnc5)cc4-c4cn(C)nc4C)C3=O)c2cc1OC]

I-65

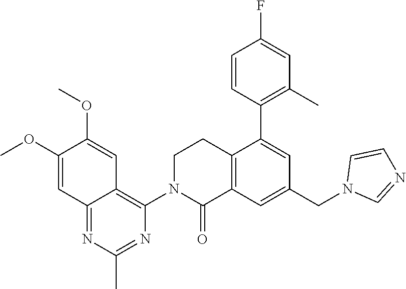 [SMILES: COc1cc2nc(C)nc(N3CCc4c(cc(Cn5ccnc5)cc4-c4ccc(F)cc4C)C3=O)c2cc1OC]

I-66

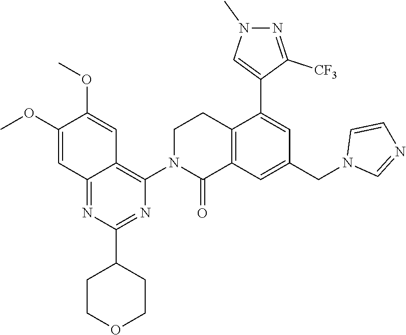 [SMILES: COc1cc2nc(C3CCOCC3)nc(N3CCc4c(cc(Cn5ccnc5)cc4-c4cn(C)nc4C(F)(F)F)C3=O)c2cc1OC]

I-67

Compound names can be assigned by using Struct=Name naming algorithm as part of CHEMDRAW® ULTRA.

Compounds may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. In the compounds of formula (I), when no specific configuration is indicated at a stereogenic center (e.g., carbon), the compounds include all possible stereoisomers.

Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes an isotopically-labeled compound (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### A. Binding to WDR5

The disclosed compounds may bind to WDR5 and prevent the association of MLL1 or other transcription factors and proteins dependent on WDR5. The compounds may bind to WDR5 and prevent oncogenic processes associated with MLL1, c-MYC, or other oncogenic proteins dependent on WDR5.

Compounds of formula (I) can bind to WDR5 resulting in a Kᵢ ranging from about 0.01 nM to about 250 μM. The compounds may have a Kᵢ of about 250 μM, about 200 μM, about 150 μM, about 100 μM, about 90 μM, about 80 μM, about 70 μM, about 60 μM, about 50 μM, about 40 μM, about 30 μM, about 20 μM, about 10 μM, about 9 μM, about 8 μM, about 7 μM, about 6 μM, about 5 μM, about 4 μM, about 3 μM, about 2 μM, about 1 μM, about 950 nM, about 900 nM, about 850 nM, about 800 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 50 nM, about 10 nM, about 5 nM, about 1 nM, about 0.3 nM, about 0.1 nM, about 0.03 nM, or about 0.01 nM. Compounds of formula (I) can bind to WDR5 resulting in a Kᵢ of less than 250 μM, less than 200 μM, less than 150 μM less than 100 μM, less than 90 μM, less than 80 μM less than 70 μM, less than 60 μM, less than 50 μM, less than 40 μM, less than 30 μM, less than 20 μM, less than 10 μM, less than 9 μM, less than 8 μM, less than 7 μM, less than 6 μM, less than 5 μM, less than 4 μM, less than 3 μM, less than 2 μM, less than 1 μM, less than 950 nM, less than 900 nM, less than 850 nM, less than 800 nM, less than 850 nM, less than 800 nM, less than 750 nM, less than 700 nM, less than 650 nM, less than 600 nM, less than 550 nM, less than 500 nM, less than 450 nM, less than 400 nM, less than 350 nM, less than 300 nM, less than 250 nM, less than 200 nM, less than 150 nM, less than 100 nM, less than 50 nM, less than 10 nM, less than 5 nM, less than 1 nM, less than 0.3 nM, less than 0.1 nM, or less than 0.03 nM.

### B. General Synthesis

Compounds of formula (I) may be prepared by synthetic processes or by metabolic processes. Preparation of the compounds by metabolic processes includes those occurring in the human or animal body (in vivo) or processes occurring in vitro.

The compounds of the present disclosure can be prepared in a number of ways well known to one skilled in the art of organic synthesis. The compounds of the present disclosure can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety by reference as to the subject matter referenced herein. Compounds of formula (I) may be also prepared by metabolic processes. Preparation of the compounds by metabolic processes includes those occurring in the human or animal body (in vivo) or processes occurring in vitro.

The compounds of the disclosure may be prepared using the exemplary reactions and techniques described in this section. The reactions are performed in solvents appropriate to the reagents and materials employed and are suitable for the transformations being effective. Also, in the description of the synthetic methods described below, it is to be understood that all proposed reaction conditions, including solvent, reaction atmosphere, reaction temperature, duration of the experiment and workup procedures, are chosen to be the conditions standard for that reaction, which should be readily recognized by one skilled in the art. One having ordinary skill in the art may adjust one or more of the conditions described herein. One skilled in the art of organic synthesis understands that the functionality present on various portions of the edict molecule must be compatible with the reagents and reactions proposed. Not all compounds of the disclosure falling into a given class may be compatible with some of the reaction conditions required in some of the methods described. Such restrictions to the substituents, which are compatible with the reaction conditions, will be readily apparent to one skilled in the art and alternate methods can be used.

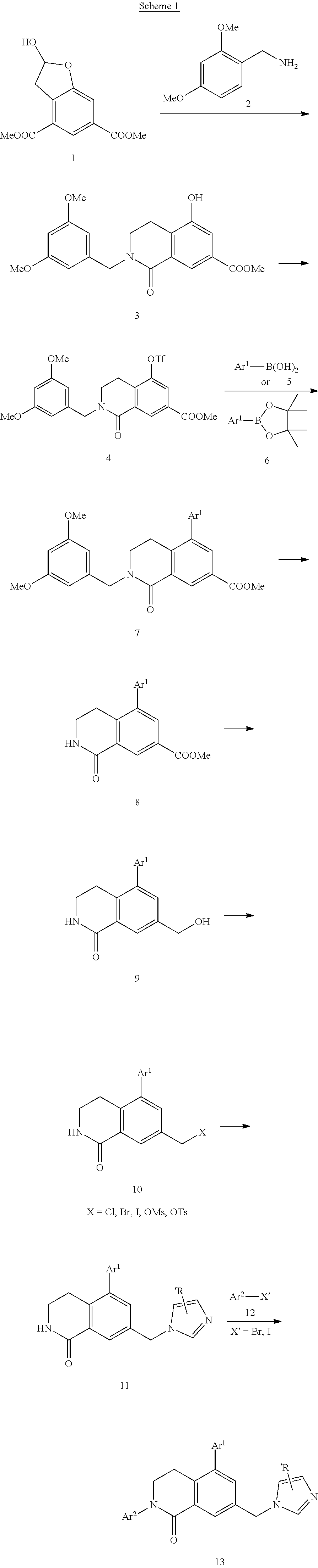

In some embodiments, compounds of Formula 13 may be synthesized by procedures illustrated in Scheme 1. Hemiacetal 1 can be coupled with (2,4-dimethoxyphenyl)methanamine 2 under reductive amination conditions, employing a reducing agent including, but not limited to, NaBH(OAc)₃ or NaCNBH₃ followed by spontaneous cyclization to yield intermediate 3. After activation of the phenol moiety of 3 as a triflate, intermediate 4 may be coupled with a variety of boronic acids 5 or borates 6, which are commercially available or can be prepared, via e.g., Suzuki-Miyaura coupling protocol to afford biaryl adducts 7 (Miyaura, N., Suzuki, A., Chem. Rev. (1995), 2457) in the presence of a catalytic Pd species, such as Pd(PPh₃)₄, PdCl₂(dppf), Pd(PPh₃)₂Cl₂, Pd(OAc)₂, Pd₂(dba)₃, and a suitable ligand, such as PPh₃, AsPh₃, etc., or other such Pd catalyst, and a base, such as Na₂CO₃, Cs₂CO₃, K₂CO₃, Ba(OH)₂ or Et₃N. The dimethoxybenzyl moiety of 7 can be removed using, but not limited to, TFA to prepare lactam 8. The methyl ester functional group of 8 may be converted to an alcohol under various reduction conditions that are routine for those skilled in the art of organic synthesis. The hydroxy group of formula 9 may be activated by converting the hydroxy group to a bromide, chloride, mesylate or tosylate group by a number of conditions that are routine for those skilled in the art of organic synthesis. The resulting intermediate 10 may be reacted with variety of nucleophiles, such as the optionally substituted imidazole, in the presence of an appropriate base, such as DIEA, TEA, Cs₂CO₃, K₂CO₃, LiOH or NaOH, to yield Intermediate 11. The lactam NH of 11 may undergo cross-coupling reactions with a variety of aryl or heteroaryl halides of formula 12, wherein X¹ is Br or I, in the presence of a catalytic Pd species, such as Pd(OAc)₂ or Pd₂(dba)₃, and a suitable ligand, such as Xantphos or BrettPhos, and a base, such as Na₂CO₃, Cs₂CO₃, or K₂CO₃, to generate compounds of formula 13. Alternatively, compounds of formula 13 can be produced using the Ullman coupling conditions in the presence of CuI and a suitable ligand, such as (trans)-1,2-N,N′-dimethylaminocyclohexane or L-proline, and a base, such as Cs₂CO₃, K₂CO₃ or K₂PO₄, in a suitable solvent, such as toluene or DMF.

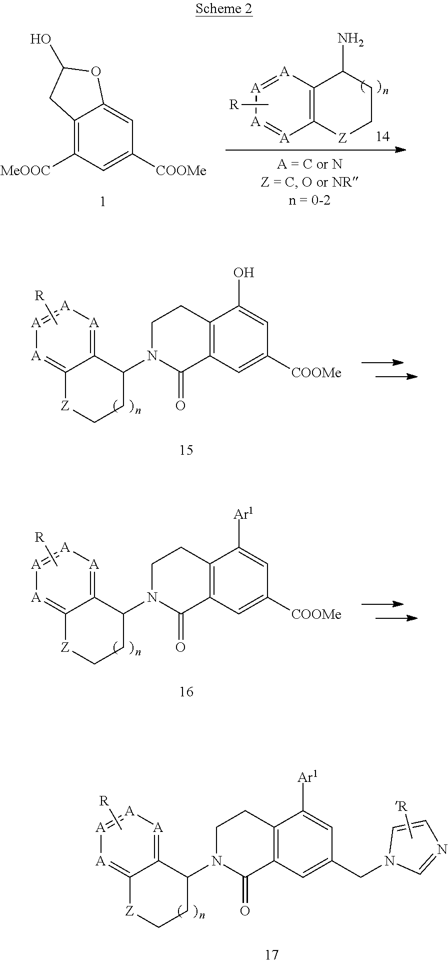

In some embodiments, compounds disclosed herein may be prepared as shown in Scheme 2. The optionally substituted, partially unsaturated, fused-bicyclic amine of formula 14 may be coupled with hemiacetal 1 under the reductive amination conditions described above to give intermediate 15. Then, intermediate 15 may be subjected to the reaction sequence from intermediate 3 to 7, illustrated in Scheme 1, to afford intermediate 16, followed by the reaction sequence from intermediate 8 to 11 to obtain compounds of formula 17.

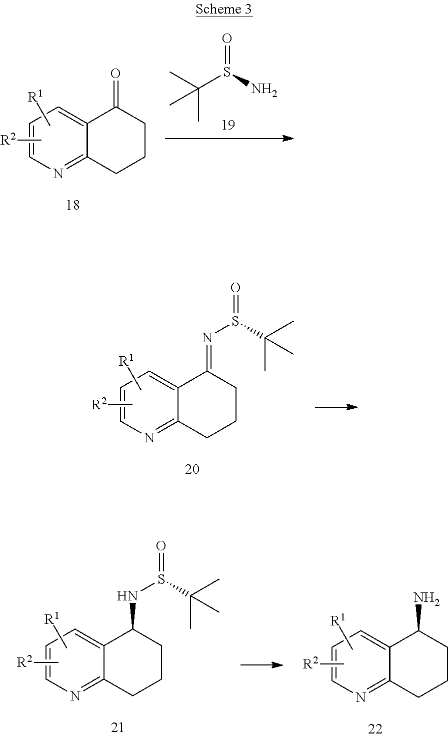

Optically pure amine intermediate of formula 22 may be prepared by procedures illustrated in Scheme 3. Suitably substituted bicyclic ketone 18 may undergo a condensation reaction with the optically pure tert-butanesulfinamide 19 using Ti(OEt)₄ as a Lewis acid and water scavenger. The resulting optically pure N-sulfinyl imine intermediate 20 may be then reduced using an appropriate hydride, such as NaBH₄ or L-Selectride, to afford the diastereomerically enriched sulfinamide 21. The tert-butanesulfinyl group may be then removed under appropriate acidic conditions to yield the optically pure bicyclic amine of formula 22.

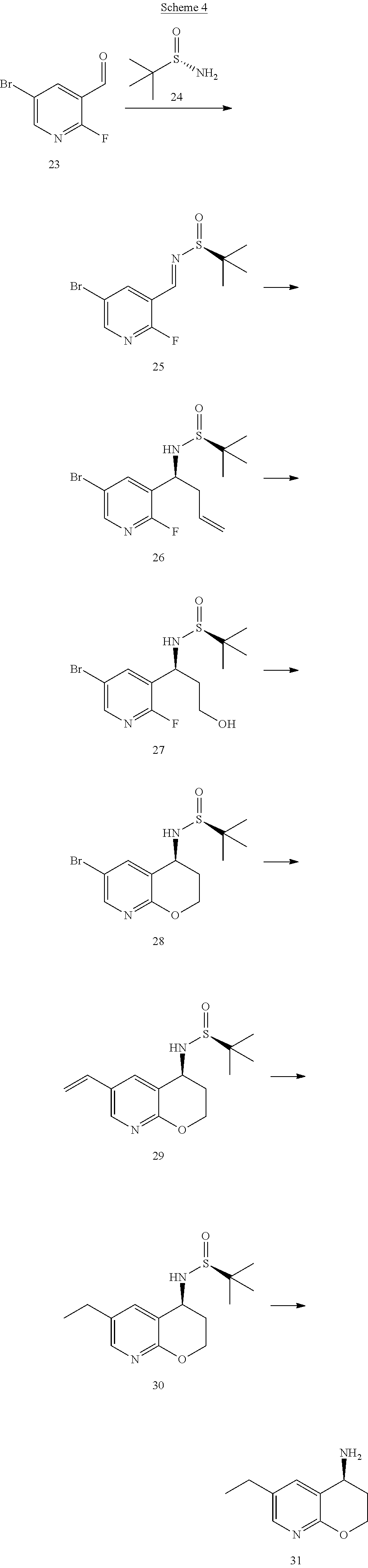

In some embodiments, the optically pure bicyclic amine of formula 31 may be used as a reagent. Further, the amine of formula 31 may be synthesized by procedures illustrated in Scheme 4 using the 5-bromo-2-fluoronicotinaldehyde of formula 23, which may be converted to the optically pure N-sulfinyl imine intermediate 25 using the condensation protocol described in Scheme 3, using CuSO₄ as a Lewis acid. Allylmagnesium bromide may be reacted with the imine functional group of intermediate 25 in stereoselective manner to yield the diastereomerically enriched sulfinamide 26. Subsequent ozonalysis, followed by the reductive work-up of Intermediate 26 in the presence of NaBH₄ may be performed. The resulting alcohol 27 may be cyclized via a SN_{Ar} reaction using, but not limited to, potassium tert-butoxide as a base to yield dihydro-pyranopyridine intermediate 28. The bromo group of intermediate 28 may be coupled with vinyl boronic acid, via e.g., Suzuki-Miyaura coupling protocol to afford adduct 29, which can be reduced under the hydrogenolysis condition to give intermediate 30 conditions that are routine for those skilled in the art of organic synthesis. The tert-butanesulfinyl group of intermediate 30 may be removed under acidic conditions to yield chiral amines 31.

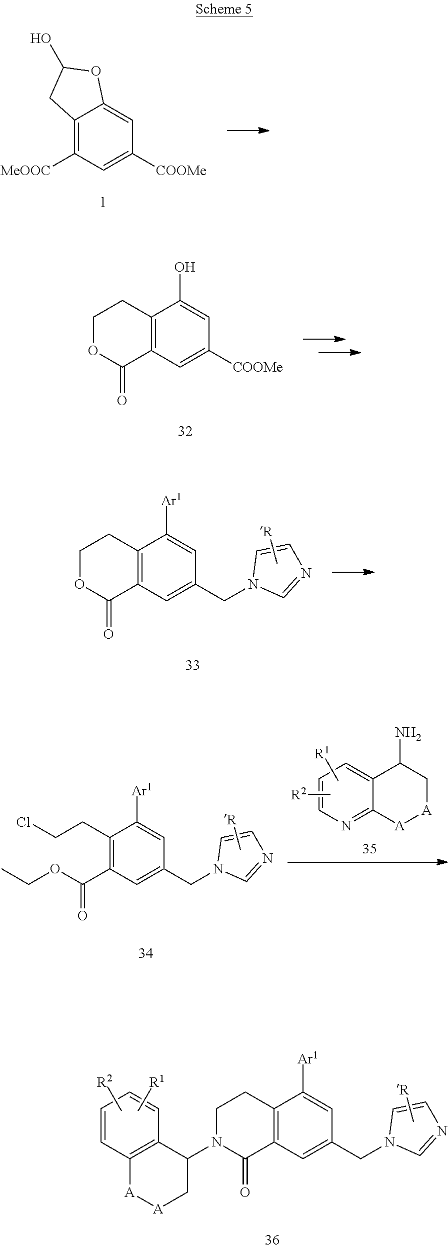

The bicyclic lactone of formula 32 may be synthesized from hemiacetal 1 by reduction followed by cyclization using, but not limited to, NaBH₄ as a reducing agent. Then, lactone 12 can be subjected to the reaction sequence illustrated in Scheme 1 from intermediate 3 to 7 followed by the reaction sequence from intermediate 8 to 11 to afford intermediate 33. The lactone moiety of intermediate 33 can be opened using, but not limited to, SOCl₂ in EtOH to give intermediate 34. Subsequent SN₂ substitution of the amine of formula 35, in the presence of tert-butylmagnesium chloride as a base, followed by cyclization yields the final compound of formula 36.

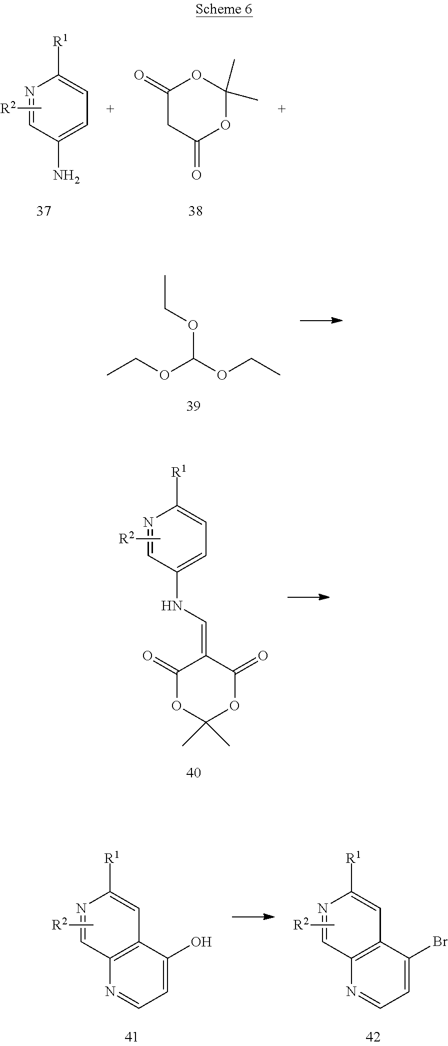

In some embodiments, 4-bromo-naphthyridine 42 was used as a reagent, and may be prepared by procedures illustrated in Scheme 6. A mixture of suitably substituted 3-amino-pyridine 37, Meldrum's acid 38, and triethyl orthoformate 39 may be heated to produce intermediate 40, which may subsequently undergo thermal cyclization to give naphthyridin-4-ol 41. 4-Bromo-naphthyridine 42 may be prepared directly from 41 using, but not limited to, PBr₃. Alternatively, bromo-naphthyridine 42 may be prepared by triflation of hydroxyl group of naphthyridin-4-ol 41 followed by bromination, using, but not limited to, LiBr.

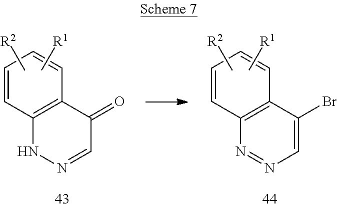

The intermediate of formula 44 may be prepared by the reaction shown in Scheme 7. The suitably substituted cinnolin-4-one 43 may be halogenated using, but not limited to, phosphorus oxybromide to yield 4-bromocinnoline 44.

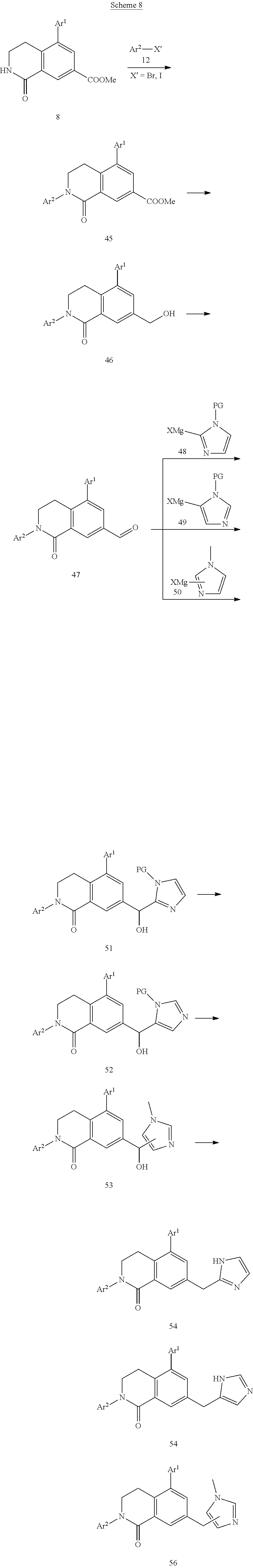

Alternatively, intermediates of formula 8 may undergo cross-coupling reactions with a variety of aryl or heteroaryl halides of formula 12 under the condition described in Scheme 1 to give intermediate 45. The methyl ester functional group of 45 may be converted to an alcohol under various reduction conditions that are routine for those skilled in the art of organic synthesis. The primary alcohol of intermediate 46 may be oxidized by appropriate reagents at a number of conditions that are routine for those skilled in the art to give aldehyde 47. A variety of N-substituted imidazolyl Grignard reagents (e.g. 48-50) such as, but not limited to, (1-trityl-1H-imidazol-5-yl)magnesium iodide, (1-trityl-1H-imidazol-2-yl)magnesium iodide or (1-methyl-1H-imidazol-2-yl)magnesium iodide may react with aldehyde 47 to generate corresponding secondary alcohols 51-53. Compounds of formula 54-56 may be produced by reduction of corresponding alcohols 51-53 using, but not limited to, triethylsilane and TFA in a polar aprotic solvent such as 1,2-dichloroethane with heat.

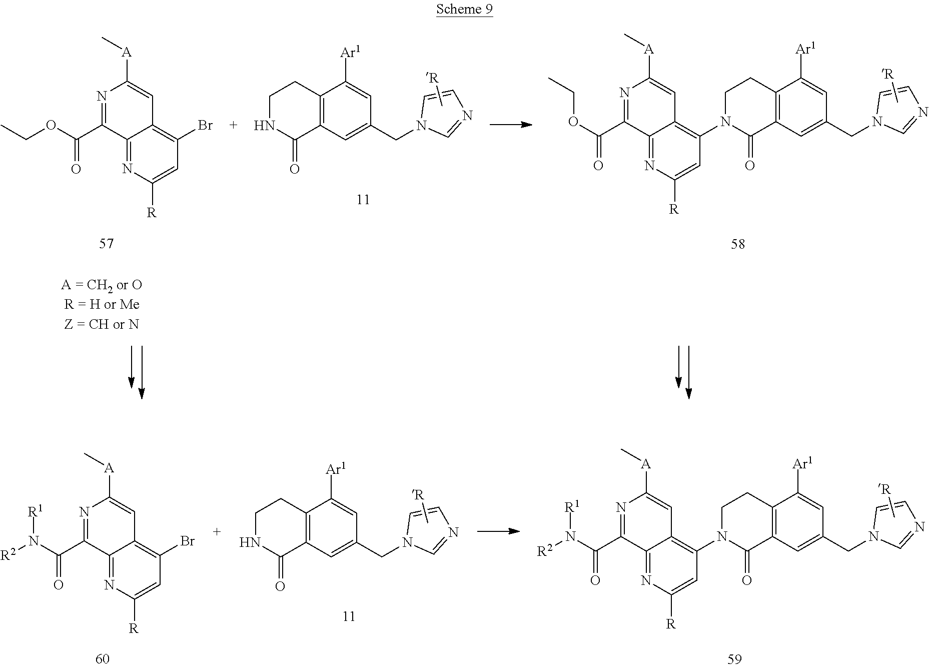

In some embodiments, compounds of Formula 59 may be synthesized by procedures illustrated in Scheme 9. Optionally substituted ethyl 4-bromo-1,7-naphthyridine-8-carboxylate 57, which was produced by the reaction sequence depicted in Scheme 6, may be coupled to lactam 11 to yield compounds of formula 58. The ester functional group of 58 may be converted to an amide to give a product of formula 59 through saponification followed by an amide coupling reaction sequence that is routine for those skilled in the art of organic synthesis. Alternatively, the same reaction sequence may be applied to ester 57 to generate intermediate 60, which may be coupled to lactam 11 to form product 59. An analogous sequence may be conducted starting from ethyl 4-bromo-quinoline-8-carboxylates to provide compounds wherein G¹ is

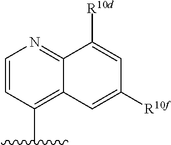

and R^{10d} is —C(O)N(R^{1a})₂ or —C(O)OR^{1a}. Ethyl 4-bromo-quinoline-8-carboxylates may be prepared using the processes of Scheme 6 starting from substituted aniline analogs of 37.

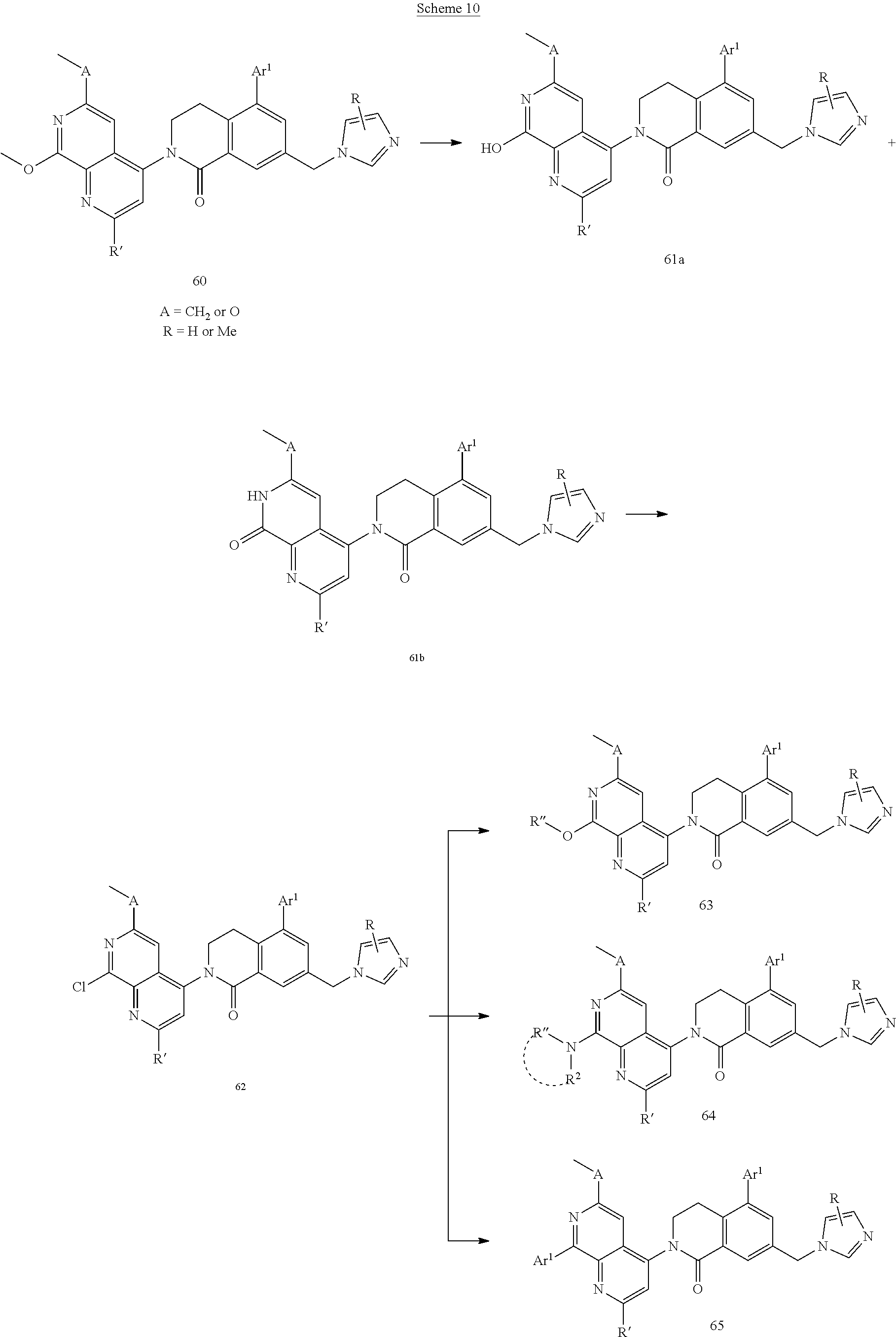

In some embodiments, compounds of Formula 63-65 may be prepared using procedures shown in Scheme 10. The 8-OMe group of compound 60 may be demethylated using, but not limited to, PBr₃ or BBrn to give a tautomeric mixture of intermediate 61a and 61b. This mixture may be treated with POC₁₋₃ to yield a versatile 8-Cl intermediate 62. Compounds of formula 63 and 64 may be produced through a SNAr reaction using an alcohol or amine as a substrate in the presence of a base using conditions that are well known for those skilled in the art of organic synthesis. Alternatively, Intermediate 62 may be subjected to Buchwald-Hartwig coupling or Suzuki-Miyaura coupling protocols that were described in Scheme I to produce a product of formula 64 or 65. Quinolin-4-yl analogs of 63-65 may be prepared using analogous processes from 8-bromoquinolin-4-yl intermediates analogous to 62.

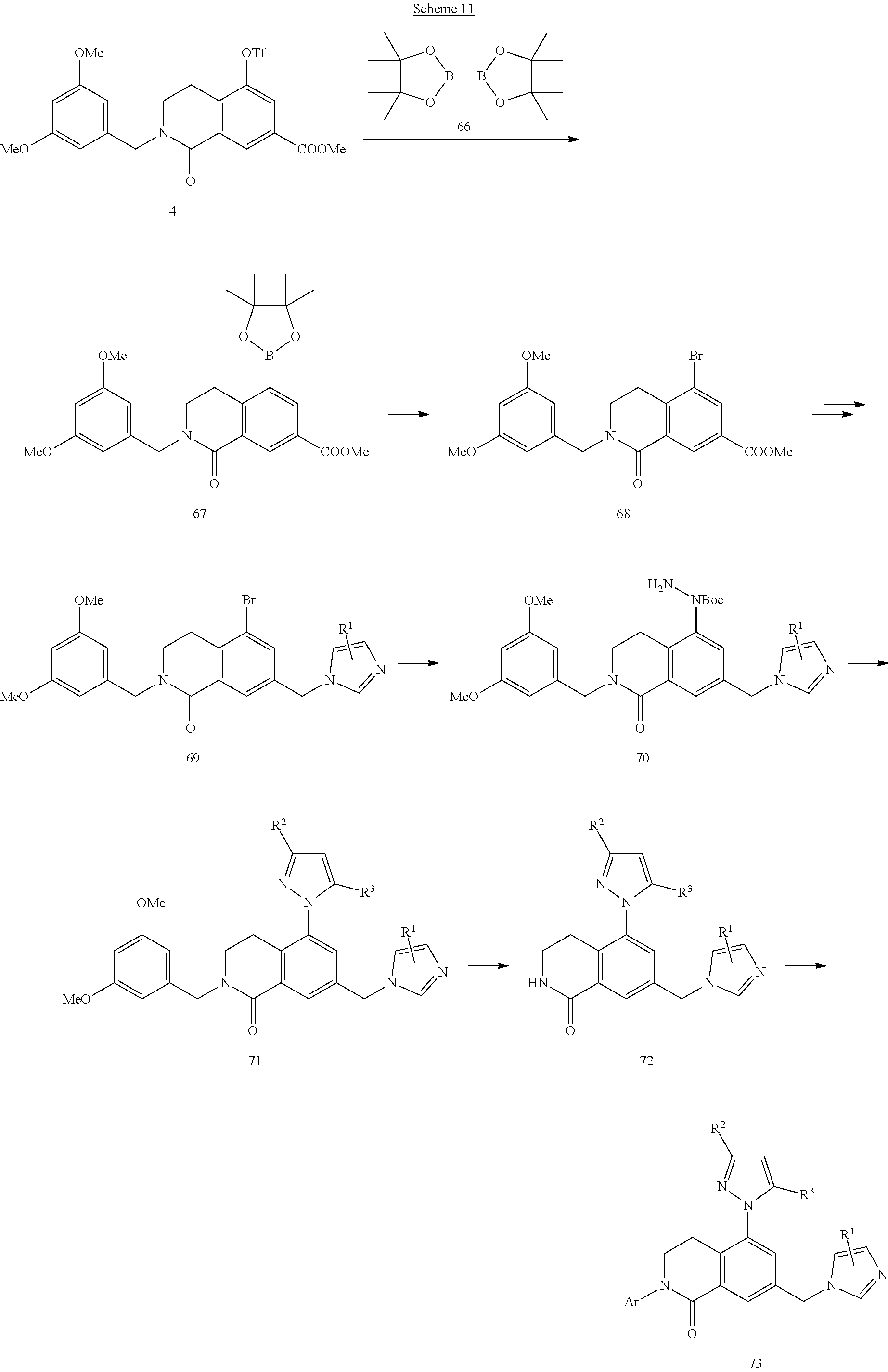

Intermediate 4 may be converted to borate 67 using, but not limited to, bis(pinacolato)diboron 66 and PdCl₂(dppf)-CH₂Cl₂, which may undergo copper(II) bromide mediated bromination to give bromide containing intermediate 68. The methyl ester functional group of 68 can be subjected to the reaction sequence illustrated in Scheme 1 from intermediate 8 to 11 to afford intermediate 69. The substituted pyrazol-1-yl moiety of intermediate 71 may be constructed through installation of Boc-hydrazine under, but not limited to, the Buchwald-Hartwig coupling condition described in Scheme 1 followed by deprotection of Boc and hetero-cyclization using conditions that are well known for those skilled in the art of organic synthesis to yield intermediate 71. Lastly, compounds of Formula 73 may be generated by removal of the dimethoxy benzyl group of 71 followed by installation of Ar-group under the Buchwald-Hartwig coupling condition described in Scheme 1.

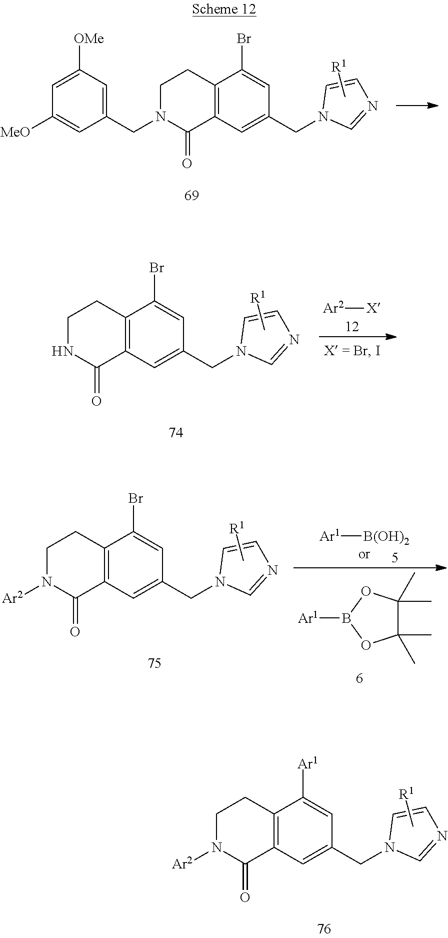

In some embodiments, compounds of Formula 76 may be prepared using intermediate 69 through the reaction sequence of removal of the the dimethoxy benzyl group, Buchwald-Hartwig coupling followed by Suzuki-Miyaura coupling under the reaction conditions described in Scheme 1.

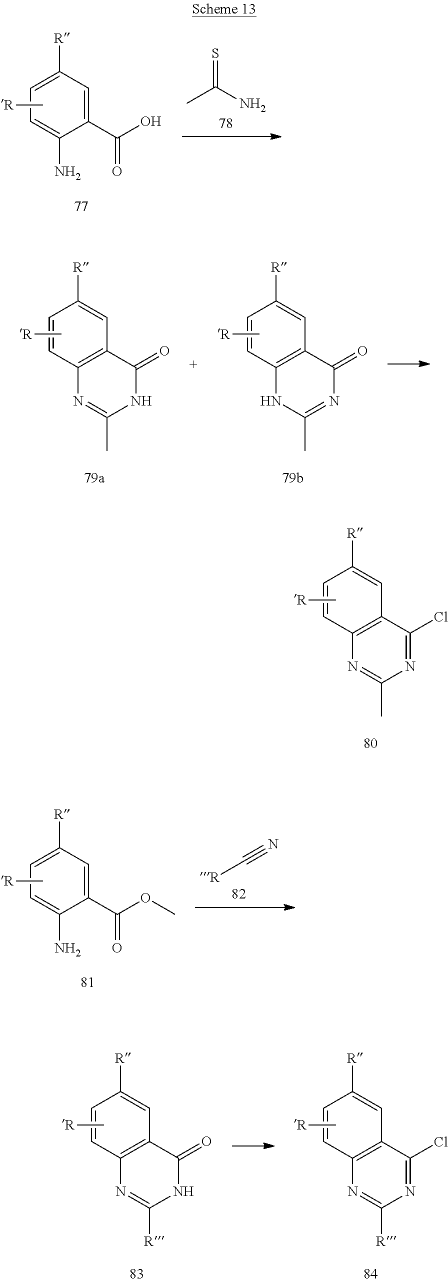

In some embodiments, substituted 2-alkyl-4-chloro-quinazoline 80 or 84 were used as reagents, which may be prepared by procedures depicted in Scheme 13. A substituted 2-amino-benzoic acid 77 and ethanethioamide 78 may be heated to produce a tautomeric mixture of 2-methylquinazolin-4(3H)-one intermediates 79a and 79b, which may be chlorinated using, but not limited to, POCl₃, to yield substituted 4-chloro-2-methyl-quinazoline 80. Alternately, methyl amino-benzoate 81 may react with alkyl-nitrile 82 to yield 2-alkylquinazolin-4(3H)-one intermediates 83, which may subsequently undergo chlorination reaction as above to give 2-alkyl-4-chloro-quinazoline 84.

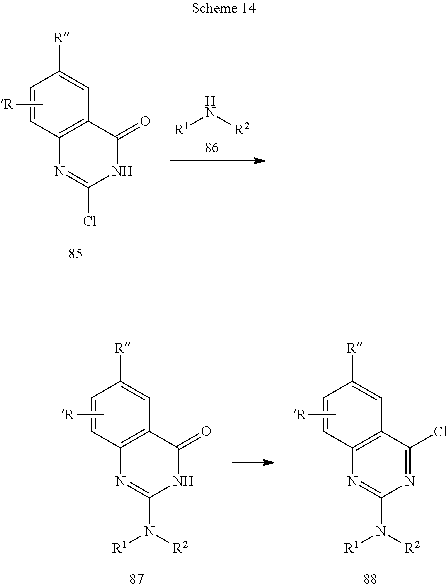

In some embodiments, substituted 2-amino-4-chloro-quinazolines 87 were used as reagents and may be prepared by procedures depicted in Scheme 14. A substituted chloro-quinazolin-4(3H)-one 85 and substituted amine 86 may be reacted to produce a substituted 2-amino-quinazolin-4(3H)-one intermediates 87 through a SN_{Ar} reaction. A subsequent chlorinatation reaction using a procedure shown in Scheme 13 may yield substituted 2-amino-4-chloro-quinazoline 88.

Precursor reagents and intermediates for core aryl or phenyl structure were either commercially available or prepared using known methods in the literature. Procedures towards key intermediates are detailed within specified examples or below.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th}ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### C. Examples

### Abbreviations

The following abbreviations are employed in the Examples and elsewhere herein:
AcOH=acetic acid
aq.=aqueous
BINAP=2,2′-bis(diphenylphosphino)-1,1′-binaphthyl
(Boc)₂O=di-tert-butyl dicarbonate
BrettPhos=2-(Dicyclohexylphosphino)3,6-dimethoxy-2′,4′,6′-triisopropyl-1,1′-biphenyl
CH₂Cl₂=methylene chloride
conc.=concentrated
Cs₂CM3=cesium carbonate
DBU=1,8-diazabicyclo[5.4.0]undec-7-ene
DCE=dichloroethane
DCM=dichloromethane
DIPEA/DIEA=N,N-diisopropylethylamine
DMA=dimethylacetamide
DMF=dimethylformamide
DMSO=dimethylsulfoxide
Dowtherm A=eutectic mixture of 26.5% diphenyl+73.5% diphenyl oxide
EDC=1-Ethyl-3-(3-dimethylaminopropyl)carbodiimide
eq. or equiv=equivalent(s)
ether=diethyl ether
Et₃N=triethylamine
EtOAc=ethyl acetate
EtOH=ethanol
g=gram(s)
h or hr=hour(s)
HATU=1-[Bis(dimethylamino)methylene]-1H-1,2,3-triazolo[4,5-b]pyridinium 3-oxid hexafluorophosphate
HCl=hydrochloric acid
Hex=hexanes
HOBt=hydroxybenzotriazole
K₂CO₃=potassiumm carbonate
KOH=potassium hydroxide
LRMS=low resolution mass spectrometry
L-selectride=Lithium tri-sec-butylborohydride
[M+H]⁺=the protonated mass of the free base of the compound
MeCN=acetonitrile
MeOH=methanol
MeONa=sodium methoxide
mg=milligram(s)
MgSO₄=magnesium sulfate
min=minute(s)
mL or ml=milliliter
mmol=millimole(s)
Na₂CO₃=sodium carbonate
NaH=sodium hydride
NaHCO₃=sodium bicarbonate
NaN₃=sodium azide
NaOH=sodium hydroxide
NBS=N-bromo succinimide
NIS=N-iodo succinimide
NMP=N-methyl-2-pyrrolidone
NMR=nuclear magnetic resonance
PdCl₂(dppf)/Pd(dppf)Cl₂=[1,1′-Bis(diphenylphosphino)ferrocene]dichloropalladium(II)
Pd₂(dba)₃=Tris(dibenzylideneacetone)dipalladium(0)
Pd(PPh₃)₄=tetrakis(triphenylphosphine)palladium(0)
Pd(PPh₃)₂Cl₂=Bis(triphenylphosphine)palladium(II) dichloride
Pd(OAc)₂=Palladium(II) acetate
Pd(t-Bu₃P)₂=Bis(tri-tert-butylphosphine)palladium(0)
PPh₃=triphenylphosphine
p-TSA=para-toluenesulfonic acid
RT or r.t.=room temperature
R_{T}=retention time (in minutes)
sat.=saturated
SPhos=2-Dicyclohexylphosphino-2′,6′-dimethoxybiphenyl
TBAF=tetra n-butyl ammonium fluoride
TEA=triethylamine
Tf₂O=trifluoromethanesulfonic anhydride
THF=tetrahydrofuran
TFA=trifluoroacetic acid
trityl=triphenylmethyl
wt.=weight
Xantphos=4,5-Bis(diphenylphosphino)-9,9-dimethylxanthene

Microwave assisted reactions are performed in a single-mode reactor: Emrys™ Optimizer microwave reactor (Personal Chemistry A.B., currently Biotage).

Hydrogenation reactions are performed using an atmospheric balloon or using a Parr hydrogenation shaker apparatus.

Normal phase flash silica gel-based column chromatography is performed using ready-to-connect cartridges from ISCO, on irregular silica gel, particle size 15-40 μm on a Combi-flash Companion chromatography system from ISCO.

Low resolution mass spectra are obtained on an Agilent 1200 series 6130 mass spectrometer. Analytical HPLC is performed on an HP1100 with UV detection at 214 and 254 nm along with ELSD detection, LC/MS (J-Sphere80—C18, 3.0×50 mm, 4.1 min gradient, 5%[0.05% TFA/CH₃CN]:95%[0.05% TFA/H₂O] to 100%[0.05% TFA/CH₃CN]. Preparative RP-HPLC purification is performed on a custom HP1100 automated purification system with collection triggered by mass detection or using a Gilson Inc. preparative UV-based system using a Phenomenex Luna C18 column (50×30 mm I.D., 5 μm) with an acetonitrile (unmodified)-water (0.1% TFA) custom gradient.

For LC-MS characterization of the compounds of the present invention, the following methods are used.
Method 1: The HPLC measurement is performed using an Agilent 1200 system comprising a binary pump with degasser, an autosampler, a column oven, a diode-array detector (DAD) and a column as specified in the respective methods below. Flow from the column is split to a SQ mass spectrometer and Polymer Labs ELSD. The MS detector is configured with an ES ionization source. Nitrogen is used as the nebulizer gas. The source temperature is maintained at 350° C. Data acquisition is performed with Agilent Chemstation software. Reversed phase HPLC is carried out on a Kinetex C18 column (2.6 μm, 2.1×30 μm) from Phenomenex, with a flow rate of 1.5 mL/min, at 45° C. The gradient conditions used are: 93% A (water+0.1% TFA), 7% B (acetonitrile), to 95% B in 1.1 minutes, returning to initial conditions at 1.11 minutes. Injection volume 1 μL. Low-resolution mass spectra (single quadruple MSD detector) are acquired in electrospray mode by scanning from 100 to 700 in 0.25 seconds, step size of 0.1 and peak width of 0.03 minutes. The capillary needle voltage is 3.0 kV and the fragmentor voltage is 100V.
Method 2: Using method 1 instrument and column conditions. The gradient conditions used are: 95% A (water+0.1% TFA), 5% B (acetonitrile), to 95% B in 2.0 minutes, returning to initial conditions at 2.11 minutes. Injection volume 1 μL. Low-resolution mass spectra (single quadruple MSD detector) are acquired in electrospray mode by scanning from 100 to 700 in 0.25 seconds, step size of 0.1 and peak width of 0.03 minutes. The capillary needle voltage is 3.0 kV and the fragmentor voltage is 100V.
Method 3: Using method 1 instrument and column conditions. The gradient conditions used are: 50% A (water+0.1% TFA), 50% B (acetonitrile), to 95% B in 2.0 minutes, returning to initial conditions at 2.11 minutes. Injection volume 1 μL. Low-resolution mass spectra (single quadruple MSD detector) are acquired in electrospray mode by scanning from 100 to 700 in 0.25 seconds, step size of 0.1 and peak width of 0.03 minutes. The capillary needle voltage is 3.0 kV and the fragmentor voltage is 100V.

¹H NMR spectra are recorded either on a Bruker DPX-400 or on a Bruker AV-500 spectrometer with standard pulse sequences, operating at 400 MHz and 500 MHz respectively. Chemical shifts (δ) are reported in parts per million (ppm) downfield from tetramethylsilane (TMS), which is used as internal standard. Coupling constants (J-values) are reported in Hz.

The following Examples are offered as illustrative as a partial scope and particular embodiments of the invention and are not meant to be limiting of the scope of the invention. Abbreviations and chemical symbols have their usual and customary meanings unless otherwise indicated. Unless otherwise indicated, the compounds described herein have been prepared, isolated and characterized using the Schemes and other methods disclosed herein or may be prepared using same.

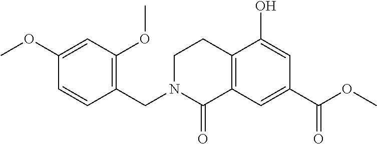

### Intermediate 1

### Methyl 2-(2,4-dimethoxybenzyl)-5-hydroxy-1-oxo-1,2,3,4-tetrahydroisoquinoline-7-carboxylate

Standard Reductive Amination Procedure: Dimethyl 2-hydroxy-2,3-dihydrobenzofuran-4,6-dicarboxylate (15.0 g, 59.5 mmol, 1 equiv) and (2,4-dimethoxyphenyl)methanamine (13.4 mL, 89.2 mmol, 1.5 equiv) were dissolved in CH₂Cl₂ (200 mL) and stirred at 30° C. for 30 min. Then sodium triacetoxyborohydride (25.2 g, 118.9 mmol, 2 equiv) was added and the reaction was stirred at 30° C. for 3 h. The reaction mixture was concentrated, dissolved in 1,4-dioxane (100 mL), and then heated at 110° C. overnight. Saturated aqueous NaHCO₃ was added and the mixture was extracted with CH₂Cl₂ (3×30 mL). The combined organic phases were dried over MgSO₄ and concentrated under reduced pressure to afford the title compound (22 g, 59.2 mmol, quant.), which was used without further purification. ¹H NMR (400 MHz, Chloroform-d) δ 8.26 (d, J=1.6 Hz, 1H), 7.71 (d, J=1.6 Hz, 1H), 7.22 (d, J=8.0 Hz, 1H), 6.39 (d, J=7.8 Hz, 2H), 4.73 (s, 2H), 3.76 (s, 3H), 3.75 (s, 3H), 3.74 (s, 3H), 3.50 (t, J=6.7 Hz, 2H), 2.94 (t, J=6.7 Hz, 2H); LCMS (ESI): Method 2: R_{T}=1.965 min, m/z=372.1 [M+H]⁺.

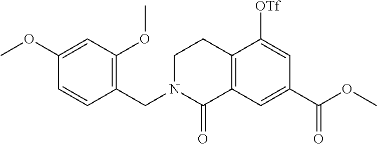

### Intermediate 2

### Methyl 2-(2,4-dimethoxybenzyl)-1-oxo-5-(((trifluoromethyl)sulfonyl)oxy)-1,2,3,4-tetrahydroisoquinoline-7-carboxylate

Standard Triflation Procedure: Phenyl triflimide (34.6 g, 96.9 mmol, 1.2 equiv) was added to a solution of methyl 2-(2,4-dimethoxybenzyl)-5-hydroxy-1-oxo-1,2,3,4-tetrahydroisoquinoline-7-carboxylate (Intermediate 1, 30.0 g, 80.8 mmol, 1 equiv) and N,N-diisopropylethylamine (35 mL, 201.9 mmol, 2.5 equiv) in THF: CH₂Cl₂ (5:1, 360 mL) at 23° C. and stirred for 14 h. Saturated aqueous NaHCO₃ was added and the mixture was extracted with CH₂Cl₂-(3×30 mL). The combined organic phases were dried over MgSO₄ and concentrated under reduced pressure. The residue was purified by flash chromatography (Combi-flash Rf, Hex/EtOAc=0-100% gradient) to afford the title compound (24.5 g, 48.7 mmol, 82% yield) as an oil. ¹H NMR (400 MHz, Chloroform-d) δ 8.80 (d, J=1.6 Hz, 1H), 8.02 (d, J=1.6 Hz, 1H), 7.33-7.27 (m, 1H), 6.49-6.43 (m, 2H), 4.74 (s, 2H), 3.95 (s, 3H), 3.82 (s, 3H), 3.80 (s, 3H), 3.59 (t, J=6.6 Hz, 2H), 3.04 (t, J=6.6 Hz, 2H); LCMS (ESI): Method 3: R_{T}=2.546 min, m/z=504.0 [M+H]⁺.

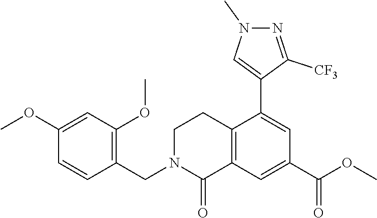

### Intermediate 3

### Methyl 2-(2,4-dimethoxybenzyl)-5-(1-methyl-3-(trifluoromethyl)-1H-pyrazol-4-yl)-1-oxo-1,2,3,4-tetrahydroisoquinoline-7-carboxylate

Standard Suzuki Coupling Procedure: Methyl 2-(2,4-dimethoxybenzyl)-1-oxo-5-(((trifluoromethyl)sulfonyl)oxy)-1,2,3,4-tetrahydroisoquinoline-7-carboxylate (Intermediate 2, 12.3 g, 24.3 mmol, 1 equiv), (1-methyl-3-(trifluoromethyl)-1H-pyrazol-4-yl)boronic acid (7.1 mg, 36.4 mmol, 1.5 equiv), potassium carbonate (8.4 g, 60.8 mmol, 2.5 equiv), and PdCl₂(dppf) (890 mg, 1.2 mmol, 0.05 equiv) were dissolved in 1,4-dioxane:water (4:1, 5 mL) under an Ar atmosphere in a sealed tube. The reaction mixture was stirred for 14 h at 90° C. then cooled to 23° C. Brine was added to the mixture, followed by extraction with EtOAc (3×20 mL). The combined organic layers were dried over MgSO₄ and concentrated under reduced pressure. The residue was purified by flash chromatography (Combi-flash Rf, Hex/EtOAc=0-100% gradient) to afford the title compound (11.0 g, 21.8 mmol, 90% yield). ¹H NMR (400 MHz, Chloroform-d) δ 8.81 (d, J=1.9 Hz, 1H), 7.99 (d, J=1.8 Hz, 1H), 7.36 (d, J=1.1 Hz, 1H), 7.31-7.27 (m, 1H), 6.45 (dd, J=6.2, 2.5 Hz, 2H), 4.73 (s, 2H), 4.00 (s, 3H), 3.92 (s, 3H), 3.80 (s, 3H), 3.79 (s, 3H), 3.46 (t, J=6.6 Hz, 2H), 2.75 (t, J=6.5 Hz, 2H); LCMS (ESI): Method 2: R_{T}=1.072 min, m/z=504.4 [M+H]⁺.

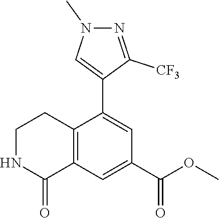 [SMILES: COC(=O)c1cc2c(c(-c3cn(C)nc3C(F)(F)F)c1)CCNC2=O]

### Intermediate 4

### Methyl 5-(1-methyl-3-(trifluoromethyl)-1H-pyrazol-4-yl)-1-oxo-1,2,3,4-tetrahydroisoquinoline-7-carboxylate

Standard TFA Deprotection Procedure: Anisole (24 mL, 218 mmol, 5 equiv.) was added to a solution of methyl 2-(2,4-dimethoxybenzyl)-5-(1-methyl-3-(trifluoromethyl)-1H-pyrazol-4-yl)-1-oxo-1,2,3,4-tetrahydroisoquinoline-7-carboxylate (Intermediate 3, 22.0 g, 43.7 mmol, 1 equiv.) in CH₂Cl₂ (50 mL) and TFA (100 mL). The reaction was stirred at room temperature overnight then concentrated under reduced pressure. The residue was dissolved in EtOAc and washed with sat. NaHCO₃. The organic layer was dried (MgSO₄) and concentrated. The residue was purified by flash chromatography (Combi-flash Rf, Hex/EtOAc=0-100% gradient followed by MeOH/CH₂Cl₂=0-10% gradient) to provide the title compound (13.5 g, 38.2 mmol, 87% yield). ¹H NMR (400 MHz, Chloroform-d) δ 8.77 (d, J=1.6 Hz, 1H), 8.05 (d, J=1.6 Hz, 1H), 7.40 (s, 1H), 6.25 (s, 1H), 4.03 (s, 3H), 3.92 (s, 3H), 3.51 (td, J=6.5, 2.8 Hz, 2H), 2.84 (t, J=6.5 Hz, 1H); LCMS (ESI): Method 2: R_{T}=1.363 min, m/z=354.1 [M+H]⁺.

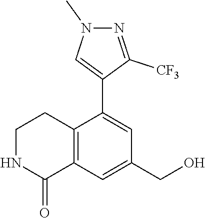 [SMILES: Cn1cc(-c2cc(CO)cc3c2CCNC3=O)c(C(F)(F)F)n1]

### Intermediate 5

### 7-(Hydroxymethyl)-5-(1-methyl-3-(trifluoromethyl)-1H-pyrazol-4-yl)-3,4-dihydroisoquinolin-1(2H)-one

Standard Methylester Reduction Procedure: Lithium triethylborohydride (4 mL, 3.9 mmol, 3 equiv) was added dropwise to a solution of methyl 5-(1-methyl-3-(trifluoromethyl)-1H-pyrazol-4-yl)-1-oxo-1,2,3,4-tetrahydroisoquinoline-7-carboxylate (Intermediate 4, 457 mg, 1.3 mmol, 1 equiv) in THE at 0° C. The reaction was stirred for 40 min, then the reaction was quenched with sat. aq. NaHCO₃. The mixture was then extracted with EtOAc. The combined organic layers were dried over MgSO₄, concentrated, and dried under reduced pressure to provide the title compound (420 mg, 1.3 mmol, quant.), which was used without further purification. LCMS (ESI): Method 2: R_{T}=1.145 min, m/z=326.1 [M+H]⁺.

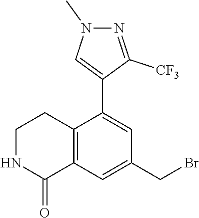 [SMILES: Cn1cc(-c2cc(CBr)cc3c2CCNC3=O)c(C(F)(F)F)n1]

### Intermediate 6

### 7-(Bromomethyl)-5-(1-methyl-3-(trifluoromethyl)-1H-pyrazol-4-yl)-3,4-dihydroisoquinolin-1(2H)-one

Standard Bromination Procedure: PBr₃ (0.2 mL, 2.6 mmol, 2 equiv) was added to a solution of 7-(hydroxymethyl)-5-(1-methyl-3-(trifluoromethyl)-1H-pyrazol-4-yl)-3,4-dihydroisoquinolin-1(2H)-one (Intermediate 5, 420 mg, 1.3 mmol, 1 equiv) in CH₂Cl₂ (10 mL) at 0° C. The reaction was warmed to room temperature and stirred overnight. Sat. aq. NaHCO₃ was added and the mixture was extracted with EtOAc. The combined organic layers were dried over MgSO₄ and concentrated to provide the title compound (440 mg, 1.3 mmol, quant.), which was used in the next step without further purification. ¹H NMR (400 MHz, Chloroform-d) δ 8.15 (d, J=2.0 Hz, 1H), 7.42 (d, J=2.0 Hz, 1H), 7.40 (d, J=1.1 Hz, 1H), 4.51 (s, 2H), 4.02 (s, 3H), 3.48 (td, J=6.5, 2.7 Hz, 2H), 2.79 (t, J=6.5 Hz, 2H); LCMS (ESI); m/z=387.9 [M+H]⁺.

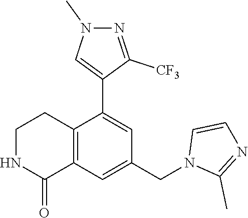 [SMILES: Cc1nccn1Cc1cc2c(c(-c3cn(C)nc3C(F)(F)F)c1)CCNC2=O]

### Intermediate 7

### 7-((2-Methyl-1H-imidazol-1-yl)methyl)-5-(1-methyl-3-(trifluoromethyl)-1H-pyrazol-4-yl)-3,4-dihydroisoquinolin-1(2H)-one

Standard Bromide Displacement Procedure: 2-Methyl-1H-imidazole (846 mg, 10.3 mmol, 4 equiv) was added to a solution of 7-(bromomethyl)-5-(1-methyl-3-(trifluoromethyl)-1H-pyrazol-4-yl)-3,4-dihydroisoquinolin-1(2H)-one (Intermediate 6, 1.0 g, 2.6 mmol, 1 equiv) in acetonitrile (15 mL) at 23° C. The reaction mixture was stirred for 12 h at 50° C., then cooled to ambient temperature, filtered and concentrated. The residue was purified by flash chromatography (Combi-flash Rf, DCM/MeOH=0-10% gradient) to afford the title compound (700 mg, 1.8 mmol, 70% yield). ¹H NMR (400 MHz, Chloroform-d) δ 8.01 (d, J=2.0 Hz, 1H), 7.32 (s, 1H), 6.95 (d, J=1.4 Hz, 1H), 6.94 (d, J=2.0 Hz, 1H), 6.85 (d, J=1.4 Hz, 1H), 5.99 (s, 1H), 5.09 (s, 2H), 4.00 (s, 3H), 3.48 (td, J=6.5, 2.8 Hz, 2H), 2.79 (t, J=6.5 Hz, 2H), 2.35 (s, 3H); LCMS (ESI): Method 2: R_{T}=0.973 min, m/z=390.0 [M+H]⁺.

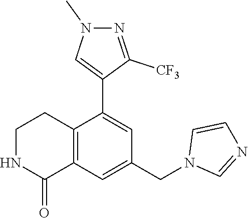 [SMILES: Cn1cc(-c2cc(Cn3ccnc3)cc3c2CCNC3=O)c(C(F)(F)F)n1]

### Intermediate 8

### 7-((1H-Imidazol-1-yl)methyl)-5-(1-methyl-3-(trifluoromethyl)-1H-pyrazol-4-yl)-3,4-dihydroisoquinolin-1(2H)-one

1H-Imidazole (0.91 g, 13 mmol, 4 equiv) was added to a solution of 7-(bromomethyl)-5-(1-methyl-3-(trifluoromethyl)-1H-pyrazol-4-yl)-3,4-dihydroisoquinolin-1(2H)-one (Intermediate 6, 1.3 g, 3.3 mmol, 1 equiv) in acetonitrile (15 mL) at 23° C. The reaction mixture was stirred for 12 h at 50° C., then cooled to ambient temperature, filtered and concentrated. The residue was purified by flash chromatography (Combi-flash Rf, DCM/MeOH=0-10% gradient) to afford the title compound (750 mg, 2.0 mmol, 60% yield). ¹H NMR (400 MHz, DMSO-d₆) δ 8.03 (brs, 1H), 8.01 (d, J=1.1 Hz, 1H), 7.81 (d, J=2.0 Hz, 1H), 7.75 (d, J=1.2 Hz, 1H), 7.26 (d, J=2.0 Hz, 1H), 7.19-7.15 (m, 1H), 6.90 (t, J=1.1 Hz, 1H), 5.25 (s, 2H), 3.96 (s, 3H), 3.26 (td, J=6.6, 2.7 Hz, 2H), 2.64 (t, J=6.5 Hz, 2H); LCMS (ESI): Method 2: R_{T}=0.979 min, m/z=376.0 [M+H]⁺.

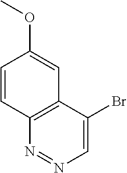

### Intermediate 9

### 4-Bromo-6-methoxycinnoline

6-Methoxycinnolin-4(1H)-one (84.0 mg, 0.477 mmol, 1 equiv) was dissolved in acetonitrile (4 mL) and stirred at room temperature. Potassium carbonate (198 mg, 1.43 mmol, 3 equiv) and phosphorus oxybromide (410 mg, 1.43 mmol, 3 equiv) were added and the reaction was placed in a preheated reaction block at 60° C. The reaction was quenched with addition of ice and water. Sat. NaHCO₃ was added to adjust the pH of the mixture to 7-9. The aqueous layer was then extracted with CH₂Cl₂ (3×20 mL). The combined organic layers were dried over MgSO₄ and concentrated under reduced pressure. The residue was purified by flash chromatography (Combi-flash Rf, Hex/EtOAc=0-100% gradient) to afford the title compound (38 mg, 0.16 mmol, 33% yield). ¹H NMR (400 MHz, Chloroform-d) δ 9.34 (s, 11H), 8.39 (d, J=9.3 Hz, 1H), 7.51 (dd, J=9.4 2.4 Hz, 1H), 7.24 (d, J=2.5 Hz, 1H), 4.04 (s, 3H), LCMS (ESI): Method 2: R_{T}=1.429 min, m/z=239.1 [M+H]⁺.

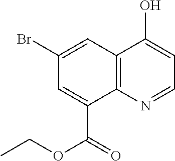 [SMILES: CCOC(=O)c1cc(Br)cc2c(O)ccnc12]

### Intermediate 10

### Ethyl 6-bromo-4-hydroxyquinoline-8-carboxylate

Step A. Preparation of ethyl 5-bromo-2-(((2,2-dimethyl-4,6-dioxo-1,3-dioxan-5-ylidene)methyl)amino)benzoate. To a solution of ethyl 2-amino-5-bromobenzoate (5.0 g, 16.6 mmol) and Meldrum's acid (1.2 equiv) in EtOH (25 mL) was added triethyl orthoformate (1 equiv). The reaction was stirred at 80° C. overnight. The reaction was cooled to 0° C., filtered, and washed with cold EtOH to yield the title compound.

Step B. Preparation of ethyl 6-bromo-4-hydroxyquinoline-8-carboxylate. Ethyl 5-bromo-2-(((2,2-dimethyl-4,6-dioxo-1,3-dioxan-5-ylidene)methyl)amino)benzoate (16.6 mmol) was added portionwise to Dowtherm A (10 mL) at 260° C. and stirred for 30 min. The reaction was cooled to room temperature, and hexanes were added. The resulting mixture was filtered, and the solid was washed with hexanes to yield the title compound (3.70 g, 12.5 mmol, 79.9% yield in two steps). ¹H NMR (400 MHz, Chloroform-) δ 8.75 (d, J=2.2 Hz 1H), 8.45 (d, J=2.3 Hz, 1H), 7.69-7.65 (m, 1H), 6.35 (dd, J=1.1 Hz, 1H), 4.46 (q, J=7.0 2H), 1.46 (t, J=7.1 3H); LCMS (ESI): m/z=295.9 [M+H]⁺.

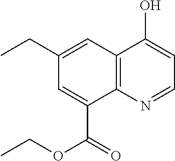 [SMILES: CCOC(=O)c1cc(CC)cc2c(O)ccnc12]

### Intermediate 11

### Ethyl 6-ethyl-4-hydroxyquinoline-8-carboxylate

A mixture of ethyl 6-bromo-4-hydroxyquinoline-8-carboxylate (Intermediate 10, 1.49 g, 5.03 mmol), triethylborane (2 equiv, 1 M THF), cesium carbonate (2 equiv), and Pd(dppf)Cl₂ (0.05 equiv) in THF (20 mL) was stirred for 3 h at 60° C. under Ar in a sealed tube. The reaction was cooled to 0° C. and quenched with 10% Y, aq. NaOH and 30% aq. H₂O₂. The resulting mixture was warmed to 23° C., brine was added, and the mixture was extracted with EtOAc (3×50 mL). The combined organic layers were dried over MgSO₄ and concentrated under reduced pressure. The residue was purified by flash chromatography (Combi-flash Rf, Hex/EtOAc=0-100% gradient followed by DCM/MeOH=0-10% gradient) to afford the title compound (700 mg, 2.85 mmol, 57% yield). ¹H NMR (400 MHz, Chloroform-d) δ 8.49 (s, 1H), 8.25 (s, 1H), 7.69 (t, J=7.7 Hz, 1H), 6.35 (d, J=4.1 Hz, 1H), 4.48 (q, J=7.1 Hz, 2H), 2.80 (q, J=7.6 Hz, 2H), 1.47 (t, J=7.1 3H), 1.35 (t, J=7.6 3H); LCMS (ESI): m/z=246.1 [M+H]⁺.

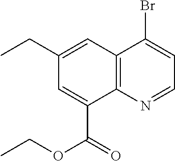 [SMILES: CCOC(=O)c1cc(CC)cc2c(Br)ccnc12]

### Intermediate 12

### Ethyl 4-bromo-6-ethylquinoline-8-carboxylate

To a solution of ethyl 6-ethyl-4-hydroxyquinoline-8-carboxylate (Intermediate 11, 610 mg, 2.49 mmol) in DMF (15 mL) was added PBr₃ (2 equiv) dropwise at 0° C. The reaction mixture was warmed to room temperature and stirred overnight. The reaction was quenched with ice, and the pH of the mixture was adjusted to 7 with NaHCO₃. The solid was filtered, washed with water, and dried to yield the title compound (630 mg, 2.04 mmol, 82.2% yield). ¹H NMR (400 MHz, Chloroform-d) δ 8.48 (s, 1H), 8.35 (s, 1H), 8.27 (t, J=7.0 Hz, 1H), 7.04 (d, J=7.0 Hz, 1H), 4.50 (q, J=7.1 Hz, 2H), 2.80 (q, J=7.6 Hz, 2H), 1.49 (t, J=7.4 3H), 1.33 (t, J=7.6 3H); LCMS (ESI): m/z=309.1 [M+H]⁺.

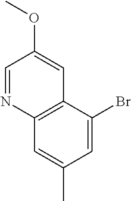

### Intermediate 13

### 5-Bromo-3-methoxy-7-methylquinoline

Step A. Preparation of 2,6-dibromo-4-methylbenzaldehyde. To a solution of 1,3-dibromo-5-methylbenzene (21.0 g, 84 mmol) in anhydrous THF (200 mL) was added 2.0 M lithium diisopropylamide solution (58.8 mL, 1.4 eq) was drop-wise at −78° C. The reaction mixture was stirred for 30 minutes then DMF (7.8 mL, 1.2 eq) was added. The reaction was stirred for 1 h at −78° C. then quenched with 1N HCl and EtOAc. The quenched mixture was extracted with EtOAc (2×200 mL), and the organic layer was washed with brine, dried over MgSO₄, filtered, and concentrated to give the title compound (23.0 g, 98%). ¹H NMR (400 MHz, Chloroform-d) δ 10.26 (s, 1H), 7.49 (s, 2H), 2.39 (s, 3H).

Step B. Preparation of 2-(2,6-dibromo-4-methylphenyl)-1,3-dioxolane. A mixture of 2,6-dibromo-4-methylbenzaldehyde (23.0 g, 83 mmol), ethane-1,2-diol (11 mL, 2.3 eq), and p-toluenesulfonic acid monohydrate (7.9 g, 0.5 eq) in anhydrous toluene (200 mL) was refluxed using a DeanStark trap until TLC showed no starting material. The reaction mixture was cooled to room temperature and concentrated. The residue was dissolved in DCM (200 mL), washed with 1N aq NaOH (50 mL) and concentrated. The residue was purified by flash chromatography (Combi-flash Rf, Hex/EtOAc=0-5% gradient) to afford the title compound (15.7 g, 59%). ¹H NMR (400 MHz, Chloroform-d) δ 7.39 (s, 2H), 6.36 (s, 1H), 4.35-4.31 (m, 2H), 4.08-4.05 (m, 2H), 2.29 (s, 3).

Step C. Preparation of N-(3-bromo-2-(1,3-dioxolan-2-yl)-5-methylphenyl)-1,1-diphenylmethanimine. A mixture of 2-(2,6-dibromo-4-methylphenyl)-1,3-dioxolane (15.7 g, 48.8 mmol), benzophenone imine (8.2 mL, 1.0 eq), cesium carbonate (31.8 g, 2.0 eq), BINAP (3.0 g, 0.1 eq), and palladium(II) acetate (0.55 g, 0.05 eq) in anhydrous toluene (200 mL) was purged with Ar then stirred for 16 h at 80° C. The reaction mixture was cooled to ambient temperature and quenched with H₂O (200 mL). The layers were separated, and the aqueous layer was extracted with EtOAc. The combined organic layer was washed with brine, dried over MgSO₄, filtered, and concentrated. The residue was triturated with DCM (200 mL). The solid was filtered to give the title compound. The filtrate was concentrated, and the residue was purified by flash chromatography (Combi-flash Rf, Hex/EtOAc=0-10% gradient) to afford the additional title compound (17.1 g, 83% yield). ¹H NMR (400 MHz, DMSO-d6) δ 7.67-7.62 (m, 2H), 7.57-7.46 (m, 3H), 7.34 (bs, 3H), 7.24 (bs, 2H), 7.00 (s, 1H), 6.16 (s, 1H), 6.14 (s, 1H), 4.02-3.98 (m, 2H), 3.88-3.84 (m, 2H), 2.01 (s, 3H).

Step D. Preparation of 2-amino-6-bromo-4-methylbenzaldehyde. To a solution of N-(3-bromo-2-(1,3-dioxolan-2-yl)-5-methylphenyl)-1,1-diphenylmethanimine (17.1 g, 40.5 mmol) in THF (100 mL) was added aq. 1N HCl (100 mL, 2.5 eq). The reaction mixture stirred for 2 h at 80° C., then was cooled to ambient temperature. The mixture was neutralized with aq. 6N NaOH. The mixture was extracted with EtOAc, and the combined organic layers were washed with brine, dried over MgSO₄, filtered, and concentrated. The residue was purified by flash chromatography (Combi-flash Rf, Hex/EtOAc=0-10% gradient) to afford the title compound (6.3 g, 73%) as a yellow oil. ¹H NMR (400 MHz, Chloroform-d) δ 10.30 (s, 1H), 6.73 (s, 1H), 6.38 (s, 1H), 2.23 (s, 3H); LCMS Method 2: >95% purity 254 nm, R_{T}=1.74 min, MS (ESI) 214.0 [M+H]⁺.

Step E. Preparation of 5-bromo-3-methoxy-7-methylquinoline. To a solution of 2-amino-6-bromo-4-methylbenzaldehyde (1.0 mmol) in ethanol was added methoxyacetaldehyde (1.2 eq) and 1M aq. NaOH solution (2.0 eq). The reaction was heated under the microwave at 110° C. for 30 min. The reaction was cooled to room temperature, poured into dichloromethane and washed with brine. The layers were separated, and the organic layer was concentrated. The residue was purified by flash chromatography (Combi-flash Rf, Hex/EtOAc=0-15% gradient) to afford the title compound. ¹H NMR (400 MHz, Chloroform-d) δ 8.63 (d, J=2.7 Hz, 1H), 7.80 (s, 1H), 7.68-7.66 (m, 2H), 3.99 (s, 3H), 2.51 (s, 3H).

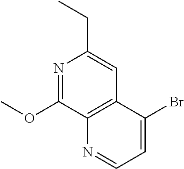

### Intermediate 14

### 4-Bromo-6-ethyl-8-methoxy-1,7-naphthyridine

Step A: Preparation of 6-ethyl-2-methoxypyridin-3-amine. Ina round bottom flask, 6-bromo-2-methoxypyridin-3-amine (1100 mg, 5.4 mmol, 1 equiv), PdCl₂(dppf)-CH₂Cl₂ adduct (221 mg, 0.27 mmol, 0.05 equiv) were dissolved in THF (20 mL) and placed under an argon atmosphere at room temperature. Diethylzinc (3.5 mL, 3.5 mmol, 0.65 equiv, 1 M Hexanes) was added dropwise and the reaction mixture was then placed in a preheated heating block and stirred for 4 h at 65° C. At 23° C., sat. NH₄Cl was added to the mixture and extracted with EtOAc (3×20 mL). The combined organic layers were dried over MgSO₄ and concentrated under reduced pressure. The title compound (645 mg, 4.2 mmol, 78% yield) was obtained after purification by column chromatography (Combi-flash Rf, Hex/EtOAc=0-70%, gradient). ¹H NMR (400 MHz, Chloroform-d) δ 7.54 (d, J=7.5 Hz, 1H), 6.54 (d, J=7.5 Hz, 1H), 3.97 (s, 3H), 3.61 (brs, 2H), 2.62 (q, J=7.6 Hz, 2H), 1.24 (t, J=7.6 Hz, 3H), LCMS (ESI): Method 2: R_{T}=1.473 min, m/z=153.2 [M+H]⁺.

Step B: Preparation of 5-(((6-ethyl-2-methoxypyridin-3-yl)amino)methylene)-2,2-dimethyl-1,3-dioxane-4,6-dione. In a round bottom flask, 6-ethyl-2-methoxypyridin-3-amine (10.5 g, 69 mmol, 1 equiv) and 2,2-dimethyl-1,3-dioxane-4,6-dione (11.9 g, 83 mmol, 1.2 equiv) were dissolved in EtOH (200 mL). Then triethoxymethane (11 mL, 69 mmol, 1 equiv) was added and the reaction was stirred at 80° C. overnight. The reaction was cooled to 0° C., filtered, and washed with cold EtOH to yield the title compound (18.0 g, 58.8 mmol, 85% yield). ¹H NMR (400 MHz, Chloroform-) δ 11.33 (d, J=14.5 Hz, 1H), 8.60 (d, J=14.6, 1H), 7.47 (d, J=7.9 Hz, 1H), 6.80 (d, J=7.9 Hz, 1H), 4.07 (s, 3H), 2.74 (q, J=7.5 Hz, 2H), 1.75 (s, 6H), 1.29 (t, J=7.5 Hz, 3H); LCMS (ESI): Method 2: R_{T}=2.070 min, m/z=307.0 [M+H]⁺.

Step C: Preparation of 6-ethyl-8-methoxy-1,7-naphthyridin-4-ol. Finely powdered 5-(((6-ethyl-2-methoxypyridin-3-yl)amino)methylene)-2,2-dimethyl-1,3-dioxane-4,6-dione (6.13 g, 20.00 mmol, 1 equiv) was added portion-wise to a vigorously stirred Dowtherm A (80 mL) at 228° C. over 15 min and then stirred for another 15 min. The reaction was immediately cooled in a room temperature water bath with constant stirring. Hexanes were added to form a precipitate, and the precipitate was filtered and washed with hexanes to obtain the title compound (2.50 g, 12.2 mmol, 61% yield). The title compound was further purified by flash column chromatography (Combi-flash Rf, DCM/MeOH=0-10%, gradient). ¹H NMR (400 MHz, Chloroform-d) δ 8.87 (brs, 1H), 7.64 (d, J=7.5 Hz, 1H), 7.52 (s, 1H), 6.38 (d, J=7.5 Hz, 1H), 4.12 (s, 3H), 2.80 (q, J=7.5 Hz, 2H), 1.31 (t, J=7.6 Hz, 3H); LCMS (ESI): Method 2: R_{T}=1.099 min, m/z=205.2 [M+H]⁺.

Step D: Preparation of 6-ethyl-8-methoxy-1,7-naphthyridin-4-yl trifluoromethanesulfonate. 6-Ethyl-8-methoxy-1,7-naphthyridin-4-ol (7.17 g, 35.0 mmol, 1 equiv) was dissolved in DMF (150 mL) and stirred at room temperature. 1,1,1-Trifluoro-N-phenyl-N-((trifluoromethyl)sulfonyl)methanesulfonamide (18.83 g, 52.7 mmol, 1.5 equiv.) followed by N,N-diisoproplyethylamine (15 mL, 87.8 mmol, 2.5 equiv) and DMAP (86 mg, 0.70 mmol, 0.02 equiv) were added and reaction was stirred at 50° C. for 4 h. The reaction was diluted with EtOAc and sat. NH₄Cl and extracted with EtOAc. The organic layer was dried over MgSO₄, filtered, and concentrated under reduced pressure. The title compound (9.89 g, 29.4 mmol, 84% yield) was obtained after purification by column chromatography (Combi-flash Rf, Hex/EtOAc=0-100%, gradient). ¹H NMR (400 MHz, Chloroform-d) δ 8.95 (d, J=5.2 Hz, 1H), 7.57 (d, J=4.8 Hz, 1H), 7.22 (s, 1H), 4.22 (s, 3H), 2.90 (q, J 7.6 Hz, 2H), 1.38 (t, J=7.5 Hz, 3H); LCMS (ESI): Method 2: R_{T}=1.861 min, m/z=337.0 [M+H]⁺.

Step E: Preparation of 4-bromo-6-ethyl-8-methoxy-1,7-naphthyridine. 6-Ethyl-8-methoxy-1,7-naphthyridin-4-yl trifluoromethanesulfonate (9.89 g, 29.4 mmol, 1 equiv) was dissolved in acetonitrile (200 mL) and stirred at room temperature. Lithium bromide (25.5 g, 294 mmol, 10 equiv) was added and reaction was stirred at 80° C. overnight. The reaction was cooled to room temperature and concentrated under reduced pressure. The reaction was diluted with EtOAc and water and extracted with EtOAc. The organic layer was dried over MgSO₄, filtered, and concentrated under reduced pressure. The title compound (6.51 g, 24.4 mmol, 83% yield) was obtained after purification by column chromatography (Combi-flash Rf, Hex/EtOAc=0-100%, gradient). ¹H NMR (400 MHz, Chloroform-d) δ 8.63 (d, J=4.7 Hz, 1H), 7.81 (d, J=4.6 Hz, 1H), 7.31 (s, 1H), 4.22 (s, 3H), 2.87 (q, J 7.2 Hz, 2H), 1.38 (t, J=7.5 Hz, 3H); LCMS (ESI): Method 2: R_{T}=1.628 min, m/z=267.0 [M+H]⁺.

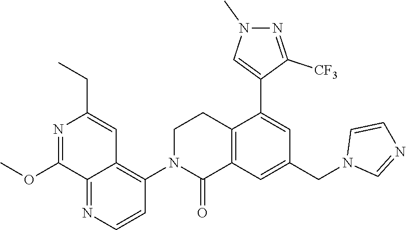 [SMILES: CCc1cc2c(N3CCc4c(cc(Cn5ccnc5)cc4-c4cn(C)nc4C(F)(F)F)C3=O)ccnc2c(OC)n1]

### Intermediate 15

### 7-((1H-Imidazol-1-yl)methyl)-2-(6-ethyl-8-methoxy-1,7-naphthyridin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-5-(1-methyl-3-(trifluoromethyl)-1H-pyrazol-4-yl)-3,4-dihydroisoquinolin-1(2H)-one (Intermediate 8, 1.0 equiv), 4-bromo-6-ethyl-8-methoxy-1,7-naphthyridine (Intermediate 14, 2.0 equiv), cesium carbonate (2.0 equiv), Xantphos (0.2 equiv), and Pd₂(dba)₃ (0.1 equiv) were dissolved in 1,4-dioxane under an Ar. The reaction mixture was stirred for 14 h at 110° C. then cooled to 23° C. Brine was added to the mixture and the mixture was extracted with EtOAc (3×50 mL). The combined organic layers were dried over MgSO₄ and concentrated under reduced pressure. The residue was purified by reversed-phase HPLC (Phenomenex Gemini C18, H₂O/CH₃CN gradient from 15-85% CH₃CN, 0.1% TFA) followed by neutralization with sat. aq. NaHCO₃ to yield the title compound. ¹H NMR (400 MHz, Chloroform-d) δ 8.93 (d, J=4.6 Hz, 1H), 8.15 (s, 1H), 7.59 (s, 1H), 7.47 (d, J=4.6 Hz, 1H), 7.41 (s, 1H), 7.18 (s, 1H), 7.11 (s, 1H), 6.95 (s, 2H), 5.20 (s, 2H), 4.22 (s, 3H), 4.02 (s, 3H), 4.00-3.96 (m, 1H), 3.84-3.78 (s, 1H), 3.19-3.11 (m, 1H), 3.00-2.94 (m, 1H), 2.81 (q, J=7.5 Hz, 2H), 1.32 (t, J=7.5 Hz, 3H); LCMS Method 2: >95%, R_{T}=1.40 min, MS (ESI) 562.0 [M+H]⁺.

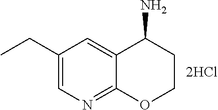

### Intermediate 16

### (S)-6-Ethyl-3,4-dihydro-2H-pyrano[2,3-b]pyridin-4-amine dihydrochloride salt

Step A: Preparation of (R,E)-N-((5-bromo-2-fluoropyridin-3-yl)methylene)-2-methylpropane-2-sulfinamide. To a solution of 5-bromo-2-fluoronicotinaldehyde (4.0 g, 20 mmol, 1 equiv) in dichloromethane (80 mL) were added (R)-2-methylpropane-2-sulfinamide (2.4 g, 20 mmol, 1 equiv) and Cs₂CM3 (9.6 g, 29 mmol, 1.5 equiv). The resulting mixture was stirred at room temperature for 36 h, and then washed with water. The organic layer was dried (Na₂SO₄), filtered, and concentrated. The residue was purified by silica gel column chromatography (Combi-flash Rf, Hex/EtOAc=0-20%, gradient) to provide the title compound (5.1 g, 17 mmol, 85% yield). LCMS (ESI): m/z=349.0 [M+H]⁺.

Step B: Preparation of (R)-N-((S)-1-(5-bromo-2-fluoropyridin-3-yl)but-3-en-1-yl)-2-methylpropane-2-sulfinamide. To a solution of (R,E)-N-((5-bromo-2-fluoropyridin-3-yl)methylene)-2-methylpropane-2-sulfinamide (1.55 g, 5.05 mmol, 1 equiv) in THF (35 mL) at −78° C. was added dropwise 1M allylzinc bromide (7.57 mL, 7.57 mmol, 1.5 equiv). The resulting mixture was stirred for 45 min, then quenched with saturated aqueous NH₄Cl solution and extracted with EtOAc. The combined organic layers were dried over Na₂SO₄, filtered, and concentrated. The residue was purified by silica gel column chromatography (Combi-flash Rf, Hex/EtOAc=0-50%, gradient) to provide the title compound (1.7 g, 4.9 mmol, 96% yield, dr=93:7). ¹H NMR (400 MHz, Chloroform-d) Major isomer: δ 8.19 (m, 1H), 7.87 (dd, J=2.4, 4.0 Hz, 1H), 5.71 (m, 1H), 5.22 (m, 2H), 4.71 (m, 1H), 3.68 (d, J=2.8 Hz, 1H), 2.69 (m, 1H), 2.54 (m, 1H), 2.05 (s, 1H), 1.23 (s, 9H); Minor isomer: δ 8.19 (m, 1H), 7.87 (dd, J=2.4, 4.0 Hz, 1H), 5.64 (m, 1H), 5.14 (m, 2H), 4.57 (m, 1H), 3.74 (d, J=7.6 Hz, 1H), 2.69 (m, 1H), 2.54 (m, 1H), 2.05 (s, 1H), 1.23 (s, 9H).

Step C: Preparation of (R)-N-((S)-1-(5-bromo-2-fluoropyridin-3-yl)-3-hydroxypropyl)-2-methylpropane-2-sulfinamide. To a solution of (R)-N-((S)-1-(5-bromo-2-fluoropyridin-3-yl)but-3-en-1-yl)-2-methylpropane-2-sulfinamide (1.76 g, 5.04 mmol, 1 equiv) in MeOH (75 mL) was bubbled 03 and the reaction progress was monitored by LCMS. Once the starting material was consumed, KF (351 mg, 6.04 mmol, 1.2 equiv) was added and the acetone/dry ice bath was replaced with an ice bath. The resulting mixture was allowed to stir at 0° C. for 30 min, then NaBH₄ (381.3 mg, 10.08 mmol, 2 equiv) was added and stirring continued for another one hour. The solvent was removed under reduced pressure and the residue was taken up in EtOAc and washed with brine/water. The organic layer was dried over Na₂SO₄, filtered, and concentrated. The residue was purified by silica gel column chromatography (Combi-flash Rf, Hex/EtOAc=0-80%, gradient) to provide the title compound (770 mg, 2.18 mmol, 43% yield). ¹H NMR (400 MHz, Chloroform-d) δ 8.18 (s, 1H), 7.91 (d, J=4.0 Hz, 1H), 4.82 (m, 2H), 3.90 (m, 2H), 2.03 (m, 2H), 1.25 (s, 9H).

Step D: Preparation of (R)-N-((S)-6-bromo-3,4-dihydro-2H-pyrano[2,3-b]pyridin-4-yl)-2-methylpropane-2-sulfinamide. To a solution of (R)-N-((S)-1-(5-bromo-2-fluoropyridin-3-yl)-3-hydroxypropyl)-2-methylpropane-2-sulfinamide (770 mg, 2.18 mmol, 1 equiv) in 1,4-dioxane (22 mL) was added tBuOK (501.4 mg, 4.47 mmol, 2.05 equiv). The resulting mixture was stirred at 53° C. for 1 h, then quenched with brine/water and extracted with EtOAc. The combined organic layers were dried over Na₂SO₄, filtered, and concentrated. The residue was purified by silica gel column chromatography (Combi-flash Rf, DCM/MeOH=0-10% gradient) to provide the title compound (698 mg, 2.09 mmol, 96% yield). LCMS (ESI): m/z=333.0 [M+H]⁺.

Step E: Preparation of (R)-2-methyl-N-((S)-6-vinyl-3,4-dihydro-2H-pyrano[2,3-b]pyridin-4-yl)propane-2-sulfinamide. To a solution of (R)-N-((S)-6-bromo-3,4-dihydro-2H-pyrano[2,3-b]pyridin-4-yl)-2-methylpropane-2-sulfinamide (227.6 mg, 0.68 mmol, 1 equiv) in EtOH (7 mL) was added potassium vinyltrifluoroborate (137 mg, 1.02 mmol, 1.5 equiv), PdCl₂(dppf) (25 mg, 34 μmol, 0.05 equiv), and Et₃N (237 μL, 1.7 mmol, 2.5 equiv). The resulting mixture was stirred in a sealed tube at 80° C. for 16 h, then concentrated. The residue was taken up in EtOAc and washed with brine/water. The organic layer was dried (Na₂SO₄), filtered, and concentrated. The residue was purified by silica gel column chromatography (Combi-flash Rf, Hex/EtOAc=50-100% gradient) to provide the title compound (113.4 mg, 0.4 mmol, 59% yield). ¹H NMR (400 MHz, Chloroform-d) δ 8.16 (d, J=2.4 Hz, 1H), 7.78 (d, J=2.0 Hz, 1H), 6.63 (dd, J=10.8, 17.6 Hz, 1H), 5.64 (d, J=17.6 Hz, 1H), 5.24 (d, J=10.8 Hz, 1H), 4.57 (m, 1H), 4.40 (m 2H), 3.53 (d, J=10.4 Hz, 1H), 2.45 (m, 1H), 2.15 (m, 1H), 1.28 (s, 9H).

Step F: Preparation of (R)-N-((S)-6-ethyl-3,4-dihydro-2H-pyrano[2,3-b]pyridin-4-yl)-2-methylpropane-2-sulfinamide. To a solution of (R)-2-methyl-N-((S)-6-vinyl-3,4-dihydro-2H-pyrano[2,3-b]pyridin-4-yl)propane-2-sulfinamide (113 mg, 0.4 mmol, 1 equiv) in EtOH (6 mL) was added 10% Pd on carbon (42 mg, 40 μmol, 0.1 equiv). The resulting mixture was stirred under H₂ atmosphere for 16 h, then filtered through a celite plug. The combined filtrate was concentrated, and the residue was purified by silica gel column chromatography (Combi-flash Rf, Hex/EtOAc=50-100% gradient) to provide the title compound (108 mg, 0.38 mmol, 95% yield). ¹H NMR (400 MHz, Chloroform-d) δ 7.96 (s, 1H), 7.51 (s, 1H), 4.53 (m, 1H), 4.35 (m, 2H), 3.56 (d, J=9.2 Hz, 1H), 2.56 (q, J=7.6 Hz, 2H), 2.41 (m, 1H), 2.12 (m, 1H), 1.25 (s, 9H), 1.19 (t, J=76 Hz, 3H).

Step G: Preparation of (S)-6-ethyl-3.4-dihydro-2H-pyrano[2,3-b]pyridin-4-amine hydrochloride salt. To a solution of (R)-N-((S)-6-ethyl-3,4-dihydro-2H-pyrano[2,3-b]pyridin-4-yl)-2-methylpropane-2-sulfinamide (155 mg, 0.55 mmol, 1 equiv) in THF (5.5 mL) at 0° C. was added 4M HCl in 1,4-dioxane (2.74 mL, 10.98 mmol, 20 equiv). The resulting mixture was allowed to warm to room temperature and stirred overnight. The mixture was concentrated, and diethyl ether was added (2 mL). The resulting suspension was filtered, and the solid was dried under vacuum to provide the title compound (136 mg, 0.54 mmol, 98% yield). LCMS (ESI): m/z=179.2 [M+H]⁺.

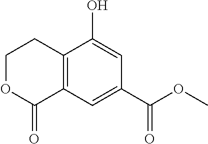 [SMILES: COC(=O)c1cc(O)c2c(c1)C(=O)OCC2]

### Intermediate 17

### Methyl 5-hydroxy-1-oxoisochromane-7-carboxylate

Dimethyl 2-hydroxy-2,3-dihydrobenzofuran-4,6-dicarboxylate (110 mg, 0.436 mmol, 1 equiv) was dissolved in EtOH (4 mL) and then NaBH₄ (20 mg, 0.523 mmol, 1.2 equiv) was added portionwise at 25° C. After completion, the reaction was concentrated under reduced pressure. The reaction mixture was diluted with EtOAc and sat. NH₄Cl and extracted with EtOAc. The organic layers were dried over MgSO₄ and concentrated under reduced pressure. The title compound (105 mg, 0.473 mmol, quant.) was used without further purification. ¹H NMR (400 MHz, DMSO-d₆) δ 10.53 (s, 1H), 7.97 (d, J=1.2 Hz, 1H), 7.65 (d, J=1.4 Hz, 1H), 4.50 (t, J=6.1 Hz, 2H), 3.86 (s, 3H), 2.97 (t, J=6.0 Hz, 2H); LCMS (ESI): Method 2: R_{T}=1.460 min, m/z=223.0 [M+H]⁺.

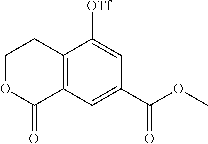 [SMILES: COC(=O)c1cc(OS(=O)(=O)C(F)(F)F)c2c(c1)C(=O)OCC2]

### Intermediate 18

### Methyl 1-oxo-5-(((trifluoromethyl)sulfonyl)oxy)isochromane-7-carboxylate

Phenyl triflimide (209 mg, 0.58 mmol, 1.3 equiv) was added to a solution of methyl 5-hydroxy-1-oxoisochromane-7-carboxylate (Intermediate 17, 100 mg, 0.45 mmol, 1 equiv) and N,N-diisopropylethylamine (0.2 mL, 1.35 mmol, 3 equiv) in THF:CH₂Cl₂ (5:1, 4.8 mL) at 23° C. and stirred for 14 h. Saturated aqueous NaHCO₃ was added and the mixture was extracted with CH₂Cl₂ (3×20 mL). The combined organic phases were dried over MgSO₄ and concentrated under reduced pressure. The residue was purified by flash chromatography (Combi-flash Rf, Hex/EtOAc=0-100%, gradient) to afford the title compound (136 mg, 0.38 mmol, 86% yield). ¹H NMR (400 MHz, Chloroform-d) δ 8.81 (s, 1H), 8.16 (s, 1H), 4.60 (t, J=6.0 Hz, 2H), 3.98 (s, 3H), 3.21 (t, J=6.0 Hz, 2H); LCMS (ESI): Method 2: R_{T}=1.724 min, m/z=355.0 [M+H]⁺.

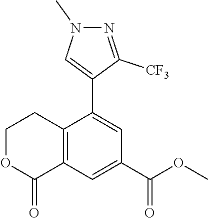 [SMILES: COC(=O)c1cc2c(c(-c3cn(C)nc3C(F)(F)F)c1)CCOC2=O]

### Intermediate 19

### Methyl 5-(1-methyl-3-(trifluoromethyl)-1H-pyrazol-4-yl)-1-oxoisochromane-7-carboxylate

Methyl 1-oxo-5-(((trifluoromethyl)sulfonyl)oxy)isochromane-7-carboxylate (Intermediate 18, 18.5 g, 52.2 mmol, 1 equiv), (1-methyl-3-(trifluoromethyl)-1H-pyrazol-4-yl)boronic acid (15.2 g, 78.3 mmol, 1.5 equiv), potassium carbonate (18.0 g, 131 mmol, 2.5 equiv), and PdCl₂(dppf) (1.91 g, 2.6 mmol, 0.05 equiv) were dissolved in 1,4-dioxane:water (4:1, 150 mL) under an argon atmosphere in a sealed tube. The reaction mixture was stirred for 1 h at 90° C. then cooled to 23° C. Brine was added to the mixture, and then the mixture was extracted with EtOAc (3×50 mL). The combined organic layers were dried over MgSO₄ and concentrated under reduced pressure. The residue was purified by flash chromatography (Combi-flash Rf, Hex/EtOAc=0-100%, gradient) to afford the title compound (16.0 g, 45 mmol, 86% yield). ¹H NMR (400 MHz, Chloroform-d) δ 8.80 (d, J=1.5 Hz, 1H), 8.14 (d, J=1.6 Hz, 1H), 7.42 (s, 1H), 4.48 (t, J=5.9 Hz, 2H), 4.04 (s, 3H), 3.95 (s, 3H), 2.90 (t, J=5.9 Hz, 2H); LCMS (ESI): Method 2: R_{T}=1.588 min, m/z=355.0 [M+H]⁺.

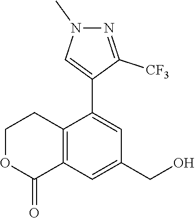 [SMILES: Cn1cc(-c2cc(CO)cc3c2CCOC3=O)c(C(F)(F)F)n1]

### Intermediate 20

### 7-(Hydroxymethyl)-5-(1-methyl-3-(trifluoromethyl)-1H-pyrazol-4-yl)isochroman-1-one

Step A: Preparation of 5-(1-methyl-3-(trifluoromethyl)-1H-pyrazol-4-yl)-1-oxoisochromane-7-carboxylic acid. Methyl 5-(1-methyl-3-(trifluoromethyl)-1H-pyrazol-4-yl)-1-oxoisochromane-7-carboxylate (Intermediate 19, 1.027 g, 2.90 mmol, 1 equiv) was dissolved in THF:water (3:1, 26.7 mL) at room temperature. Then lithium hydroxide (139 mg, 5.8 mmol, 2 equiv) was added and the reaction was stirred overnight. LCMS detected hydrolysis of the lactone and 2 M HCl was added to until pH<2 and stirred for 2 hours until the lactone was detected by LCMS. The mixture pH was adjusted to a pH of 4-5 with sat. NaHCO₃, and the reaction mixture was extracted with CH₂Cl₂ (3×30 mL). The organic layers were dried over MgSO₄ and concentrated under reduced pressure to yield the title compound (0.972 g, 2.86 mmol, 99% yield). ¹H NMR (400 MHz, DMSO-dc) δ 8.49 (d, J=1.6 Hz, 1H), 8.12 (s, 1H), 8.02 (d, J=1.4 Hz, 1H), 4.47 (t, J=5.9 Hz, 2H), 3.99 (s, 3H), 2.93 (t, J=5.9 Hz, 2H); LCMS (ESI): Method 2: R_{T}=1.354 min, m/z=341.0 [M+H]⁺.

Step B: Preparation of 7-(Hydroxymethyl)-5-(1-methyl-3-(trifluoromethyl)-1H-pyrazol-4-yl)isochroman-1-one. 5-(1-Methyl-3-(trifluoromethyl)-1H-pyrazol-4-yl)-1-oxoisochromane-7-carboxylic acid (855.3 mg, 1 equiv, 2.514 mmol) was dissolved in CH₂Cl₂ (10 mL) and CDI (489.1 mg, 1.2 equiv, 3.016 mmol) was added at 25° C. The slurry dissolved almost immediately and the reaction was stirred for 3 h. The reaction was concentrated under reduced pressure then redissolved in THF (8 mL) and cooled to 0° C. NaBH₄ (190.2 mg, 2 equiv, 5.0 mmol) was added followed by water (2 mL) dropwise. The reaction was quenched by addition of MeOH (1 mL) then extracted with CH₂Cl₂. The organic layer was washed with sat. NH₄CL, dried over MgSO₄ and concentrated under reduced pressure. The residue was purified by flash chromatography (Combi-flash Rf, Hex/EtOAc=0-100% gradient, then DCM/MeOH=0-10% gradient) to afford the title compound (580.2 mg, 1.78 mmol, 71% yield). ¹H NMR (400 MHz, DMSO-d₆) δ 8.05 (s, 1H), 7.96 (s, 1H), 7.47 (s, 1H), 5.39 (t, J=5.7 Hz, 1H), 4.56 (d, J=5.5 Hz, 2H), 4.42 (t, J=5.9 Hz, 2H), 3.98 (s, 3H), 2.81 (t, J=5.9, 2H); LCMS (ES1): Method 2: R_{T}=1.374 min, m/z=327.1 [M+H]⁺.

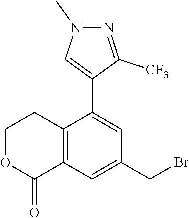 [SMILES: Cn1cc(-c2cc(CBr)cc3c2CCOC3=O)c(C(F)(F)F)n1]

### Intermediate 21

### 7-(Bromomethyl)-5-(1-methyl-3-(trifluoromethyl)-1H-pyrazol-4-yl)isochroman-1-one

PBr₃ (5.2 mL, 55 mmol, 2 equiv) was added to a solution of 7-(hydroxymethyl)-5-(1-methyl-3-(trifluoromethyl)-1H-pyrazol-4-yl)isochroman-1-one (Intermediate 20, 9.0 g, 28 mmol, 1 equiv) in CH₂Cl₂ (100 mL) at 0° C. The reaction was warmed to room temperature and stirred overnight. Sat. aq. NaHCO₃ was added, and the mixture was extracted with EtOAc. The combined organic layers were dried over MgSO₄ and concentrated under reduced pressure. The residue was purified by flash chromatography (Combi-flash Rf, Hex/EtOAc=0-100% gradient) to afford the title compound (7.57 g, 19.5 mmol, 71% yield). ¹H NMR (400 MHz, Chloroform-d) δ 8.17 (d, J=1.9 Hz, 1H), 7.51 (d, J=1.9 Hz, 1H), 7.42 (s, 1H), 4.50 (s, 2H), 4.45 (t, J=6.0 Hz, 2H), 4.03 (s, 3H), 2.84 (t, J=6.0 Hz, 2H); LCMS (ESI): Method 2: R_{T}=1.983 min, m/z=388.9 [M+H]⁺.

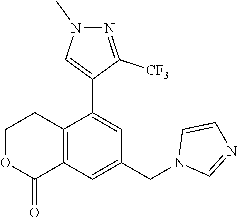 [SMILES: Cn1cc(-c2cc(Cn3ccnc3)cc3c2CCOC3=O)c(C(F)(F)F)n1]

### Intermediate 22

### 7-((1H-Imidazol-1-yl)methyl)-5-(1-methyl-3-(trifluoromethyl)-1H-pyrazol-4-yl)isochroman-1-one

Imidazole (3.97 g, 58.4 mmol, 3 equiv) was added to a solution of 7-(bromomethyl)-5-(1-methyl-3-(trifluoromethyl)-1H-pyrazol-4-yl)isochroman-1-one (Intermediate 21, 7.57 g, 19.5 mmol, 1 equiv) in acetonitrile (100 mL) at 23° C. The reaction mixture was stirred at 50° C., then cooled to ambient temperature, filtered, and concentrated. The residue was extracted with CH₂Cl₂. The combined organic layers were then washed with sat. aq. NH₄Cl, dried over MgSO₄, and concentrated under reduced pressure. The residue was purified by flash chromatography (Combi-flash Rf, Hex/EtOAc=0-100% gradient, then DCM/MeOH=0-10% gradient) to afford the title compound (5.83 g, 15.5 mmol, 80% yield). ¹H NMR (400 MHz, Chloroform-d) δ 8.05 (d, J=1.6 Hz, 1H), 7.55 (s, 1H), 7.35 (s, 1H), 7.17 (d, J=1.7 Hz, 1H), 7.10 (s, 1H), 6.91 (s, 1H), 5.17 (s, 2H), 4.45 (t, J=6.0 Hz, 2H), 4.01 (s, 3H), 2.84 (t, J=6.0 Hz, 2H); LCMS (ESI): Method 2: R_{T}=1.167 min, m/z=377.0 [M+H]⁺.

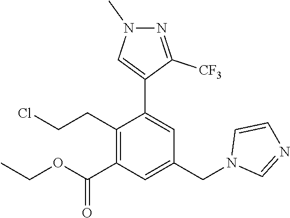 [SMILES: CCOC(=O)c1cc(Cn2ccnc2)cc(-c2cn(C)nc2C(F)(F)F)c1CCCl]

### Intermediate 23

### Ethyl 5-((1H-imidazol-1-yl)methyl)-2-(2-chloroethyl)-3-(1-methyl-3-(trifluoromethyl)-1H-pyrazol-4-yl)benzoate

To a solution of 7-((1H-imidazol-1-yl)methyl)-5-(1-methyl-3-(trifluoromethyl)-1H-pyrazol-4-yl)isochroman-1-one (Intermediate 22,254 mg, 0.68 mmol, 1 equiv) in EtOH (3.5 mL) at room temperature was added SOCl₂ (790 μL, 10.8 mmol, 16 equiv). The resulting mixture was stirred in a sealed tube at room temperature for 24 h then quenched with saturated aqueous NaHCO₃ solution. The mixture was extracted with EtOAc. The combined organic layers were dried over Na₂SO₄, filtered, and concentrated. The residue was purified by silica gel column chromatography (Combi-flash Rf, DCM/MeOH=0-10% gradient) to provide the title compound (293 mg, 0.67 mmol, 98% yield). ¹H NMR (400 MHz, Chloroform-d) δ 7.75 (d, J=2.0 Hz, 1H), 7.56 (s, 1H), 7.39 (s, 1H), 7.11 (s, 1H), 7.09 (d, J=2.0 Hz, 1H), 6.90 (m, 1H), 5.14 (s, 2H), 4.40 (q, J=7.2 Hz, 2H), 4.03 (s, 3H), 3.54 (t, J=8.0 Hz, 2H), 3.30 (t, J=8.0 Hz, 2H), 1.42 (t, J=7.2 Hz, 3H).

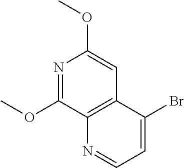

### Intermediate 24

### 4-Bromo-6,8-dimethoxy-1,7-naphthyridine

Step A. Preparation of 5-(((2,6-dimethoxypyridin-3-yl)amino)methylene)-2,2-dimethyl-1,3-dioxane-4,6-dione. The title compound (8.48 g, 27.5 mmol, 85% yield) was prepared following the procedure described for Intermediate 15 Step B, substituting 2,6-dimethoxypyridin-3-amine (5.00 g, 32.4 mmol, 1 equiv). ¹H NMR (400 MHz, Chloroform-d) δ 11.28 (d, J=14.2 Hz, 1H), 8.52 (d, J=14.6 Hz, 1H), 7.52 (d, J=8.5 Hz, 1H), 6.39 (d, J=8.4 Hz, 1H), 4.06 (s, 3H), 3.94 (s, 3H), 1.75 (s, 6H).

Step B. Preparation of 6,8-dimethoxy-1,7-naphthyridin-4-ol. The title compound (412 mg, 0.100 mmol, 4% yield) was prepared following the procedure described for Intermediate 15 Step C, substituting 5-(((2,6-dimethoxypyridin-3-yl)amino)methylene)-2,2-dimethyl-1,3-dioxane-4,6-dione (8.48 g, 27.5 mmol, 1 equiv). LCMS (ESI): Method 2: R_{T}=1.062 min, m/z=207.1 [M+H]⁺.

Step C. Preparation of 4-bromo-6,8-dimethoxy-1,7-naphthyridine. To a solution of 6,8-dimethoxy-1,7-naphthyridin-4-ol (211 mg, 1.02 mmol, 1 equiv) in DMF (4 mL) was added PBr₃ (0.19 mL, 2.05 mmol, 2 equiv) dropwise at 0° C. The reaction mixture was warmed to room temperature and stirred overnight. The reaction was quenched with ice and brought to pH 7-9 with sat. aq. NaHCO₃. The mixture was extracted with EtOAc. The combined organic layers were dried over MgSO₄, concentrated under reduced pressure. The residue was purified by flash chromatography (Combi-flash Rf, Hex/EtOAc=0-100% gradient) to afford the title compound (152 mg, 0.566 mmol, 55% yield). ¹H NMR (400 MHz, Chloroform-d) δ 8.51 (d, J=4.6 Hz, 1H), 7.78 (d, J=4.6 Hz, 1H), 6.79 (s, 1H), 4.24 (s, 3H), 4.04 (s, 3H); LCMS (ESI): Method 2: R_{T}=1.682 min, m/z=269.0 [M+H]⁺.

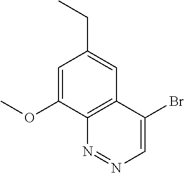

### Intermediate 25

### 4-Bromo-6-ethyl-8-methoxycinnoline

Step A. Preparation of 1-(2-amino-5-ethyl-3-methoxyphenyl)ethan-1-one. A mixture of 1-(2-amino-5-bromo-3-methoxyphenyl)ethan-1-one (527 mg, 2.16 mmol, 1 equiv), triethylborane (4.3 mL, 4.3 mmol, 2 equiv, 1 M THF), cesium carbonate (1.41 g, 4.32 mmol, 2 equiv), and Pd(dppf)Cl₂ (79 mg, 108 μmol, 0.05 equiv) in THF (10 mL) was stirred for 3 h at 60° C. under Ar in a sealed tube. The reaction was cooled to 23° C. and quenched by addition of 1:1 (v/v) AcOH:water solution. The mixture was extracted with EtOAc (3×30 mL). The combined organic layers were dried over MgSO₄ and concentrated under reduced pressure. The residue was purified by flash chromatography (Combi-flash Rf, Hex/EtOAc=0-100% gradient, then DCM/MeOH=0-10% gradient) to afford the title compound (366 mg, 1.89 mmol, 88% yield). ¹H NMR (400 MHz, Chloroform-d) δ 7.13 (d, J=1.5 Hz, 1H), 6.72 (d, J=1.5 Hz, 1H), 3.87 (s, 3H), 2.57 (q, J=7.6 Hz, 2H), 2.56 (s, 3H), 1.23 (t, J=7.6 Hz, 3H); LCMS (ESI): Method 2: R^{T}=0.643 min, m/z=194.2 [M+H]⁺.

Step B. Preparation of 6-ethyl-8-methoxycinnolin-4-ol. 1-(2-Amino-5-ethyl-3-methoxyphenyl)ethan-1-one (352 mg, 1.82 mmol, 1 equiv) was taken up in conc. HCl (5 mL) and stirred at 0° C. A solution of sodium nitrite (188 mg, 2.73 mmol, 1.5 equiv) in water (0.5 mL) was added dropwise at 0° C. The reaction was stirred for 1 h at 0° C. and allowed to warm to room temperature. The pH was adjusted to pH 7-9 by addition of solid NaHCO₃. The reaction mixture was extracted with CH₂Cl₂ (3×20 mL). The combined organic layers were dried over MgSO₄ and concentrated under reduced pressure. The residue was purified by flash chromatography (Combi-flash Rf, Hex/EtOAc=0-100% gradient, then DCM/MeOH=0-10% gradient) to afford the title compound (271 mg, 1.33 mmol, 73% yield). ¹H NMR (400 MHz, Chloroform-d) δ 10.35 (brs, 1H), 7.84 (s, 1H), 7.63 (s, 1H), 6.94 (d, J=1.4 Hz, 1H), 4.03 (s, 3H), 2.75 (q, J=7.6 Hz, 2H), 1.30 (t, J=7.6 Hz, 3H); LCMS (ESI): Method 2: R_{T}=1.135 min, m/z=205.2 [M+H]⁺.

Step C. Preparation of 4-Bromo-6-ethyl-8-methoxycinnoline. Phosphorus oxybromide (1.14 g, 3.98 mmol, 3 equiv) and 6-ethyl-8-methoxycinnolin-4-ol (271 mg, 1.33 mmol, 1 equiv) were dissolved in acetonitrile (10 mL). The reaction mixture was stirred at 60° C. until completion. The reaction was quenched with ice and brought to pH 7-9 with sat. aq. NaHCO₃. The mixture was extracted with EtOAc. The combined organic layers were dried over MgSO₄, concentrated under reduced pressure. The residue was purified by flash chromatography (Combi-flash Rf, Hex/EtOAc=0-100% gradient, then DCM/MeOH=0-10% gradient) to afford the title compound (119 mg, 0.445 mmol, 34% yield). ¹H NMR (400 MHz, Chloroform-d) δ 9.44 (s, 1H), 7.44 (s, 1H), 7.03 (d, J=1.2 Hz, 1H), 4.18 (s, 3H), 2.90 (q, J=7.5 Hz, 2H), 1.39 (t, J=7.6 Hz, 3H); LCMS (ESI): Method 2: R_{T}=1.696 min, m/z=267.0 [M+H]⁺.

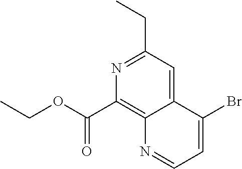 [SMILES: CCOC(=O)c1nc(CC)cc2c(Br)ccnc12]

### Intermediate 26

### Ethyl 4-bromo-6-ethyl-1,7-naphthyridine-8-carboxylate

Step A. Preparation of ethyl 3-amino-6-ethylpicolinate. The title compound (2 g, quant.) was prepared following the procedure described for Intermediate 25 Step A, substituting ethyl 3-amino-6-bromopicolinate (2.5 g, 10.2 mmol, 1 equiv). ¹H NMR (400 MHz, Chloroform-d) δ 7.13 (d, J=8.3 Hz, 1H), 7.01 (d, J=8.4 Hz, 1H), 5.60 (brs, 2H), 4.44 (q, J=7.1 Hz, 2H), 2.78 (q, J=7.6 Hz, 2H), 1.44 (t, J=7.1 Hz, 3H), 1.26 (t, J=7.5 Hz, 3H).

Step B. Preparation of ethyl 3-(((2,2-dimethyl-4,6-dioxo-1,3-dioxan-5-ylidene)methyl)amino)-6-ethylpicolinate. The title compound (2.44 g, 7.01 mmol, 62% yield) was prepared following the procedure described for Intermediate 15, Step B, substituting ethyl 3-amino-6-ethylpicolinate (2.2 g, 11.3 mmol, 1 equiv). ¹H NMR (400 MHz, Chloroform-d) δ 8.67 (d, J=14.1 Hz, 1H), 7.81 (d, J=8.7 Hz, 1H), 7.46 (d, J=8.6 Hz, 1H), 4.60 (q, J=7.1 Hz, 2H), 2.94 (q, J=7.6 Hz, 2H), 1.76 (s, 6H), 1.48 (t, J=7.1 Hz, 3H), 1.35 (t, J=7.6 Hz, 3H), LCMS (ESI): Method 2: R_{T}=1.833 min, m/z=349.0 [M+H]⁺.

Step C. Preparation of ethyl 6-ethyl-4-hydroxy-1,7-naphthyridine-8-carboxylate. The title compound (641 mg, 2.6 mmol, 37% yield) was prepared following the procedure described for Intermediate 15 Step C, substituting ethyl 3-(((2,2-dimethyl-4,6-dioxo-1,3-dioxan-5-ylidene)methyl)amino)-6-ethylpicolinate (2.44 g, 7.01 mmol, 1 equiv). LCMS (ESI); Method 2: R_{T}=1.129 min, m/z=247.2 [M+H]⁺.

Step D. Preparation of ethyl 4-bromo-6-ethyl-1,7-naphthyridine-8-carboxylate. The title compound (101 mg, 0.327 mmol, 13% yield) was prepared following the procedure described for Intermediate 24 Step C, substituting ethyl 6-ethyl-4-hydroxy-1,7-naphthyridine-8-carboxylate (641 mg, 2.6 mmol, 1 equiv). ¹H NMR (400 MHz, Chloroform-d) δ 8.76 (d, J=4.6 Hz, 1H), 7.87-7.86 (m, 2H), 4.61 (q, J=7.2 Hz, 2H), 3.09 (q, J=7.6 Hz, 2H), 1.47 (t, J=7.2 Hz, 3H), 1.43 (t, J=7.6 Hz, 3H); LCMS (ESI): Method 2: R_{T}=1.609 min, m/z=309.0 [M+H]⁺.

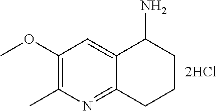

### Intermediate 27

### 3-Methoxy-2-methyl-5,6,7,8-tetrahydroquinolin-5-amine dihydrochloride salt

Step A. Preparation of 7,8-dihydro-6H-spiro[quinoline-5,2′-[1,3]dioxolane]. To a solution of 7,8-dihydroquinolin-5(6H)-one (4.1 g, 28 mmol, 1 equiv) and ethane-1,2-diol (112 mmol, 4 equiv) in toluene (60 mL) was added p-TSA (6.59 g, 34.7 mmol, 1.25 equiv). The resulting mixture was refluxed with a Dean Stark trap for 24 h. Toluene was removed under reduced pressure and the residue was taken up in EtOAc and washed with 1M NaOH. The organic layer was dried over Na₂SO₄, filtered, and concentrated. The residue was purified by silica gel column chromatography (Combi-flash Rf, Hex/EtOAc=0-50% gradient) to provide the title compound (4.9 g, 26 mmol, 91% yield). ¹H NMR (400 MHz, Chloroform-d) δ 8.49 (dd, J=1.2, 4.8 Hz, 1H), 7.79 (dd, J=2.0, 8.0 Hz, 1H), 7.16 (dd, J=4.8, 8.0 Hz, 1H), 4.17 (m, 4H), 2.97 (t, J=6.4 Hz, 2H), 2.04 (m, 2H), 1.98 (m, 2H).

Step B. Preparation of 7,8-dihydro-6H-spiro[quinoline-5,2′-[1,3]dioxolan]-3-ol. To a solution of 7,8-dihydro-6H-spiro[quinoline-5,2′-[1,3]dioxolane] (3.76 g, 19.7 mmol, 1 equiv) in THF (37 mL) were added 4,4,4′,4′,5,5,5′,5′-octamethyl-2,2′-bi(1,3,2-dioxaborolane) (7.99 g, 31.5 mmol, 1.6 equiv), 4,4′-di-tert-butyl-2,2′-bipyridine (496 mg, 1.85 mmol, 0.094 equiv), and (1,5-cyclooctadiene)(methoxy)iridium(I) dimer (613 mg, 0.92 mmol, 0.047 equiv). The resulting mixture was stirred in a sealed tube at 78° C. for 20 h then cooled to 0° C. The reaction was quenched with MeOH and concentrated. The residue was taken up in dichloromethane (100 mL), cooled to 0° C., and 30% hydrogen peroxide (12.9 mL, 126 mmol, 6.4 equiv) was added. The mixture was warmed to room temperature and stirred for 16 h. The reaction was quenched with sat. aq. Na₂SO₃. The resulting mixture was extracted with dichloromethane. The combined organic layers were dried over Na₂SO₄, filtered, and concentrated. The residue was purified by silica gel column chromatography (Combi-flash Rf, Hex/EtOAc=50-100% gradient) to provide the title compound (3.01 g, 14.5 mmol, 74% yield). ¹H NMR (400 MHz, Chloroform-d) δ 8.19 (d, J=3.2 Hz, 1H), 7.32 (d, J=2.8 Hz, 1H), 4.16 (m 4H), 2.90 (t, J=6.4 Hz, 2H), 2.03 (m, 2H), 1.92 (m, 2H).

Step C. Preparation of 2-iodo-7,8-dihydro-6H-spiro[quinoline-5,2′-[1,3]dioxolan]-3-ol. To a suspension of 7,8-dihydro-6H-spiro[quinoline-5,2′-[1,3]dioxolan]-3-ol (690 mg, 3.3 mmol, 1 equiv) in water (49 mL) was added Na₂CO₃ (706 mg, 6.7 mmol, 2 equiv). The resulting mixture was stirred at room temperature for 30 min to form a homogeneous solution. Once a homogeneous solution was achieved, iodine (845.2 mg 3.33 mmol, 1 equiv) was added. The resulting mixture was stirred at room temperature for 16 h then quenched with 4M HCl. The mixture was basified with sat. aq. NaHCO₃ and extracted with EtOAc. The combined organic layers were dried (Na₂SO₄), filtered, and concentrated. The residue was purified by silica gel column chromatography (Combi-flash Rf, Hex/EtOAc=0-100% gradient) to provide the title compound (985 mg, 2.95 mmol, 88% yield). ¹H NMR (400 MHz, Chloroform-d) δ 7.15 (s, 1H), 4.14 (m, 4H), 2.80 (t, J=6.4 Hz, 2H), 1.97 (m, 4H).

Step D. Preparation of 2-iodo-3-methoxy-7,8-dihydro-6H-spiro[quinoline-5,2′-[1,3]dioxolane]. To a solution of 2-iodo-7,8-dihydro-6H-spiro[quinoline-5,2′-[1,3]dioxolan]-3-ol (406 mg, 1.22 mmol, 1 equiv) in THE (8 mL) at 0° C. was added 1M NaHMDS (1.6 mL, 1.6 mmol, 1.3 equiv). The ice bath was removed, and the mixture was stirred at room temperature for 50 min then concentrated under reduced pressure. The residue was dissolved in DMF (8 mL) and iodomethane (85 μL, 1.4 mmol, 1.1 equiv) was added. The resulting mixture was stirred at room temperature overnight then concentrated under reduced pressure. The residue was taken up in chloroform and washed with brine/water. The organic layer was dried over Na₂SO₄, filtered, and concentrated. The residue was purified by silica gel column chromatography (Combi-flash Rf, Hex/EtOAc=30-100% gradient) to provide the title compound (370 mg, 1.07 mmol, 87% yield). ¹H NMR (400 MHz, Chloroform-d) ¹H NMR (400 MHz, Chloroform-d) δ 7.06 (s, 1H), 4.15 (m, 4H), 2.91 (t, J=6.4 Hz, 2H), 1.95 (m, 4H).

Step E. Preparation of 3-methoxy-2-methyl-7,8-dihydro-6H-spiro[quinoline-5,2′-[1,3]dioxolane]. To a solution of 2-iodo-3-methoxy-7,8-dihydro-6H-spiro[quinoline-5,2′-[1,3]dioxolane] (145.7 mg, 0.42 mmol, 1 equiv) in THF (4 mL) at −78° C. was added 1.7M 1-BuLi (519 μL, 0.88 mmol, 2.1 equiv). The mixture was stirred for 15 min then iodomethane (53 μL, 0.84 mmol, 2 equiv) was added. The reaction mixture was warmed to 0° C. and stirred for 45 min then quenched with sat. aq. NH₄Cl solution. The resulting mixture was extracted with EtOAc. The combined organic layers were dried over Na₂SO₄, filtered, and concentrated. The residue was purified by silica gel column chromatography (Combi-flash Rf, Hex/EtOAc=0-50% gradient) to provide the title compound (79 mg, 0.33 mmol, 78% yield). ¹H NMR (400 MHz, Chloroform-d) δ 7.16 (s, 1H), 4.15 (m, 4H), 3.84 (s, 3H), 2.86 (t, J=6.4 Hz, 2H), 2.44 (s, 3H), 1.99 (m, 2H), 1.94 (m, 2H).

Step F. Preparation of 3-methoxy-2-methyl-7,8-dihydroquinolin-5(6H)-one. To a solution of 3-methoxy-2-methyl-7,8-dihydro-6H-spiro[quinoline-5,2′-[1,3]dioxolane] (135 mg, 574 μmol, 1 equiv) in acetone (1.5 mL) was added 2M HCl (1.5 mL, 30 mmol, 5 equiv). The mixture was stirred at 45° C. for 16 h, then neutralized with sat. aq. NaHCO₃ and extracted with chloroform. The combined organic layers were dried over Na₂SO₄, filtered, and concentrated. The residue was purified by silica gel column chromatography (Combi-flash Rf, Hex/EtOAc=0-100% gradient) to provide the title compound (104.6 mg, 547 μmol, 95% yield). LCMS (ESI): m/z=192.2 [M+H]⁺.

Step G. Preparation of 3-methoxy-2-methyl-5,6,7,8-tetrahydroquinolin-5-ol. To a solution of 3-methoxy-2-methyl-7,8-dihydroquinolin-5(6H)-one (62 mg, 0.32 mmol, 1 equiv) in THF (3 mL) at 0° C. was added 1M L-selectride (973 μL, 0.97 mmol, 3 equiv). The mixture was warmed to room temperature and stirred for 16 h. The reaction was quenched with sat. aq. NaHCO₃, and the resulting mixture was extracted with EtOAc. The combined organic layers were dried over Na₂SO₄, filtered, and concentrated. The residue was purified by silica gel column chromatography (Combi-flash Rf, DCM/MeOH=0-10% gradient) to provide the title compound (60.9 mg, 0.32 mmol, 97% yield). LCMS (ESI): m/z=194.3 [M+H]⁺.

Step H. Preparation of 5-azido-3-methoxy-2-methyl-5,6,7,8-tetrahydroquinoline. To a solution of 3-methoxy-2-methyl-5,6,7,8-tetrahydroquinolin-5-ol (60.9 mg, 0.32 mmol, 1 equiv) in THF (4 mL) at room temperature were added DBU (71 μL, 0.47 mmol, 1.5 equiv) and diphenylphosphoryl azide (102 μL, 0.47 mmol, 1.5 equiv). The reaction mixture was stirred at room temperature overnight then quenched with water. The resulting mixture was extracted with EtOAc. The combined organic layers were dried over Na₂SO₄, filtered, and concentrated. The residue was purified by silica gel column chromatography (Combi-flash Rf, Hex/EtOAc=0-60% gradient) to provide the title compound (69 mg, 0.32 mmol, 100% yield). LCMS (ESI): m/z=219.2 [M+H]⁺.

Step 1. Preparation of tert-butyl (3-methoxy-2-methyl-5,6,7,8-tetrahydroquinolin-5-yl)carbamate. To a solution of 5-azido-3-methoxy-2-methyl-5,6,7,8-tetrahydroquinoline (69 mg, 0.32 mmol, 1 equiv) in THF (3.5 mL) were added PPh₃ (116 mg, 0.44 mmol, 1.4 equiv) and water (0.35 mL). The mixture was stirred in a sealed tube at 50° C. overnight. Sat. aq. NaHCO₃ solution (2.5 mL) and (Boc)₂₀ (89.7 mg, 0.41 mmol, 1.3 equiv) were added. The resulting mixture was stirred at room temperature for 5 h then extracted with EtOAc. The combined organic layers were dried (Na₂SO₄), filtered, and concentrated. The residue was purified by silica gel column chromatography (Combi-flash Rf, Hex/EtOAc=0-50% gradient) to provide the desired product (80 mg, 0.27 mmol, 87% yield). LCMS (ESI): m/z=293.2 [M+H]⁺.

Step J. Preparation of 3-methoxy-2-methyl-5,6,7,8-tetrahydroquinolin-5-amine dihydrochloride salt. To a solution of tert-butyl (3-methoxy-2-methyl-5,6,7,8-tetrahydroquinolin-5-yl)carbamate (80 mg, 0.27 mmol, 1 equiv) in DCM (1 mL) at 0° C. was added 4M HCl in 1,4-dioxane (1.71 mL, 6.84 mmol, 25 equiv). The mixture was warmed to room temperature, stirred for 48 h, and then concentrated. The residue was washed with Et₂O and dried under vacuum to provide the title compound (73 mg, 0.27 mmol, 100% yield); LCMS (ESI): m/z=193.2 [M+H]⁺.

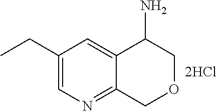

### Intermediate 28

### 3-Ethyl-5,8-dihydro-6H-pyrano[3,4-b]pyridin-5-amine dihydrochloride salt

Step A. Preparation of 3-ethyl-6H-pyrano[3,4-b]pyridin-5(8H)-one. To a solution of 2H-pyran-3,5(4H,6H)-dione (308 mg, 2.7 mmol, 1 equiv) in toluene (5 mL) were added 2-ethylacrolein (343 μL, 3.5 mmol, 1.3 equiv), NH₄OAc (416 mg, 5.4 mmol, 2 equiv), 4 Å molecular sieves (0.5 g), and acetic acid (1.2 mL). The resulting mixture was stirred in a sealed tube at 115° C. for 16 h. The mixture was cooled to ambient temperature, diluted with EtOAc and filtered. The filtrate was washed with sat. aq. NaHCO₃, dried over Na₂SO₄, filtered, and concentrated. The residue was purified by silica gel column chromatography (Combi-flash Rf, Hex/EtOAc=0-100% gradient) to provide the title compound (194 mg, 1.1 mmol, 40% yield). ¹H NMR (400 MHz, Chloroform-d) δ 8.59 (d, J=2.4 Hz, 1H), 8.12 (d, J=2.4 Hz, 1H), 4.95 (s, 2H), 4.39 (s, 2H), 2.74 (q, J=7.6 Hz, 2H), 1.29 (t, J=7.6 Hz, 3H).

Step B. Preparation of 3-ethyl-5,8-dihydro-6H-pyrano[3,4-b]pyridin-5-ol. To a solution of 3-ethyl-6H-pyrano[3,4-b]pyridin-5(8H)-one (172 mg, 1.0 mmol, 1 equiv) in EtOH (5 mL) at 0° C. was added NaBH₄ (80 mg, 2.1 mmol, 2.1 equiv). The resulting mixture was warmed to room temperature and stirred for 1.5 h. The solvent (EtOH) was removed under reduced pressure, and the residue was taken up in EtOAc and washed with sat. aq. NaHCO₃ and brine. The organic layer was dried over Na₂SO₄, filtered, and concentrated. The residue was purified by silica gel column chromatography (Combi-flash Rf, Hex/EtOAc=0-60% gradient) to provide the title compound (66 mg, 368 μmol, 38% yield); LCMS (ESI): m/z=180.2 [M+H]⁺.

Step C. Preparation of 5-azido-3-ethyl-5,8-dihydro-6H-pyrano[3,4-b]pyridine. To a solution of 3-ethyl-5,8-dihydro-6H-pyrano[3,4-b]pyridin-5-ol (66 mg, 368 μmol, 1 equiv) in THF (4 mL) at room temperature was added DBU (86 μL, 0.56 mmol, 1.5 equiv) and diphenylphosphoryl azide (130 μL, 0.56 mmol, 1.5 equiv). The reaction was stirred at room temperature overnight, and then quenched with water. The resulting mixture was extracted with EtOAc. The combined organic layers were dried over Na₂SO₄, filtered, and concentrated. The residue was purified by silica gel column chromatography (Combi-flash Rf, Hex/EtOAc=0-60% gradient) to provide the title compound (70 mg, 340 μmol, 93% yield). LCMS (ESI): m/z=205.2 [M+H]⁺.

Step D. Preparation of 3-ethyl-5,8-dihydro-6H-pyrano[3,4-b]pyridin-5-amine dihydrochloride salt. The title compound (101 mg, 0.327 mmol, 13% yield) was prepared following the procedure described for Intermediate 27 Steps I and J, substituting 5-azido-3-ethyl-5,8-dihydro-6H-pyrano[3,4-b]pyridine (641 mg, 2.6 mmol, 1 equiv). ¹H NMR (400 MHz, Chloroform-d) δ 8.76 (d, J=4.6 Hz, 1H), 7.87-7.86 (m, 2H), 4.61 (q, J=7.2 Hz, 2H), 3.09 (q, J=7.6 Hz, 2H), 1.47 (t, J=7.2 Hz, 3H), 1.43 (t, J=7.6 Hz, 3H); LCMS (ESI): Method 2: R_{T}=1.609 min, m/z=309.0 [M+H]⁺.

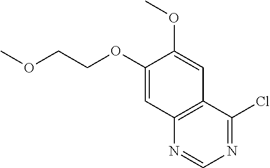 [SMILES: COCCOc1cc2ncnc(Cl)c2cc1OC]

### Intermediate 29

### 4-Chloro-6-methoxy-7-(2-methoxyethoxy)quinazoline

To a stirred suspension of triphenylphosphine (polymer bound, 3 mmol/g) (560 mg, 2.14 mmol, 3 equiv) and 2-methoxyethan-1-ol (0.06 mL, 0.71 mmol, 1 equiv) in CH₂Cl₂ (5 mL/g of resin (3.5 mL) at 0° C., was added DIAD (0.4 mL, 2.14 mmol, 3 equiv) followed by 4-chloro-6-methoxyquinazolin-7-ol (150 mg, 0.71 mmol, 1 equiv). The reaction mixture was stirred and allowed to warm to room temperature. The reaction mixture was filtered, washed with CH₂Cl₂. The filtrate was concentrated under reduced pressure. The residue was purified by flash column chromatography (Combi-flash Rf, Hex/EtOAc=0-100% gradient) to give the title compound (161 mg, 0.599 mmol, 83% yield). ¹H NMR (400 MHz, Chloroform-d) δ 8.87 (s, 1H), 7.39 (s, 1H), 7.37 (s, 1H), 4.37-4.34 (m, 2H), 4.05 (s, 3H), 3.90-3.87 (m, 2H) 3.48 (s, 3H); LCMS (ESI) Method 2: R_{T}=1.262 min, m/z=269.1 [M+H]⁺.

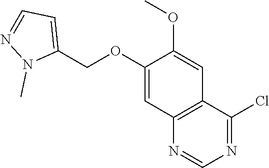 [SMILES: COc1cc2c(Cl)ncnc2cc1OCc1ccnn1C]

### Intermediate 30

### 4-Chloro-6-methoxy-7-((1-methyl-1H-pyrazol-5-yl)methoxy)quinazoline

The title compound (49 mg, 0.16 mmol, 23% yield) was prepared following the procedure described for Intermediate 29, substituting ethyl (1-methyl-1H-pyrazol-5-yl)methanol (80 mg, 0.71 mmol, 1 equiv). ¹H NMR (400 MHz, Chloroform-d) δ 8.89 (s, 1H), 7.47-7.46 (m, 2H), 7.42 (s, 1H), 6.44 (d, J=1.8 Hz, 1H), 5.29 (s, 2H), 4.04 (s, 3H), 3.97 (s, 3H); LCMS (ESI) Method 2: R_{T}=1.304 min, m/z=305.1 [M+H]⁺.

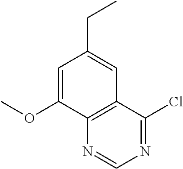

### Intermediate 31

### 4-Chloro-6-ethyl-8-methoxyquinazoline

Step A. Preparation of 6-bromo-8-methoxyquinazolin-4-ol. A mixture of 2-amino-5-bromo-3-methoxybenzoic acid (2.000 g, 8.13 mmol, 1 equiv) and formamidine acetate (8.462 g, 81.28 mmol, 10 equiv) was stirred at 150° C. The cooled mixture was poured into ice water. The solid was collect by filtration, washed with water, and dried to afford the title compound (1.83 g, 7.18 mmol, 88% yield). LCMS (ESI) Method 2: R_{T}=1.063 min, m/z=255.0 [M+H]⁺.

Step B. Preparation of 6-ethyl-8-methoxyquinazolin-4-ol. The title compound (400 mg, 1.96 mmol, quant.) was prepared following the procedure described for Intermediate 14 Step A, substituting 6-bromo-8-methoxyquinazolin-4-ol (500 mg, 1.96 mmol, 1 equiv). LCMS (ESI) Method 2: R_{T}=1.025 min, m/z=205.2 [M+H]⁺.

Step C. Preparation of 4-chloro-6-ethyl-8-methoxyquinazoline. POC₁₋₃ (0.064 mL, 0.687 mmol, 1.5 equiv) was added dropwise to a mixture of 6-ethyl-8-methoxyquinazolin-4-ol (100 mg, 0.458 mmol, 1 equiv) and N,N-diisopropylethylamine (0.12 mL, 0.687 mmol, 1.5 equiv) in anisole (4 mL) while keeping the temperature between 0-10° C. in an ice water bath. The mixture was stirred at room temperature for 1 h and then heated to 95° C. The reaction was cooled to room temperature and quenched with ice water. The pH was adjusted to 7-9 with addition of sat. aq. NaHCO₃. The mixture was extracted with EtOAc. The combined organic layers were dried over MgSO₄, concentrated under reduced pressure. The residue was purified by flash column chromatography (Combi-flash Rf, Hex/EtOAc=0-100% gradient) to give the title compound (30 mg, 0.13 mmol, 29% yield). ¹H NMR (400 MHz, Chloroform-d) δ 9.00 (s, 1H), 7.62 (s, 1H), 7.16 (s, 1H), 4.10 (s, 3H), 2.88 (q, J=7.6 Hz, 2H), 1.38 (t, J=7.6 Hz, 3H).

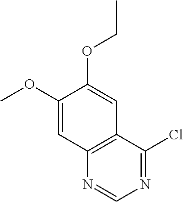 [SMILES: CCOc1cc2c(Cl)ncnc2cc1OC]

### Intermediate 32

### 4-Chloro-6-ethoxy-7-methoxyquinazoline

The title compound (80 mg, 0.34 mmol, 47% yield) was prepared following the procedure described for Intermediate 29, using 4-chloro-7-methoxyquinazolin-6-ol (150 mg, 0.71 mmol, 1 equiv) and ethanol (0.042 mL, 0.71 mmol, 1 equiv). ¹H NMR (400 MHz, Chloroform-d) δ 8.86 (s, 1H), 7.38 (s, 1H), 7.35 (s, 1H), 4.29 (q, J=7.0 Hz, 2H), 4.06 (s, 3H), 1.58 (t, J=7.0 Hz, 3H); LCMS (ESI) Method 2: R_{T}=1.383 min, n: =239.1 [M+H]⁺.

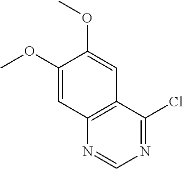

### Intermediate 33

### 4-Chloro-7-ethoxy-6-methoxyquinazoline

The title compound was prepared following the procedure described for Intermediate 29, substituting ethanol (0.083 mL, 1.42 mmol, 2 equiv). ¹H NMR (400 MHz, Chloroform-d) δ 8.86 (s, 1H), 7.39 (s, 1H), 7.33 (s, 1H), 4.29 (q, J=7.0 Hz, 2H), 4.06 (s, 3H), 1.58 (t, J=7.0 Hz, 3H); LCMS (ESI) Method 2: R_{T}=1.367 min, m/z=239.1 [M+H]⁺.

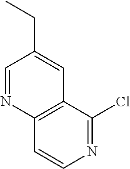

### Intermediate 34

### 5-Chloro-3-ethyl-1,6-naphthyridine

Step A. Preparation of 3-ethyl-1,6-naphthyridin-5-ol. The title compound (150 mg, 0.86 mmol, 97% yield) was prepared following the procedure described for Intermediate 14 Step A, substituting 3-bromo-1,6-naphthyridin-5-ol (200 mg, 0.889 mmol, 1 equiv). LCMS (ESI) Method 2: R_{T}=0.844 min, m, =175.2 [M+H]⁺.

Step B. Preparation of 5-chloro-3-ethyl-1,6-naphthyridine. POCl₃ (2 mL) was added dropwise to 3-ethyl-1,6-naphthyridin-5-ol (200 mg, 1.15 mmol, 1 equiv). The mixture was stirred at 90° C. The reaction was cooled to room temperature and quenched with ice water. The pH was adjusted to 7-9 with addition of sat. aq. NaHCO₃. The mixture was extracted with EtOAc. The combined organic layers were dried over MgSO₄, and concentrated under reduced pressure. The residue was purified by flash column chromatography (Combi-flash Rf, Hex/EtOAc=0-100% gradient) to give the title compound (50 mg, 0.26 mmol, 23% yield). ¹H NMR (400 MHz, Chloroform-d) δ 8.99 (d, J=1.8 Hz, 1H), 8.44 (d, J=5.8 Hz, 1H), 8.37 (s, 1H), 7.85 (d, J=5.8 Hz, 1H), 2.92 (q, J=7.6 Hz, 2H), 1.39 (t, J=7.6 Hz, 3H); LCMS (ESI) Method 2: R_{T}=1.615 min, m/z=193.1 [M+H]⁺.

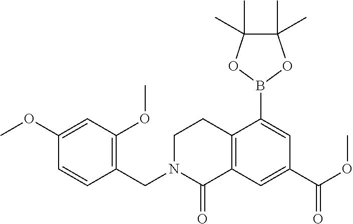

### Intermediate 35

### Methyl 2-(2,4-dimethoxybenzyl)-1-oxo-5-(4,4,5,5-tetramethyl-1,3,2-dioxaborolan-2-yl)-1,2,3,4-tetrahydroisoquinoline-7-carboxylate

Methyl 2-(2,4-dimethoxybenzyl)-1-oxo-5-(((trifluoromethyl)sulfonyl)oxy)-1,2,3,4-tetrahydroisoquinoline-7-carboxylate (Intermediate 2, 500 mg, 0.99 mmol, 1 equiv), Bis(pinacolato)diboron (504 mg, 1.99 mmol, 2 equiv), KOAc (2.92 mg, 2.98 mmol, 3 equiv), and PdCl₂(dppf)-CH₂Cl₂ (36 mg, 0.05 mmol, 0.05 equiv) were dissolved in 1,4-dioxane (10 mL) and placed under an Ar atmosphere. The reaction mixture was stirred at 100° C. overnight. Brine was added to the cooled mixture and extracted with EtOAc. The combined organic layers were dried over MgSO₄ and concentrated under reduced pressure. The residue was purified by flash chromatography (Combi-flash Rf, EtOAc/Hex=0-50% gradient) to afford the title compound (481 mg, 1.0 mmol, quant.). ¹H NMR (400 MHz, Chloroform-d) δ 8.86 (d, J=2.0 Hz, 1H), 8.55 (d, J=2.0 Hz, 1H), 7.27 (d, J=7.8 Hz, 1H), 6.45 (s, 1H), 6.44 (dd, J=7.8, 2.4 Hz, 1H), 4.75 (s, 2H), 3.92 (s, 3H), 3.82 (s, 3H), 3.79 (s, 3H), 3.50 (t, J=6.6 Hz, 2H), 3.30 (t, J=6.6 Hz, 2H), 1.33 (s, 12H); LCMS (ESI) Method 2: R_{T}=2.200 min, m/z=482.1 [M+H]⁺.

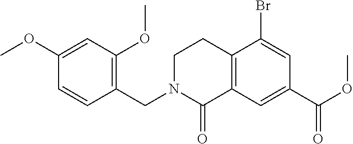

### Intermediate 36

### Methyl 5-bromo-2-(2,4-dimethoxybenzyl)-1-oxo-1,2,3,4-tetrahydroisoquinoline-7-carboxylate

A mixture of methyl 2-(2,4-dimethoxybenzyl)-1-oxo-5-(4,4,5,5-tetramethyl-1,3,2-dioxaborolan-2-yl)-1,2,3,4-tetrahydroisoquinoline-7-carboxylate (Intermediate 35, 479 mg, 0.99 mmol, 1 equiv), copper(II) bromide (665 mg, 2.98 mmol, 3 equiv) and MeOH/H₂O (1:1 v/v, 100 mL) was stirred and refluxed at 80° C. The mixture was cooled to room temperature and diluted with EtOAc. The layers were separated, and the aqueous layer was extracted with EtOAc. The combined organic layers were dried over MgSO₄ and concentrated under reduced pressure. The residue was purified by flash chromatography (Combi-flash Rf, EtOAc/Hex=0-50% gradient) to afford the title compound (373 mg, 0.86 mmol, 87% yield). ¹H NMR (400 MHz, Chloroform-d) δ 8.73 (d, J=1.6 Hz, 1H), 8.31 (d, J=1.7 Hz, 1H), 7.28 (d, J=8.8 Hz, 1H), 6.46 (s, 1H), 6.45 (dd, J=8.2, 2.4 Hz, 1H), 4.73 (s, 2H), 3.93 (s, 3H), 3.82 (s, 3H), 3.79 (s, 3H), 3.56 (t, J=6.7 Hz, 2H), 3.05 (t, J=6.7 Hz, 2H); LCMS (ESI) Method 2: R_{T}=2.075 min, m/z=433.9 [M+H]⁺.

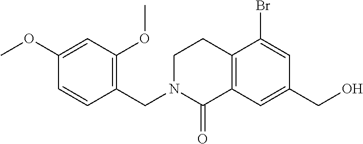 [SMILES: COc1ccc(CN2CCc3c(Br)cc(CO)cc3C2=O)c(OC)c1]

### Intermediate 37

### 5-Bromo-2-(2,4-dimethoxybenzyl)-7-(hydroxymethyl)-3,4-dihydroisoquinolin-1(2H)-one

The title compound (2.79 g, 6.87 mmol, 66% yield) was prepared following the procedure described for Intermediate 5, using methyl 5-bromo-2-(2,4-dimethoxybenzyl)-1-oxo-1,2,3,4-tetrahydroisoquinoline-7-carboxylate (Intermediate 36, 4.5 g, 10 mmol, 1 equiv). ¹H NMR (400 MHz, Chloroform-d) δ 8.03 (s, 1H), 7.69 (d, J=1.2 Hz, 1H), 7.25 (d, J=8.2 Hz, 1H), 6.46 (s, 1H), 6.45 (dd, J=8.2, 2.4 Hz, 1H), 4.71 (s, 2H), 4.69 (s, 2H), 3.82 (s, 3H), 3.79 (s, 3H), 3.52 (t, J=6.7 Hz, 2H), 2.98 (t, J=6.7 Hz, 2H); LCMS (ESI) Method 2: R_{T}=1.832 min, m/z=406.0 [M+H]⁺.

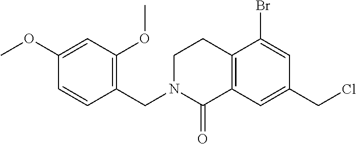 [SMILES: COc1ccc(CN2CCc3c(Br)cc(CCl)cc3C2=O)c(OC)c1]

### Intermediate 38

### 5-Bromo-7-(chloromethyl)-2-(2,4-dimethoxybenzyl)-3,4-dihydroisoquinolin-1(2H)-one

Thionyl chloride (0.8 mL, 10.3 mmol, 1.5 equiv) was added to a solution of 5-bromo-2-(2,4-dimethoxybenzyl)-7-(hydroxymethyl)-3,4-dihydroisoquinolin-1(2H)-one (Intermediate 37, 2.79 g, 6.87 mmol, 1 equiv) in CH₂Cl₂ (60 mL) at 0° C. The reaction was warmed to room temperature and stirred overnight. Sat. aq. NaHCO₃ was added and the mixture was extracted with CH₂Cl₂. The combined organic layers were dried over MgSO₄ and concentrated to provide the title compound (2.5 g, 5.9 mmol, 86% yield), which was used in the next step without further purification. ¹H NMR (400 MHz, Chloroform-d) δ 8.11 (d, J=1.7 Hz, 1H), 7.70 (d, J=1.8 Hz, 1H), 7.26 (d, J=8.3 Hz, 1H), 6.46 (s, 1H), 6.45 (dd, J=8.2, 2.4 Hz, 1H), 4.72 (s, 2H), 4.56 (s, 2H), 3.82 (s, 3H), 3.80 (s, 3H), 3.54 (t, J=6.7 Hz, 2H), 3.00 (t, J=6.7 Hz, 2H): LCMS (ESI) Method 2: R_{T}=2.125 min, m/z=423.9 [M+H]⁺.

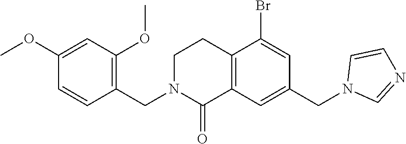 [SMILES: COc1ccc(CN2CCc3c(Br)cc(Cn4ccnc4)cc3C2=O)c(OC)c1]

### Intermediate 39

### 7-((1H-Imidazol-1-yl)methyl)-5-bromo-2-(2,4-dimethoxybenzyl)-3,4-dihydroisoquinolin-1(2H)-one

The title compound (1.83 g, 4.0 mmol, 68% yield) was prepared following the procedure described for Intermediate 7, using 5-bromo-7-(chloromethyl)-2-(2,4-dimethoxybenzyl)-3,4-dihydroisoquinolin-1(2H)-one (Intermediate 38, 2.5 g, 5.9 mmol, 1 equiv), 1H-imidazole (1.2 g, 17.7 mmol, 4 equiv), and cesium carbonate (3.84 g, 11.8 mmol, 2 equiv). ¹H NMR (400 MHz, Chloroform-d) δ 8.03 (s, 1H), 7.79-7.73 (m, 1H), 7.41 (s, 1H), 7.25 (d, J=6.1 Hz, 1H), 7.14 (s, 1H), 6.93 (s, 1H), 6.46 (s, 1H), 6.45 (dd, J=8.4, 2.4 Hz, 1H), 5.14 (s, 2H), 4.71 (s, 2H), 3.82 (s, 3H), 3.79 (s, 3H), 3.55 (t, J=6.7 Hz, 2H), 2.99 (t, J=6.7 Hz, 2H); LCMS (ESI) Method 2: R_{T}=1.439 min, m/z=456.0 [M+H]⁺.

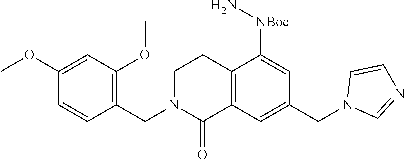 [SMILES: COc1ccc(CN2CCc3c(cc(Cn4ccnc4)cc3N(N)C(=O)OC(C)(C)C)C2=O)c(OC)c1]

### Intermediate 40

### Tert-butyl 1-(7-((1H-imidazol-1-yl)methyl)-2-(2,4-dimethoxybenzyl)-1-oxo-1,2,3,4-tetrahydroisoquinolin-5-yl)hydrazine-1-carboxylate

The title compound (180 mg, 0.36 mmol, 25% yield) was prepared following the Buchwald coupling procedure described for Intermediate 15, using 7-((1H-imidazol-1-yl)methyl)-5-bromo-2-(2,4-dimethoxybenzyl)-3,4-dihydroisoquinolin-1(2H)-one (Intermediate 39, 650 mg, 1.4 mmol, 1 equiv) and tert-butyl hydrazinecarboxylate (376 mg, 2.85 mmol, 2 equiv). ¹H NMR (400 MHz, DMSO-d₆) δ 8.82 (s, 1H), 7.68 (s, 1H), 7.41 (s, 1H), 7.24 (s, 1H), 7.09 (s, 1H), 7.06 (d, J=8.3 Hz, 1H), 6.89 (s, 1H), 6.65 (s, 1H), 6.57 (d, J=2.3 Hz, 1H), 6.47 (dd, J=8.3, 2.3 Hz, 1H), 5.15 (s, 2H), 4.55 (s, 2H), 3.79 (s, 3H), 3.74 (s, 3H), 3.41 (t, J=6.6 Hz, 2H), 2.70 (t, J=6.9 Hz, 2H), 1.41 (s, 9H); LCMS (ESI) Method 2: R_{T}=1.400 min, m/z=508.1 [M+H]⁺.

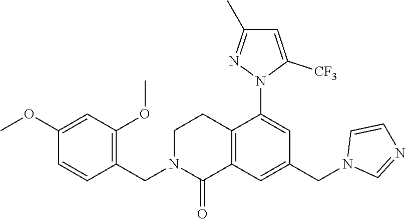 [SMILES: COc1ccc(CN2CCc3c(cc(Cn4ccnc4)cc3-n3nc(C)cc3C(F)(F)F)C2=O)c(OC)c1]

### Intermediate 41

### 7-((1H-Imidazol-1-yl)methyl)-2-(2,4-dimethoxybenzyl)-5-(3-methyl-5-(trifluoromethyl)-1H-pyrazol-1-yl)-3,4-dihydroisoquinolin-1(2H)-one

A 4 M HCl solution in 1,4-dioxane (1.6 mL, 6.46 mmol, 20 equiv) was added dropwise to a solution of tert-butyl 1-(7-((1H-imidazol-1-yl)methyl)-2-(2,4-dimethoxybenzyl)-1-oxo-1,2,3,4-tetrahydroisoquinolin-5-yl)hydrazine-1-carboxylate (Intermediate 40, 164 mg, 0.323 mmol, 1 equiv) in CH₂Cl₂ (2 mL) at room temperature and stirred for 1 h. The reaction mixture was concentrated under reduced pressure. The residue was dissolved in EtOH (1 mL) and DIPEA (0.17 mL, 0.97 mmol, 3 equiv) was added dropwise at room temperature. The reaction mixture was cooled to 0° C. and (Z)-1,1,1-trifluoro-4-methoxypent-3-en-2-one (0.05 mL, 0.36 mmol, 1.1 equiv) was added dropwise. The reaction mixture was allowed to warm to room temperature and then refluxed at 80° C. overnight. The reaction mixture was concentrated under reduced pressure. The residue was purified by flash chromatography (Combi-flash Rf, MeOH/CH₂Cl₂=0-10% gradient) to afford the title compound (118 mg, 0.23 mmol, 70% yield). LCMS (ESI) Method 2: R_{T}=1.555 min, m/z=526.0 [M+H].

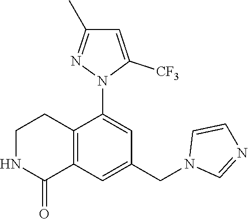 [SMILES: Cc1cc(C(F)(F)F)n(-c2cc(Cn3ccnc3)cc3c2CCNC3=O)n1]

### Intermediate 42

### 7-((1H-Imidazol-1-yl)methyl)-5-(3-methyl-S-(trifluoromethyl)-1H-pyrazol-1-yl)-3,4-dihydroisoquinolin-1(2H)-one

The title compound (56 mg, 0.15 mmol, 71% yield) was prepared following the TFA deprotection procedure described for Intermediate 4, using 7-((1H-imidazol-1-yl)methyl)-2-(2,4-dimethoxybenzyl)-5-(3-methyl-5-(trifluoromethyl)-1H-pyrazol-1-yl)-3,4-dihydroisoquinolin-1(2H)-one (Intermediate 41, 110 mg, 0.21 mmol, 1 equiv). LCMS (ESI) Method 2: R_{T}=1.165 min, m/z=376.0 [M+H]⁺.

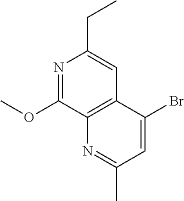

### Intermediate 43

### 4-Bromo-6-ethyl-8-methoxy-2-methyl-1,7-naphthyridine

Step A: Preparation of 5-(1-((6-ethyl-2-methoxypyridin-3-yl)amino)ethylidene)-2,2-dimethyl-1,3-dioxane-4,6-dione. In a dry flask, Meldrum's acid (30 g, 0.21 mol, 1 equiv) was added, followed by pyridine (30 mL) and triethylorthoacetate (60 mL, 0.32 mol, 1.5 equiv). The reaction mixture was heated at 80° C. for 20 min. The reaction mixture was cooled quickly to room temperature and the solvents were removed by rotary evaporator at bath temperature less than 42° C. The viscous material was heated with ligroin for 5 min at 80° C. and the ligroin was decanted. The process was repeated twice more. The 5-(1-ethoxyethylidene)-2,2-dimethyl-1,3-dioxane-4,6-dione was used without further purification (45 g, 0.21 mol, quant.). The ligroin fractions were concentrated down to give additional pure material. ¹H NMR (400 MHz, Chloroform-d) δ 4.41 (q, J=6.8 Hz, 2H), 2.74 (s, 3H), 1.72 (s, 6H), 1.53 (t, J=7.2 Hz, 3H); LCMS (ESI): m/z=214.3 [M+H]⁺. In a dry flask, 6-ethyl-2-methoxypyridin-3-amine (Intermediate 14 Step A, 15 g, 0.099 mol, 1 equiv) was dissolved in anhydrous toluene (30 mL). 5-(1-Ethoxyethylidene)-2,2-dimethyl-1,3-dioxane-4,6-dione (45 g, 0.21 mol, 2.1 equiv) was added and the reaction mixture was heated at 110° C. for 4 h. After cooling to room temperature, a solid was formed, which was filtered and washed with cold ethanol to isolate the title compound (13.85 g, 0.043 mol, 44% yield). ¹H NMR (400 MHz, DMSO-<₆) δ 7.73 (d, J=7.6 Hz, 1H), 6.98 (d, J=8.0 Hz, 1H), 3.93 (s, 3H), 2.73 (q, J=7.6 Hz, 2H), 2.45 (s, 3H), 1.66 (s, 6H), 1.25 (t, J=7.6 Hz, 3H), LCMS (ESI) Method 2: R_{T}=1.906 min, m/z=321.1 [M+H]⁺.

Step B: Preparation of 6-ethyl-8-methoxy-2-methyl-1,7-naphthyridin-4-ol. Finely powdered 5-(1-((6-ethyl-2-methoxypyridin-3-yl)amino)ethylidene)-2,2-dimethyl-1,3-dioxane-4,6-dione (3.8 g, 12 mmol, 1 equiv) was added portionwise to a vigorously stirred diphenyl ether (40 mL) at 230° C. in 15 min and then stirred for another 15 min. Then, the reaction was immediately cooled in room temperature water bath with constant stirring. In 2 min water was replaced by cold water. Hexanes was then added, and the precipitate was filtered and washed with hexanes to obtain the title compound (2.16 g, 9.9 mmol, 83% yield) as an off-white solid. LCMS (ESI) Method 2: R_{T}=1.386 min, m/z=219.1 [M+H]⁺.

Step C: Preparation of 6-ethyl-8-methoxy-2-methyl-1,7-naphthyridin-4-yl trifluoromethanesulfonate. 6-Ethyl-8-methoxy-2-methyl-1,7-naphthyridin-4-ol (1.0 g, 4.58 mmol, 1 equiv) was dissolved in DMF (20 mL) and stirred at room temperature. 1,1,1-trifluoro-N-phenyl-N-((trifluoromethyl)sulfonyl)methanesulfonamide (2.46 g, 6.87 mmol, 1.5 equiv) followed by N,N-diisoproplyethylamine (2 mL, 11.5 mmol, 2.5 equiv) and DMAP (11.2 mg, 0.092 mmol, 0.02 equiv) were added and reaction was stirred at 50° C. for 4 h. The reaction was diluted with EtOAc and sat. NH₄Cl and extracted with EtOAc. The organic layer was dried over MgSO₄, filtered, and concentrated under reduced pressure. The title compound (1.6 g, 1.5 mmol, quant.) was obtained after purification by column chromatography (Combi-flash Rf, EtOAc/Hex=0-20%, gradient). ¹H NMR (400 MHz, Chloroform-d) δ 7.43 (s, 1H), 7.15 (s, 1H), 4.22 (s, 3H), 2.87 (q, J=7.5 Hz, 2H), 2.84 (s, 3H), 1.36 (t, J=7.5 Hz, 3H); LCMS (ESI) Method 2. R_{T}=2.106 min, m/z=351.0 [M+H]⁺.

Step D: Preparation of 4-bromo-6-ethyl-8-methoxy-2-methyl-1,7-naphthyridine. 6-Ethyl-8-methoxy-2-methyl-1,7-naphthyridin-4-yl trifluoromethanesulfonate (1.6 g, 1.5 mmol, 1 equiv) was dissolved in acetonitrile (40 mL) and stirred at room temperature. Lithium bromide (4.0 g, 46 mmol, 10 equiv) was added and reaction was stirred at 80° C. until completion in 3 h. The reaction was cooled to room temperature and concentrated under reduced pressure. The reaction was diluted with EtOAc and water and extracted with EtOAc. The organic layer was dried over MgSO₄, filtered, and concentrated under reduced pressure. The title compound (1.12 g, 3.97 mmol, 87% yield) was obtained after purification by column chromatography (Combi-flash Rf, EtOAc/Hex=0-50%, gradient). ¹H NMR (400 MHz, Chloroform-d) δ 7.72 (s, 1H), 7.27 (s, 1H), 4.21 (s, 3H), 2.85 (q, J=7.5 Hz, 2H), 2.75 (s, 3H), 1.37 (t, J=7.5 Hz, 3H); LCMS (ESI) Method 2: R_{T}=1.598 min, m/z=281.1 [M+H]⁺.

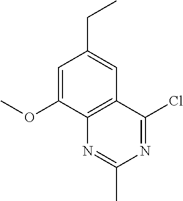

### Intermediate 44

### 4-Chloro-6-ethyl-8-methoxy-2-methylquinazoline

Step A: Preparation of 6-bromo-8-methoxy-2-methylquinazolin-4(3H)-one. Ethanethioamide (376 mg, 5.0 mmol, 5 equiv) was added to a solution of 2-amino-5-bromo-3-methoxybenzoic acid (246 mg, 1.0 mmol, 1 equiv) in ethanol (2 mL). The reaction mixture was stirred at 85° C. under an argon atmosphere for 7 h. The reaction was cooled to room temperature and the solvent was removed under reduced pressure. The residue was suspended in water, filtered, and dried. The title compound (112 mg, 0.42 mmol, 42% yield) was used without further purification. LCMS (ESI) Method 2: R_{T}=1.384 min, m/z=269.1 [M+H]⁺.

Step B: Preparation of 6-ethyl-8-methoxy-2-methylquinazolin-4(3H)-one and 6-ethyl-8-methoxy-2-methylquinazolin-4(1H)-one. In a round bottom flask, 6-bromo-8-methoxy-2-methylquinazolin-4(3H)-one (269 mg, 1.0 mmol, 1 equiv), PdCl₂(dppf)-CH₂Cl₂ adduct (40 mg, 0.05 mmol, 0.05 equiv) were dissolved in THF (5 mL) and placed under an argon atmosphere at room temperature. Diethylzinc (1 mL, 1.0 mmol, 1 equiv, 1 M Hexanes) was added dropwise and the reaction mixture was then placed in a preheated heating block and stirred for 30 min at 65° C. At 23° C., the mixture was filtered and concentrated. The residue was suspended in EtOAc (10 mL), then hexanes (20 mL) was added. The precipitate was filtered to obtain the title compound (142 mg, 0.65 mmol, 65% yield). ¹H NMR (400 MHz, DMSO-d₆) δ 7.70 (d, J=2.1 Hz, 0.5H), 7.44 (s, 1H), 7.43 (s, 0.5H), 7.17 (d, J=1.4 Hz, 1H), 3.90 (s, 1.5H), 3.87 (s, 3H), 2.70 (q, J=7.6 Hz, 2H), 2.33 (s, 1.5H), 2.32 (s, 3H), 1.23 (t, J=7.6 Hz, 3H); LCMS (ESI) Method 2: R_{T}=0.263 min, m, =219.1 [M+H]⁺.

Step C: Preparation of 4-chloro-6-ethyl-8-methoxy-2-methylquinazoline. POCl₃ (1.5 mL) was added dropwise to a mixture of 6-ethyl-8-methoxy-2-methylquinazolin-4(3H)-one and 6-ethyl-8-methoxy-2-methylquinazolin-4(1H)-one (300 mg, 1.37 mmol, 1 equiv) in a reaction vial. The mixture was stirred at 90° C. until completion. The reaction was cooled to room temperature and concentrated under reduced pressure. The residue was dissolved in EtOAc and washed with sat. NaHCO₃. The combined organic layers were dried over MgSO₄, concentrated under reduced pressure. The title compound (364 mg, 1.54 mmol, quant.) was used without further purification. ¹H NMR (400 MHz, Chloroform-d) δ 7.57 (s, 1H), 7.10 (d, J=1.2 Hz, 1H), 4.08 (s, 3H), 2.88 (s, 3H), 2.84 (q, J=6.8 Hz, 2H), 1.36 (t, J=7.6 Hz, 3H); LCMS (ESI) Method 2: R_{T}=1.639 min, m/z=237.1 [M+H]⁺.

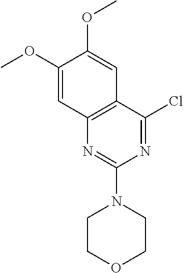 [SMILES: COc1cc2nc(N3CCOCC3)nc(Cl)c2cc1OC]

### Intermediate 45

### 4-(4-Chloro-6,7-dimethoxyquinazolin-2-yl)morpholine

Step A: Preparation of 6,7-dimethoxy-2-morpholinoquinazolin-4(3H)-one. 2-Chloro-6,7-dimethoxyquinazolin-4(3H)-one (100 mg, 0.42 mmol, 1 equiv), and morpholine (0.06 mL, 0.70 mmol, 1.7 equiv) were dissolved in EtOH (2 mL) and stirred at 80° C. until completion. The reaction was cooled to 0° C., filtered, and washed with cold EtOH to yield 6,7-dimethoxy-2-morpholinoquinazolin-4(3H)-one (112 mg, 0.38 mmol, 92% yield). ¹H NMR (400 MHz, DMSO-d₆) δ 11.19 (brs, 1H), 7.28 (s, 1H), 6.78 (s, 1H), 3.84 (s, 3H), 3.80 (s, 3H), 3.66 (t, J=4.7 Hz, 4H), 3.53 (t, J=4.8 Hz, 4H); LCMS (ESI) Method 2: R_{T}=1.056 min, m/z=292.1 [M+H]⁺.

Step B: Preparation of 4-(4-chloro-6,7-dimethoxyquinazolin-2-yl)morpholine. POCl₃ (0.5 mL) was added dropwise to 6,7-dimethoxy-2-morpholinoquinazolin-4(3H)-one (50 mg, 0.17 mmol, 1 equiv) in a reaction vial. The mixture was stirred at 90° C. until completion. The reaction was cooled to room temperature and concentrated under reduced pressure. The residue was dissolved in EtOAc and washed with sat. NaHCO₃. The combined organic layers were dried over MgSO₄, concentrated under reduced pressure. The title compound (54 mg, 0.17 mmol, quant.) was used without further purification. ¹H NMR (400 MHz, Chloroform-d) δ 7.26 (s, 1H), 7.21 (s, 1H), 4.03 (s, 3H), 3.99 (s, 3H), 3.93 (brs, 4H), 3.81 (t, J=4.8 Hz, 4H); LCMS (ESI) Method 2: R_{T}=1.529 min, m/z=310.0 [M+H]⁺.

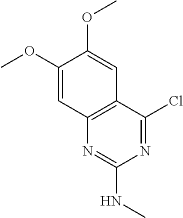 [SMILES: CNc1nc(Cl)c2cc(OC)c(OC)cc2n1]

### Intermediate 46

### 4-Chloro-6,7-dimethoxy-N-methylquinazolin-2-amine

Step A: Preparation of 6,7-dimethoxy-2-(methylamino)quinazolin-4(3H)-one. 2-Chloro-6,7-dimethoxyquinazolin-4(3H)-one (300 mg, 1.25 mmol, 1 equiv), potassium carbonate (431 mg, 3.12 mmol, 2.5 equiv) and methylamine hydrochloride (421 mg, 6.23 mmol, 5 equiv) was dissolved in EtOH (2 mL) and stirred at 80° C. until completion. The reaction was cooled to 0° C., filtered, and washed with cold EtOH to yield 6,7-dimethoxy-2-(methylamino)quinazolin-4(3H)-one (265 mg, 1.13 mmol, 90% yield). LCMS (ESI) Method 2: R_{T}=1.024 min, m/z=236.1 [M+H]⁺.

Step B: Preparation of 4-chloro-6,7-dimethoxy-N-methylquinazolin-2-amine. The title compound (55 mg, 0.21 mmol, quant.) was prepared following the procedure described for Intermediate 44 Step C, substituting 6,7-dimethoxy-2-(methylamino)quinazolin-4(3H)-one (50 mg, 0.21 mmol, 1 equiv). ¹H NMR (400 MHz, Chloroform-d) δ 8.66 (s, 1H), 7.28 (s, 1H), 7.13 (s, 1H), 4.08 (s, 3H), 4.01 (s, 3H), 3.19 (d, J=4.8 Hz, 3H); LCMS (ESI) Method 2: R_{T}=1.256 min, m/z=254.1 [M+H].

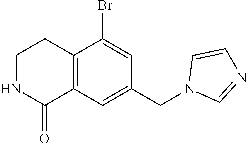 [SMILES: O=C1NCCc2c(Br)cc(Cn3ccnc3)cc21]

### Intermediate 47

### 7-((1H-Imidazol-1-yl)methyl)-5-bromo-3,4-dihydroisoquinolin-1(2H)-one

The title compound (230 mg, 0.75 mmol, 86% yield) was prepared following the TFA deprotection procedure described for Intermediate 4, using 7-((1H-imidazol-1-yl)methyl)-5-bromo-2-(2,4-dimethoxybenzyl)-3,4-dihydroisoquinolin-1(2H)-one (Intermediate 39, 400 mg, 0.88 mmol, 1 equiv). ¹H NMR (400 MHz, DMSO-d₆) δ 8.10 (s, 1H), 7.88-7.80 (m, 1H), 7.77 (s, 1H), 7.71 (d, J=1.8 Hz, 1H), 7.31-7.20 (m, 1H), 6.99-6.89 (m, 1H), 5.24 (s, 2H), 3.37 (td, J=6.6, 2.7 Hz, 2H), 2.93 (t, J=6.6 Hz, 2H); LCMS (ESI) Method 2: R_{T}=1.102 min, m/z=306.0 [M+H]⁺.

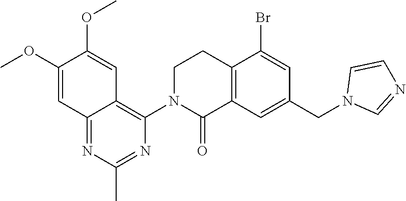 [SMILES: COc1cc2nc(C)nc(N3CCc4c(Br)cc(Cn5ccnc5)cc4C3=O)c2cc1OC]

### Intermediate 48

### 7-((1H-Imidazol-1-yl)methyl)-5-bromo-2-(6,7-dimethoxy-2-methylquinazolin-4-yl)-3,4-dihydroisoquinolin-1(2H)-one

The title compound (280 mg, 0.55 mmol, 75% yield) was prepared following the Buchwald coupling procedure described for Intermediate 15, using 7-((1H-imidazol-1-yl)methyl)-5-bromo-3,4-dihydroisoquinolin-1(2H)-one (Intermediate 47, 244 mg, 0.73 mmol, 1 equiv) and 4-chloro-6,7-dimethoxy-2-methylquinazoline (210 mg, 0.88 mmol, 1.2 equiv). LCMS (ESI) Method 2: R_{T}=1.282 min, m/z=508.0 [M+H]⁺.

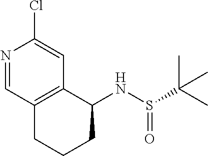 [SMILES: CC(C)(C)[S@](=O)N[C@H]1CCCc2cnc(Cl)cc21]

### Intermediate 49

### (R)-N-((S)-3-chloro-5,6,7,8-tetrahydroisoquinolin-5-yl)-2-methylpropane-2-sulfinamide

Step A: Preparation of (S,E)-N-((5-bromo-2-chloropyridin-4-yl)methylene)-2-methylproprane-2-sulfinamide. A suspension of (S)-2-methylpropane-2-sulfinamide (6.60 g, 54.4 mmol, 1.2 equiv) and Cs₂CM3 (14.8 g, 45.4 mmol, 1 equiv) in anhydrous dichloromethane (70 mL) was stirred at room temperature for 10 min followed by the addition of 5-bromo-2-chloroisonicotinaldehyde (10.0 g, 45.4 mmol, 1 equiv). The mixture was stirred 42° C. until reaction completion. The reaction was mixture was filtered. The filtrate was concentrated and was purified by flash chromatography (Combi-flash Rf, EtOAc/Hex=0-20% gradient) to provide (S,E)-N-((5-bromo-2-chloropyridin-4-yl)methylene)-2-methylpropane-2-sulfinamide (7.49 g, 23.1 mmol, 51% yield). ¹H NMR (400 MHz, Chloroform-d) δ 8.85 (s, 1H), 8.63 (s, 1H), 7.85 (s, 1H), 1.30 (s, 10H); LCMS (ESI): m/z=323.2 [M+H]⁺.

Step B: Preparation of (S,E)-N-((2-chloro-5-vinylpyridin-4-yl)methylene)-2-methylpropane-2-sulfinamide. To an oven dried 2-dram vial equipped with a magnetic stir bar, (S,E)-N-((5-bromo-2-chloropyridin-4-yl)methylene)-2-methylpropane-2-sulfinamide (1.00 g, 3.09 mmol, 1 equiv), PdC12(dppf) (452 mg, 0.62 mmol, 0.2 equiv), K₃PO₄ (3.28 g, 15.4 mmol, 5 equiv), and potassium vinyltrifluoroborate (455 mg, 3.40 mmol, 1.1 equiv) were suspended in THF (10 mL) and H₂O (2.4 mL). The resulting suspension was heated to 75° C. and stirred until reaction completion. The reaction mixture was cooled to room temperature followed by dilution with CH₂Cl₂ and H₂O. The layers were separated. The aqueous layer was extracted with CH₂Cl₂ (2×). The combined organic layers were dried over anhydrous Na₂SO₄, filtered, and concentrated under reduced pressure. The crude material was purified by flash chromatography (Combi-flash Rf, EtOAc/Hex=0-30% gradient) to provide (S,E)-N-((2-chloro-5-vinylpyridin-4-yl)methylene)-2-methylpropane-2-sulfinamide (452 mg, 3.09 mmol, 54% yield). ¹H NMR (400 MHz, Chloroform-d) δ 8.82 (s, 1H), 8.59 (d, J=0.7 Hz, 1H), 7.77 (s, 1H), 7.21-7.06 (m, 1H), 5.75 (dd, J=17.4, 0.8 Hz, 1H), 5.61 (dd, J 11.1, 0.7 Hz, 1H), 1.29 (s, 10H); LCMS (ESI): m/z=271.2 [M+H]⁺.

Step C: Preparation of (S)-N-((S)-1-(2-chloro-5-vinylpyridin-4-yl)but-3-en-1-yl)-2-methylpropane-2-sulfinamide. To a suspension of granular zinc (1.97 g, 30 mmol, 1.2 equiv) in THF (25.5 mL), allyl bromide (2.21 mL, 25.5 mmol, 1 equiv) was added dropwise. The resulting suspension was stirred at 40° C. until most of the zinc was consumed. In a separate oven dried 200 mL round-bottom flask equipped with a magnetic stir bar, the freshly prepared 1M allylzinc bromide solution (14.7 mL, 14.7 mmol, 1.5 equiv) was added dropwise to a −78° C. solution of (S,E)-N-((2-chloro-5-vinylpyridin-4-yl)methylene)-2-methylpropane-2-sulfinamide (2.66 g, 9.82 mmol, 1 equiv) in anhydrous THF (48 mL). The resulting mixture was stirred at −78° C. for 1 h, and then quenched with saturated aqueous NH₄Cl. The mixture was warmed to room temperature then extracted with EtOAc. The combined organic layers were dried (Na₂SO₄) and concentated. The residue was purified by flash chromatography (Combi-flash Rf, EtOAc/Hex=0-50% gradient) to provide (S)-N-((S)-1-(2-chloro-5-vinylpyridin-4-yl)but-3-en-1-yl)-2-methylpropane-2-sulfinamide (2.68 g, 9.82 mmol, 88% yield). ¹H NMR (400 MHz, Chloroform-d) δ 8.43 (s, 1H), 7.33 (s, 1H), 6.94 (dd, J=17.3, 11.1 Hz, 1H), 5.79-5.59 (m, 2H), 5.51 (dd, J=11.0, 1.0 Hz, 1H), 5.36-5.15 (m, 2H), 4.90-4.72 (m, 1H), 3.79 (s, 1H), 2.60 (dt, J=14.5, 5.8 Hz, 1H), 2.49-2.28 (m, 1H), 1.25 (s, 10H); LCMS (ESI): m/z=313.3 [M+H]⁺.

Step D: Preparation of (S)-N-((S)-3-chloro-5,6-dihydroisoquinolin-5-yl)-2-methylpropane-2-sulfinamide. To a solution of (S)-N-((S)-1-(2-chloro-5-vinylpyridin-4-yl)but-3-en-1-yl)-2-methylpropane-2-sulfinamide (1.63 g, 5.21 mmol, 1 equiv) in CH₂Cl₂ (45 mL), benzylidene[1,3-bis(2,4,6-trimethylphenyl)-2-imidazolidinylidene]dichloro(tricyclohexyl phosphine)ruthenium (Grubbs II catalyst, 221 mg, 0.26 mmol, 0.05 equiv) was added. A vent needle was added, and the resulting solution was heated to 40° C. and stirred until reaction completion, which was typically 1 h. The reaction was cooled to room temperature then concentrated under reduced pressure. The residue was purified by flash chromatography (Combi-flash Rf, EtOAc/Hex=0-100% gradient) to provide (S)-N-((S)-3-chloro-5,6-dihydroisoquinolin-5-yl)-2-methylpropane-2-sulfinamide (1.40 g, 5.21 mmol, 94% yield). ¹H NMR (400 MHz, Chloroform-d) δ 8.11 (s, 1H), 7.35 (s, 1H), 6.54 (d, J=9.8 Hz, 1H), 6.15 (dt, J=9.2, 4.3 Hz, 1H), 4.53 (q, J=8.3 Hz, 1H), 3.35 (d, J=9.5 Hz, 1H), 2.88 (dt, J=16.9, 5.6 Hz, 1H), 2.64-2.42 (m, 1H), 1.26 (s, 10H); LCMS (ESI): m/z=285.2 [M+H]⁺.

Step E: Preparation of (S)-N-((S)-3-chloro-5,6,7,8-tetrahydroisoquinolin-5-yl)-2-methylpropane-2-sulfinamide. To a solution of (S)-N-((S)-3-chloro-5,6-dihydroisoquinolin-5-yl)-2-methylpropane-2-sulfinamide (1.40 g, 4.92 mmol, 1 equiv) in MeOH (10 mL) was added platinum(IV) oxide (112 mg, 0.49 mmol, 0.1 equiv). The flask was evacuated and backfilled with H₂ (3×). The solution was stirred until reaction completion. The reaction mixture was filtered over celite. The filter cake was washed with EtOAc. The filtrated was concentrated under reduced pressure to provide the desired product (1.39 g, 4.85 mmol, 99% yield), which was used without further purification. ¹H NMR (400 MHz, Chloroform-d) δ 8.14 (s, 1H), 7.36 (s, 1H), 4.39 (q, J=8.1 Hz, 1H), 3.33 (d, J=10.0 Hz, 1H), 2.74 (t, J=6.1 Hz, 2H), 2.52-2.33 (m, 1H), 1.97 (qd, J=7.2, 3.8 Hz, 1H), 1.91-1.75 (m, 2H), 1.30 (s, 9H); LCMS (ESI): m/z=287.2 [M+H]⁺.

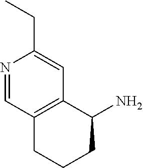

### Intermediate 50

### (S)-3-Ethyl-5,6,7,8-tetrahydroisoquinolin-5-amine

Step A: Preparation of (S)-N-((S)-3-ethyl-5,6,7,8-tetrahydroisoquinolin-5-yl)-2-methylpropane-2-sulfinamide. To a 0° C. suspension of (S)-N-((S)-3-chloro-5,6,7,8-tetrahydroisoquinolin-5-yl)-2-methylpropane-2-sulfinamide (Intermediate 49, 1.00 g, 3.49 mmol, 1 equiv) and PdCl₂(dppf) (128 mg, 0.17 mmol, 0.05 equiv) in THF (30 mL), diethylzinc (1 M in hexanes, 4.2 mL, 4.18 mmol, 1.2 equiv) was added dropwise. The resulting mixture was heated to 60° C. and stirred until reaction completion. The reaction mixture was cooled then diluted with EtOAc and H₂O. The layers were separated. The aqueous layer was extracted with EtOAc (2×). The combined organic layers were dried over anhydrous Na₂SO₄, filtered, and concentrated under reduced pressure. The residue was purified by flash chromatography (Combi-flash Rf, MeOH/CH₂Cl₂=0-10% gradient) to provide the desired product (980 mg, 3.49 mmol, quant.). ¹H NMR (400 MHz, Chloroform-d) δ 8.28 (s, 1H), 7.21 (s, 1H), 4.39 (s, 1H), 3.36 (d, J=9.8 Hz, 1H), 2.88-2.66 (m, 4H), 2.48-2.33 (m, 1H), 2.05-1.77 (m, 2H), 1.32-1.17 (m, 14H); LCMS (ESI): m-z=281.3 [M+H]⁺.

Step B: Preparation of (S)-3-ethyl-5,6,7,8-tetrahydroisoquinolin-5-amine. To a solution of (S)-N-((S)-3-ethyl-5,6,7,8-tetrahydroisoquinolin-5-yl)-2-methylpropane-2-sulfinamide (100 mg, 0.55 mmol, 1 equiv) in THF (6 mL) at 0° C. was added 4 M HCl in 1,4-dioxane (1.8 mL, 7.13 mmol, 20 equiv). The resulting mixture was allowed to warm to room temperature and stirred overnight. The reaction was concentrated in vacuo. The residue was suspended in CH₂Cl₂ and washed with saturated aqueous Na₂CO₃. The aqueous layer was extracted with CH₂Cl₂ (2×). The combined organic layers were dried over anhydrous Na₂SO₄, filtered, and concentrated under reduced pressure to provide (S)-3-ethyl-5,6,7,8-tetrahydroisoquinolin-5-amine (41 mg, 0.36 mmol, 65% yield): ¹H NMR (400 MHz, Chloroform-d) δ 8.26 (s, 1H), 3.97 (s, 1H), 2.91-2.61 (m, 5H), 2.09 (t, J=6.6 Hz, 1H), 1.94 (d, J=6.4 Hz, OH), 1.89-1.57 (m, 8H), 1.30 (d, J=7.6 Hz, 3H); LCMS (ESI): m/z=177.4 [M+H]⁺.

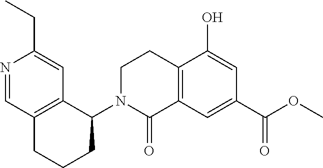 [SMILES: CCc1cc2c(cn1)CCC[C@@H]2N1CCc2c(O)cc(C(=O)OC)cc2C1=O]

### Intermediate 51

### Methyl (S)-3′-ethyl-5-hydroxy-1-oxo-3,4,5′,6′,7′,8′-hexahydro-1H-[2,5′-biisoquinoline]-7-carboxylate

The title compound (243 mg, 0.64 mmol, 40% yield) was prepared following the procedure described for Intermediate 1, using dimethyl 2-hydroxy-2,3-dihydrobenzofuran-4,6-dicarboxylate (400 mg, 1.6 mmol, 1 equiv) and (S)-3-ethyl-5,6,7,8-tetrahydroisoquinolin-5-amine (Intermediate 50, 310 mg, 1.7 mmol, 1.1 equiv). ¹H NMR (400 MHz, Chloroform-d) δ 8.41 (d, J=1.5 Hz, 1H), 8.33 (s, 1H), 7.66 (d, J=1.6 Hz, 1H), 7.04 (s, 1H), 6.05 (dd, J=10.4, 5.8 Hz, 1H), 4.01-3.83 (m, 5H), 3.40 (ddd, J=12.4, 10.3, 4.7 Hz, 1H), 3.20 (dt, J=12.0, 5.5 Hz, 1H), 3.04 (dt, J=16.7, 5.1 Hz, 1H), 2.95-2.66 (m, 6H), 2.26-2.08 (m, 3H), 1.98-1.76 (m, 2H), 1.25 (q, J=7.3 Hz, 4H); LCMS (ESI): m/z=381.4 [M+H]⁺.

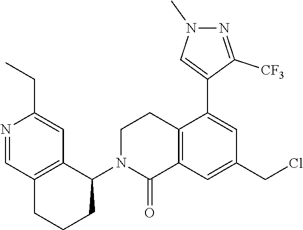 [SMILES: CCc1cc2c(cn1)CCC[C@@H]2N1CCc2c(cc(CCl)cc2-c2cn(C)nc2C(F)(F)F)C1=O]

### Intermediate 52

### (S)-7-(Chloromethyl)-3′-ethyl-5-(1-methyl-3-(trifluoromethyl)-1H-pyrazol-4-yl)-3,4,5′,6′,7′,8′-hexahydro-1H-[2,5′-biisoquinolin]-1-one

Step A: Preparation of methyl (S)-3′-ethyl-1-oxo-5-(((trifluoromethyl)sulfonyl) oxy)-3,4,5′,6′,7′,8′-hexahydro-1H-[2,5′-biisoquinoline]-7-carboxylate. The title compound (311 mg, 0.63 mmol, 40% yield) was prepared following the procedure described for Intermediate 2, using methyl (S)-3′-ethyl-5-hydroxy-1-oxo-3,4,5′,6′,7′,8′-hexahydro-1H-[2,5′-biisoquinoline]-7-carboxylate (Intermediate 51, 240 mg, 0.63 mmol, 1 equiv). ¹H NMR (400 MHz, Chloroform-d) 8.87 (d, J=1.6 Hz, 1H), 8.34 (s, 1H), 8.08 (d, J=1.6 Hz, 1H), 6.94 (s, 1H), 6.03 (dd, J=10.5, 6.0 Hz, 1H), 3.97 (d, J=12.4 Hz, 4H), 3.42 (ddd, J=12.6, 9.9, 4.7 Hz, 1H), 3.30-3.17 (m, 1H), 3.15-2.91 (m, 2H), 2.76 (td, J=16.0, 6.9 Hz, 5H), 2.25-2.07 (m, 2H), 1.97-1.72 (m, 2H), 1.25 (dt, J=9.6, 7.4 Hz, 8H); LCMS (ESI): m/z=513.3 [M+H]⁺.

Step B: Preparation of methyl (S)-3′-ethyl-5-(1-methyl-4-(trifluoromethyl)-1H-pyrazol-3-yl)-1-oxo-3,4,5′,6′,7′,8′-hexahydro-1H-[2,5′-biisoquinoline]-7-carboxylate. The title compound (150 mg, 0.29 mmol, 48% yield) was prepared following the procedure described for Intermediate 3, using methyl (S)-3′-ethyl-1-oxo-5-(((trifluoromethyl)sulfonyl) oxy)-3,4,5′,6′,7′,8′-hexahydro-JH-[2,5′-biisoquinoline]-7-carboxylate (310 mg, 0.61 mmol, 1 equiv). ¹H NMR (400 MHz, Chloroform-d) δ 8.86 (d, J=1.9 Hz, 1H), 8.33 (s, 1H), 8.06 (d, J=1.9 Hz, 1H), 7.39 (s, 1H), 6.05 (t, J=8.4 Hz, 1H), 4.02 (s, 3H), 3.95 (s, 3H), 3.32 (s, 1H), 3.10 (dt, J=12.5, 5.5 Hz, 1H), 2.76 (dd, J=15.7, 9.3 Hz, 7H), 2.28-2.07 (m, 2H), 1.99-1.73 (m, 2H), 1.26 (t, J=7.1 Hz, 3H); LCMS (ESI): m/z=513.4 [M+H]⁺.

Step C: Preparation of (S)-3′-ethyl-7-(hydroxymethyl)-5-(1-methyl-4-(trifluoromethyl)-1H-pyrazol-3-yl)-3,4,5′,6′,7′,8′-hexahydro-1H-[2,5′-biisoquinolin]-1-one. The title compound (109 mg, 0.29 mmol, 77% yield) was prepared following the procedure described for Intermediate 5, using methyl (S)-3′-ethyl-5-(1-methyl-4-(trifluoromethyl)-JH-pyrazol-3-yl)-1-oxo-3,4,5′,6′,7′,8′-hexahydro-1H-[2,5′-biisoquinoline]-7-carboxylate (150 mg, 0.29 mmol, 1 equiv). ¹H NMR (400 MHz, Chloroform-d)δ 8.31 (s, 1H), 8.20 (d, J 1.9 Hz, 1H), 7.43 (d, J=1.9 Hz, 1H), 7.36 (s, 1H), 6.99 (s, 1H), 6.04 (dd, J=10.6, 6.0 Hz, 1H), 4.78 (d, J=5.6 Hz, 2H), 4.00 (s, 3H), 3.29 (ddd, J=12.4, 8.9, 5.7 Hz, 1H), 3.08 (dt, J=11.9, 5.5 Hz, 1H), 2.89-2.56 (m, 7H), 2.23-2.00 (m, 2H), 1.98-1.67 (m, 3H), 1.23 (t, J=7.6 Hz, 3H); LCMS (ESI): m/z=485.4 [M+H]⁺.

Step D: Preparation of (S)-7-(chloromethyl)-3′-ethyl-5-(1-methyl-4-(trifluoromethyl)-1H-pyrazol-3-yl)-3,4,5′,6′,7′,8′-hexahydro-1H-[2,5′-biisoquinolin]-1-one. The title compound (84 mg, 0.17 mmol, 96% yield) was prepared following the procedure described for Intermediate 38, using (S)-3′-ethyl-7-(hydroxymethyl)-5-(1-methyl-4-(trifluoromethyl)-1H-pyrazol-3-yl)-3,4,5′,6′,7′,8′-hexahydro-JH-[2,5′-biisoquinolin]-1-one (84 mg, 0.17 mmol, 1 equiv) and thionyl chloride (0.19 mL, 2.6 mmol, 15 equiv). LCMS (ESI): m/z=503.3 [M+H]⁺.

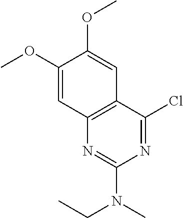 [SMILES: CCN(C)c1nc(Cl)c2cc(OC)c(OC)cc2n1]

### Intermediate 53

### 4-Chloro-N-ethyl-6,7-dimethoxy-N-methylquinazolin-2-amine

Step A: Preparation of 2-(ethyl(methyl)amino)-6,7-dimethoxyquinazolin-4(3H)-one. Chloro-6,7-dimethoxyquinazolin-4(3H)-one (100 mg, 0.42 mmol, 1 equiv), and N-methylethanamine (0.06 mL, 0.70 mmol, 1.7 equiv) was dissolved in EtOH (2 mL) and stirred at 80° C. until completion. The reaction was cooled to 0° C., filtered, and washed with cold EtOH to yield 2-(ethyl(methyl)amino)-6,7-dimethoxyquinazolin-4(3H)-one (100 mg, 0.38 mmol, 91% yield). ¹H NMR (400 MHz, DMSO-d₆) δ 10.93 (s, 1H), 7.25 (s, 1H), 6.71 (s, 1H), 3.84 (s, 3H), 3.78 (s, 3H), 3.56 (q, J=7.0 Hz, 2H), 3.02 (s, 3H), 1.08 (t, J=7.0 Hz, 3H); LCMS (ESI) Method 2: R_{T}=1.096 min, m/z=264.1 [M+H]⁺.

Step B: Preparation of 4-chloro-N-ethyl-6,7-dimethoxy-N-methylquinazolin-2-amine. The title compound (83 mg, 0.29 mmol, 78% yield) was prepared following the procedure described for Intermediate 44 Step C, substituting 2-(ethyl(methyl)amino)-6,7-dimethoxyquinazolin-4(3H)-one (100 mg, 0.38 mmol, 1 equiv). LCMS (ESI) Method 2: R_{T}=1.425 min, m/z=282.1 [M+H]⁺.

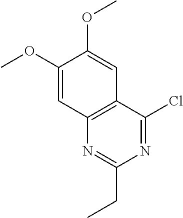 [SMILES: CCc1nc(Cl)c2cc(OC)c(OC)cc2n1]

### Intermediate 54

### 4-Chloro-2-ethyl-6,7-dimethoxyquinazoline

Step A: Preparation of 2-ethyl-6,7-dimethoxyquinazolin-4(3H)-one. Methyl 2-amino-4,5-dimethoxybenzoate (100 mg, 0.47 mmol, 1 equiv), propiononitrile (0.11 mL, 1.42 mmol, 3 equiv), and 4 M HCl in 1,4-dioxane (1 mL) were added to a reaction vial and the resulting heterogenous mixture was stirred at 115° C. until completion. The reaction mixture was cooled and poured into cold saturated aqueous NaHCO₃ solution (10 mL). The precipitate was filtered, washed with water, and air-dried to afford 2-ethyl-6,7-dimethoxyquinazolin-4(3H)-one (100 mg, 0.43 mmol, 90% yield), which was used without purification. ¹H NMR (400 MHz, DMSO-d₆) δ 7.40 (s, 1H), 7.07 (s, 1H), 3.89 (s, 3H), 3.85 (s, 3H), 2.59 (q, J=7.5 Hz, 2H), 1.23 (t, J=7.5 Hz, 3H); LCMS (ESI) Method 2: R_{T}=0.331 min, m/z=235.2 [M+H]⁺.

Step B: Preparation of 4-chloro-2-ethyl-6,7-dimethoxyquinazoline. POCl₃ (0.5 mL) was added dropwise to 2-ethyl-6,7-dimethoxyquinazolin-4(3H)-one (100 mg, 0.43 mmol, 1 equiv) in a reaction vial. The mixture was stirred at 90° C. until completion. The reaction was cooled to room temperature and concentrated under reduced pressure. The residue was dissolved in EtOAc and washed with sat. NaHCO₃. The combined organic layers were dried over MgSO₄, concentrated under reduced pressure. The title compound (64 mg, 0.25 mmol, 59% yield) was used without further purification. LCMS (ESI) Method 2: R_{T}=1.524 min, m/z=253.0 [M+H]⁺.

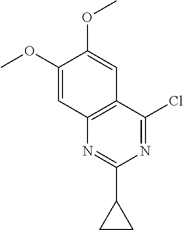 [SMILES: COc1cc2nc(C3CC3)nc(Cl)c2cc1OC]

### Intermediate 55

### 4-Chloro-2-cyclopropyl-6,7-dimethoxyquinazoline

Step A: Preparation of 2-cyclopropyl-6,7-dimethoxyquinazolin-4(3H)-one. The title compound (27 mg, 0.11 mmol, 23% yield) was prepared following the procedure described for Intermediate 54 Step A, using methyl 2-amino-4,5-dimethoxybenzoate (100 mg, 0.47 mmol, 1 equiv) and cyclopropanecarbonitrile (0.11 mL, 1.42 mmol, 3 equiv). LCMS (ESI) Method 2: R_{T}=0.245 min, m/z=247.1 [M+H]⁺.

Step B: Preparation of 4-chloro-2-cyclopropyl-6,7-dimethoxyquinazoline. The title compound (17 mg, 0.064 mmol, 63% yield) was prepared following the procedure described for Intermediate 44 Step C, substituting 2-cyclopropyl-6,7-dimethoxyquinazolin-4(3H)-one (25 mg, 0.10 mmol, 1 equiv). LCMS (ESI) Method 2: R_{T}=1.609 min, m/z=265.1 [M+H]⁺.

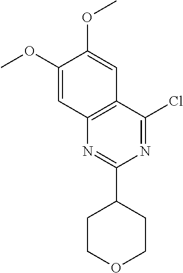 [SMILES: COc1cc2nc(C3CCOCC3)nc(Cl)c2cc1OC]

### Intermediate 56

### 4-Chloro-6,7-dimethoxy-2-(tetrahydro-2H-pyran-4-yl)quinazoline

Step A: Preparation of 6,7-dimethoxy-2-(tetrahydro-2H-pyran-4-yl)quinazolin-4(3H)-one. Methyl 2-amino-4,5-dimethoxybenzoate (400 mg, 1.89 mmol, 1 equiv), tetrahydro-2H-pyran-4-carbonitrile (2.1 mL, 18.9 mmol, 3 equiv), and 4 M HCl in 1,4-dioxane (10 mL) were added to a reaction vial and the resulting heterogenous mixture was stirred at 100° C. until completion. The reaction mixture was cooled and poured into cold saturated aqueous NaHCO₃ solution (20 mL). The precipitate was filtered, washed with water, and air-dried to afford 6,7-dimethoxy-2-(tetrahydro-2H-pyran-4-yl)quinazolin-4(3H)-one (546 mg, 1.88 mmol, 99% yield), which was used without purification. LCMS (ESI) Method 2: R_{T}=1.139 min, m/z=291.1 [M+H]⁺.

Step B: Preparation of 4-chloro-6,7-dimethoxy-2-(tetrahydro-2H-pyran-4-yl)quinazoline. POCl₃ (1 mL) was added dropwise to 6,7-dimethoxy-2-(tetrahydro-2H-pyran-4-yl)quinazolin-4(3H)-one (300 mg, 1.03 mmol, 1 equiv) in a reaction vial. The mixture was stirred at 90° C. until completion. The reaction was cooled to room temperature and concentrated under reduced pressure. The residue was dissolved in EtOAc and washed with sat. NaHCO₃. The combined organic layers were dried over MgSO₄, concentrated under reduced pressure. The title compound (298 mg, 0.93 mmol, 90° % yield) was used without further purification. ¹H NMR (400 MHz, Chloroform-d) δ 7.37 (s, 1H), 7.31 (s, 1H), 4.11 (ddd, J=11.4, 4.3, 2.0 Hz, 2H), 4.07 (s, 3H), 4.06 (s, 3H), 3.89 (ddd, J=12.1, 6.3, 3.6 Hz, 1H), 3.66-3.50 (m, 4H), 3.19 (tt, J=11.3, 4.2 Hz, 1H), 2.86 (tt, J=8.1, 4.4 Hz, 1H); LCMS (ESI) Method 2: R_{T}=1.571 min, m/z=309.1 [M+H]⁺.

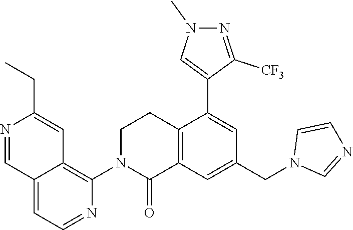 [SMILES: CCc1cc2c(N3CCc4c(cc(Cn5ccnc5)cc4-c4cn(C)nc4C(F)(F)F)C3=O)nccc2cn1]

### Example 1

### 7-((1H-Imidazol-1-yl)methyl)-2-(3-ethyl-1,6-naphthyridin-5-yl)-5-(1-methyl-3-(trifluoromethyl)-1H-pyrazol-4-yl)-3,4-dihydroisoquinolin-1(2H)-one

The title compound (68 mg, 0.13 mmol, 80% yield) was prepared following the procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 5-chloro-3-ethyl-1,6-naphthyridine (Intermediate 34, 46 mg, 0.24 mmol, 1.5 equiv). ¹H NMR (400 MHz, Chloroform-d) δ 8.98 (d, J=2.1 Hz, 1H), 8.58-8.47 (m, 2H), 8.19 (s, 1H), 7.96 (s, 1H), 7.90 (d, J=5.5 Hz, 1H), 7.48 (s, 1H), 7.31 (s, 1H), 7.29 (s, 1H), 7.05 (s, 1H), 5.37 (s, 2H), 4.36 (brs, 1H), 4.04 (s, 3H), 3.90 (brs, 1H), 3.06 (brs, 2H), 2.87 (q, J=7.6 Hz, 2H), 1.33 (t, J=7.6 Hz, 3H); LCMS (ESI) Method 2: >95%, R_{T}=1.326 min, m/z=532.0 [M+H]⁺.

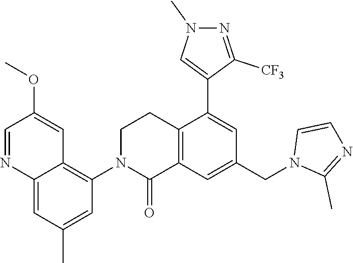 [SMILES: COc1cnc2cc(C)cc(N3CCc4c(cc(Cn5ccnc5C)cc4-c4cn(C)nc4C(F)(F)F)C3=O)c2c1]

### Example 2

### 2-(3-Methoxy-7-methylquinolin-5-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one

The title compound (57.9 mg, 103 μmol, 67% yield) was prepared following the Buchwald coupling procedure described for Intermediate 15, using 7-((2-methyl-1H-imidazol-1-yl)methyl)-5-(1-methyl-3-(trifluoromethyl)-1H-pyrazol-4-yl)-3,4-dihydroisoquinolin-1(2H)-one (Intermediate 7, 60 mg, 0.15 mmol, 1 equiv) and 5-bromo-3-methoxy-7-methylquinoline (Intermediate 13, 78 mg, 0.31 mmol, 2 equiv). ¹H NMR (400 MHz, Chloroform-d) δ 8.67 (d, J=2.7 Hz, 1H), 8.13 (s, 1H), 7.86 (s, 1H), 7.39 (s, 1H), 7.33 (s, 1H), 7.27 (d, J=2.7 Hz, 1H), 7.01 (s, 1H), 6.97 (s, 1H), 6.89 (s, 1H), 5.13 (s, 2H), 4.02 (s, 311), 3.99 (td, J=11.5, 4.5 Hz, 1H), 3.88 (s, 3H), 3.75 (dt, J=12.5, 5.6 Hz, 1H), 3.15-3.06 (m, 1H), 2.97 (dt, J=16.5, 4.8 Hz, 1H), 2.54 (s, 3H), 2.38 (s, 3H); ¹⁹F NMR (376 MHz, Chloroform-d) 5-60.16; LCMS (ESI) Method 2: >95%, R_{T}=1.437 min, m/z=561.0 [M+H]⁺.

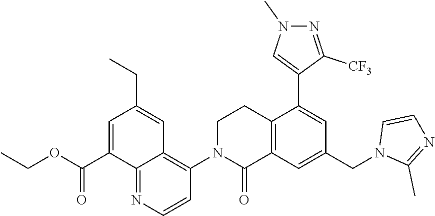 [SMILES: CCOC(=O)c1cc(CC)cc2c(N3CCc4c(cc(Cn5ccnc5C)cc4-c4cn(C)nc4C(F)(F)F)C3=O)ccnc12]

### Example 3

### Ethyl 6-ethyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxylate

The title compound (155 mg, 0.252 mmol, 98% yield) was prepared following the Buchwald coupling procedure described for Intermediate 15, using 7-((2-methyl-1H-imidazol-1-yl)methyl)-5-(1-methyl-3-(trifluoromethyl)-1H-pyrazol-4-yl)-3,4-dihydroisoquinolin-1(2H)-one (Intermediate 7, 100 mg, 0.257 mmol, 1 equiv) and ethyl 4-bromo-6-ethylquinoline-8-carboxylate (Intermediate 12, 150 mg, 0.488 mmol, 1.9 equiv). ¹H NMR (400 MHz, Chloroform-d) δ 9.03 (d, J=4.6 Hz, 1H), 8.12 (d, J=1.8 Hz, 1H), 7.89 (d, J=1.9 Hz, 1H), 7.72-7.71 (m, 1H), 7.40 (s, 1H), 7.36 (d, J=4.6 Hz, 1H), 7.03 (d, J=1.8 Hz, 1H), 6.96 (d, J=1.3 Hz, 1H), 6.88 (d, J=1.3 Hz, 1H), 5.13 (s, 2H), 4.54 (q, J=7.1 Hz, 2H), 4.02 (s, 3H), 4.00 (td, J=11.2, 4.2 Hz, 1H), 3.78 (dt, J=12.3, 5.3 Hz, 1H), 3.19-3.11 (m, 1H), 2.97 (dt, J=16.5, 4.7 Hz, 1H), 2.84 (q, J=7.5 Hz, 2H), 2.37 (s, 3H), 1.46 (t, J=7.1 Hz, 3H), 1.31 (t, J=7.6 Hz, 3H); LCMS (ESI) Method 2: R_{T}=1.374 min, m-=617.1 [M+H]⁺.

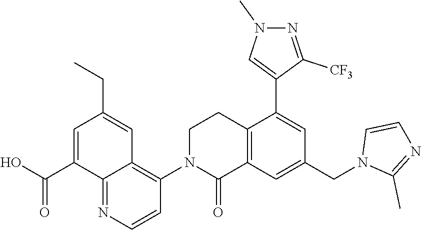 [SMILES: CCc1cc(C(=O)O)c2nccc(N3CCc4c(cc(Cn5ccnc5C)cc4-c4cn(C)nc4C(F)(F)F)C3=O)c2c1]

### Example 4

### 6-Ethyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxylic acid

Ethyl 6-ethyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxylate (140 mg, 0.227 mmol, 1 equiv) was dissolved in THF (2 mL) at room temperature. Water (0.7 mL) and LiOH (10.9 mg, 0.454 mmol. 2 equiv) were added and the reaction mixture was stirred overnight. The reaction was concentrated and was purified by silica gel chromatography (Combi-flash Rf, DCM/MeOH=0-10%, gradient) to yield the title compound (129 mg, 0.220 mmol, 97% yield). ¹H NMR (400 MHz, DMSO-d₆) δ 9.13 (d, J=4.9 Hz, 1H), 8.51 (d, J=1.9 Hz, 1H), 8.10 (s, 1H), 8.08 (d, J=1.9 Hz, 1H), 7.90 (d, J=1.6 Hz, 1H), 7.87 (d, J=4.9 Hz, 1H), 7.26 (d, J=1.4 Hz, 1H), 7.22 (d, J=1.2 Hz, 1H), 6.85 (d, J=1.1 Hz, 1H), 5.30 (s, 2H), 4.15 (td, J=11.6, 4.2 Hz, 1H), 4.00 (s, 3H), 3.86 (dt, J=12.0, 5.1 Hz, 1H), 3.26-3.20 (m, 1H), 2.95-2.90 (m, 1H), 2.89 (q, J=7.6 Hz, 2H), 2.27 (s, 3H), 1.25 (t, J=7.6 Hz, 3H); LCMS (ESI) Method 2: R_{T}=1.385 min, m/z=589.0 [M+H]⁺.

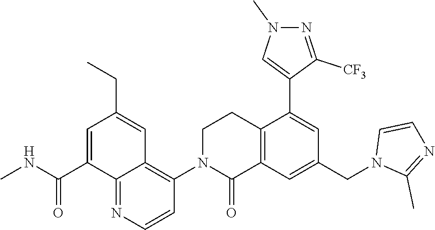 [SMILES: CCc1cc(C(=O)NC)c2nccc(N3CCc4c(cc(Cn5ccnc5C)cc4-c4cn(C)nc4C(F)(F)F)C3=O)c2c1]

### Example 5

### 6-Ethyl-N-methyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxamide

To a solution of 6-ethyl-4-(7-((2-methyl-1H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxylic acid (Example 4, 60 mg, 0.10 mmol, 1 equiv) in DMF (1 mL) was added HATU (58 mg, 0.15 mmol, 1.5 equiv). After stirring at room temperature for 10 min, the reaction was cooled to 0° C. Methylamine hydrochloride (69 mg, 1.0 mmol, 10 equiv) and N,N-diisopropylethylamine (0.036 mL, 0.20 mmol, 2 equiv) were added then stirred for additional 2 h at 0° C. and allowed to warm to room temperature. The reaction mixture was diluted with sat. NH₄Cl and extracted with CH₂Cl₂ (3×20 mL). The organic layer was then dried over MgSO₄, filtered, and concentrated. The residue was purified by reversed-phase HPLC (Phenomenex Gemini C18, H₂O/CH₃CN gradient from 15-80% CH₃CN, 0.1% TFA) followed by neutralization with sat. aq. NaHCO₃ to yield the title compound (16.7 mg, 0.028 mmol, 27% yield). ¹H NMR (400 MHz, Chloroform-d) δ 11.05 (d, J=4.5 Hz, 1H), 8.93 (d, J=4.7 Hz, 1H), 8.81 (d, J=1.7 Hz, 1H), 8.12 (s, 1H), 7.75 (s, 1H), 7.41-7.39 (m, 2H), 7.05 (s, 1H), 6.98 (s, 1H), 6.89 (s, 1H), 5.14 (s, 2H), 4.02 (s, 3H), 4.01 (td, J=11.3, 4.1 Hz, 1H), 3.82 (dt, J=12.4, 5.3 Hz, 1H), 3.22-3.12 (m, 4H), 2.99 (dt, J=16.5, 4.5 Hz, 1H), 2.87 (q, J=7.5 Hz, 2H), 2.39 (s, 3H), 1.32 (t, J=7.6 Hz, 3H); ¹⁹F NMR (376 MHz, Chloroform-d) δ −60.10; LCMS (ESI) Method 2: >95%, R_{T}=1.372 min, m/z=602.1 [M+H]⁺.

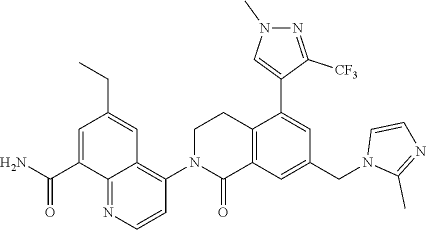 [SMILES: CCc1cc(C(N)=O)c2nccc(N3CCc4c(cc(Cn5ccnc5C)cc4-c4cn(C)nc4C(F)(F)F)C3=O)c2c1]

### Example 6

### 6-Ethyl-4-(7-((2-methyl-11H-imidazol-1-yl)methyl)-5-(1-methyl-3-(trifluoromethyl)-1H-pyrazol-4-yl)-1-oxo-3,4-dihydroisoquinolin-2(1H)-yl)quinoline-8-carboxamide

The title compound (22.5 mg, 0.038 mmol, 38% yield) was prepared following the procedure described for Example 5, substituting ammonium chloride (55 mg, 1.0 mmol, 10 equiv). ¹H NMR (400 MHz, Chloroform-d) δ 10.90 (d, J=4.8 Hz, 1H), 8.94 (d, J=4.6 Hz, 1H), 8.80 (s, 1H), 8.12 (s, 1H), 7.80 (s, 1H), 7.42-7.41 (m, 2H), 7.05 (s, 1H), 6.97 (s, 1H), 6.88 (s, 1H), 6.08 (d, J=4.5 Hz, 1H), 5.14 (s, 2H), 4.06-3.99 (m, 4H), 3.82 (dt, J=12.3, 5.2 Hz, 1H), 3.22-3.14 (m, 1H), 2.99 (dt, J=16.4, 4.3 Hz, 1H), 2.88 (q, J=7.6 Hz, 2H), 2.38 (s, 3H), 1.32 (t, J=7.6 Hz, 3H); ¹⁹F NMR (376 MHz, Chloroform-d) δ −60.10; LCMS (ESI) Method 2: >95%, R_{T}=1.361 min, m/z=588.1 [M+H]⁺.

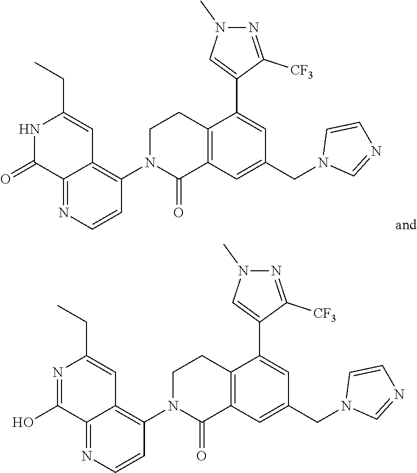 [SMILES: CCc1cc2c(N3CCc4c(cc(Cn5ccnc5)cc4-c4cn(C)nc4C(F)(F)F)C3=O)ccnc2c(=O)[nH]1 ; "and" ; CCc1cc2c(N3CCc4c(cc(Cn5ccnc5)cc4-c4cn(C)nc4C(F)(F)F)C3=O)ccnc2c(O)n1]

### Example 7

### 4-(7-((1H-Imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridin-8(7H)-one and 7-((1H-imidazol-1-yl)methyl)-2-(6-ethyl-8-hydroxy-1,7-naphthyridin-4-yl)-5-(1-methyl-3-(trifluoromethyl)-1H-pyrazol-4-yl)-3,4-dihydroisoquinolin-1(2H)-one mixture

PBr₃ (0.25 mL, 2.67 mmol, 1.5 equiv) was added to a solution of 7-((1H-imidazol-1-yl)methyl)-2-(6-ethyl-8-methoxy-1,7-naphthyridin-4-yl)-5-(1-methyl-3-(trifluoromethyl)-1H-pyrazol-4-yl)-3,4-dihydroisoquinolin-1(2H)-one (Intermediate 14, 1.00 g, 1.78 mmol, 1 equiv) in DMF (6 mL) at 25° C. The reaction was stirred overnight. Sat. aq. NaHCO₃ was added and the mixture was extracted with EtOAc. The combined organic layers were dried over MgSO₄, concentrated under reduced pressure. The residue was purified by flash chromatography (Combi-flash Rf, DCM/MeOH=0-20% gradient) to afford the inseparable mixture of title compounds (661 mg, 1.21 mmol, 68% yield). ¹H NMR (400 MHz, Chloroform-d) δ 11.24-10.78 (m, 1H), 8.88 (d, J=4.6 Hz, 1H), 8.15 (s, 1H), 7.63 (s, 1H), 7.47 (d, J=4.7 Hz, 1H), 7.41 (s, 1H), 7.19 (s, 1H), 7.11 (s, 1H), 6.95 (s, 1H), 6.21 (s, 1H), 5.21 (s, 2H), 4.03 (s, 3H), 3.99 (td, J=11.6, 4.2 Hz, 1H), 3.76 (dt, J=12.1, 5.2 Hz, 1H), 3.16-3.07 (m, 1H), 2.97 (dt, J=16.5, 4.6 Hz, 1H), 2.74 (q, J=7.3 Hz, 2H), 1.30 (t, J=7.5 Hz, 3H); ¹⁹F NMR (376 MHz, Chloroform-d) δ −60.11; LCMS (ESI) Method 2: >95%, R_{T}=1.228 min, m/z=548.1 [M+H]⁺.

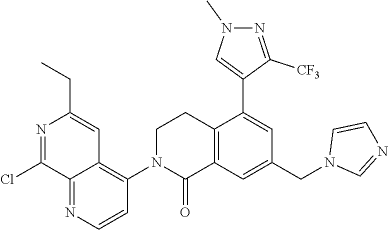 [SMILES: CCc1cc2c(N3CCc4c(cc(Cn5ccnc5)cc4-c4cn(C)nc4C(F)(F)F)C3=O)ccnc2c(Cl)n1]

### Example 8

### 7-((1H-Imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one

Excess POCl₃ (1.0 mL, 11.0 mmol) was added to the mixture of 4-(7-((1H-Imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridin-8(7H)-one and 7-((1H-imidazol-1-yl)methyl)-2-(6-ethyl-8-hydroxy-1,7-naphthyridin-4-yl)-5-(1-methyl-3-(trifluoromethyl)-1H-pyrazol-4-yl)-3,4-dihydroisoquinolin-1(2H)-one (Example 7, 150 mg, 274 μmol, 1 equiv) and the reaction was heated at 80° C. for 2 h. The reaction was cooled to room temperature, and ice was added to quench the reaction. The quenched mixture was neutralized with sat. aq. NaHCO₃ and extracted with CH₂Cl₂. The organic layer was dried over MgSO₄ and concentrated under reduced pressure to give the title compound (110 mg, 0.194 mmol, 70.9% yield). ¹H NMR (400 MHz, CDCl₃) δ 9.04 (d, J=4.2 Hz, 1H), 8.12 (s, 1H), 7.48 (q, J=6.8 Hz, 2H), 7.31 (q, J=11.8 Hz, 4H), 7.07 (s, 1H), 5.38 (s, 2H), 3.98 (s, 4H), 3.77 (d, J=7.5 Hz, 1H), 3.14 (s, 1H), 2.97 (d, J=15.6 Hz, 11H), 2.87 (q, J=7.4 Hz, 2H), 1.28 (t, J=7.4 Hz, 3H); LCMS (ESI) Method 2: >95%, R_{T}=1.650 min, m/z=566.0 [M+H]⁺.

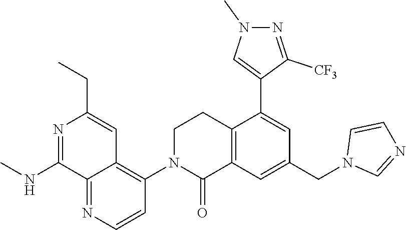 [SMILES: CCc1cc2c(N3CCc4c(cc(Cn5ccnc5)cc4-c4cn(C)nc4C(F)(F)F)C3=O)ccnc2c(NC)n1]

### Example 9

### 7-((1H-Imidazol-1-yl)methyl)-2-(6-ethyl-8-(methylamino)-1,7-naphthyridin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 50 mg, 0.088 mmol, 1 equiv) was dissolved in acetonitrile (1 mL) and stirred at room temperature. Then potassium carbonate (0.12 g, 0.88 mmol, 10 equiv) and methylamine hydrochloride (60 mg, 0.88 mmol, 10 equiv) were added, and the reaction was stirred until completion. The reaction mixture was diluted with sat. aq. NH₄Cl and extracted with CH₂Cl₂ (3×20 mL). The combined organic layer was dried over MgSO₄, filtered, and concentrated. The residue was purified by reversed-phase HPLC (Phenomenex Gemini C18, H₂O/CH₃CN gradient from 15-80% CH₃CN, 0.1% TFA) followed by neutralization with sat. aq. NaHCO₃ to yield the title compound (15.3 mg, 0.027 mmol, 31% yield). ¹H NMR (400 MHz, Chloroform-d) δ 8.66 (d, J=4.7 Hz, 1H), 8.16 (d, J=1.9 Hz, 1H), 7.59 (s, 1H), 7.42-7.40 (m, 2H), 7.17 (d, J=1.7 Hz, 1H), 7.08 (s, 1H), 6.95 (s, 1H), 6.91 (brs, 1H), 6.52 (s, 1H), 5.20 (s, 2H), 4.03 (s, 3H), 3.91 (td, J=11.2, 4.2 Hz, 1H), 3.83-3.78 (m, 1H), 3.22 (d, J=3.9 Hz, 3H), 3.21-3.09 (m, 1H), 2.95 (dt, J=16.8, 4.6 Hz, 1H), 2.76 (q, J=7.3 Hz, 2H), 1.31 (t, J=7.5 Hz, 3H); ¹⁹F NMR (376 MHz, Chloroform-d) δ-60.10; LCMS (ESI) Method 2: >95%, R_{T}=1.332 min, m/z=561.1 [M+H]⁺.

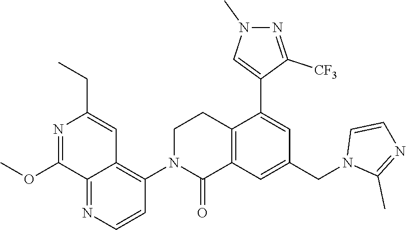 [SMILES: CCc1cc2c(N3CCc4c(cc(Cn5ccnc5C)cc4-c4cn(C)nc4C(F)(F)F)C3=O)ccnc2c(OC)n1]

### Example 10

### 2-(6-Ethyl-8-methoxy-1,7-naphthyridin-4-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one

The title compound (44 mg, 76 μmol, 74% yield) was prepared following the Buchwald coupling procedure described for Intermediate 15, using 7-((2-methyl-1H-imidazol-1-yl)methyl)-5-(1-methyl-3-(trifluoromethyl)-1H-pyrazol-4-yl)-3,4-dihydroisoquinolin-1(2H)-one (Intermediate 7, 40 mg, 0.10 mmol, 1 equiv) and 4-bromo-6-ethyl-8-methoxy-1,7-naphthyridine (Intermediate 14.44 mg, 0.16 mmol, 1.6 equiv). ¹H NMR (400 MHz, Chloroform-d) δ 8.93 (d, J=4.6 Hz, 1H), 8.12 (d, J=1.7 Hz, 1H), 7.48 (d, J=4.6 Hz, 1H), 7.40 (s, 1H), 7.03 (d, J=1.8 Hz, 1H), 6.97 (s, 1H), 6.95 (s, 1H), 6.89 (s, 1H), 5.14 (s, 2H), 4.22 (s, 3H), 4.02 (s, 3H), 3.99 (td, J=11.2, 4.2 Hz, 1H), 3.81 (dt, J=12.2, 5.3 Hz, 1H), 3.19-3.11 (m, 1H), 2.97 (dt, J=16.5, 4.6 Hz, 1H), 2.81 (q, J=7.5 Hz, 2H), 2.38 (s, 3H), 1.33 (t, J=7.5 Hz, 3H); ¹⁹F NMR (376 MHz, Chloroform-d) δ −60.09; LCMS (ESI) Method 2: >95%, R_{T}=1.555 min, m/z=576.0 [M+H]⁺.

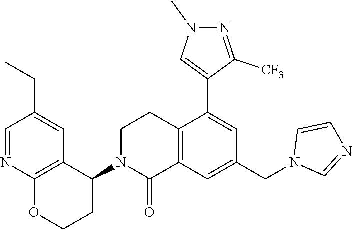 [SMILES: CCc1cnc2c(c1)[C@@H](N1CCc3c(cc(Cn4ccnc4)cc3-c3cn(C)nc3C(F)(F)F)C1=O)CCO2]

### Example 11

### (S)-7-((1H-Imidazol-1-yl)methyl)-2-(6-ethyl-3,4-dihydro-2H-pyrano[2,3-b]pyridin-4-yl)-5-(1-methyl-3-(trifluoromethyl)-1H-pyrazol-4-yl)-3,4-dihydroisoquinolin-1(2H)-one

To a suspension of (S)-6-ethyl-3,4-dihydro-2H-pyrano[2,3-b]pyridin-4-amine dihydrochloride salt (Intermediate 16, 56 mg, 222 μmol, 2.0 equiv) in THF (2.5 mL) at −78° C. was added 1M tert-butylmagnesium chloride (666 μL, 0.66 mmol, 6.0 equiv). The acetone/dry ice bath was removed and the reaction mixture was allowed to warm to 0° C. and stir for 20 min. Ethyl 5-((1H-imidazol-1-yl)methyl)-2-(2-chloroethyl)-3-(1-methyl-3-(trifluoromethyl)-1H-pyrazol-4-yl)benzoate (Intermediate 23, 45 mg, 111 μmol, 1 equiv) was added as a solution in THF (4 mL). The reaction was warmed to room temperature and stirred for 16 h, then quenched with sat. aq. NH₄Cl solution. The mixture was extracted with EtOAc. The combined organic layers were dried (Na₂SO₄), filtered, and concentrated. The residue was purified by reversed-phase HPLC (Phenomenex Gemini C18, H₂O/CH₃CN gradient from 30-90% CH₃CN, 0.1% TFA) to yield the title compound (30 mg, 56 μmol, 50% yield). ¹H NMR (400 MHz, Chloroform-d) δ 8.12 (d, J=2.0 Hz, 1H), 7.99 (d, J=2.0 Hz, 1H), 7.57 (s, 1H), 7.32 (s, 2H), 7.12 (d, J=2.0 Hz, 1H), 7.10 (s, 1H), 6.94 (m, 1H), 6.21 (t, J=8.4 Hz, 1H), 5.19 (s, 2H), 5.50 (m, 1H), 4.35 (m, 1H), 3.99 (s, 3H), 3.28 (m, 1H), 3.09 (m, 1H), 2.68 (m, 2H), 2.53 (m, 2H), 2.16 (m, 2H), 1.16 (t, J=7.6 Hz, 3H); LCMS (ESI): >95%, m/z=537.4 [M+H]⁺.

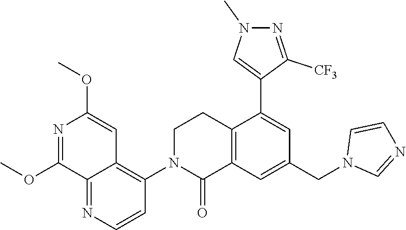 [SMILES: COc1cc2c(N3CCc4c(cc(Cn5ccnc5)cc4-c4cn(C)nc4C(F)(F)F)C3=O)ccnc2c(OC)n1]

### Example 12

### 7-((1H-Imidazol-1-yl)methyl)-2-(6,8-dimethoxy-1,7-naphthyridin-4-yl)-5-(1-methyl-3-(trifluoromethyl)-1H-pyrazol-4-yl)-3,4-dihydroisoquinolin-1(2H)-one

The title compound (189 mg, 0.337 mmol, 97% yield) was prepared following the Buchwald coupling procedure described for Example 1, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 130 mg, 0.346 mmol, 1 equiv) and 4-bromo-6,8-dimethoxy-1,7-naphthyridine (Intermediate 24, 149 mg, 0.554 mmol, 1.6 equiv). ¹H NMR (400 MHz, Chloroform-d) δ 8.79 (d, J=4.5 Hz, 1H), 7.74 (s, 1H), 7.43 (d, J=4.5 Hz, 1H), 7.41 (s, 1H), 7.20 (s, 1H), 7.15 (s, 1H), 6.98 (s, 1H), 6.43 (s, 1H), 5.19 (s, 2H), 4.24 (s, 3H), 4.03 (s, 3H), 3.99-3.93 (m, 4H), 3.80 (dt, J=12.3, 5.3 Hz, 1H), 3.18-3.10 (m, 1H), 2.96 (dt, J=16.5, 4.5 Hz, 1H); ¹⁹F NMR (376 MHz, Chloroform-d) 5-60.08; LCMS (ESI) Method 2: >95%, R_{T}=1.867 min, m/z=564.0 [M+H]⁺.

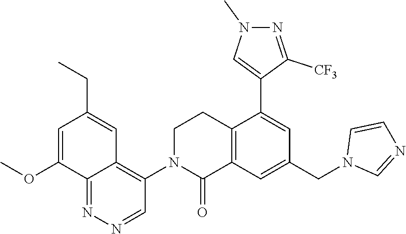 [SMILES: CCc1cc(OC)c2nncc(N3CCc4c(cc(Cn5ccnc5)cc4-c4cn(C)nc4C(F)(F)F)C3=O)c2c1]

### Example 13

### 7-((1H-Imidazol-1-yl)methyl)-2-(6-ethyl-8-methoxycinnolin-4-yl)-5-(1-methyl-3-(trifluoromethyl)-1H-pyrazol-4-yl)-3,4-dihydroisoquinolin-1(2H)-one

The title compound (50 mg, 0.089 mmol, 61% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 55 mg, 0.15 mmol, 1 equiv) and 4-bromo-6-ethyl-8-methoxycinnoline (Intermediate 25, 59 mg, 0.22 mmol, 1.5 equiv). ¹H NMR (400 MHz, Chloroform-d) δ 9.24 (s, 1H), 8.16 (d, J=1.8 Hz, 1H), 7.74 (s, 1H), 7.52 (s, 1H), 7.22 (d, J=1.9 Hz, 1H), 7.14 (s, 1H), 7.12 (s, 1H), 6.99 (d, J=1.1 Hz, 1H), 6.98 (s, 1H), 5.23 (s, 2H), 4.18 (s, 3H), 4.09-4.00 (m, 4H), 3.89-3.83 (m, 1H), 3.18-3.10 (m, 1H), 3.01 (dt, J=16.4, 4.9 Hz, 1H), 2.84 (q, J=7.6 Hz, 2H), 1.32 (t, J=7.6 Hz, 3H); ¹¹F NMR (376 MHz, Chloroform-d) 5-60.06; LCMS (ESI) Method 2: >95%, R_{T}=1.740 min, m/z=562.0 [M+H]⁺.

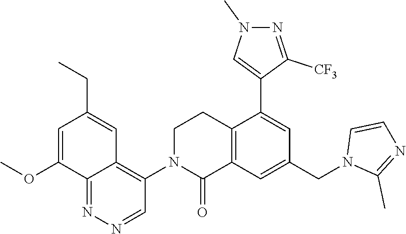 [SMILES: CCc1cc(OC)c2nncc(N3CCc4c(cc(Cn5ccnc5C)cc4-c4cn(C)nc4C(F)(F)F)C3=O)c2c1]

### Example 14

### 2-(6-Ethyl-8-methoxycinnolin-4-yl)-7-((2-methyl-1H-imidazol-1-yl)methyl)-5-(1-methyl-3-(trifluoromethyl)-1H-pyrazol-4-yl)-3,4-dihydroisoquinolin-1(2H)-one

The title compound (62.5 mg, 0.109 mmol, 77% yield) was prepared following the Buchwald coupling procedure described for Intermediate 15, using 7-((2-methyl-1H-imidazol-1-yl)methyl)-5-(1-methyl-3-(trifluoromethyl)-1H-pyrazol-4-yl)-3,4-dihydroisoquinolin-(2H)-one (Intermediate 7, 55 mg, 0.14 mmol, 1 equiv) and 4-bromo-6-ethyl-8-methoxycinnoline (Intermediate 25, 57 mg, 0.21 mmol, 1.5 equiv). ¹H NMR (400 MHz, Chloroform-d) δ 9.23 (s, 1H), 8.12 (d, J=1.7 Hz, 1H), 7.40 (s, 1H), 7.12 (s, 1H), 7.04 (d, J=1.8 Hz, 1H), 6.99-6.98 (m, 2H), 6.90 (d, J=1.3 Hz, 1H), 5.14 (s, 2H), 4.18 (s, 3H), 4.08-3.99 (m, 4H), 3.88-3.82 (m, 1H), 3.18-3.10 (m, 1H), 3.00 (dt, J=16.8, 4.9 Hz, 1H), 2.84 (q, J=7.6 Hz, 2H), 2.40 (s, 3H), 1.33 (t, J=7.6 Hz, 3H); ¹⁹F NMR (376 MHz, Chloroform-d) δ −60.06; LCMS (ESI) Method 2: >95%, R_{T}=1.753 min, m % z=576.1 [M+H]⁺.

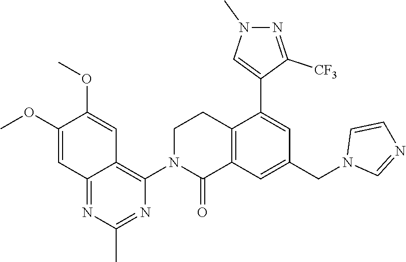 [SMILES: COc1cc2nc(C)nc(N3CCc4c(cc(Cn5ccnc5)cc4-c4cn(C)nc4C(F)(F)F)C3=O)c2cc1OC]

### Example 15

### 7-((1H-Imidazol-1-yl)methyl)-2-(6,7-dimethoxy-2-methylquinazolin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv), 4-chloro-6,7-dimethoxy-2-methylquinazoline (76 mg, 0.32 mmol, 2 equiv), cesium carbonate (104 mg, 0.32 mmol, 2 equiv), Xantphos (28 mg, 0.048 mmol, 0.3 equiv), and Pd₂(dba)₃ (15 mg, 0.016 mmol, 0.1 equiv) were dissolved in 1,4-dioxane (1 mL) under an Ar. The reaction mixture was stirred for 14 h at 115° C. then cooled to 23° C. Brine was added to the mixture and the mixture was extracted with CH₂Cl₂ (3×20 mL). The combined organic layers were dried over MgSO₄ and concentrated under reduced pressure. The residue was purified by reversed-phase HPLC (Phenomenex Gemini C18, H₂O/CH₃CN gradient from 15-70% CH₃CN, 0.1% TFA) followed by neutralization with sat. aq. NaHCO₃ to obtain the title compound (61.3 mg, 0.106 mmol, 66% yield). ¹H NMR (400 MHz, DMSO-d₆) δ 8.08 (s, 1H), 7.99 (s, 1H), 7.79 (s, 1H), 7.39 (s, 1H), 7.34 (s, 1H), 7.22 (s, 1H), 7.06 (s, 1H), 6.91 (s, 1H), 5.31 (s, 2H), 3.99 (s, 4H), 3.98 (s, 4H), 3.79 (s, 3H), 3.01 (brs, 2H), 2.67 (s, 3H); ¹⁹F NMR (376 MHz, Chloroform-d) δ −58.24; LCMS (ESI) Method 2: >95%, R_{T}=1.663 min, m/z=578.1 [M+H]⁺.

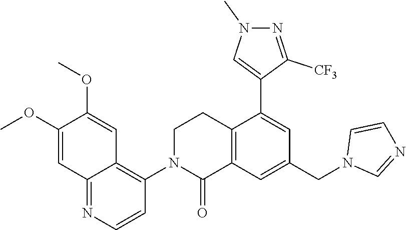 [SMILES: COc1cc2nccc(N3CCc4c(cc(Cn5ccnc5)cc4-c4cn(C)nc4C(F)(F)F)C3=O)c2cc1OC]

### Example 16

### 7-((1H-Imidazol-1-yl)methyl)-2-(6,7-dimethoxyquinolin-4-yl)-5-(1-methyl-3-(trifluoromethyl)-1H-pyrazol-4-yl)-3,4-dihydroisoquinolin-1(2H)-one

The title compound (61.4 mg, 0.109 mmol, 68% yield) was prepared following the Buchwald coupling procedure described for Intermediate 15, substituting 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-bromo-6,7-dimethoxyquinoline (86 mg, 0.32 mmol, 2 equiv). ¹H NMR (400 MHz, Chloroform-d) δ 8.78 (d, J=4.8 Hz, 1H), 8.18 (s, 1H), 7.64 (s, 1H), 7.50 (s, 1H), 7.41 (s, 1H), 7.22 (d, J=4.7 Hz, 1H), 7.19 (s, 1H), 7.12 (s, 1H), 7.00 (s, 1H), 6.96 (s, 1H), 5.21 (s, 2H), 4.05 (s, 3H), 4.02-3.97 (m, 4H), 3.94 (s, 3H), 3.83-3.77 (m, 1H), 3.18-3.10 (m, 1H), 3.01-2.94 (m, 1H); ¹¹F NMR (376 MHz, Chloroform-d) δ-60.16; LCMS (ESI) Method 2: >95%, R_{T}=1.615 min, m/z=563.1 [M+H]⁺.

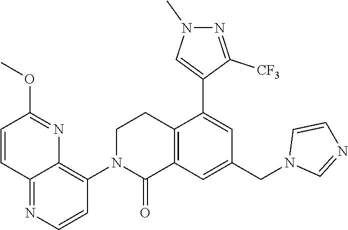 [SMILES: COc1ccc2nccc(N3CCc4c(cc(Cn5ccnc5)cc4-c4cn(C)nc4C(F)(F)F)C3=O)c2n1]

### Example 17

### 7-((1H-Imidazol-1-yl)methyl)-2-(6-methoxy-1,5-naphthyridin-4-yl)-5-(1-methyl-3-(trifluoromethyl)-1H-pyrazol-4-yl)-3,4-dihydroisoquinolin-1(2H)-one

The title compound (67.8 mg, 0.127 mmol, 68% yield) was prepared following the Buchwald coupling procedure described for Intermediate 15, substituting 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 70 mg, 0.186 mmol, 1 equiv) and 8-bromo-2-methoxy-1,5-naphthyridine (67 mg, 0.28 mmol, 1.5 equiv). ¹H NMR (400 MHz, Chloroform-d) δ 8.84 (d, J=4.7 Hz, 1H), 8.26 (d, J=9.1 Hz, 1H), 7.72 (brs, 1H), 7.66 (d, J=4.7 Hz, 1H), 7.41 (s, 1H), 7.16 (s, 1H), 7.14 (s, 1H), 6.97 (s, 1H), 5.22 (s, 2H), 4.08 (t, J=6.2 Hz, 2H), 4.03 (s, 3H), 3.95 (s, 3H), 3.08 (t, J=6.3 Hz, 2H); ¹⁹F NMR (376 MHz, Chloroform-d) 6-60.28; LCMS (ESI) Method 2: >95%, R_{T}=1.421 min, m/z=534.0 [M+H]⁺.

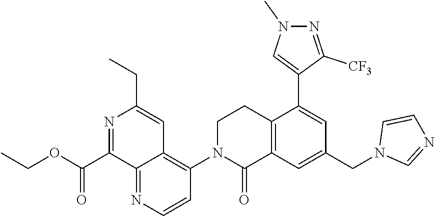 [SMILES: CCOC(=O)c1nc(CC)cc2c(N3CCc4c(cc(Cn5ccnc5)cc4-c4cn(C)nc4C(F)(F)F)C3=O)ccnc12]

### Example 18

### Ethyl 4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridine-8-carboxylate

The title compound (130 mg, 0.214 mmol, 90% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 90 mg, 0.24 mmol, 1 equiv) and ethyl 4-bromo-6-ethyl-1,7-naphthyridine-8-carboxylate (Intermediate 26, 111 mg, 0.36 mmol, 1.5 equiv). ¹H NMR (400 MHz, Chloroform-d) δ 9.04 (d, J=4.6 Hz, 1H), 8.16 (s, 1H), 7.76 (s, 1H), 7.51-7.49 (m, 2H), 7.43 (s, 1H), 7.23 (s, 1H), 7.14 (s, 1H), 6.98 (s, 1H), 5.23 (s, 2H), 4.61 (qd, J=7.2 Hz, 2.4 Hz, 2H), 4.09-4.00 (m, 4H), 3.83-3.77 (m, 1H), 3.22-3.14 (m, 1H), 3.03-2.98 (m, 3H), 1.48 (t, J=7.2 Hz, 3H), 1.36 (t, J=7.6 Hz, 3H); LCMS (ESI) Method 2: >95%, R_{T}=1.362 min, m/z=604.0 [M+H]⁺.

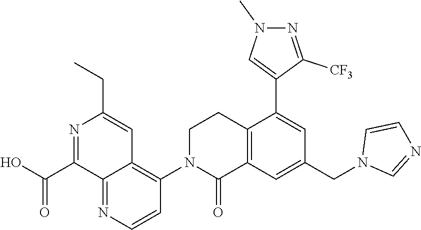 [SMILES: CCc1cc2c(N3CCc4c(cc(Cn5ccnc5)cc4-c4cn(C)nc4C(F)(F)F)C3=O)ccnc2c(C(=O)O)n1]

### Example 19

### 4-(7-((1H-Imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridine-8-carboxylic acid

The title compound (122 mg, 0.213 mmol, quant.) was prepared following the procedure described for Example 4, using ethyl 4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridine-8-carboxylate (Example 18, 128 mg, 0.214 mmol, 1 equiv). LCMS (ESI) Method 2: >95%, R_{T}=1.637 min, m/z=576.0 [M+H]⁺.

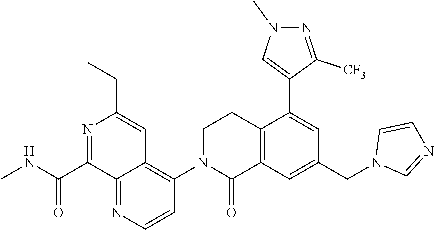 [SMILES: CCc1cc2c(N3CCc4c(cc(Cn5ccnc5)cc4-c4cn(C)nc4C(F)(F)F)C3=O)ccnc2c(C(=O)NC)n1]

### Example 20

### 4-(7-((1H-Imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-N-methyl-1,7-naphthyridine-8-carboxamide

The title compound (40 mg, 0.068 mmol, 33% yield) was prepared following the procedure described for Example 5, substituting 4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-6-ethyl-1,7-naphthyridine-8-carboxylic acid (Example 19, 120 mg, 0.208 mmol, 1 equiv). ¹H NMR (400 MHz, Chloroform-d) δ 9.18 (d, J=4.4 Hz, 1H), 9.06 (d, J=4.6 Hz, 1H), 8.15 (s, 1H), 7.77 (s, 1H), 7.54-7.53 (m, 2H), 7.44 (s, 1H), 7.24 (s, 1H), 7.15 (s, 1H), 6.98 (s, 1H), 5.24 (s, 2H), 4.07 (td, J=11.7, 4.0 Hz, 1H), 4.03 (s, 3H), 3.82 (dt, J=12.0, 5.2 Hz, 1H), 3.23-3.15 (m, 4H), 3.07 (q, J=7.6 Hz, 2H), 3.00 (dt, J=16.4, 4.6 Hz, 1H), 1.37 (t, J=7.6 Hz, 3H); ¹⁹F NMR (376 MHz, Chloroform-d) δ −60.08: LCMS (ESI) Method 2: >95%, R_{T}=1.411 min, m/z=589.0 [M+H]⁺.

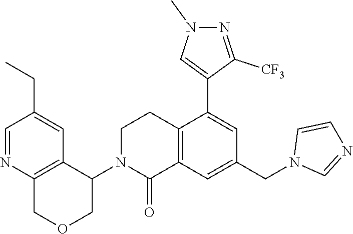 [SMILES: CCc1cnc2c(c1)C(N1CCc3c(cc(Cn4ccnc4)cc3-c3cn(C)nc3C(F)(F)F)C1=O)COC2]

### Example 21

### 7-((1H-Imidazol-1-yl)methyl)-2-(3-ethyl-5,8-dihydro-6H-pyrano[3,4-b]pyridin-5-yl)-5-(1-methyl-3-(trifluoromethyl)-1H-pyrazol-4-yl)-3,4-dihydroisoquinolin-1(2H)-one

The title compound (16 mg, 30 μmol, 28% yield) was prepared following the procedure described for Example 11, using ethyl 5-((1H-imidazol-1-yl)methyl)-2-(2-chloroethyl)-3-(1-methyl-3-(trifluoromethyl)-1H-pyrazol-4-yl)benzoate (Intermediate 23, 45 mg, 111 μmol, 1 equiv) and 3-ethyl-5,8-dihydro-6H-pyrano[3,4-b]pyridin-5-amine dihydrochloride salt (Intermediate 28, 54 mg, 215 μmol, 2.0 equiv). ¹H NMR (400 MHz, Chloroform-d) δ 8.36 (d, J=2.0 Hz, 1H), 8.13 (d, J=2.0 Hz, 1H), 7.86 (s, 1H), 7.45 (s, 1H), 7.31 (s, 1H), 7.17 (s, 1H), 7.15 (d, J=2.0 Hz, 1H), 7.00 (s, 1H), 5.96 (brs, 1H), 5.24 (s, 2H), 4.87 (d, J=16.0 Hz, 1H), 4.71 (d, J=16.0 Hz, 1H), 4.18 (dd, J=2.4, 12.4 Hz, 1H), 4.05 (dd, J=4.0, 12.4 Hz, 1H), 3.98 (s, 3H), 3.60 (m, 1H), 3.15 (m, 1H), 2.62 (m, 4H), 1.22 (t, J=7.6 Hz, 3H); LCMS (ESI): >95%, m/z=537.4 [M+H]⁺.

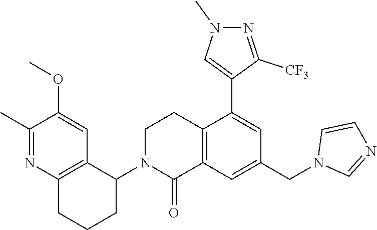 [SMILES: COc1cc2c(nc1C)CCCC2N1CCc2c(cc(Cn3ccnc3)cc2-c2cn(C)nc2C(F)(F)F)C1=O]

### Example 22

### 7-((1H-Imidazol-1-yl)methyl)-2-(3-methoxy-2-methyl-5,6,7,8-tetrahydroquinolin-5-yl)-5-(1-methyl-3-(trifluoromethyl)-1H-pyrazol-4-yl)-3,4-dihydroisoquinolin-1(2H)-one

Step A. Preparation of methyl 5-hydroxy-2-(3-methoxy-2-methyl-5,6,7,8-tetrahydroquinolin-5-yl)-1-oxo-1,2,3,4-tetrahydroisoquinoline-7-carboxylate To a suspension of 3-methoxy-2-methyl-5,6,7,8-tetrahydroquinolin-5-amine dihydrochloride salt (Intermediate 27, 20 mg, 75 μmol, 1 equiv) in dichloromethane (1 mL) at 30° C. was added DIPEA (0.037 mL, 21 μmol, 3 equiv). The mixture was stirred for 15 min, then dimethyl 2-hydroxy-2,3-dihydrobenzofuran-4,6-dicarboxylate (19 mg, 75 μmol, 1 equiv) and NaBH(OAc)₃ (24 mg, 113 μmol, 1.5 equiv) were added sequentially. The reaction was stirred at 30° C. for 2 h, then concentrated. The residue was dissolved in 1,4-dioxane (10 mL) and heated at 90° C. for 1 h. The mixture was diluted with water and extracted with EtOAc. The combined organic layers were dried (Na₂SO₄) and concentrated to provide the title compound, which was used without further purification.

Step B. Preparation of methyl 2-(3-methoxy-2-methyl-5,6,7,8-tetrahydroquinolin-5-yl)-1-oxo-5-(((trifluoromethyl)sulfonyl)oxy)-1,2,3,4-tetrahydroisoquinoline-7-carboxylate. The title compound (28.3 mg, 53.5 μmol, 71% yield over two steps) was prepared following the procedure described for Intermediate 2, using methyl 5-hydroxy-2-(3-methoxy-2-methyl-5,6,7,8-tetrahydroquinolin-5-yl)-1-oxo-1,2,3,4-tetrahydroisoquinoline-7-carboxylate (30 mg, 75 μmol, 1 equiv). ¹H NMR (400 MHz, Chloroform-d) δ 8.87 (s, 1H), 8.07 (s, 1H), 6.85 (s, 1H), 6.08 (dd, J=6.0, 9.6 Hz, 1H), 3.99 (s, 3H), 3.72 (s, 3H), 3.45 (m, 1H), 3.24 (m, 1H), 3.06 (m, 1H), 2.92 (m, 3H), 2.45 (s, 3H), 2.15 (m, 2H), 1.87 (m, 1H), 1.75 (m, 1H).

Step C. Preparation of methyl 2-(3-methoxy-2-methyl-5,6,7,8-tetrahydroquinolin-5-yl)-5-(1-methyl-3-(trifluoromethyl)-1H-pyrazol-4-yl)-1-oxo-1,2,3,4-tetrahydroisoquinoline-7-carboxylate. The title compound (33 mg, 53.5 μmol, 100% yield) was prepared following the procedure described for Intermediate 3, using methyl 2-(3-methoxy-2-methyl-5,6,7,8-tetrahydroquinolin-5-yl)-1-oxo-5-(((trifluoromethyl)sulfonyl)oxy)-1,2,3,4-tetrahydroisoquinoline-7-carboxylate (28.3 mg, 53.5 μmol, 1 equiv). LCMS (ESI): m/z=529.2 [M+H]⁺.

Step D. Preparation of 7-(hydroxymethyl)-2-(3-methoxy-2-methyl-5,6,7,8-tetrahydroquinolin-5-yl)-5-(1-methyl-3-(trifluoromethyl)-1H-pyrazol-4-yl)-3,4-dihydroisoquinolin-1(2H)-one. The title compound (25.8 mg, 51.5 μmol, 96% yield) was prepared following the procedure described for Intermediate 5, using methyl 2-(3-methoxy-2-methyl-5,6,7,8-tetrahydroquinolin-5-yl)-5-(1-methyl-3-(trifluoromethyl)-1H-pyrazol-4-yl)-1-oxo-1,2,3,4-tetrahydroisoquinoline-7-carboxylate (28.3 mg, 53.5 μmol, 1 equiv). ¹H NMR (400 MHz, Chloroform-d) δ 8.21 (s, 1H), 7.42 (d, J=2.0 Hz, 1H), 7.35 (s, 1H), 6.91 (s, 1H), 6.09 (dd, J=6.0, 9.6 Hz, 1H), 4.78 (s, 2H), 4.00 (s, 3H), 3.72 (s, 3H), 3.29 (m, 1H), 3.06 (m, 1H), 2.90 (m, 2H), 2.65 (m, 2H), 2.45 (s, 3H), 2.09 (m, 2H), 1.89 (m, 1H), 1.78 (m, 1H).

Step E. Preparation of 7-(chloromethyl)-2-(3-methoxy-2-methyl-5,6,7,8-tetrahydroquinolin-5-yl)-5-(1-methyl-3-(trifluoromethyl)-1H-pyrazol-4-yl)-3,4-dihydroisoquinolin-1(2H)-one. The title compound (26.8 mg, 51.5 μmol, 100% yield) was prepared following the procedure described for Intermediate 6, using 7-(hydroxymethyl)-2-(3-methoxy-2-methyl-5,6,7,8-tetrahydroquinolin-5-yl)-5-(1-methyl-3-(trifluoromethyl)-1H-pyrazol-4-yl)-3,4-dihydroisoquinolin-1(2H)-one (25.8 mg, 51.5 μmol, 1 equiv) and thionyl chloride (2.0 equiv). LCMS (ESI): m-z=519.2 [M+H]⁺.

Step F. Preparation of 7-((1H-imidazol-1-yl)methyl)-2-(3-methoxy-2-methyl-5,6,7,8-tetrahydroquinolin-5-yl)-5-(1-methyl-3-(trifluoromethyl)-1H-pyrazol-4-yl)-3,4-dihydroisoquinolin-1(2H)-one. The title compound (23.5 mg, 42.7 μmol, 82% yield) was prepared following the procedure described for Intermediate 7, using 7-(chloromethyl)-2-(3-methoxy-2-methyl-5,6,7,8-tetrahydroquinolin-5-yl)-5-(1-methyl-3-(trifluoromethyl)-1H-pyrazol-4-yl)-3,4-dihydroisoquinolin-1(2H)-one (26.7 mg, 51.5 μmol, 1 equiv) and 1H-imidazole (206 μmol, 4 equiv). ¹H NMR (400 MHz, Chloroform-d) δ 8.14 (d, J=2.0 Hz, 1H), 7.65 (s, 1H), 7.32 (s, 1H), 7.12 (m, 2H), 7.00 (s, 1H), 6.91 (s, 1H), 6.07 (m, 1H), 5.20 (s, 2H), 3.99 (s, 3H), 3.73 (s, 3H), 3.30 (m, 1H), 3.06 (m, 1H), 2.89 (m, 2H), 2.65 (m, 2H), 2.46 (s, 3H), 2.11 (m, 2H), 1.89 (m, 1H), 1.76 (m, 11H); LCMS (ESI): >95%, m/z=551.5 [M+H]⁺.

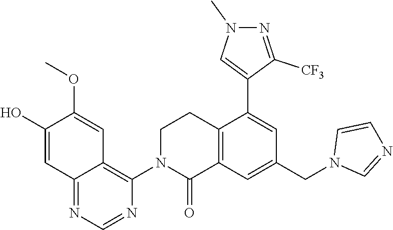 [SMILES: COc1cc2c(N3CCc4c(cc(Cn5ccnc5)cc4-c4cn(C)nc4C(F)(F)F)C3=O)ncnc2cc1O]

### Example 23

### 7-((1H-Imidazol-1-yl)methyl)-2-(7-hydroxy-6-methoxyquinazolin-4-yl)-5-(1-methyl-3-(trifluoromethyl)-1H-pyrazol-4-yl)-3,4-dihydroisoquinolin-1(2H)-one

The title compound (9 mg, 0.02 mmol, 10% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-chloro-6-methoxyquinazolin-7-ol (51 mg, 0.24 mmol, 1.5 equiv). ¹H NMR (400 MHz, DMSO-d₆) δ 10.92 (s, 1H), 8.93 (s, 1H), 8.09 (s, 1H), 8.02 (d, J=1.4 Hz, 1H), 7.99 (brs, 1H), 7.42 (s, 1H), 7.30 (brs, 1H), 7.26 (s, 1H), 7.10 (s, 1H), 7.02 (brs, 1H), 5.35 (s, 2H), 4.05-3.96 (m, 5H), 3.82 (s, 3H), 3.02 (brs, 2H); ¹⁹F NMR (376 MHz, DMSO-d₆) δ −58.21; LCMS (ESI) Method 2: >95%, R_{T}=1.125 min, m/z=550.0 [M+H]⁺.

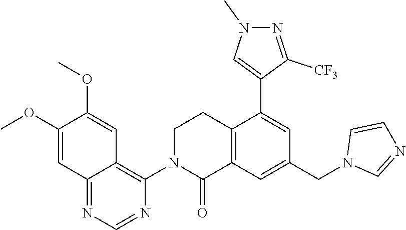 [SMILES: COc1cc2ncnc(N3CCc4c(cc(Cn5ccnc5)cc4-c4cn(C)nc4C(F)(F)F)C3=O)c2cc1OC]

### Example 24

### 7-((1H-Imidazol-1-yl)methyl)-2-(6,7-dimethoxyquinazolin-4-yl)-5-(1-methyl-3-(trifluoromethyl)-1H-pyrazol-4-yl)-3,4-dihydroisoquinolin-1(2H)-one

The title compound (61 mg, 0.11 mmol, 68% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-chloro-6,7-dimethoxyquinazoline (54 mg, 0.24 mmol, 1.5 equiv). ¹H NMR (400 MHz, Chloroform-d) 9.03 (s, 1H), 8.18 (d, J=1.7 Hz, 1H), 7.93-7.91 (m, 1H), 7.44 (s, 1H), 7.36 (s, 1H), 7.23 (s, 1H), 7.17 (s, 1H), 7.00-6.99 (m, 2H), 5.19 (s, 2H), 4.15 (brs, 2H), 4.07 (s, 3H), 4.04 (s, 3H), 3.94 (s, 3H), 3.06 (t, J=6.2 Hz, 2H); ¹⁹F NMR (376 MHz, Chloroform-d) 6-60.09; LCMS (ESI) Method 2: >95%, R_{T}=1.212 min, m/z=564.0 [M+H]⁺.

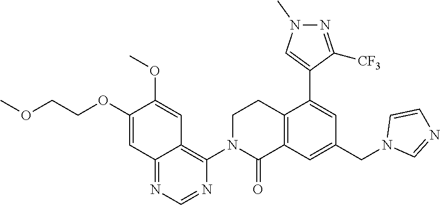 [SMILES: COCCOc1cc2ncnc(N3CCc4c(cc(Cn5ccnc5)cc4-c4cn(C)nc4C(F)(F)F)C3=O)c2cc1OC]

### Example 25

### 7-((1H-Imidazol-1-yl)methyl)-2-(6-methoxy-7-(2-methoxyethoxy)quinazolin-4-yl)-5-(1-methyl-3-(trifluoromethyl)-1H-pyrazol-4-yl)-3,4-dihydroisoquinolin-1(2H)-one

The title compound (61 mg, 0.10 mmol, 63% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-chloro-6-methoxy-7-(2-methoxyethoxy)quinazoline (Intermediate 29, 65 mg, 0.24 mmol, 1.5 equiv). ¹H NMR (400 MHz, Chloroform-d) δ 9.02 (s, 1H), 8.18 (d, J=1.5 Hz, 1H), 7.99 (s, 1H), 7.44 (s, 1H), 7.36 (s, 1H), 7.23 (d, J=1.6 Hz, 1H), 7.19 (s, 1H), 7.02 (s, 1H), 6.98 (s, 1H), 5.27 (s, 2H), 4.36 (t, J=4.7 Hz, 2H), 4.15 (brs, 2H), 4.04 (s, 3H), 3.91 (s, 3H), 3.89 (t, J=4.7 Hz, 2H), 3.48 (s, 3H), 3.06 (t, J=6.0 Hz, 2H); LCMS (ESI) Method 2: >95%, R_{T}=1.225 min, m/z=608.1 [M+H]⁺.

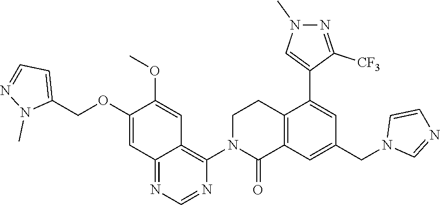 [SMILES: COc1cc2c(N3CCc4c(cc(Cn5ccnc5)cc4-c4cn(C)nc4C(F)(F)F)C3=O)ncnc2cc1OCc1ccnn1C]

### Example 26

### 7-((1H-Imidazol-1-yl)methyl)-2-(6-methoxy-7-((1-methyl-1H-pyrazol-5-yl)methoxy)quinazolin-4-yl)-5-(1-methyl-3-(trifluoromethyl)-1H-pyrazol-4-yl)-3,4-dihydroisoquinolin-1(2H)-one

The title compound (42 mg, 0.11 mmol, 61% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 40 mg, 0.11 mmol, 1 equiv) and 4-chloro-6-methoxy-7-((1-methyl-1H-pyrazol-5-yl)methoxy)quinazoline (Intermediate 30, 49 mg, 0.16 mmol, 1.5 equiv). ¹H NMR (400 MHz, Chloroform-d) δ 9.03 (s, 1H), 8.17 (s, 1H), 8.06-7.93 (m, 1H), 7.46 (s, 2H), 7.44 (s, 1H), 7.24 (s, 1H), 7.20-7.17 (m, 1H), 7.02-6.99 (m, 2H), 6.44 (d, J=1.6 Hz, 1H), 5.29-5.16 (m, 4H), 4.16 (brs, 2H), 4.04 (s, 3H), 3.96 (s, 3H), 3.90 (s, 3H), 3.06 (t, J=6.0 Hz, 2H); LCMS (ESI) Method 2. >95%, R_{T}=1.249 min, m/z=644.1 [M+H]⁺.

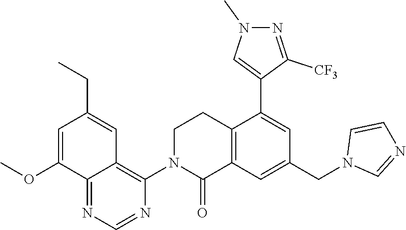 [SMILES: CCc1cc(OC)c2ncnc(N3CCc4c(cc(Cn5ccnc5)cc4-c4cn(C)nc4C(F)(F)F)C3=O)c2c1]

### Example 27

### 7-((1H-imidazol-1-yl)methyl)-2-(6-ethyl-8-methoxyquinazolin-4-yl)-5-(1-methyl-3-(trifluoromethyl)-1H-pyrazol-4-yl)-3,4-dihydroisoquinolin-1(2H)-one

The title compound (25 mg, 0.045 mmol, 56% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 30 mg, 0.08 mmol, 1 equiv) and 4-chloro-6-ethyl-8-methoxyquinazoline (Intermediate 31, 27 mg, 0.12 mmol, 1.5 equiv). ¹H NMR (400 MHz, Chloroform-d) δ 9.16 (s, 1H), 8.19 (d, J=1.7 Hz, 1H), 7.89 (s, 1H), 7.44 (s, 1H), 7.23 (d, J=1.5 Hz, 1H), 7.20 (s, 1H), 7.17 (s, 1H), 7.09 (d, J=1.1 Hz, 1H), 7.00 (s, 1H), 5.25 (s, 2H), 4.13 (brs, 2H), 4.10 (s, 3H), 4.04 (s, 3H), 3.07 (brs, 2H), 2.81 (q, J=7.5 Hz, 2H), 1.30 (t, J=7.6 Hz, 3H); LCMS (ESI) Method 2: >95%, R_{T}=1.333 min, m z=562.0 [M+H]⁺.

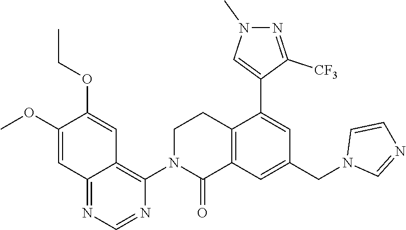 [SMILES: CCOc1cc2c(N3CCc4c(cc(Cn5ccnc5)cc4-c4cn(C)nc4C(F)(F)F)C3=O)ncnc2cc1OC]

### Example 28

### 7-((1H-Imidazol-1-yl)methyl)-2-(6-ethoxy-7-methoxyquinazolin-4-yl)-5-(1-methyl-3-(trifluoromethyl)-1H-pyrazol-4-yl)-3,4-dihydroisoquinolin-1(2H)-one

The title compound (25 mg, 0.043 mmol, 27% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-chloro-6-ethoxy-7-methoxyquinazoline (Intermediate 32, 57 mg, 0.24 mmol, 1.5 equiv). ¹H NMR (400 MHz, Chloroform-d) δ 9.01 (s, 1H), 8.18 (s, 1H), 8.11-7.94 (m, 1H), 7.44 (s, 1H), 7.35 (s, 1H), 7.23 (s, 1H), 7.21-7.17 (m, 1H), 7.03-7.00 (m, 1H), 6.98 (s, 1H), 5.27 (s, 2H), 4.16-4.11 (m, 4H), 4.06 (s, 3H), 4.04 (s, 3H), 3.05 (t, J=5.9 Hz, 2H), 1.51 (t, J=7.0 Hz, 3H); LCMS (ESI) Method 2: >95%, R_{T}=1.490 min, m/z=578.1 [M+H]⁺.

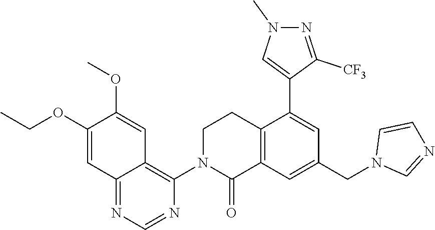 [SMILES: CCOc1cc2ncnc(N3CCc4c(cc(Cn5ccnc5)cc4-c4cn(C)nc4C(F)(F)F)C3=O)c2cc1OC]

### Example 29

### 7-((1H-Imidazol-1-yl)methyl)-2-(7-ethoxy-6-methoxyquinazolin-4-yl)-5-(1-methyl-3-(trifluoromethyl)-1H-pyrazol-4-yl)-3,4-dihydroisoquinolin-1(2H)-one

The title compound (37 mg, 0.064 mmol, 40% yield) was prepared following the Buchwald coupling procedure described for Intermediate 15, using 7-((1H-imidazol-1-yl)methyl)-5-(l-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-chloro-7-ethoxy-6-methoxyquinazoline (Intermediate 33, 76 mg, 0.32 mmol, 2 equiv). ¹H NMR (400 MHz, Chloroform-d) δ 9.01 (s, 1H), 8.18 (s, 1H), 7.91-7.81 (m, 1H), 7.43 (s, 1H), 7.34 (s, 1H), 7.22 (s, 1H), 7.16 (s, 1H), 6.99 (s, 1H), 6.98 (s, 1H), 5.25 (s, 2H), 4.31 (q, J=7.0 Hz, 2H), 4.14 (brs, 2H), 4.03 (s, 3H), 3.93 (s, 3H), 3.05 (t, J=6.2 Hz, 2H), 1.58 (t, J=7.0 Hz, 3H); LCMS (ESI) Method 2. >95%, R_{T}=1.283 min, m % z=578.1 [M+H]⁺.

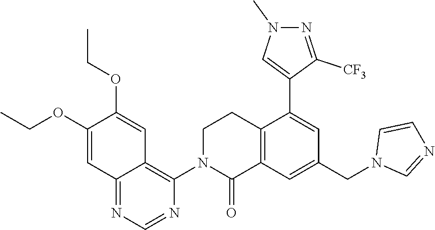 [SMILES: CCOc1cc2ncnc(N3CCc4c(cc(Cn5ccnc5)cc4-c4cn(C)nc4C(F)(F)F)C3=O)c2cc1OCC]

### Example 30

### 7-((1H-Imidazol-1-yl)methyl)-2-(6,7-diethoxyquinazolin-4-yl)-5-(1-methyl-3-(trifluoromethyl)-1H-pyrazol-4-yl)-3,4-dihydroisoquinolin-1(2H)-one

The title compound (68 mg, 0.11 mmol, 72% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-chloro-6,7-diethoxyquinazoline (61 mg, 0.24 mmol, 1.5 equiv). ¹H NMR (400 MHz, DMSO-d₆) δ 8.99 (s, 1H), 8.09 (s, 1H), 8.00 (d, J=1.6 Hz, 1H), 7.79 (s, 1H), 7.40 (d, J=1.4 Hz, 1H), 7.39 (s, 1H), 7.22 (s, 1H), 7.09 (s, 1H), 6.91 (s, 1H), 5.32 (s, 2H), 4.29 (q, J=7.0 Hz, 2H), 4.10-4.01 (m, 4H), 3.99 (s, 3H), 3.00 (brs, 2H), 1.44 (t, J=7.0 Hz, 3H), 1.35 (t, J=6.9 Hz, 3H); LCMS (ESI) Method 2: >95%, R_{T}=1.502 min, m/z=592.0 [M+H]⁺.

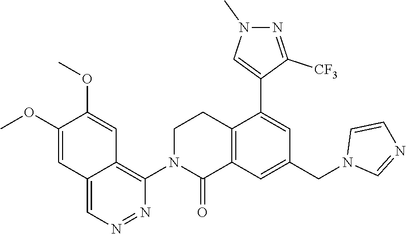 [SMILES: COc1cc2cnnc(N3CCc4c(cc(Cn5ccnc5)cc4-c4cn(C)nc4C(F)(F)F)C3=O)c2cc1OC]

### Example 31

### 7-((1H-Imidazol-1-yl)methyl)-2-(6,7-dimethoxyphthalazin-1-yl)-5-(1-methyl-3-(trifluoromethyl)-1H-pyrazol-4-yl)-3,4-dihydroisoquinolin-1(2H)-one

The title compound (65 mg, 0.12 mmol, 72% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 1-chloro-6,7-dimethoxyphthalazine (54 mg, 0.24 mmol, 1.5 equiv). ¹H NMR (400 MHz, DMSO-d₆) δ 9.46 (s, 1H), 8.10 (s, 1H), 7.98 (d, J=1.4 Hz, 1H), 7.79 (s, 1H), 7.66 (s, 1H), 7.40 (s, 1H), 7.22 (s, 1H), 7.19 (s, 1H), 6.91 (s, 1H), 5.32 (s, 2H), 4.20-4.05 (m, 1H), 4.00 (s, 3H), 3.99 (s, 3H), 3.94-3.84 (m, 4H), 3.16 (brs, 1H), 2.95 (brs, 1H); LCMS (ESI) Method 2: >95%, R_{T}=1.108 min, m/z=564.0 [M+H]⁺.

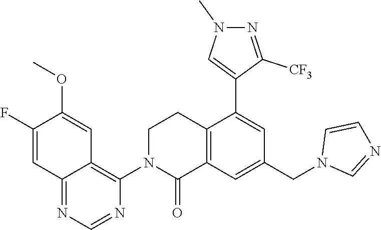 [SMILES: COc1cc2c(N3CCc4c(cc(Cn5ccnc5)cc4-c4cn(C)nc4C(F)(F)F)C3=O)ncnc2cc1F]

### Example 32

### 7-((1H-Imidazol-1-yl)methyl)-2-(7-fluoro-6-methoxyquinazolin-4-yl)-5-(l-methyl-3-(trifluoromethyl)-1H-pyrazol-4-yl)-3,4-dihydroisoquinolin-1(2H)-one

The title compound (28 mg, 0.051 mmol, 32% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv) and 4-chloro-7-fluoro-6-methoxyquinazoline (51 mg, 0.24 mmol, 1.5 equiv). ¹H NMR (400 MHz, Chloroform-d) δ 9.08 (s, 1H), 8.17 (d, 0.1=1.7 Hz, 1H), 7.91 (s, 1H), 7.70 (d, J=11.2 Hz, 1H), 7.44 (s, 1H), 7.25 (d, J=1.7 Hz, 1H), 7.17 (s, 1H), 7.12 (d, J=8.8 Hz, 1H), 7.00 (s, 1H), 5.27 (s, 2H), 4.17 (brs, 2H), 4.04 (s, 3H), 3.95 (s, 3H), 3.07 (t, J=6.2 Hz, 2H); ¹⁹F NMR (376 MHz, Chloroform-d) δ −60.08, −120.25; LCMS (ESI) Method 2: >95%, R_{T}=1.314 min, m/z=552.0 [M+H]⁺.

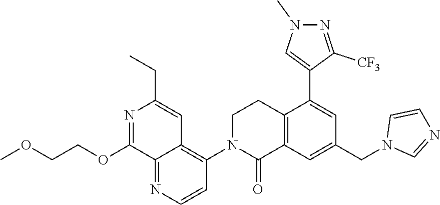 [SMILES: CCc1cc2c(N3CCc4c(cc(Cn5ccnc5)cc4-c4cn(C)nc4C(F)(F)F)C3=O)ccnc2c(OCCOC)n1]

### Example 33

### 7-((1H-Imidazol-1-yl)methyl)-2-(6-ethyl-8-(2-methoxyethoxy)-1,7-naphthyridin-4-yl)-5-(1-methyl-3-(trifluoromethyl)-1H-pyrazol-4-yl)-3,4-dihydroisoquinolin-1(2H)-one

Sodium hydride (9.4 mg, 90% w/w, 0.35 mmol, 4 equiv) was added to a solution of 2-methoxyethanol (1 mL) at 0° C. and stirred for 15 min. 7-((1H-Imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8.50 mg, 0.088 mmol, 1 equiv) was added. The reaction mixture was then warmed to room temperature and stirred overnight. Sat. aq. NaHCO₃ was added and the mixture was extracted with EtOAc. The combined organic layers were dried over MgSO₄, concentrated under reduced pressure. The residue was purified by flash chromatography (Combi-flash Rf, CH₂Cl₂/MeOH=0-10% gradient) and reversed-phase HPLC (Phenomenex Gemini C18, H₂O/CH₃CN gradient from 15-80% CH₃CN, 0.1% TFA) followed by neutralization with sat. aq. NaHCO₃ to yield the title compound (23 mg, 0.038 mmol, 43% yield). ¹H NMR (400 MHz, Chloroform-d) δ 8.95 (d, J=4.6 Hz, 1H), 8.18 (s, 1H), 8.01 (brs, 1H), 7.47 (d, J=4.5 Hz, 1H), 7.45 (s, 1H), 7.24-7.21 (m, 2H), 7.04 (brs, 1H), 6.95 (s, 1H), 5.28 (s, 2H), 4.81 (t, J=5.2 Hz, 2H), 4.04 (s, 3H), 4.01-3.90 (m, 3H), 3.84-3.78 (m, 1H), 3.47 (s, 3H), 3.21-3.12 (m, 1H), 3.01-2.95 (m, 1H), 2.79 (q, J=7.4 Hz, 2H), 1.31 (t, J=7.5 Hz, 3H); ¹⁹F NMR (376 MHz, Chloroform-d) δ −60.06; LCMS (ESI) Method 2: >95%, R_{T}=1.500 min, m/z=606.1 [M+H]⁺.

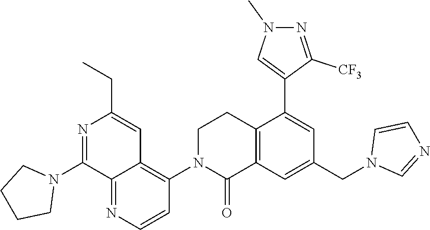 [SMILES: CCc1cc2c(N3CCc4c(cc(Cn5ccnc5)cc4-c4cn(C)nc4C(F)(F)F)C3=O)ccnc2c(N2CCCC2)n1]

### Example 34

### 7-((1H-Imidazol-1-yl)methyl)-2-(6-ethyl-8-(pyrrolidin-1-yl)-1,7-naphthyridin-4-yl)-5-(1-methyl-3-(trifluoromethyl)-1H-pyrazol-4-yl)-3,4-dihydroisoquinolin-1(2H)-one

The title compound (22 mg, 37 μmol, 49% yield) was prepared following the Buchwald coupling procedure described for Intermediate 15, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 42.4 mg, 0.075 mmol, 1 equiv) and pyrrolidine (8.00 mg, 11 μL, 113 μmol, 1.5 equiv). ¹H NMR (400 MHz, CDCl₃) δ 8.77 (d, J=4.6 Hz, 1H), 8.15 (d, J=1.6 Hz, 1H), 7.77 (s, 1H), 7.44 (t, J=4.1 Hz, 2H), 7.22 (d, J=1.6 Hz, 1H), 7.15 (s, 1H), 6.99 (d, J=2.9 Hz, 1H), 6.54 (s, 1H), 5.24 (s, 2H), 4.33 (d, J=38.6 Hz, 4H), 4.03 (s, 3H), 3.96 (m, 1H), 3.76 (m, 1H), 3.04 (m, 4H), 2.07 (t, J=6.1 Hz, 4H), 1.32 (t, J=7.5 Hz, 3H); LCMS (ESI) Method 2: >95%, R_{T}=1.40 min, m/z=601.1 [M+H]⁺.

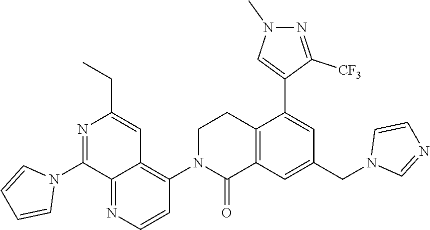 [SMILES: CCc1cc2c(N3CCc4c(cc(Cn5ccnc5)cc4-c4cn(C)nc4C(F)(F)F)C3=O)ccnc2c(-n2cccc2)n1]

### Example 35

### 7-((1H-Imidazol-1-yl)methyl)-2-(6-ethyl-8-(1H-pyrrol-1-yl)-1,7-naphthyridin-4-yl)-5-(1-methyl-3-(trifluoromethyl)-1H-pyrazol-4-yl)-3,4-dihydroisoquinolin-1(2H)-one

The title compound (24 mg, 40 μmol, 54% yield) was prepared following the Buchwald coupling procedure described for Intermediate 15, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 42.4 mg, 0.075 mmol, 1 equiv) and 1H-pyrrole (7.5 mg, 7.8 μL, 112.5 μmol, 1.5 equiv). ¹H NMR (400 MHz, CDCl₃) δ 9.01 (d, J=4.5 Hz, 1H), 8.64 (s, 1H), 8.20 (d, J=1.6 Hz, 1H), 8.15 (t, J=2.3 Hz, 2H), 7.51 (t, J=3.9 Hz, 2H), 7.35 (d, J=1.5 Hz, 1H), 7.29 (s, 1H), 7.24 (s, 1H), 7.10 (s, 1H), 6.41 (t, J=2.3 Hz, 2H), 5.39 (s, 2H), 4.04 (m, 4H), 3.85 (m, 1H), 3.21 (m, 1H), 3.03 (m, 1H), 2.92 (q, J=7.5 Hz, 2H), 1.36 (t, J=7.5 Hz, 3H); LCMS (ESI) Method 2: >95%, R_{T}=1.74 min, m z=597.0 [M+H]⁺.

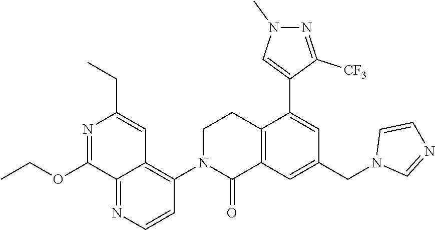 [SMILES: CCOc1nc(CC)cc2c(N3CCc4c(cc(Cn5ccnc5)cc4-c4cn(C)nc4C(F)(F)F)C3=O)ccnc12]

### Example 36

### 7-((1H-Imidazol-1-yl)methyl)-2-(8-ethoxy-6-ethyl-1,7-naphthyridin-4-yl)-5-(1-methyl-3-(trifluoromethyl)-1H-pyrazol-4-yl)-3,4-dihydroisoquinolin-1(2H)-one

The title compound (20 mg, 0.035 mmol, 39% yield) was prepared following the procedure described for Example 33, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 50 mg, 0.088 mmol, 1 equiv) and ethanol (1 mL). ¹H NMR (400 MHz, Chloroform-d) δ 9.01-8.96 (m, 1H), 8.19 (s, 1H), 7.93-7.80 (m, 1H), 7.50-7.48 (m, 1H), 7.46 (s, 1H), 7.25 (s, 1H), 7.19 (s, 1H), 7.02 (s, 1H), 6.90 (s, 1H), 5.27 (s, 2H), 4.76-4.68 (m, 2H), 4.06 (s, 3H), 4.05-3.99 (m, 1H), 3.86-3.82 (m, 1H), 3.22-3.11 (m, 1H), 3.04-2.96 (m, 1H), 2.85-2.78 (m, 2H), 1.61-1.58 (m, 3H), 1.37-1.31 (m, 3H); ¹⁹F NMR (376 MHz, Chloroform-d) δ −60.08; LCMS (ESI) Method 2: >95%, R_{T}=1.573 min, m/z=576.0 [M+H]⁺.

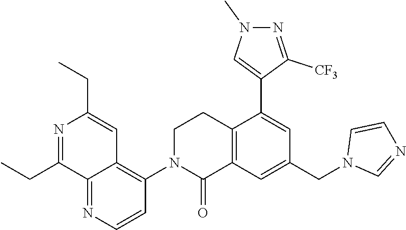 [SMILES: CCc1cc2c(N3CCc4c(cc(Cn5ccnc5)cc4-c4cn(C)nc4C(F)(F)F)C3=O)ccnc2c(CC)n1]

### Example 37

### 7-((1H-Imidazol-1-yl)methyl)-2-(6,8-diethyl-1,7-naphthyridin-4-yl)-5-(1-methyl-3-(trifluoromethyl)-1H-pyrazol-4-yl)-3,4-dihydroisoquinolin-1(2H)-one

The title compound (28.00 mg, 50.04 μmol, 67% yield) was prepared following the procedure described for Intermediate 14 Step A, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 42.4 mg, 0.075 mmol, 1 equiv) and diethylzinc (75.0 μL 1.0 M, 75.0 μmol, 1 equiv) ¹H NMR (400 MHz, CDCl₃) δ 9.01 (d, J=4.5 Hz, 1H), 8.55 (s, 1H), 8.19 (s, 1H), 7.50 (d, J=4.2 Hz, 2H), 7.34 (s, 1H), 7.29 (d, J=8.1 Hz, 2H), 7.09 (s, 1H), 5.38 (s, 2H), 4.04 (s, 4H), 3.83 (t, J=6.0 Hz, 1H), 3.60 (m, 2H), 3.20 (q, J=7.2 Hz, 1H), 3.00 (m, 3H), 1.46 (t, J=7.5 Hz, 3H), 1.36 (t, J=7.5 Hz, 3H); LCMS (ESI) Method 2: >95%, R_{T}=1.377 min, m/z=560.1 [M+H]⁺.

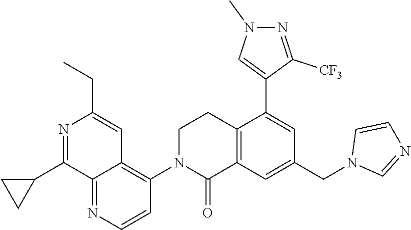 [SMILES: CCc1cc2c(N3CCc4c(cc(Cn5ccnc5)cc4-c4cn(C)nc4C(F)(F)F)C3=O)ccnc2c(C2CC2)n1]

### Example 38

### 7-((1H-Imidazol-1-yl)methyl)-2-(8-cyclopropyl-6-ethyl-1,7-naphthyridin-4-yl)-5-(1-methyl-3-(trifluoromethyl)-1H-pyrazol-4-yl)-3,4-dihydroisoquinolin-1(2H)-one

The title compound (25 mg, 44 μmol, 58% yield) was prepared following the Suzuki reaction described for Intermediate 3, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 42.4 mg, 0.075 mmol, 1 equiv) and cyclopropylboronic acid (7.1 mg, 83 μmol, 1.1 equiv). ¹H NMR (400 MHz, CDCl₃) δ 8.99 (d, J=4.5 Hz, 1H), 8.92 (s, 1H), 8.21 (s, 1H), 7.52 (s, 1H), 7.48 (d, J=4.5 Hz, 1H), 7.38 (s, 1H), 7.33 (s, 1H), 7.13 (s, 2H), 5.42 (s, 2H), 4.04 (m, 4H), 3.82 (m, 1H), 3.57 (m, 1H), 3.19 (m, 1H), 3.02 (q, J=7.2 Hz, 1H), 2.86 (q, J=7.4 Hz, 2H), 1.30 (q, J=11.2 Hz, 5H), 1.18 (t, J=6.3 Hz, 2H); LCMS (ESI) Method 2: >95%, R_{T}=1.588 min, m/z=572.1 [M+H]⁺.

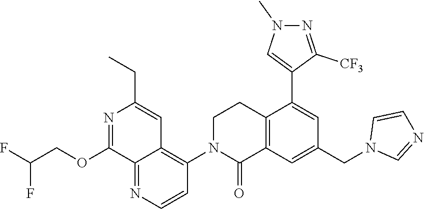 [SMILES: CCc1cc2c(N3CCc4c(cc(Cn5ccnc5)cc4-c4cn(C)nc4C(F)(F)F)C3=O)ccnc2c(OCC(F)F)n1]

### Example 39

### 7-((1H-Imidazol-1-yl)methyl)-2-(8-(2,2-difluoroethoxy)-6-ethyl-1,7-naphthyridin-4-yl)-5-(1-methyl-3-(trifluoromethyl)-1H-pyrazol-4-yl)-3,4-dihydroisoquinolin-1(2H)-one

The title compound (27 mg, 0.044 mmol, 50% yield) was prepared following the procedure described for Example 33, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 50 mg, 0.088 mmol, 1 equiv) and 2,2-difluoroethan-1-ol (1 mL). ¹H NMR (400 MHz, Chloroform-d) δ 8.99 (d, J=4.6 Hz, 1H), 8.16 (d, J=1.3 Hz, 1H), 7.83 (brs, 1H), 7.50 (d, J=4.6 Hz, 1H), 7.43 (s, 1H), 7.23 (s, 1H), 7.16 (s, 1H), 7.03 (s, 1H), 7.00 (s, 1H), 6.37 (tt, J=55.8, 4.4 Hz, 1H), 5.25 (s, 2H), 4.90-4.81 (m, 2H), 4.06-3.99 (m, 4H), 3.81 (dt, J=12.2, 5.1 Hz, 1H), 3.21-3.12 (m, 1H), 2.99 (dt, J=16.4, 4.5 Hz, 1H), 2.81 (q, J=7.5 Hz, 2H), 1.32 (t, J=7.5 Hz, 3H): ¹⁹F NMR (376 MHz, Chloroform-d) δ −60.08, −124.98 (d, J=30.5 Hz); LCMS (ESI) Method 2: >95%, R_{T}=1.569 min, m/z=612.0 [M+H]⁺.

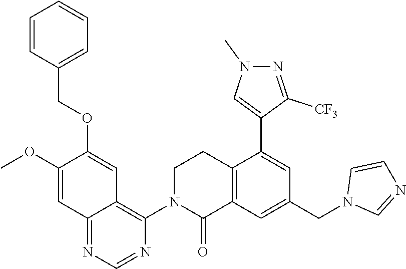 [SMILES: COc1cc2ncnc(N3CCc4c(cc(Cn5ccnc5)cc4-c4cn(C)nc4C(F)(F)F)C3=O)c2cc1OCc1ccccc1]

### Example 40

### 7-((1H-Imidazol-1-yl)methyl)-2-(6-(benzyloxy)-7-methoxyquinazolin-4-yl)-5-(1-methyl-3-(trifluoromethyl)-1H-pyrazol-4-yl)-3,4-dihydroisoquinolin-1(2H)-one

The title compound (735 mg, 1.15 mmol, 94% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 460 mg, 1.23 mmol, 1 equiv) and 6-(benzyloxy)-4-chloro-7-methoxyquinazoline (553 mg, 1.84 mmol, 1.5 equiv). ¹H NMR (400 MHz, Chloroform-d) δ 9.00 (s, 1H), 8.17 (d, J=1.3 Hz, 1H), 7.79 (brs, 1H), 4.43 (s, 1H), 7.37-7.34 (m, 4H), 7.25-7.17 (m, 5H), 7.05 (s, 1H), 5.24 (s, 2H), 5.20 (s, 2H), 4.07-4.02 (m, 8H), 2.96 (t, J=6.3 Hz, 2H); LCMS (ESI) Method 2: >95%, R_{T}=1.545 min, m/z=640.0 [M+H]⁺.

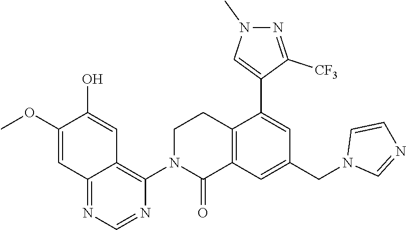 [SMILES: COc1cc2ncnc(N3CCc4c(cc(Cn5ccnc5)cc4-c4cn(C)nc4C(F)(F)F)C3=O)c2cc1O]

### Example 41

### 7-((1H-Imidazol-1-yl)methyl)-2-(6-hydroxy-7-methoxyquinazolin-4-yl)-5-(1-methyl-3-(trifluoromethyl)-1H-pyrazol-4-yl)-3,4-dihydroisoquinolin-1(2H)-one

To a solution of 7-((1H-imidazol-1-yl)methyl)-2-(6-(benzyloxy)-7-methoxyquinazolin-4-yl)-5-(1-methyl-3-(trifluoromethyl)-1H-pyrazol-4-yl)-3,4-dihydroisoquinolin-1(2H)-one (Example 40, 706 mg, 1.10 mmol, 1 equiv) in MeOH (30 mL) was added Pd/C (210 mg, 10% w/w, 197 μmol, 0.18 equiv) at room temperature. The reaction was degassed and placed under a hydrogen atmosphere. The reaction was heated to 50° C. and stirred until completion. The reaction mixture was filter through celite and concentrated under reduced pressure to yield the title compound (510 mg, 0.928 mmol, 84% yield). ¹H NMR (400 MHz, DMSO-d₆) δ 10.22 (s, 1H), 8.95 (s, 1H), 8.12 (s, 1H), 8.01 (s, 1H), 7.80 (s, 1H), 7.44 (s, 1H), 7.39 (s, 1H), 7.23 (s, 1H), 7.05 (s, 1H), 6.92 (s, 1H), 5.32 (s, 2H), 4.05-3.96 (m, 8H), 2.98 (brs, 2H); LCMS (ESI) Method 2: >95%, R_{T}=1.392 min, m/z=550.0 [M+H]⁺.

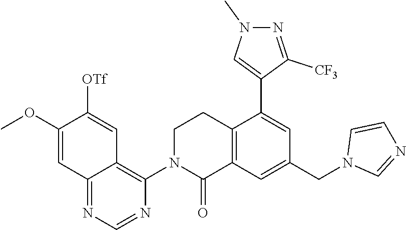 [SMILES: COc1cc2ncnc(N3CCc4c(cc(Cn5ccnc5)cc4-c4cn(C)nc4C(F)(F)F)C3=O)c2cc1OS(=O)(=O)C(F)(F)F]

### Example 42

### 4-(7-((1H-Imidazol-1-yl)methyl)-5-(1-methyl-3-(trifluoromethyl)-1H-pyrazol-4-yl)-1-oxo-3,4-dihydroisoquinolin-2(1H)-yl)-7-methoxyquinazolin-6-yl trifluoromethanesulfonate

To a solution of 7-((1H-imidazol-1-yl)methyl)-2-(6-hydroxy-7-methoxyquinazolin-4-yl)-5-(1-methyl-3-(trifluoromethyl)-1H-pyrazol-4-yl)-3,4-dihydroisoquinolin-1(2H)-one (Example 41, 300 mg, 546 μmol, 1 equiv) and pyridine (0.2 mL, 2.18 mmol, 4 equiv) in CH₂Cl₂ (3 mL) was added Tf₂O (1.64 mL, 1.64 mmol, 3 equiv, 1 M CH₂Cl₂) dropwise at 0° C. The reaction mixture was stirred at 0° C. for 1 h then diluted with CH₂Cl₂. The reaction was quenched with sat. aq. NaHCO₃ and extracted with CH₂Cl₂. The combined organic layer was dried over MgSO₄, filtered, and concentrated under reduced pressure. The residue was purified by column chromatography (Combi-flash Rf, CH₂Cl₂/MeOH=0-10% gradient) to yield the title compound (370 mg, 0.542 mmol, 99% yield). ¹H NMR (400 MHz, Chloroform-d) δ 9.17 (s, 1H), 8.34-8.08 (m, 2H), 7.73 (s, 1H), 7.52 (s, 1H), 7.45 (s, 1H), 7.30-7.20 (m, 2H), 7.05-7.02 (m, 1H), 5.37-5.32 (m, 2H), 4.21 (t, J=6.0 Hz, 2H), 4.13 (s, 3H), 4.06 (s, 3H), 3.08 (t, J=5.8 Hz, 2H); LCMS (ESI) Method 2: >95%, R_{T}=1.636 min, m/z=681.9 [M+H]⁺.

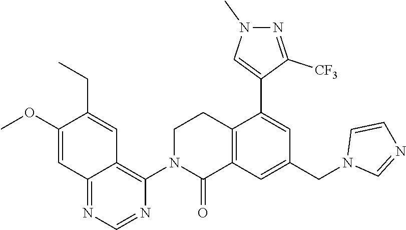 [SMILES: CCc1cc2c(N3CCc4c(cc(Cn5ccnc5)cc4-c4cn(C)nc4C(F)(F)F)C3=O)ncnc2cc1OC]

### Example 43

### 7-((1H-Imidazol-1-yl)methyl)-2-(6-ethyl-7-methoxyquinazolin-4-yl)-5-(1-methyl-3-(trifluoromethyl)-1H-pyrazol-4-yl)-3,4-dihydroisoquinolin-1(2H)-one

The title compound (31 mg, 0.055 mmol, 63% yield) was prepared following the procedure described for Intermediate 11, using 4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-7-methoxyquinazolin-6-yl trifluoromethanesulfonate (Example 42, 60 mg, 0.088 mmol, 1 equiv), triethylborane (0.18 mmol, 2 equiv, 1 M THF), and cesium acetate (34 mg, 0.18 mmol, 2 equiv). ¹H NMR (400 MHz, Chloroform-d) δ 9.05 (s, 1H), 8.18 (s, 1H), 7.78 (brs, 1H), 7.56 (s, 1H), 7.42 (s, 1H), 7.31, (s, 1H), 7.21-7.14 (m, 2H), 7.00 (brs, 1H), 5.23 (s, 2H), 4.14 (brs, 2H), 4.03 (s, 3H), 4.02 (s, 3H), 3.05 (t, J=6.0 Hz, 2H), 2.78 (q, J=7.4 Hz, 2H), 1.23 (t, J=7.5 Hz, 3H); LCMS (ESI) Method 2: >95%, R_{T}=1.487 min, m/z=562.0 [M+H]⁺.

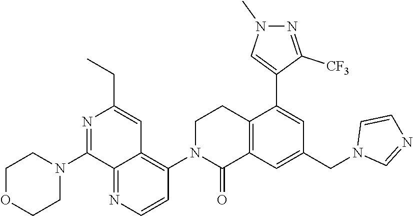 [SMILES: CCc1cc2c(N3CCc4c(cc(Cn5ccnc5)cc4-c4cn(C)nc4C(F)(F)F)C3=O)ccnc2c(N2CCOCC2)n1]

### Example 44

### 7-((1H-Imidazol-1-yl)methyl)-2-(6-ethyl-8-morpholino-1,7-naphthyridin-4-yl)-5-(1-methyl-3-(trifluoromethyl)-1H-pyrazol-4-yl)-3,4-dihydroisoquinolin-1(2H)-one

The title compound (27.0 mg, 44 μmol, 58% yield) was prepared following the Buchwald coupling procedure described for Intermediate 15, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 42.5 mg, 0.075 mmol, 1 equiv) and morpholine (9.8 mg, 9.7 μL, 113 μmol, 1.5 equiv). ¹H NMR (400 MHz, CDCl₃) δ 9.14 (s, 1H), 8.81 (t, J=4.5 Hz, 1H), 8.21 (s, 1H), 7.54 (t, J=7.3 Hz, 1H), 7.47 (s, 1H), 7.42 (s, 1H), 7.39 (s, 1H), 7.18 (d, J=6.2 Hz, 1H), 6.80 (s, 1H), 5.47 (s, 2H), 4.22 (s, 3H), 4.05 (s, 4H), 3.97 (m, 6H), 3.81 (t, J=6.0 Hz, 1H), 3.19 (q, J=9.0 Hz, 1H), 2.99 (m, 2H), 1.29 (q, J=7.9 Hz, 3H); LCMS (ESI) Method 2: >95%, R_{T}=1.387 min, m/z=617.1 [M+H]⁺.

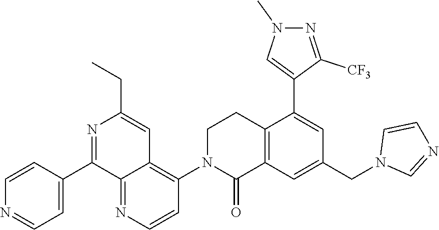 [SMILES: CCc1cc2c(N3CCc4c(cc(Cn5ccnc5)cc4-c4cn(C)nc4C(F)(F)F)C3=O)ccnc2c(-c2ccncc2)n1]

### Example 45

### 7-((1H-Imidazol-1-yl)methyl)-2-(6-ethyl-8-(pyridin-4-yl)-1,7-naphthyridin-4-yl)-5-(1-methyl-3-(trifluoromethyl)-1H-pyrazol-4-yl)-3,4-dihydroisoquinolin-1(2H)-one

The title compound (28 mg, 46 μmol, 61% yield) was prepared following the Suzuki reaction described for Intermediate 3, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 42.4 mg, 0.075 mmol, 1 equiv) and pyridin-4-ylboronic acid (14 mg, 113 μmol, 1.5 equiv). ¹H NMR (400 MHz, CDCl₃) δ 9.04 (d, J=4.5 Hz, 1H), 8.80 (q, J=2.1 Hz, 2H), 8.45 (s, 1H), 8.21 (q, J=3.7 Hz, 3H), 7.54 (t, J=4.1 Hz, 1H), 7.51 (d, J=5.0 Hz, 2H), 7.35 (d, J=1.6 Hz, 1H), 7.26 (s, 1H), 7.09 (s, 1H), 5.38 (s, 2H), 4.13 (m, 1H), 4.05 (s, 3H), 3.87 (m, 1H), 3.23 (m, 1H), 3.06 (q, J=7.5 Hz, 3H), 1.41 (t, J=7.5 Hz, 3H); LCMS (ESI) Method 2: >95%, R_{T}=1.315 min, m/z=609.0 [M+H]⁺.

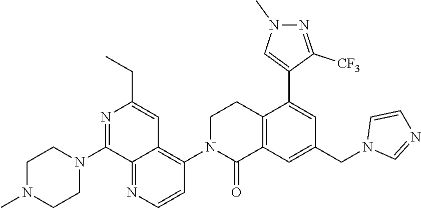 [SMILES: CCc1cc2c(N3CCc4c(cc(Cn5ccnc5)cc4-c4cn(C)nc4C(F)(F)F)C3=O)ccnc2c(N2CCN(C)CC2)n1]

### Example 46

### 7-((1H-Imidazol-1-yl)methyl)-2-(6-ethyl-8-(4-methylpiperazin-1-yl)-1,7-naphthyridin-4-yl)-5-(1-methyl-3-(trifluoromethyl)-1H-pyrazol-4-yl)-3,4-dihydroisoquinolin-1(2H)-one

The title compound (28 mg, 44 μmol, 59% yield) was prepared following the Buchwald coupling procedure described for Intermediate 15, using 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 42.5 mg, 0.075 mmol, 1 equiv) and 1-methylpiperazine (11 mg, 13 μL, 113 μmol, 1.5 equiv). ¹H NMR (400 MHz, CDCl₃) δ 8.80 (d, J=4.6 Hz, 1H), 8.13 (d, J=1.8 Hz, 1H), 7.62 (s, 1H), 7.43 (t, J=4.6 Hz, 2H), 7.20 (d, J=1.8 Hz, 1H), 7.12 (s, 1H), 6.96 (s, 1H), 6.92 (s, 1H), 5.21 (s, 2H), 4.03 (s, 4H), 3.97 (m, J=5.5 Hz, 2H), 3.80 (m, 1H), 3.50 (t, J=16.8 Hz, 2H), 3.17 (m, 3H), 2.98 (q, J=7.3 Hz, 2H), 2.83 (s, 3H), 2.76 (q, J=7.5 Hz, 2H), 1.28 (t, J=7.5 Hz, 3H); LCMS (ESI) Method 2: >95%, R_{T}=1.267 min, m-z=630.1 [M+H]⁺.

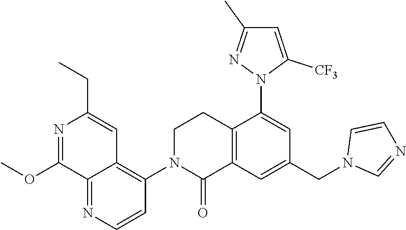 [SMILES: CCc1cc2c(N3CCc4c(cc(Cn5ccnc5)cc4-n4nc(C)cc4C(F)(F)F)C3=O)ccnc2c(OC)n1]

### Example 47

### 7-((1H-Imidazol-1-yl)methyl)-2-(6-ethyl-8-methoxy-1,7-naphthyridin-4-yl)-5-(3-methyl-5-(trifluoromethyl)-1H-pyrazol-1-yl)-3,4-dihydroisoquinolin-1(2H)-one

The title compound (25 mg, 0.045 mmol, 33% yield) was prepared following the Buchwald coupling procedure described for Intermediate 15, using 7-((1H-imidazol-1-yl)methyl)-5-(3-methyl-5-(trifluoromethyl)-1H-pyrazol-1-yl)-3,4-dihydroisoquinolin-1(2H)-one (Intermediate 42, 50 mg, 0.13 mmol, 1 equiv) and 4-bromo-6-ethyl-8-methoxy-1,7-naphthyridine (Intermediate 14, 39 mg, 0.15 mmol, 1.1 equiv). ¹H NMR (400 MHz, Chloroform-d) δ 9.07 (brs, 1H), 8.94 (d, J=4.6 Hz, 1H), 8.33 (s, 1H), 7.48 (d, J=4.5 Hz, 1H), 7.47 (s, 1H), 7.35 (brs, 1H), 7.06 (brs, 1H), 6.91 (s, 1H), 6.67 (s, 1H), 5.53 (s, 2H), 4.23 (s, 3H), 4.04 (td, J=11.6, 3.6 Hz, 1H), 3.83 (dt, J=12.4, 5.4 Hz, 1H), 3.18-3.08 (m, 1H), 2.81 (q, 0.1=7.5 Hz, 2H), 2.77-2.70 (m, 1H), 2.38 (s, 3H), 1.32 (t, J=7.5 Hz, 3H); ¹¹F NMR (376 MHz, Chloroform-d) δ −58.57; LCMS (ESI) Method 2: >95%, R_{T}=1.472 min, m/z=562.0 [M+H]⁺.

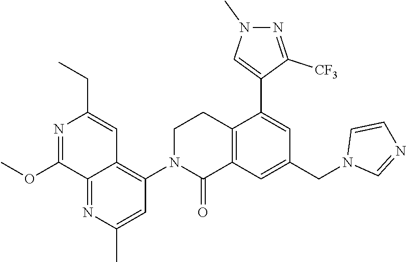 [SMILES: CCc1cc2c(N3CCc4c(cc(Cn5ccnc5)cc4-c4cn(C)nc4C(F)(F)F)C3=O)cc(C)nc2c(OC)n1]

### Example 48

### 7-((1H-Imidazol-1-yl)methyl)-2-(6-ethyl-8-methoxy-2-methyl-1,7-naphthyridin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-5-(1-methyl-3-(trifluoromethyl)-1H-pyrazol-4-yl)-3,4-dihydroisoquinolin-1(2H)-one (Intermediate 8, 50 mg, 0.13 mmol, 1.0 equiv), 4-bromo-6-ethyl-8-methoxy-2-methyl-1,7-naphthyridine (Intermediate 43, 41 mg, 0.15 mmol 2.0 equiv), cesium carbonate (87 mg, 0.27 mmol, 2 equiv), Xantphos (12 mg, 0.02 mmol, 0.15 equiv), and Pd₂(dba)₃ (6 mg, 0.007 mmol, 0.05 equiv) were dissolved in 1,4-dioxane under an Ar. The reaction mixture was stirred for 14 h at 115° C. then cooled to 23° C. Brine was added to the mixture and the mixture was extracted with CH₂Cl₂ (3×20 mL). The combined organic layers were dried over MgSO₄ and concentrated under reduced pressure. The residue was purified by reversed-phase HPLC (Phenomenex Gemini C18, H₂O/CH₃CN gradient from 15-70% CH₃CN, 0.1% TFA) followed by neutralization with sat. aq. NaHCO₃ to obtain the title compound (65 mg, 0.11 mmol, 85% yield). ¹H NMR (400 MHz, DMSO-d₆) δ 8.08 (s, 1H), 7.95 (s, 1H), 7.78 (s, 1H), 7.67 (s, 1H), 7.41 (s, 1H) 7.21 (s, 1H), 7.05 (s, 1H), 6.91 (s, 1H), 5.31 (s, 2H), 4.06 (s, 3H), 4.03-3.95 (m, 4H), 3.78-3.72 (m, 1H), 3.18-3.09 (m, 1H), 2.87 (dt, J=16.4, 4.5 Hz, 1H), 2.73 (q, J=7.5 Hz, 2H), 2.65 (s, 3H), 1.24 (t, J=7.5 Hz, 3H); ¹⁹F NMR (376 MHz, DMSO-d₆) δ −58.21; LCMS (ESI) Method 2: >95%, R_{T}=1.256 min, m/z=576.0 [M+H]⁺.

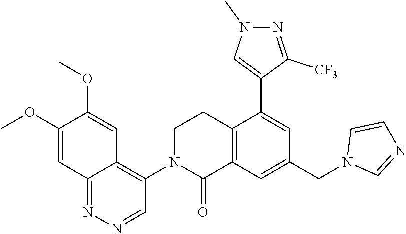 [SMILES: COc1cc2nncc(N3CCc4c(cc(Cn5ccnc5)cc4-c4cn(C)nc4C(F)(F)F)C3=O)c2cc1OC]

### Example 49

### 7-((1H-Imidazol-1-yl)methyl)-2-(6,7-dimethoxycinnolin-4-yl)-5-(1-methyl-3-(trifluoromethyl)-1H-pyrazol-4-yl)-3,4-dihydroisoquinolin-1(2H)-one

The title compound (47 mg, 0.084 mmol, 63% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 50 mg, 0.13 mmol, 1 equiv) and 4-chloro-6,7-dimethoxycinnoline (45 mg, 0.20 mmol, 1.5 equiv). ¹H NMR (400 MHz, Chloroform-d) δ 9.07 (s, 1H), 8.17 (d, J=1.5 Hz, 1H), 8.08 (s, 1H), 7.80 (s, 1H), 7.46 (s, 1H), 7.27 (s, 1H), 7.19 (s, 1H), 7.03 (s, 1H), 6.89 (s, 1H), 5.29 (s, 2H), 4.15-4.08 (m, 1H), 4.11 (s, 3H), 4.03 (s, 3H), 3.99 (s, 3H), 3.88-3.81 (m, 1H), 3.19-3.10 (m, 1H), 3.03 (dt, J=16.4, 4.4 Hz, 1H); ¹⁹F NMR (376 MHz, Chloroform-d) δ −60.07; LCMS (ESI) Method 2: >95%, R_{T}=1.123 min, m/z=564.0 [M+H]⁺.

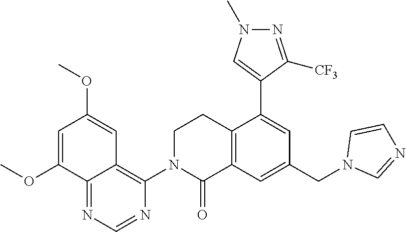 [SMILES: COc1cc(OC)c2ncnc(N3CCc4c(cc(Cn5ccnc5)cc4-c4cn(C)nc4C(F)(F)F)C3=O)c2c1]

### Example 50

### 7-((1H-Imidazol-1-yl)methyl)-2-(6,8-dimethoxyquinazolin-4-yl)-5-(1-methyl-3-(trifluoromethyl)-1H-pyrazol-4-yl)-3,4-dihydroisoquinolin-1(2H)-one

The title compound (55 mg, 0.097 mmol, 73% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 50 mg, 0.13 mmol, 1 equiv) and 4-chloro-6,8-dimethoxyquinazoline (45 mg, 0.20 mmol, 1.5 equiv). ¹H NMR (400 MHz, DMSO-d₆) δ 9.03 (s, 1H), 8.09 (s, 1H), 7.99 (d, J=1.7 Hz, 1H), 7.78 (s, 1H), 7.40 (d, J=1.4 Hz, 1H), 7.22 (s, 1H), 7.08 (d, J=2.4 Hz, 1H), 6.91 (s, 1H), 6.71 (d, J=2.4 Hz, 1H), 5.32 (s, 2H), 4.00 (brs, 2H), 3.99 (s, 6H), 3.80 (s, 3H), 3.00 (brs, 2H); ¹⁹F NMR (376 MHz, DMSO-dc) 6-58.22; LCMS (ESI) Method 2: >95%, R_{T}=1.327 min, m/z=564.0 [M+H]⁺.

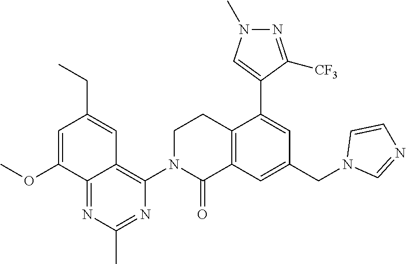 [SMILES: CCc1cc(OC)c2nc(C)nc(N3CCc4c(cc(Cn5ccnc5)cc4-c4cn(C)nc4C(F)(F)F)C3=O)c2c1]

### Example 51

### 7-((1H-Imidazol-1-yl)methyl)-2-(6-ethyl-8-methoxy-2-methylquinazolin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-5-(1-methyl-3-(trifluoromethyl)-1H-pyrazol-4-yl)-3,4-dihydroisoquinolin-1(2H)-one (Intermediate 8,400 mg, 1.07 mmol, 1.0 equiv), 4-chloro-6-ethyl-8-methoxy-2-methylquinazoline (Intermediate 44, 328 mg, 1.39 mmol, 1.3 equiv), cesium carbonate (694 mg, 2.13 mmol, 2 equiv), Xantphos (93 mg, 0.16 mmol, 0.15 equiv), and Pd₂(dba)₃ (49 mg, 0.053 mmol, 0.05 equiv) were dissolved in 1,4-dioxane (6 mL) under an Ar. The reaction mixture was stirred for 14 h at 115° C. then cooled to 23° C. Brine was added to the mixture and the mixture was extracted with CH₂Cl₂ (3×50 mL). The combined organic layers were dried over MgSO₄ and concentrated under reduced pressure. The residue was purified by reversed-phase HPLC (Phenomenex Gemini C18, H₂O/CH₃CN gradient from 15-70% CHzCN, 0.1% TFA) followed by neutralization with sat. aq. NaHCO₃ to obtain the title compound (215 mg, 0.37 mmol, 35% yield). ¹H NMR (400 MHz, DMSO-d₆) δ 8.10 (s, 1H), 7.98 (d, J=1.8 Hz, 1H), 7.78 (s, 1H), 7.40 (d, J=1.7 Hz, 1H), 7.28 (d, J=1.5 Hz, 1H), 7.22 (s, 1H), 7.19 (d, J=1.3 Hz, 1H), 6.91 (s, 1H), 5.32 (s, 2H), 3.99 (brs, 8H), 3.00 (brs, 2H), 2.720 (q, J=7.6 Hz, 2H), 2.719 (s, 3H), 1.21 (t, J=7.6 Hz, 3H); ¹⁹F NMR (376 MHz, DMSO-d₆) δ −58.25; LCMS (ESI) Method 2: >95%, R_{T}=1.391 min, m/z=576.1 [M+H]⁺.

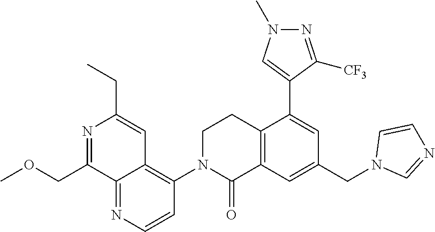 [SMILES: CCc1cc2c(N3CCc4c(cc(Cn5ccnc5)cc4-c4cn(C)nc4C(F)(F)F)C3=O)ccnc2c(COC)n1]

### Example 52

### 7-((1H-Imidazol-1-yl)methyl)-2-(6-ethyl-8-(methoxymethyl)-1,7-naphthyridin-4-yl)-5-(1-methyl-3-(trifluoromethyl)-1H-pyrazol-4-yl)-3,4-dihydroisoquinolin-1(2H)-one

In a sealed tube, 7-((1H-imidazol-1-yl)methyl)-2-(8-chloro-6-ethyl-1,7-naphthyridin-4-yl)-5-(1-methyl-3-(trifluoromethyl)-1H-pyrazol-4-yl)-3,4-dihydroisoquinolin-1(2H)-one (Example 8, 30 mg, 0.053 mmol, 1 equiv), tributyl(methoxymethyl)stannane (0.03 mL, 0.098 mmol, 1.9 equiv), and Pd(PPh₃)₄ (7.1 mg, 0.008 mmol, 0.1 equiv) were dissolved in DMF (0.5 mL) and placed under an argon atmosphere. The reaction mixture was then placed in a preheated heating block and stirred at 115° C. At 23° C., brine was added to the mixture and extracted with CH₂Cl₂. The combined organic layers were dried over MgSO₄ and concentrated under reduced pressure. The residue was purified by reversed-phase HPLC (Phenomenex Gemini C18, MeCN/H₂O=15-70% gradient, 0.1% TFA) followed by neutralization with sat. aq. NaHCO₃ to yield the title compound (16 mg, 0.028 mmol, 52% yield). ¹H NMR (400 MHz, Chloroform-d) δ 9.00 (d, J=4.6 Hz, 1H), 8.24-8.15 (m, 2H), 7.50 (d, J=4.6 Hz, 1H), 7.47 (s, 1H), 7.37 (s, 1H), 7.29 (d, J=2.0 Hz, 1H), 7.22 (s, 1H), 7.04 (s, 1H), 5.34 (dd, J=60.0, 12.8 Hz, 1H), 5.30 (s, 2H), 4.07-4.01 (m, 1H), 4.04 (m, 3H), 3.87-3.76 (m, 1H), 3.61 (s, 3H), 3.19 (ddd, J=16.2, 10.8, 5.1 Hz, 1H), 3.02 (q, J=7.6 Hz, 2H), 3.01-2.97 (m, 1H), 1.36 (t, J=7.5 Hz, 3H); ¹⁹F NMR (376 MHz, Chloroform-d) δ −60.05; LCMS (ESI) Method 2: >95%, R_{T}=1.226 min, m/z=576.0 [M+H]⁺.

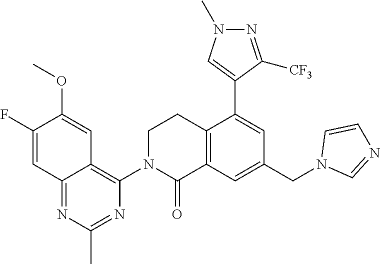 [SMILES: COc1cc2c(N3CCc4c(cc(Cn5ccnc5)cc4-c4cn(C)nc4C(F)(F)F)C3=O)nc(C)nc2cc1F]

### Example 53

### 7-((1H-Imidazol-1-yl)methyl)-2-(7-fluoro-6-methoxy-2-methylquinazolin-4-yl)-5-(1-methyl-3-(trifluoromethyl)-1H-pyrazol-4-yl)-3,4-dihydroisoquinolin-1(2H)-one

The title compound (450 mg, 0.8 mmol, quant.) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 300 mg, 0.8 mmol, 1 equiv) and 4-chloro-7-fluoro-6-methoxy-2-methylquinazoline (362 mg, 1.60 mmol, 2 equiv). ¹H NMR (400 MHz, DMSO-d₆) δ 8.08 (d, J=1.2 Hz, 1H), 7.99 (d, J=1.9 Hz, 1H), 7.82 (s, 1H), 7.78 (d, J=2.3 Hz, 1H), 7.41 (d, J=1.9 Hz, 1H), 7.35 (d, J=9.1 Hz, 1H), 7.21 (d, J=1.3 Hz, 1H), 6.91 (t, J=1.1 Hz, 1H), 5.32 (s, 2H), 4.10-4.01 (m, 2H), 4.00 (s, 3H), 3.88 (s, 3H), 3.06-3.00 (m, 2H), 2.71 (s, 3H); ¹⁹F NMR (376 MHz, DMSO-d₆) δ −58.22, −121.58; LCMS (ESI) Method 2: >95%, R_{T}=1.422 min, m/z=566.0 [M+H]⁺.

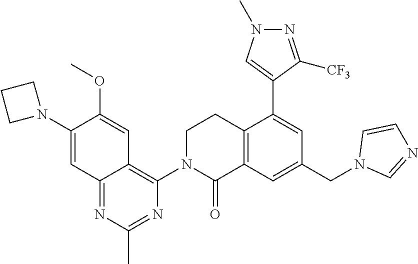 [SMILES: COc1cc2c(N3CCc4c(cc(Cn5ccnc5)cc4-c4cn(C)nc4C(F)(F)F)C3=O)nc(C)nc2cc1N1CCC1]

### Example 54

### 7-((1H-Imidazol-1-yl)methyl)-2-(7-(azetidin-1-yl)-6-methoxy-2-methylquinazolin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-2-(7-fluoro-6-methoxy-2-methylquinazolin-4-yl)-5-(1-methyl-3-(trifluoromethyl)-1H-pyrazol-4-yl)-3,4-dihydroisoquinolin-1(2H)-one (Example 53, 60 mg, 0.11 mmol, 1 equiv) was dissolved in DMSO (1 mL) and stirred at room temperature. Then potassium carbonate (29 mg, 0.21 mmol, 2 equiv) and azetidine (0.04 mL, 0.59 mmol, 5.6 equiv) were added, and the reaction was stirred at 35° C. until completion. The reaction mixture was diluted with sat. aq. NH₄Cl and extracted with CH₂Cl₂ (3×20 mL). The combined organic layer was dried over MgSO₄, filtered, and concentrated. The residue was purified by reversed-phase HPLC (Phenomenex Gemini C18, MeCN/H₂O=15-80% gradient, 0.1% TFA) followed by neutralization with sat. aq. NaHCO₃ to yield the title compound (45 mg, 0.074 mmol, 70% yield). ¹H NMR (400 MHz, DMSO-d₆) δ 8.08 (d, J=1.2 Hz, 1H), 7.96 (d, J=1.9 Hz, 1H), 7.79 (s, 1H), 7.38 (d, J=1.9 Hz, 1H), 7.22 (s, 1H), 6.91 (s, 1H), 6.86 (s, 1H), 6.49 (s, 1H), 5.31 (s, 2H), 4.14 (t, J=7.4 Hz, 4H), 3.99 (s, 3H), 3.93 (brs, 2H), 3.73 (s, 3H), 2.98 (brs, 2H), 2.58 (s, 3H), 2.32 (p, J=7.4 Hz, 3H); ¹¹F NMR (376 MHz, DMSO-d₆) δ −58.27; LCMS (ESI) Method 2: >95%, R_{T}=1.338 min, m % z=603.0 [M+H]⁺.

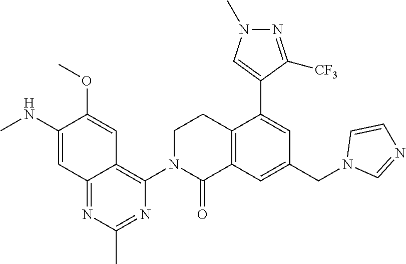 [SMILES: CNc1cc2nc(C)nc(N3CCc4c(cc(Cn5ccnc5)cc4-c4cn(C)nc4C(F)(F)F)C3=O)c2cc1OC]

### Example 55

### 7-((1H-Imidazol-1-yl)methyl)-2-(6-methoxy-2-methyl-7-(methylamino)quinazolin-4-yl)-5-(1-methyl-3-(trifluoromethyl)-1H-pyrazol-4-yl)-3,4-dihydroisoquinolin-1(2H)-one

The title compound (34 mg, 0.058 mmol, 55% yield) was prepared following the procedure described for Example 54, using 7-((1H-Imidazol-1-yl)methyl)-2-(7-fluoro-6-methoxy-2-methylquinazolin-4-yl)-5-(1-methyl-3-(trifluoromethyl)-1H-pyrazol-4-yl)-3,4-dihydroisoquinolin-1(2H)-one (Example 53, 60 mg, 0.53 mmol, 5 equiv) and methylamine hydrochloride (36 mg, 1.60 mmol, 2 equiv). ¹H NMR (400 MHz, DMSO-d₆) δ 8.08 (s, 1H), 7.97 (d, J=1.9 Hz, 1H), 7.78 (t, 0.1=1.2 Hz, 1H), 7.38 (d, J=1.9 Hz, 1H), 7.21 (t, J=1.3 Hz, 1H), 6.90 (t, J=1.1 Hz, 1H), 6.86 (s, 1H), 6.63 (s, 1H), 6.56 (q, J=4.9 Hz, 1H), 5.31 (s, 2H), 3.99 (s, 3H), 3.92 (brs, 2H), 3.82 (s, 3H), 2.98 (brs, 2H), 2.86 (d, J=4.9 Hz, 3H), 2.58 (s, 3H); ¹⁹F NMR (376 MHz, DMSO-d₆) δ −58.26; LCMS (ESI) Method 2: >95%, R_{T}=1.256 min, m/z=577.0 [M+H]⁺.

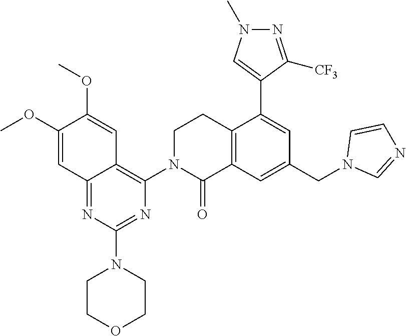 [SMILES: COc1cc2nc(N3CCOCC3)nc(N3CCc4c(cc(Cn5ccnc5)cc4-c4cn(C)nc4C(F)(F)F)C3=O)c2cc1OC]

### Example 56

### 7-((1H-Imidazol-1-yl)methyl)-2-(6,7-dimethoxy-2-morpholinoquinazolin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-5-(1-methyl-3-(trifluoromethyl)-1H-pyrazol-4-yl)-3,4-dihydroisoquinolin-1(2H)-one (Intermediate 8, 45 mg, 0.12 mmol, 1 equiv), 4-(4-chloro-6,7-dimethoxyquinazolin-2-yl)morpholine (Intermediate 45, 53 mg, 0.17 mmol, 1.4 equiv), cesium carbonate (78 mg, 0.24 mmol, 2 equiv), Xantphos (10 mg, 0.018 mmol, 0.15 equiv), and Pd₂(dba)₃ (5.5 mg, 0.006 mmol, 0.05 equiv) were dissolved in 1,4-dioxane (1 mL) under an Ar. The reaction mixture was stirred for 14 h at 115° C. then cooled to 23° C. Brine was added to the mixture and the mixture was extracted with CH₂Cl₂ (3×20 mL). The combined organic layers were dried over MgSO₄ and concentrated under reduced pressure. The residue was purified by reversed-phase HPLC (Phenomenex Gemini Cl₈, H₂O/CH₃CN gradient from 15-70% CH₃CN, 0.1% TFA) followed by neutralization with sat. aq. NaHCO₃ to obtain the title compound (60 mg, 0.092 mmol, 77% yield). ¹H NMR (400 MHz, DMSO-d₆) δ 8.08 (s, 1H), 7.98 (d, J=1.9 Hz, 1H), 7.78 (s, 1H), 7.39 (d, J=1.9 Hz, 1H), 7.21 (d, J=1.4 Hz, 1H), 6.99 (s, 1H), 6.92 (s, 1H), 6.90 (s, 1H), 5.31 (s, 2H), 3.99 (s, 3H), 3.96 (brs, 2H), 3.92 (s, 3H), 3.74-3.72 (m, 4H), 3.71 (s, 3H), 3.68-3.65 (m, 4H), 2.98 (brs, 2H); ⁹F NMR (376 MHz, DMSO-d₆) δ −58.23; LCMS (ESI) Method 2: >95%, R_{T}=1.343 min, m/z=649.0 [M+H]⁺.

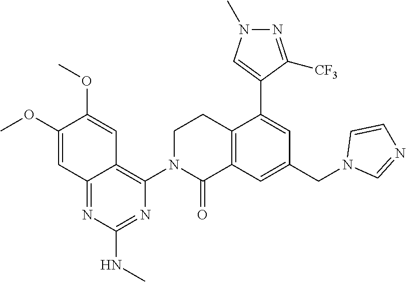 [SMILES: CNc1nc(N2CCc3c(cc(Cn4ccnc4)cc3-c3cn(C)nc3C(F)(F)F)C2=O)c2cc(OC)c(OC)cc2n1]

### Example 57

### 7-((1H-Imidazol-1-yl)methyl)-2-(6,7-dimethoxy-2-(methylamino)quinazolin-4-yl)-5-(1-methyl-3-(trifluoromethyl)-1H-pyrazol-4-yl)-3,4-dihydroisoquinolin-1(2H)-one

The title compound (42 mg, 0.071 mmol, 53% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 50 mg, 0.13 mmol, 1 equiv) and 4-chloro-6,7-dimethoxy-N-methylquinazolin-2-amine (Intermediate 46.60 mg, 0.24 mmol, 1.8 equiv). ¹H NMR (400 MHz, DMSO-d_{f}) δ 8.08 (s, 1H), 7.98 (d, J=2.0 Hz, 1H), 7.78 (s, 1H), 7.38 (s, 1H), 7.21 (s, 1H), 7.01 (d, J=5.0 Hz, 1H), 6.97 (s, 1H), 6.90 (s, 1H), 6.87 (s, 1H), 5.31 (s, 2H), 3.99 (s, 3H), 3.91 (s, 3H), 3.89 (brs, 2H), 3.69 (s, 3H), 2.97 (brs, 2H), 2.86 (d, 0.1=4.7 Hz, 3H); ¹¹F NMR (376 MHz, DMSO-d₆) 5-58.27; LCMS (ESI) Method 2: >95%, R_{T}=1.292 min, m/z=593.1 [M+H]⁺.

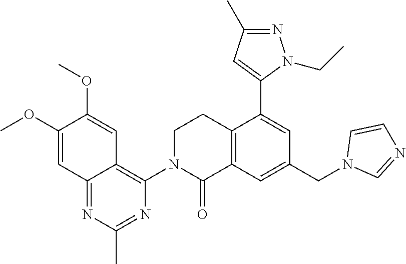 [SMILES: CCn1nc(C)cc1-c1cc(Cn2ccnc2)cc2c1CCN(c1nc(C)nc3cc(OC)c(OC)cc13)C2=O]

### Example 58

### 7-((1H-Imidazol-1-yl)methyl)-2-(6,7-dimethoxy-2-methylquinazolin-4-yl)-5-(1-ethyl-3-methyl-1H-pyrazol-5-yl)-3,4-dihydroisoquinolin-1(2H)-one

7-((1H-Imidazol-1-yl)methyl)-5-bromo-2-(6,7-dimethoxy-2-methylquinazolin-4-yl)-3,4-dihydroisoquinolin-1(2H)-one (Intermediate 48, 50 mg, 0.098 mmol, 1 equiv), 1-ethyl-3-methyl-5-(4,4,5,5-tetramethyl-1,3,2-dioxaborolan-2-yl)-1H-pyrazole (46 mg, 0.20 mmol, 2 equiv), K₂CO₃ (34 mg, 0.25 mmol, 2.5 equiv), and PdC12(dppf)-CH₂Cl₂ adduct (4 mg, 0.005 mmol, 0.05 equiv), were dissolved in 1,4-dioxane/H₂O (4.1, 1 mL). The reaction mixture was placed under an argon atmosphere heated to 80° C. for 14 h and then cooled to room temperature. The reaction mixture was diluted with water and extracted with CH₂Cl₂. The combined organic layers were washed with brine, dried over MgSO₄, filtered, and concentrated under reduced pressure. The residue was purified by preparative reversed-phase HPLC (Phenomenex Gemini C18, MeCN/1-H₂O=15-70% gradient, 0.1% TFA) followed by neutralization with sat. aqu. NaHCO₃ to yield the title compound (35 mg, 0.065 mmol, 66% yield). ¹H NMR (400 MHz, DMSO-d₆) δ 8.03 (d, J=1.9 Hz, 1H), 7.82 (s, 1H), 7.46 (d, J=1.9 Hz, 1H), 7.34 (s, 1H), 7.26 (d, J=1.2 Hz, 1H), 7.09 (s, 1H), 6.91 (d, J=1.2 Hz, 1H), 6.14 (s, 1H), 5.33 (s, 2H), 4.00 (brs, 2H), 3.98 (s, 3H), 3.84 (q, J=7.3 Hz, 2H), 3.80 (s, 3H), 2.99 (brs, 2H), 2.67 (s, 3H), 2.21 (s, 3H), 1.20 (t, J=7.1 Hz, 3H); LCMS (ESI) Method 2: >95%, R_{T}=1.218 min, m/z=538.0 [M+H]⁺.

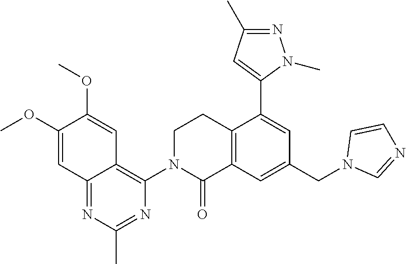 [SMILES: COc1cc2nc(C)nc(N3CCc4c(cc(Cn5ccnc5)cc4-c4cc(C)nn4C)C3=O)c2cc1OC]

### Example 59

### 7-((1H-Imidazol-1-yl)methyl)-2-(6,7-dimethoxy-2-methylquinazolin-4-yl)-5-(1,3-dimethyl-1H-pyrazol-5-yl)-3,4-dihydroisoquinolin-1(2H)-one

The title compound (28 mg, 0.053 mmol, 54% yield) was prepared following the coupling procedure described for Example 58, using 7-((1H-imidazol-1-yl)methyl)-5-bromo-2-(6,7-dimethoxy-2-methylquinazolin-4-yl)-3,4-dihydroisoquinolin-1(2H)-one (Intermediate 48, 50 mg, 0.098 mmol, 1 equiv) and (1,3-dimethyl-1H-pyrazol-5-yl)boronic acid (21 mg, 0.15 mmol, 1.5 equiv). ¹H NMR (400 MHz, DMSO-d₆) δ 8.02 (d, J=1.9 Hz, 1H), 7.82 (s, 1H), 7.53 (d, J=1.8 Hz, 1H), 7.34 (s, 1H), 7.27 (s, 1H), 7.08 (s, 1H), 6.91 (s, 1H), 6.17 (s, 1H), 5.32 (s, 2H), 4.01 (brs, 2H), 3.98 (s, 3H), 3.79 (s, 3H), 3.60 (s, 3H), 3.02 (brs, 2H), 2.67 (s, 3H), 2.20 (s, 3H); LCMS (ESI) Method 2: >95%, R_{T}=1.245 min, m/z=524.1 [M+H]⁺.

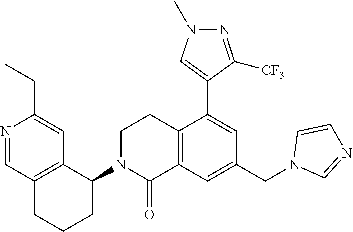 [SMILES: CCc1cc2c(cn1)CCC[C@@H]2N1CCc2c(cc(Cn3ccnc3)cc2-c2cn(C)nc2C(F)(F)F)C1=O]

### Example 60

### (S)-7-((1H-Imidazol-1-yl)methyl)-3′-ethyl-5-(1-methyl-4-(trifluoromethyl)-1H-pyrazol-3-yl)-3,4,5′,6′,7′,8′-hexahydro-1H-[2,5′-biisoquinolin]-1-one

The title compound (71 mg, 0.13 mmol, 80% yield) was prepared following the coupling procedure described for Intermediate 7, using (S)-7-(chloromethyl)-3′-ethyl-5-(1-methyl-4-(trifluoromethyl)-JH-pyrazol-3-yl)-3,4,5′,6′,7′,8′-hexahydro-/H-[2,5′-biisoquinolin]-1-one (Intermediate 52, 84 mg, 0.17 mmol, 1 equiv) and 1H₁-imidazole (28 mg, 0.42 mmol, 2.5 equiv). ¹H NMR (400 MHz, Chloroform-d) δ 8.31 (s, 1H), 8.14 (d, J=2.0 Hz, 1H), 7.87 (s, 1H), 7.60 (s, 1H), 7.39-7.30 (m, 1H), 7.22-7.06 (m, 5H), 6.96 (d, J=4.6 Hz, 2H), 6.02 (dd, 0.1=10.7, 5.9 Hz, 1H), 5.19 (s, 2H), 3.99 (s, 3H), 3.29 (ddd, J=12.4, 9.0, 5.7 Hz, 1H), 3.08 (dt, J=12.1, 5.4 Hz, 1H), 2.88-2.51 (m, 5H), 2.26-1.98 (m, 2H), 1.98-1.64 (m, 2H), 1.23 (t, J=7.6 Hz, 3H); ¹⁹F NMR (376 MHz, Chloroform-d) 5-60.19; LCMS (ESI): m/z=535.4 [M+H]⁺.

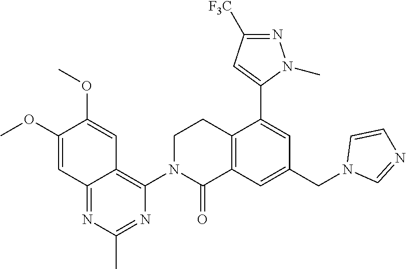 [SMILES: COc1cc2nc(C)nc(N3CCc4c(cc(Cn5ccnc5)cc4-c4cc(C(F)(F)F)nn4C)C3=O)c2cc1OC]

### Example 61

### 7-((1H-Imidazol-1-yl)methyl)-2-(6,7-dimethoxy-2-methylquinazolin-4-yl)-5-(1-methyl-3-(trifluoromethyl)-1H-pyrazol-5-yl)-3,4-dihydroisoquinolin-1(2H)-one

The title compound (39 mg, 0.068 mmol, 69% yield) was prepared following the coupling procedure described for Example 58, using 7-((1H-imidazol-1-yl)methyl)-5-bromo-2-(6,7-dimethoxy-2-methylquinazolin-4-yl)-3,4-dihydroisoquinolin-1(2H₁)-one (Intermediate 48, 50 mg, 0.098 mmol, 1 equiv) and (1-methyl-3-(trifluoromethyl)-1H-pyrazol-5-yl)boronic acid (38 mg, 0.20 mmol, 2 equiv). ¹H NMR (400 MHz, DMSO-d₆) δ 8.09 (d, J=1.9 Hz, 1H), 7.83 (s, 1H), 7.70 (d, J=1.9 Hz, 1H), 7.35 (s, 1H), 7.29 (s, 1H), 7.08 (s, 1H), 6.95 (s, 1H), 6.91 (s, 1H), 5.33 (s, 2H), 4.02 (brs, 2H), 3.98 (s, 3H), 3.80 (s, 3H), 3.79 (s, 2H), 3.04 (brs, 2H), 2.67 (s, 3H); ¹⁹F NMR (376 MHz, DMSO-d₆) δ −60.32; LCMS (ESI) Method 2: >95%, R_{T}=1.409 min, m/z=578.0 [M+H]⁺.

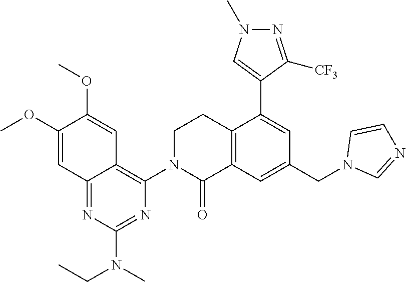 [SMILES: CCN(C)c1nc(N2CCc3c(cc(Cn4ccnc4)cc3-c3cn(C)nc3C(F)(F)F)C2=O)c2cc(OC)c(OC)cc2n1]

### Example 62

### 7-((1H-Imidazol-1-yl)methyl)-2-(2-(ethyl(methyl)amino)-6,7-dimethoxyquinazolin-4-yl)-5-(1-methyl-3-(trifluoromethyl)-1H-pyrazol-4-yl)-3,4-dihydroisoquinolin-1(2H)-one

The title compound (22 mg, 0.037 mmol, 53% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 25 mg, 0.067 mmol, 1 equiv) and 4-chloro-N-ethyl-6,7-dimethoxy-N-methylquinazolin-2-amine (Intermediate 53, 24 mg, 0.087 mmol, 1.3 equiv). ¹H NMR (400 MHz, DMSO-d₆) δ 8.08 (s, 1H), 7.99 (d, J=1.9 Hz, 1H), 7.78 (s, 1H), 7.39 (s, 1H), 7.21 (s, 1H), 6.94 (s, 1H), 6.90 (s, 1H), 6.88 (s, 1H), 5.31 (s, 2H), 3.99 (s, 3H), 3.95 (brs, 2H), 3.92 (s, 3H), 3.70 (s, 3H), 3.67 (q, J=7.0 Hz, 2H), 3.12 (s, 3H), 2.98 (brs, 2H), 1.11 (t, J=7.0 Hz, 3H); ¹⁹F NMR (376 MHz, DMSO-d₆) δ −58.20; LCMS (ESI) Method 2: >95%, R_{T}=1.359 min, m/z=621.0 [M+H]⁺.

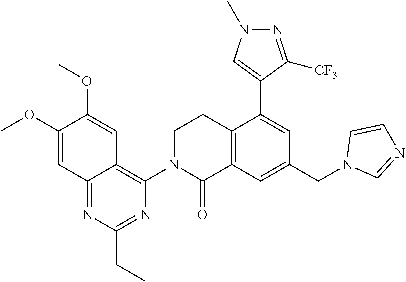 [SMILES: CCc1nc(N2CCc3c(cc(Cn4ccnc4)cc3-c3cn(C)nc3C(F)(F)F)C2=O)c2cc(OC)c(OC)cc2n1]

### Example 63

### 7-((1H-Imidazol-1-yl)methyl)-2-(2-ethyl-6,7-dimethoxyquinazolin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-5-(1-methyl-3-(trifluoromethyl)-1H-pyrazol-4-yl)-3,4-dihydroisoquinolin-1(2H)-one (Intermediate 8, 60 mg, 0.16 mmol, 1 equiv), 4-chloro-2-ethyl-6,7-dimethoxyquinazoline (Intermediate 54, 61 mg, 0.24 mmol, 1.5 equiv), cesium carbonate (104 mg, 0.32 mmol, 2 equiv), Xantphos (14 mg, 0.024 mmol, 0.15 equiv), and Pd₂(dba)₃ (7.3 mg, 0.008 mmol, 0.05 equiv) were dissolved in 1,4-dioxane (1 mL) under an Ar. The reaction mixture was stirred for 14 h at 115° C. then cooled to 23° C. Brine was added to the mixture and the mixture was extracted with CH₂Cl₂ (3×20 mL). The combined organic layers were dried over MgSO₄ and concentrated under reduced pressure. The residue was purified by reversed-phase HPLC (Phenomenex Gemini C18, H₂O/CH₃CN gradient from 15-70% CH₃CN, 0.1% TFA) followed by neutralization with sat. aq. NaHCO₃ to obtain the title compound (62 mg, 0.10 mmol, 65% yield). ¹H NMR (400 MHz, DMSO-d₆) δ 8.09 (d, J=1.1 Hz, 1H), 7.99 (d, J=1.9 Hz, 1H), 7.78 (t, J=1.1 Hz, 1H), 7.39 (d, J=1.9 Hz, 1H), 7.35 (s, 1H), 7.21 (t, J=1.2 Hz, 1H), 7.07 (s, 1H), 6.90 (t, J=1.1 Hz, 1H), 5.31 (s, 2H), 4.01 (brs, 2H), 3.99 (s, 3H), 3.98 (s, 3H), 3.79 (s, 3H), 2.95 (q, J=7.6 Hz, 2H), 1.33 (t, J=7.6 Hz, 3H); LCMS (ESI) Method 2: >95%, R_{T}=1.395 min, m/z=592.0 [M+H]⁺.

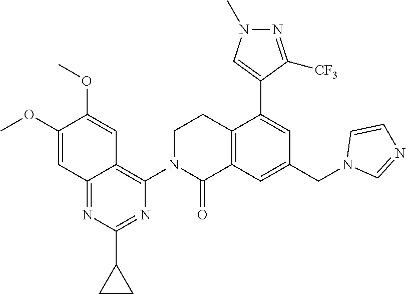 [SMILES: COc1cc2nc(C3CC3)nc(N3CCc4c(cc(Cn5ccnc5)cc4-c4cn(C)nc4C(F)(F)F)C3=O)c2cc1OC]

### Example 64

### 7-((1H-Imidazol-1-yl)methyl)-2-(2-cyclopropyl-6,7-dimethoxyquinazolin-4-yl)-5-(1-methyl-3-(trifluoromethyl)-1H-pyrazol-4-yl)-3,4-dihydroisoquinolin-1(2H)-one

The title compound (16 mg, 0.026 mmol, 48% yield) was prepared following the Buchwald coupling procedure described for Intermediate 15, using 7-((1H-imidazol-1-yl)methyl)-5-(1-methyl-3-(trifluoromethyl)-1H-pyrazol-4-yl)-3,4-dihydroisoquinolin-1(2H)-one (Intermediate 8, 20 mg, 0.053 mmol, 1 equiv) and 4-chloro-2-cyclopropyl-6,7-dimethoxyquinazoline (intermediate 55, 18 mg, 0.069 mmol, 1.3 equiv). ¹H NMR (400 MHz, DMSO-d₆) δ 8.09 (d, J=1.1 Hz, 1H), 7.99 (d, J=1.9 Hz, 1H), 7.79 (s, 1H), 7.40 (d, J=1.9 Hz, 1H), 7.27 (s, 1H), 7.22 (s, 1H), 7.04 (s, 1H), 6.91 (s, 1H), 5.31 (s, 2H), 3.99 (s, 3H), 3.98 (brs, 2H), 3.97 (s, 3H), 3.77 (s, 3H), 3.00 (brs, 2H), 2.23 (tt, J=7.6, 5.3 Hz, 1H), 1.04 (ddd, J=7.7, 3.9, 2.4 Hz, 4H); LCMS (ESI) Method 2: >95%, R_{T}=1.418 min, m/z=604.0 [M+H]⁺.

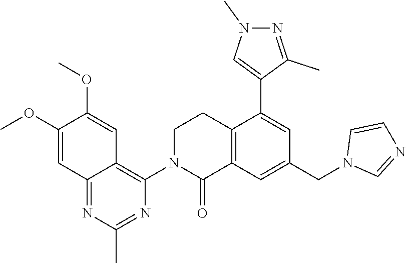 [SMILES: COc1cc2nc(C)nc(N3CCc4c(cc(Cn5ccnc5)cc4-c4cn(C)nc4C)C3=O)c2cc1OC]

### Example 65

### 7-((1H-Imidazol-1-yl)methyl)-2-(6,7-dimethoxy-2-methylquinazolin-4-yl)-5-(1,3-dimethyl-1H-pyrazol-4-yl)-3,4-dihydroisoquinolin-1(2H)-one

The title compound (52 mg, 0.099 mmol, quant.) was prepared following the coupling procedure described for Example 58, using 7-((1H-imidazol-1-yl)methyl)-5-bromo-2-(6,7-dimethoxy-2-methylquinazolin-4-yl)-3,4-dihydroisoquinolin-1(2H₁)-one (Intermediate 48, 50 mg, 0.098 mmol, 1 equiv) and 1,3-dimethyl-4-(4,4,5,5-tetramethyl-1,3,2-dioxaborolan-2-yl)-1H-pyrazole (44 mg, 0.20 mmol, 2 equiv). ¹H NMR (400 MHz, DMSO-d₆) δ 7.90 (d, J=1.9 Hz, 1H), 7.80 (d, J=1.2 Hz, 1H), 7.76 (s, 1H), 7.38 (d, J=2.0 Hz, 1H), 7.34 (s, 1H), 7.25 (t, J=1.3 Hz, 1H), 7.06 (s, 1H), 6.91 (t, J=1.1 Hz, 1H), 5.30 (s, 2H), 4.00 (brs, 2H), 3.98 (s, 3H), 3.82 (s, 3H), 3.79 (s, 3H), 3.10 (brs, 2H), 2.67 (s, 3H), 2.11 (s, 3H); LCMS (ESI) Method 2: >95%, R_{T}=1.203 min, m/z=524.1 [M+H]⁺.

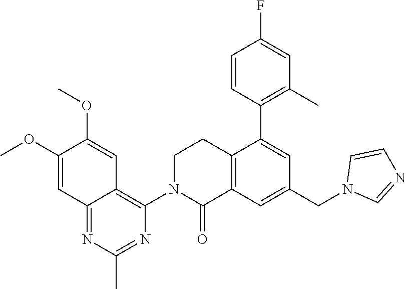 [SMILES: COc1cc2nc(C)nc(N3CCc4c(cc(Cn5ccnc5)cc4-c4ccc(F)cc4C)C3=O)c2cc1OC]

### Example 66

### 7-((1H-Imidazol-1-yl)methyl)-2-(6,7-dimethoxy-2-methylquinazolin-4-yl)-5-(4-fluoro-2-methylphenyl)-3,4-dihydroisoquinolin-1(2H)-one

The title compound (12 mg, 0.022 mmol, 56% yield) was prepared following the coupling procedure described for Example 58, using 7-(1H-imidazol-1-yl)methyl)-5-bromo-2-(6,7-dimethoxy-2-methylquinazolin-4-yl)-3,4-dihydroisoquinolin-1(2H)-one (Intermediate 48, 20 mg, 0.039 mmol, 1 equiv) and (4-fluoro-2-methylphenyl)boronic acid (12 mg, 0.079 mmol, 2 equiv). ¹H NMR (400 MHz, DMSO-ds) δ 7.97 (d, J=1.9 Hz, 1H), 7.80 (t, J=1.1 Hz, 1H), 7.36 (d, J=1.9 Hz, 1H), 7.34 (s, 1H), 7.25 (t, J=1.3 Hz, 1H), 7.25-7.20 (m, 2H), 7.13 (td, J=8.5, 2.7 Hz, 1H), 7.08 (s, 1H), 6.90 (t, J=1.1 Hz, 1H), 5.31 (s, 2H), 3.98 (brs, 2H), 3.97 (s, 3H), 3.80 (s, 3H), 2.95 (brs, 2H), 2.66 (s, 3H), 2.09 (s, 3H); LCMS (ESI) Method 2: >95%, R_{T}=1.483 min, m/z=538.0 [M+H]⁺.

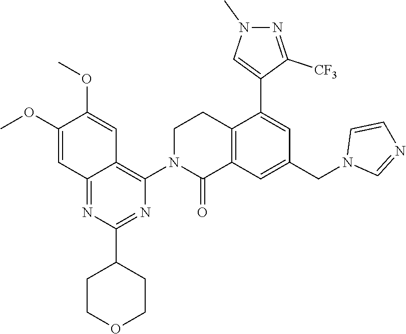 [SMILES: COc1cc2nc(C3CCOCC3)nc(N3CCc4c(cc(Cn5ccnc5)cc4-c4cn(C)nc4C(F)(F)F)C3=O)c2cc1OC]

### Example 67

### 7-((1H-Imidazol-1-yl)methyl)-2-(6,7-dimethoxy-2-(tetrahydro-2H-pyran-4-yl)quinazolin-4-yl)-5-(1-methyl-3-(trifluoromethyl)-1H-pyrazol-4-yl)-3,4-dihydroisoquinolin-1(2H)-one

7-((1H-Imidazol-1-yl)methyl)-5-(1-methyl-3-(trifluoromethyl)-1H-pyrazol-4-yl)-3,4-dihydroisoquinolin-1(2H)-one (intermediate 8, 260 mg, 0.69 mmol, 1 equiv), 4-chloro-6,7-dimethoxy-2-(tetrahydro-2H-pyran-4-yl)quinazoline (Intermediate 56, 278 mg, 0.90 mmol, 1.3 equiv), cesium carbonate (451 mg, 1.39 mmol, 2 equiv), Xantphos (60 mg, 0.10 mmol, 0.15 equiv), and Pd₂(dba)₃ (32 mg, 0.035 mmol, 0.05 equiv) were dissolved in 1,4-dioxane (4 mL) under an Ar. The reaction mixture was stirred for 14 h at 115° C. then cooled to 23° C. Brine was added to the mixture and the mixture was extracted with CH₂Cl₂ (3×40 mL). The combined organic layers were dried over MgSO₄ and concentrated under reduced pressure. The residue was purified by reversed-phase HPLC (Phenomenex Gemini C18, H₂O/CH₃CN gradient from 15-70% CH₃CN, 0.1% TFA) followed by neutralization with sat. aq. NaHCO₃ to obtain the title compound (390 mg, 0.60 mmol, 87% yield). ¹H NMR (400 MHz, DMSO-d₆) δ 8.09 (d, J=1.1 Hz, 1H), 8.00 (d, J=1.9 Hz, 1H), 7.79 (d, J=1.1 Hz, 1H), 7.40 (d, J=1.9 Hz, 1H), 7.35 (s, 1H), 7.21 (t, J=1.3 Hz, 1H), 7.08 (s, 1H), 6.91 (t, J=1.1 Hz, 1H), 5.31 (s, 2H), 4.03 (brs, 2H), 3.99 (s, 6H), 3.95 (dt, J=11.4, 2.6 Hz, 2H), 3.79 (s, 3H), 3.48 (td, J=11.3, 3.0 Hz, 2H), 3.12 (tt, J=10.9, 4.5 Hz, 1H), 3.02 (brs, 2H), 1.97-1.80 (m, 4H); ¹F NMR (376 MHz, DMSO-d₆) δ −58.22; LCMS (ESI) Method 2: >95%, R_{T}=1.402 min, m/z=648.0 [M+H]⁺.

### 3. Pharmaceutical Compositions

The disclosed compounds may be incorporated into pharmaceutical compositions suitable for administration to a subject (such as a patient, which may be a human or non-human).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention [e.g., a compound of formula (T)] are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The routes by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGTT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound [e.g., a compound of formula (I)], and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% A to about 0.5%, particularly, about 0.001% to about 0.1%.

Suitable pH adjusting additives include HCl or NaOH in amounts sufficient to adjust the pH of a topical pharmaceutical composition.

### 4. Methods of Treatment

Mixed lineage leukemia (MLL) presents a heterogeneous group of acute myeloid leukemia and acute lymphoblastic leukemia bearing features of more than one hematopoietic cell lineages. MLL accounts for about 80% of infant acute leukemia cases (Tomizawa, 2007) and 10% of all acute leukemia cases (Marschalek, 2011). MLL leukemia patients have a poor prognosis with overall 5-year survival ratio around 35% (Dimartino, 1999; Pui, 2003; Tomizawa, 2007).

MLL is composited of heterogeneous cell lineages with different molecular biology, cell biology and immunology features. However, MLL does share a common feature, which involves the chromosomal rearrangement of Mixed Lineage Leukemia (MLL) gene. MLL gene locates on chromosome 11q23 and the encoded MLL protein is a homolog of Drosophila trithorax (Trx) (Thachuk, 1992). Wild type MLL binds to regulatory regions of homeox (HOX) genes (Milne, 2005) through the amino terminal fragment while the catalytic C-terminal domain catalyzes the Histone 3 lysine 4 (H3K4) methylation via interaction with WDR5 and up regulates target genes transcription (Nakamura, 2002; Yokoyama, 2004; Milne, 2002). Wild type MLL in conjunction with WDR5 is required for maintenance HOX genes expression and is widely expressed not only during embryo development but also in adult tissues including myeloid and lymphoid cells (Butler, 1997; Yu, 1998). Reciprocal translocations of MLL gene result in-frame fusion of 5′-end MLL with the 3′-end of another partner gene. A common feature of MLL1 abnormality in leukemia is the preservation of one wild-type MLL1 allele. Currently, more than 80 partner genes have been identified, with MLL-AF4, MLL-AF9 and MLL-ENL being the three most frequently found fusion genes (Pui, 2003; herein incorporated by reference in its entirety). Expression of MLL fusion proteins promotes over expression of target genes such as HOXA9 and MEIS1, which blocks differentiation, enhances blast expansion and ultimately leads to leukemic transformation (Caslini, 2007; Yokoyama, 2005). The numerous chromosomal translocation of MLL gene and partner genes diversity add to the complexity to MLL leukemia treatment, though HOX9 and MEIS1 overexpression are commonly observed among MLL leukemia patients, each rearrangement leading to distinct dysregulated target gene expression patterns and downstream events (Slany, 2009). Clinical studies reveal that MLL of different chromosomal translocations are associated with different prognosis and are treated differently under current protocols (Tamai, 2010; Balgobind, 2011; Pigazzi, 2011).

Intrinsic HMT activity of MLL1 is extremely low and requires a complex assembly of WDR5, RbBP5, ASH2L, and DPY30 protein partners for effective H3K4 trimethylation, so called WRAD complex. The binding of MLL1 to WDR5 (WD40 repeat protein 5) is particularly critical for HMT activity and occurs through a conserved arginine containing motif on MLL1 called the “Win” or WDR5 interaction motif. Thus, targeting inhibitors of the MLL1-WDR5 interaction at the WIN site in order to block MLL1 methyltransferase activity could represent a promising therapeutic strategy for treating MLL leukemia patients. Peptidomimetics have been discovered that bind tightly to WDR5 at the MLL site, inhibit MLL1 methyltransferase activity, and block proliferation of MLL1 cells by inducing cell-cycle arrest, apoptosis, and myeloid differentiation (Cao, et al. Molecular Cell, 2014, 53, 247-261.) In addition, altered gene expression patterns similar to MLL1 deletion are observed, supporting a role for MLL1 activity in regulating MLL1-dependent leukemia transcription. Thus, interruption of the WDR5-MLL1 interaction may be a useful strategy for treating patients with MLL leukemias. The molecules described herein will target this interaction and could provide an attractive therapeutic approach to develop novel drugs for leukemias with translocations of MLL gene and other leukemias with upregulation of target genes. It also appreciated that WDR5 has been implicated in other cancer types and may utilize the WIN-site for other chromatin regulatory complexes outside and/or overlapping with WRAD complex. As such the WIN-site inhibitors described herein may have utility in multiple cancer types through mechanisms of action involving both direct competitive WIN-site antagonism, or through allosteric inhibition of higher complexes wherein WDR5 is dependent for their proliferative activity and tumor formation. Examples include breast cancer (Dai, X. et al. PLoS One, 2015), MYC-driven tumor types (Thomas, et al. Molecular Cell, 2015), bladder cancer (Chen, X. et al. Nature, Scientific Reports, 2015), neuroblastoma (Sun, Y. et al. Cancer Research, 2015), and pancreatic cancer (Carugo, A. et al. Cell Reports, 2016).

The disclosed compounds and compositions may be used in methods for treatment of MLL related cancers. The methods of treatment may comprise administering to a subject in need of such treatment a composition comprising a therapeutically effective amount of the compound of formula (I).

In one aspect, disclosed is a method of treating cancer, the method comprising administration of a therapeutically effective amount of a compound of formula (I), or a pharmaceutically acceptable salt thereof to a subject in need thereof.

In certain embodiments, the cancer being treated is associated with dysfunction of MLL.

In certain embodiments, the cancer is at least one of leukemia, ovarian cancer, breast cancer, colorectal cancer, pancreatic cancer, gastric cancer, stomach cancer, lung cancer, cervical cancer, uterine cancer, cancers of the blood, and cancers of the lymphatic system.

In another aspect, disclosed is a method of disrupting the protein-protein interaction between WDR5 and MLL1, the method comprising administration of a therapeutically effective amount of a compound of formula (I), or a pharmaceutically acceptable salt thereof to a subject in need thereof.

The compositions can be administered to a subject in need thereof to bind WDR5 and modulate MLL, to treat a variety of diverse cancers. The present disclosure is directed to methods for administering the composition to inhibit the protein-protein interaction between WDR5 its binding partners such chromatin, cognate transcription and other regulatory factors, including for example the histone methyltransferase MLL1.

The compositions may be useful for treating certain cancers in humans and animals related to MLL dysfunction. Treatment of such cancers can be effected by modulating MLL1 in a subject, by administering a compound or composition of the invention, either alone or in combination with another active agent as part of a therapeutic regimen to a subject in need thereof.

Disruption of the the interaction between WDR5 and its binding partners (such as MLL1) can lead to treatment and reduction of cancer or tumor growth, and/or reduce metastasis of cancerous or tumor cells. Accordingly, the disclosed compositions can be used in methods that treat and/or prevent cancer or tumors in a subject administered the composition. The method can treat cancer or tumor based growth and can be any type of cancer such as, but not limited to, leukemia (mixed-lineage leukemia), ovarian cancer, breast cancer, colorectal cancer, pancreatic cancer, gastric cancer, stomach cancer, lung cancer, cervical cancer, uterine cancer, cancers of the blood, and cancers of the lymphatic system.

In some embodiments, the administered composition to a subject in need thereof can mediate reduction, clearance or prevention of additional growth of tumor cells by disrupting the ability of MLL1, another transcription factor, or chromatin to associate with WDR5, thereby reducing growth/proliferation of tumor cells, but does not have an effect on normal cells.

In some embodiments, the administered composition can increase tumor free survival, reduce tumor mass, slow tumor growth, increase tumor survival, or a combination thereof in the subject. The administered composition can reduce tumor volume in the subject in need thereof. The administered composition can increase tumor free survival in the subject after administration of the composition.

In some embodiments, the composition can be administered to clear or eliminate the cancer or tumor expressing the one or more oncogenes without damaging or causing illness or death in the subject administered the composition.

### A. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

### B. Combination Therapies

Additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds. For example, the compound of Formula (I) can be combined with a variety of different anti-cancer drugs such as chemotherapeutics, anti-tumor agents, and anti-proliferative agents.

Further, the compound of formula (I) can be combined with the following, but not limited to, actinomycins, alkylating agents, anthracyclines, antifolates, antiestrogen agents, anti-metabolites, anti-androgens, antimicrotubule agents, aromatase inhibitors, bleomycins, bromodomain inhibitors, Ca²⁺ adenosine triphosphate (ATP)ase inhibitors, cytosine analogs, deltoids/retinoids, dihydrofolate reductase inhibitors, deoxyribonucleic acid (DNA) topoisomerase inhibitors, dopaminergic neurotoxins, glucocorticoids, histone deacetylase inhibitors, hormonal therapies, immunotherapeutic agents, inosine monophosphate (IMP) dehydrogenase inhibitors, isoprenylation inhibitors, luteinizing hormone-releasing hormone agonists, mammalian target of rapamycin (mtor) inhibitors, multi-drug resistance (MDR) inhibitors, mitomycins, photodynamic therapies, proteasome inhibitors, platinum containing compounds, radiation, receptor tyrosine kinase inhibitors, ribonucleotide reductase inhibitors, thrombospondin mimetics, uracil analogs, vinca alkaloids, vitamin D3 analogs, □-radiation, DOT1L inhibitors, agents targeting epigenetic mechanisms, or an additional chemotherapeutic agent such as N—Ac-Sar-Gly-Val-D-allolle-Thr-Nva-Ile-Arg-Pro-NHCH2CH3 or a salt thereof, actinomycin D, AG13736, 17-allylamino-17-demethoxygeldanamycin, 9-aminocamptothecin, N-(4-(3-amino-1H-indazol-4-yl)phenyl}-N-(2-fluoro-5-methylphenyl)urea or a salt thereof, N-(4-(4-aminothieno[2,3-d]pyrimidin-5-yl)phenyl}-N-(2-fluoro-5-(trifluoromethyl)phenyl)urea or a salt thereof, temozolomide, nedaplatin, satraplatin, triplatin tetranitrate, procarbazine, altretamine, mitozolomide, anastozole, AP-23573, asparaginase, azacitidine, bevacizurnab, bicalutamide, bleomycin a2, bleomycin b2, bortezemib, busulfan, campathecins, carboplatin, carmustine (BCNU), CB1093, cetuximab, CHOP (C: Cytoxan® (cyclophosphamide); H: Adriamycin® (hydroxydoxorubicin); O: Vincristine (Oncovin®); P: prednisone), chlorambucil, CHIR258, cisplatin, CNF-101, CNF-1001, CNF-2024, CP547632, crisnatol, cytarabine, cyclophosphamide, cytosine arabinoside, daunorubicin, dacarbazine, dactinomycin, dasatinib, daunorubicin, deferoxamine, demethoxyhypocrellin A, depsipeptide, dexamethasone, 17-dimethylaminoethylamino-17-demethoxygeldanamycin, docetaxel, doxifluridine, doxorubicin, EB 1089, epothilone D, epirubicin, 5-ethynyl-1-13-D-ribofuranosylimidazole-4-carboxamide (EICAR), erlotinib, etoposide, everolimus, 5-fluorouracil (5-FU), floxuridine, fludarabine, flutamide, gefitinib, geldanamycin, gemcitabine, goserelin, N-(2-(4-hydroxyanilino}-3-pyridinyl}-4-methoxybenzenesulfonamide or a salt thereof, hydroxyurea, idarubicin, ifosfamide, imatinab, interferon-a, interferon-y, IPI-504, irinotecan, KH 1060, lapatanib, leucovorin calcium, LAQ824, leuprolide acetate, letrozole, lomustine (CCNU), lovastatin, megestrol, melphalan, mercaptopurine, methotrexate, 1-methyl-4-phyenylpyridinium, MG132, mitomycin, mitoxantrone, MLN518, MLN4924, MS-275, mycophenolic acid, mitomycin C, nitrosoureas, oprelvekin, oxaliplatin, paclitaxel, PARP inhibitors (e.g., rucaparib, niraparib, olaparib, iniparib, talazoparib, and veliparib), PD98059, peplomycin, photosensitizer Pc4, phtalocyanine, pirarubicin, plicamycin, prednisone, procarbizine, PTK787, PU24FC1, PU3, radicicol, raloxifene, rapamycin, ratitrexed, retinoids such as pheuretinide, ribavirin, rituximab (Rituxin®), sorafenib, staurosporine, steroids such as dexamethasone and prednisone, suberoylanilide hydroxamic acid, tamoxifen, taxol, temozolamide, teniposide, thapsigargin, thioguanine, thrombospondin-1, tiazofurin, topotecan, trapoxin, trastuzumab, treosulfan, trichostatin A, trimetrexate, trofosfamide, tumor necrosis factor, valproic acid, VER49009, verapamil, vertoporfin, vinblastine, vincristine, vindesine, vinorelbine vitamin D3, VX-680, zactima, ZK-EPO, zorubicin, bevacizumab, enzastaurin, temsirolimus, cilengitide, lapatinib, sunitinib, axitinib, pazopanib, vemurafenib, dabrafenib, JQ1 or combinations thereof.

The disclosed compounds may be included in kits comprising the compound [e.g., one or more compounds of formula (I)], a systemic or topical composition described above, or both; and information, instructions, or both that use of the kit will provide treatment for medical conditions in mammals (particularly humans). The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans).

### 5. Biological Activity

The in vitro modulation of WDR5 protein was determined as follows.

### MLL Peptide Binding Assay

### General

Provided compounds of the present invention can be demonstrated to compete for binding with fluorescently labeled peptides derived from relevant MLL protein.

### Time Resolved-Fluorescence Energy Transfer Competition Assay

A Time-Resolved Fluorescence Resonance Energy Transfer (TR-FRET) assay that measures the displacement of the 10mer-Thr-FAM probe in response to compound treatment was performed for compounds wherein the IC₅₀ from FPA assay using 10mer-Thr-FAM was below the lower assay IC₅₀ limit˜nM. Excess 10mer-Thr-FAM probe was utilized with His-tagged WDR5 in conjunction with a commercial anti-His antibody containing a Terbium label. The LanthaScreen™ Elite Tb-anti-HIS Antibody from ThermoFisher Scientific was used for this purpose. This Tb-anti-HIS has an excitation/emission of 340 nm and 490 nm, respectively. The 10mer-Thr-FAM probe when bound to WDR5 will undergo a FRET interaction with the Tb-anti-HIS and emit at 520 nm. The ratio of the 520 and 495 signals are then utilized to generate a dose-response curve to calculate an IC₅₀ value. By virtue of FRET there is little to no background fluorescence interference from 10mer-Thr-FAM probe allowing an excess of the probe to be used permitting an increase in the lower limit of the calculated Kᵢ when testing against highly potent inhibitors with Kᵢ<<1 nM. WDR5-His Tag (A23, residues 24-334) is expressed and purified in our lab in sufficient quantities for screening. 10mer-Thr-FAM peptide is used at 150 nM. WDR5-His tag protein is used at 2 nM. A source plate is prepared using an Echo Liquid Handler, which distributes the compounds to the assay plate (white, flat-bottom; OptiPlate) in a 10-point, 5-fold dilution schemes with a top concentration of 5 μM, in a final volume of 20 μL. A final target (WDR5)/Tb-Ab concentration of 2 nM/1 nM is dispensed from appropriate stock solutions, respectively. The final DMSO concentration in each well of the assay plate is 1% or lower. The plate is covered, shielded from light, and incubated for 60 minutes at room temperature with rocking. 10mer-Thr-FAM and Anti-His terbium antibody fluorescence is then measured on a Biotek Cytation 3 at excitation wavelength of 340 nm, and emission wavelengths of 495 nm and 520 nm. Working buffer conditions contain 1× Phosphate Buffered Saline, 300 mM NaCl, 0.5 mM TCEP, 0.1% CHAPS, at pH 7.2. TR-FRET signal is plotted and IC₅₀ and Kᵢ values are calculated according to the formula of Wang Z. FEBS Lett (1996) 3, 245.

$${K}_{i} = {[ I ]}_{50} / ( {[ L ]}_{50} / {K}_{d} + {[ P ]}_{0} / {K}_{d} + 1 )$$

where [1]₅₀ is the concentration of the free inhibitor at 50% inhibition, [L]₅₀ is the concentration of the free labeled ligand at 50% inhibition, [P]₀ is the concentration of the free protein at 0% inhibition, K_{d} represents the dissociation constant of the FITC-MLL or 10mer-Thr-FAM probe for WDR5. Total fluorescence is also measured, to rule out compounds that are inherently fluorescent or able to act as quenchers in the assay.
TR-FRET binding assay

TABLE 2
Kᵢ for Exemplified Compounds for Inhibition
of WDR5 by TR-FRET assay

| Example | Kᵢ (nM) |
|---|---|
| 2 | <0.02 |
| 5 | <0.02 |
| 6 | <0.02 |
| 7 | <0.02 |
| 9 | <0.02 |
| 10 | <0.02 |
| 11 | <0.02 |
| 12 | <0.02 |
| 13 | <0.02 |
| 14 | <0.02 |
| 15 | <0.02 |
| 16 | <0.02 |
| 17 | 0.03 |
| 20 | <0.02 |
| 21 | <0.02 |
| 22 | <0.02 |
| 23 | 0.025 |
| 24 | <0.02 |
| 25 | 0.036 |
| 26 | <0.02 |
| 27 | <0.02 |
| 28 | <0.02 |
| 29 | 0.032 |
| 30 | <0.02 |
| 31 | <0.02 |
| 32 | <0.02 |
| 33 | <0.02 |
| 34 | <0.02 |
| 35 | <0.02 |
| 36 | <0.02 |
| 37 | <0.02 |
| 39 | <0.02 |
| 43 | <0.02 |
| 44 | <0.02 |
| 45 | <0.02 |
| 46 | <0.02 |
| 47 | 0.024 |
| 48 | <0.02 |
| 49 | <0.02 |
| 50 | 0.022 |
| 51 | 0.022 |
| 52 | 0.03 |
| 53 | <0.02 |
| 54 | <0.02 |
| 55 | <0.02 |
| 56 | <0.02 |
| 57 | 0.023 |
| 58 | 0.03 |
| 59 | <0.02 |
| 60 | 0.53 |
| 61 | 0.031 |
| 62 | <0.02 |
| 63 | <0.02 |
| 64 | <0.02 |
| 65 | 0.023 |
| 66 | 0.02 |
| 67 | 0.025 |

Among other things, these data demonstrate the utility of representative compounds as selective inhibitors of the activity of WDR5 protein to bind peptides from relevant MLL domain.

Additional WDR5 inhibitor compounds and the K; for inhibition of WDR5 in the TR-FRET assay are shown below.

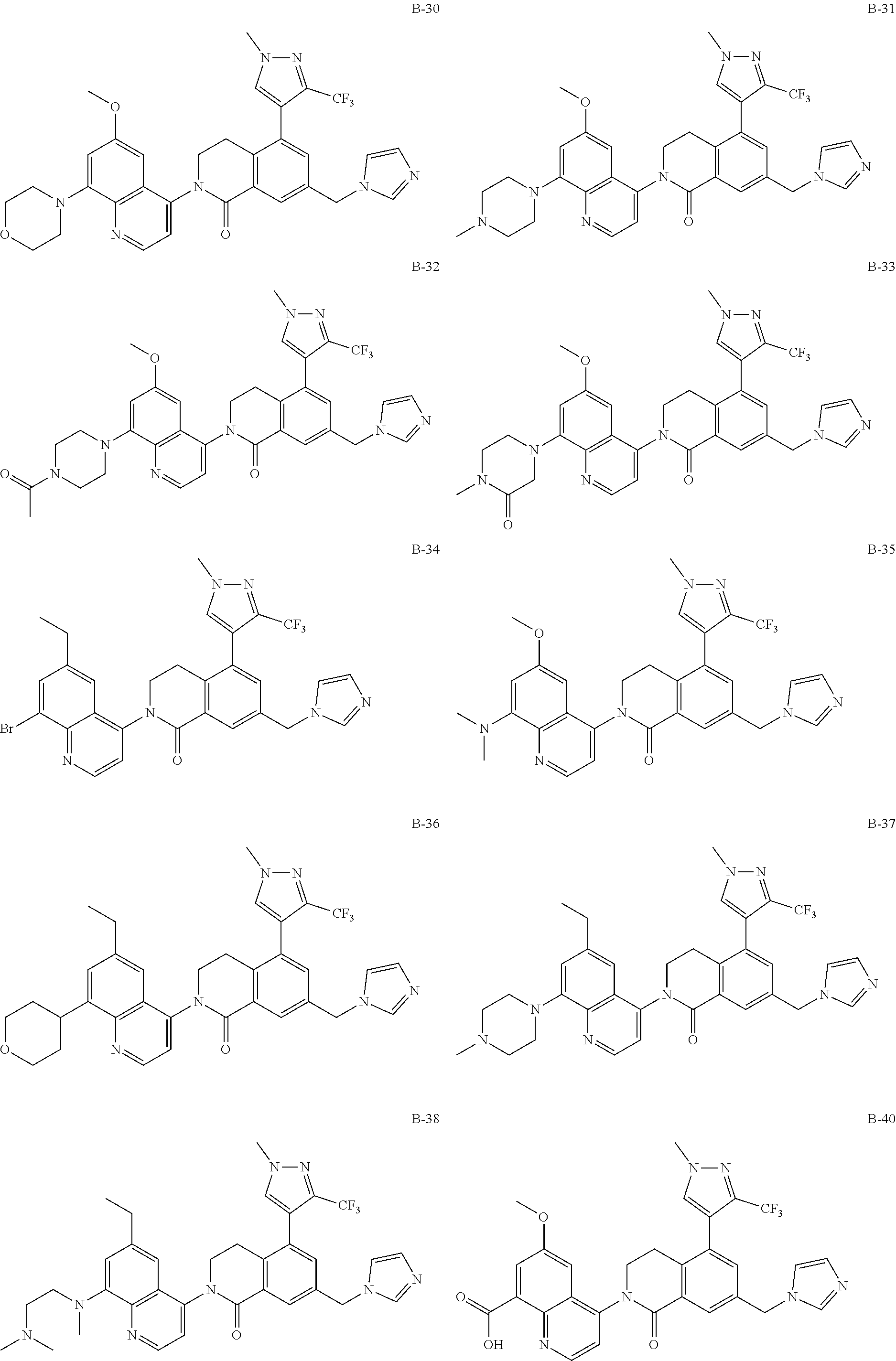

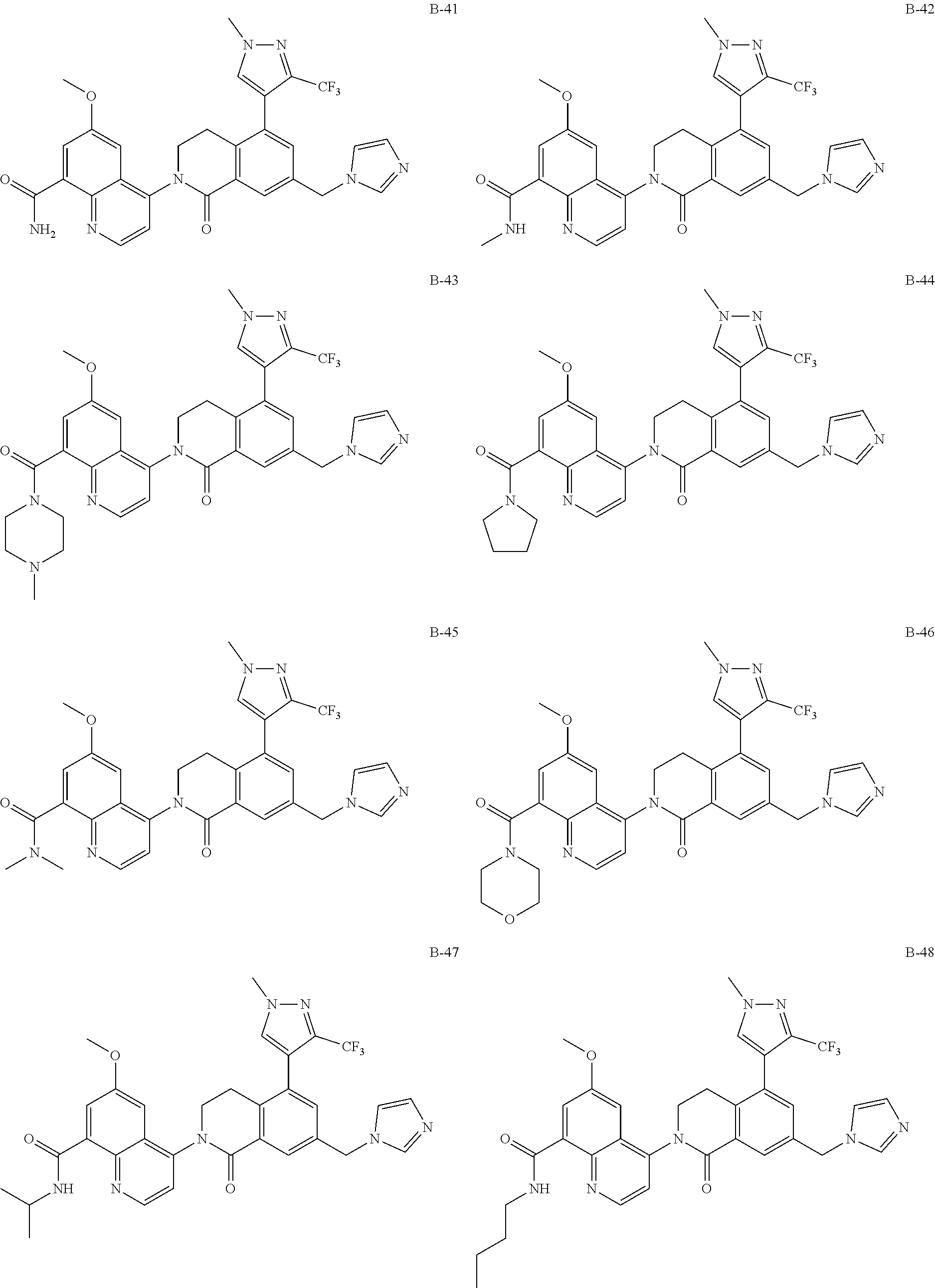

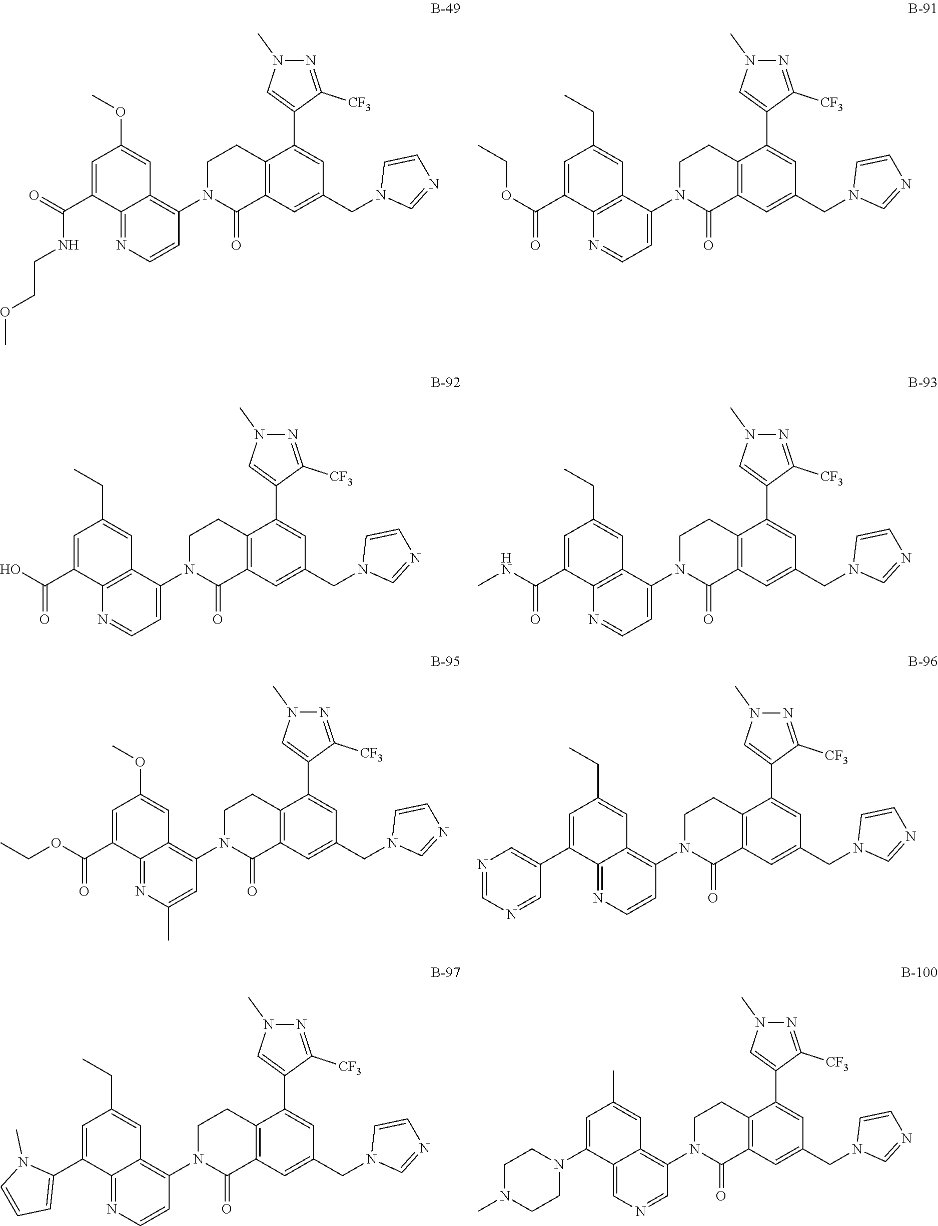

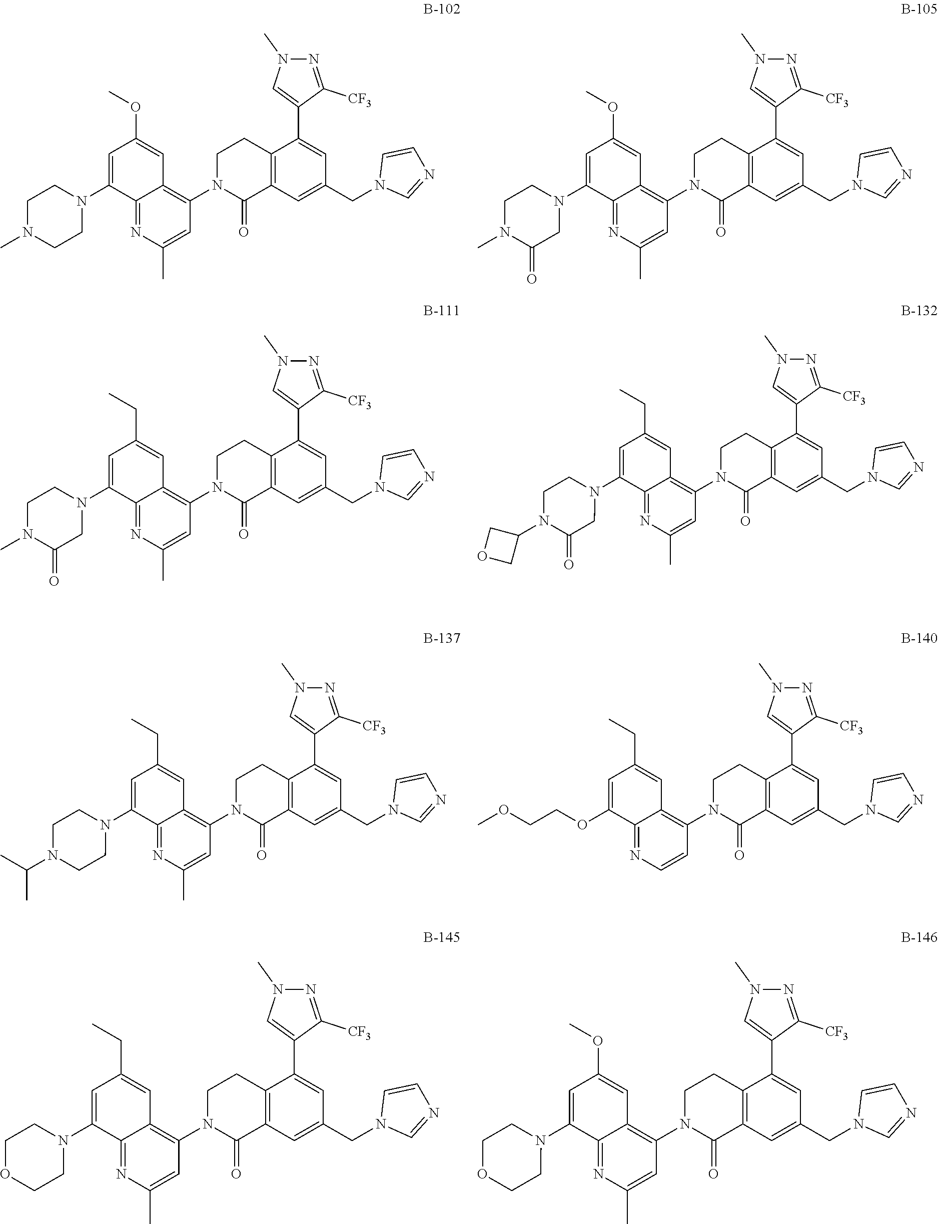

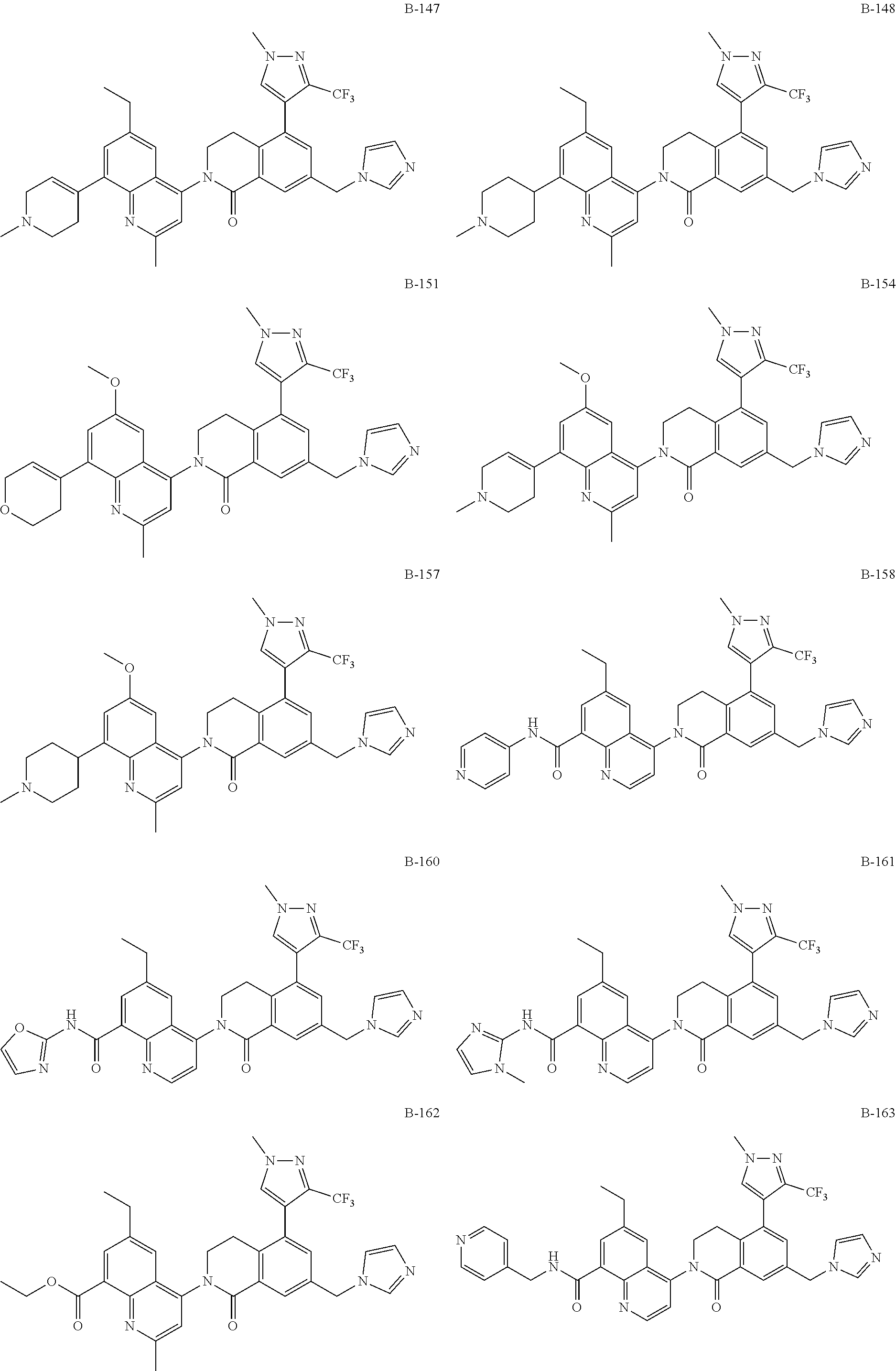

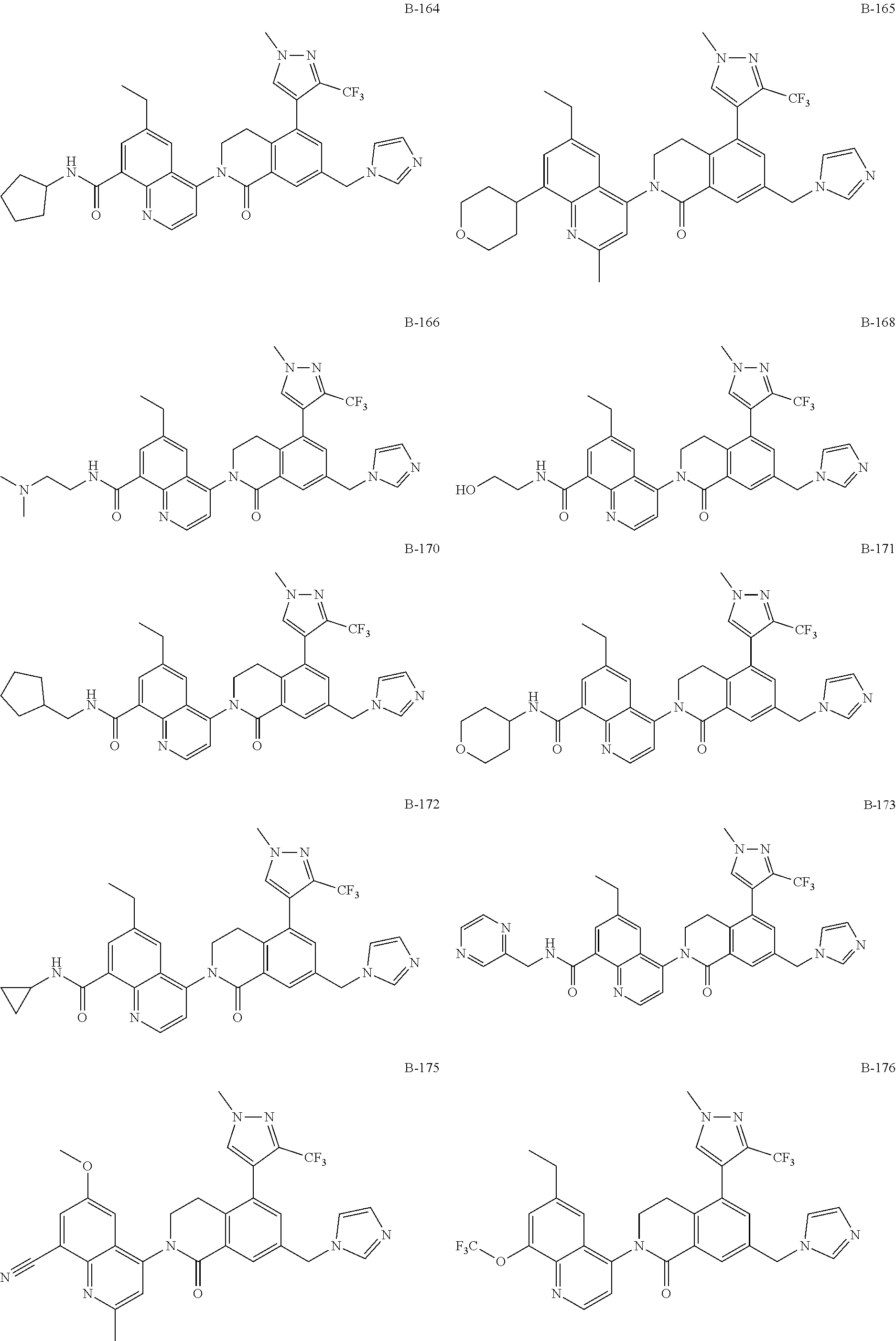

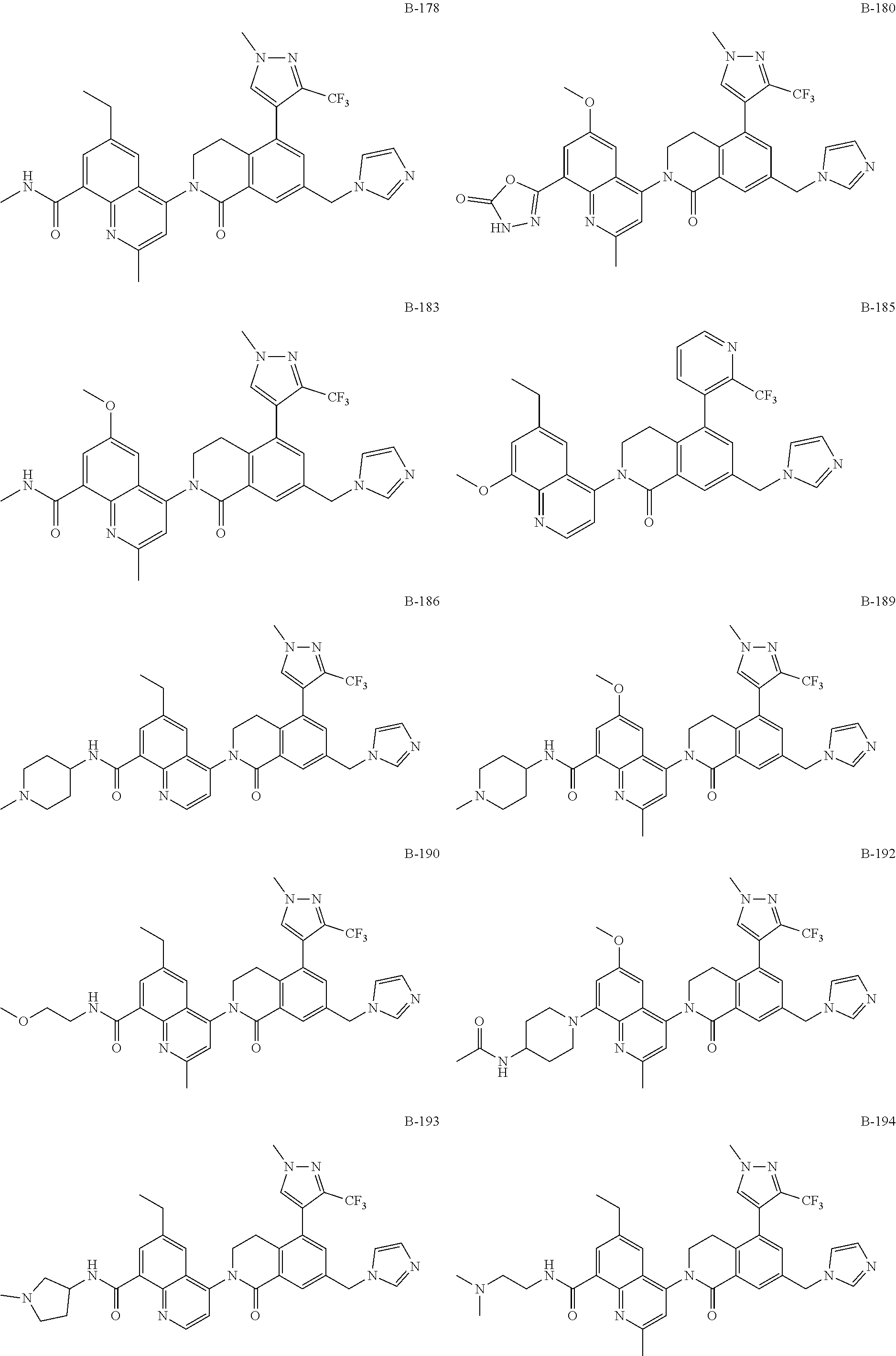

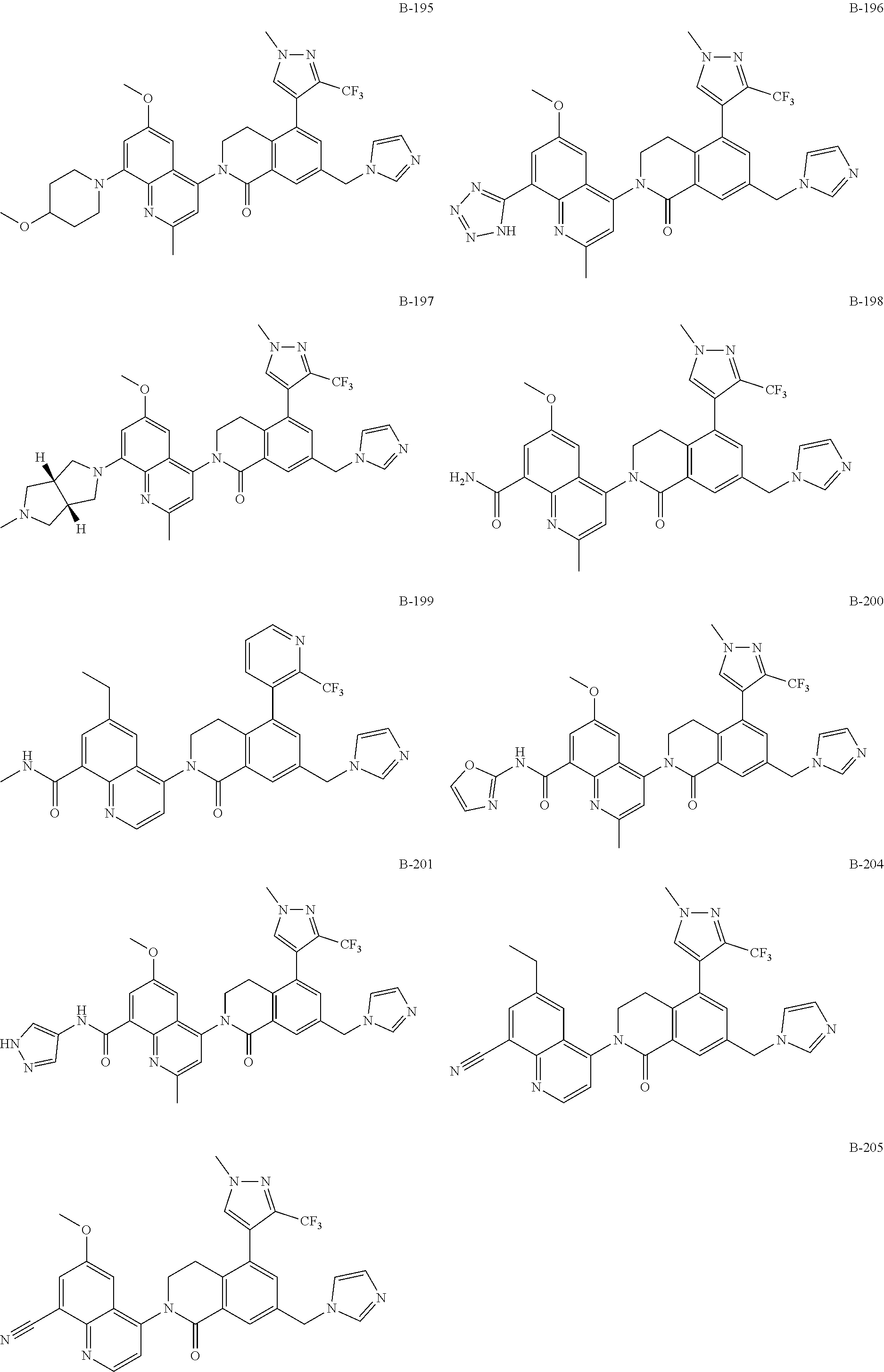

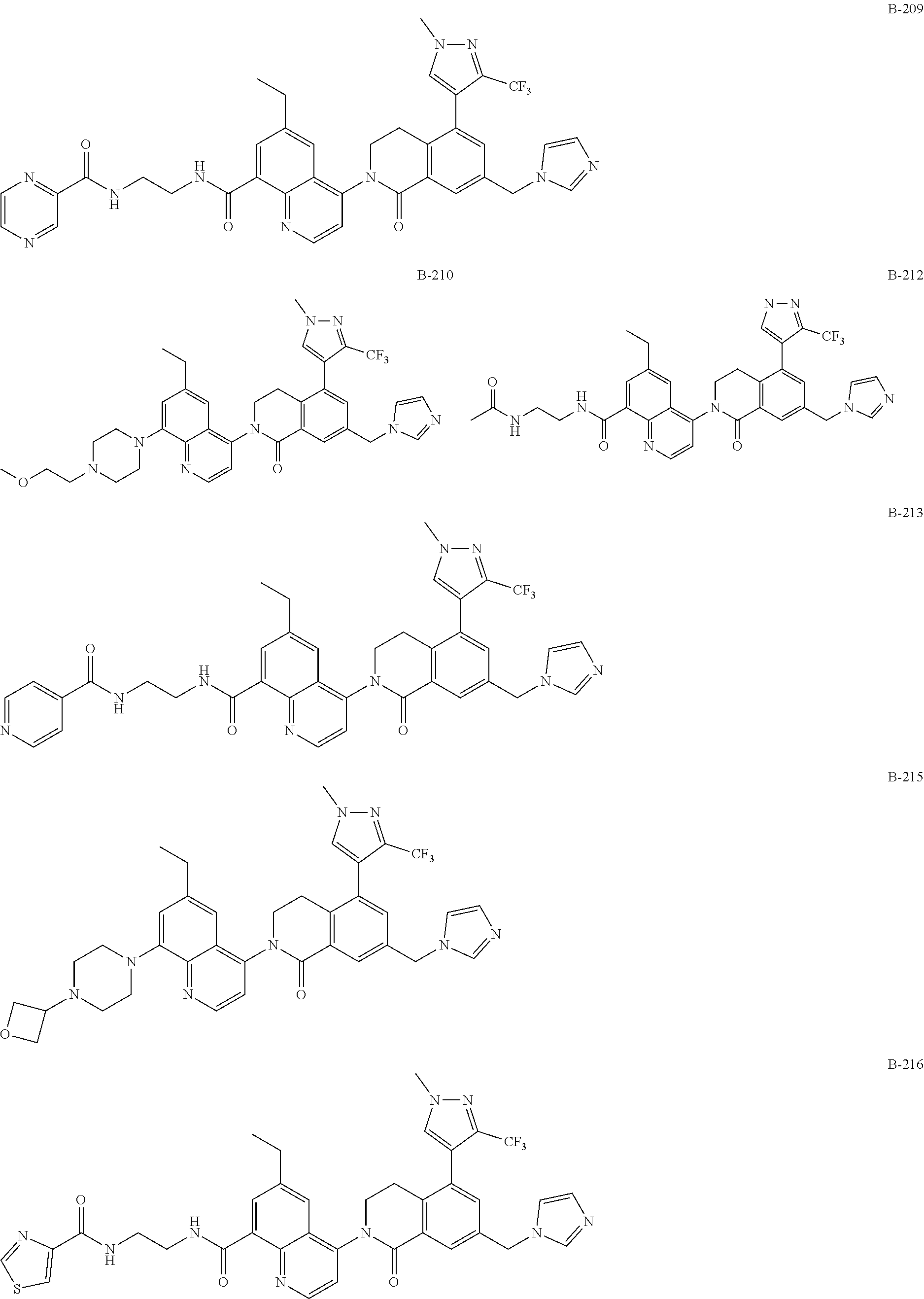

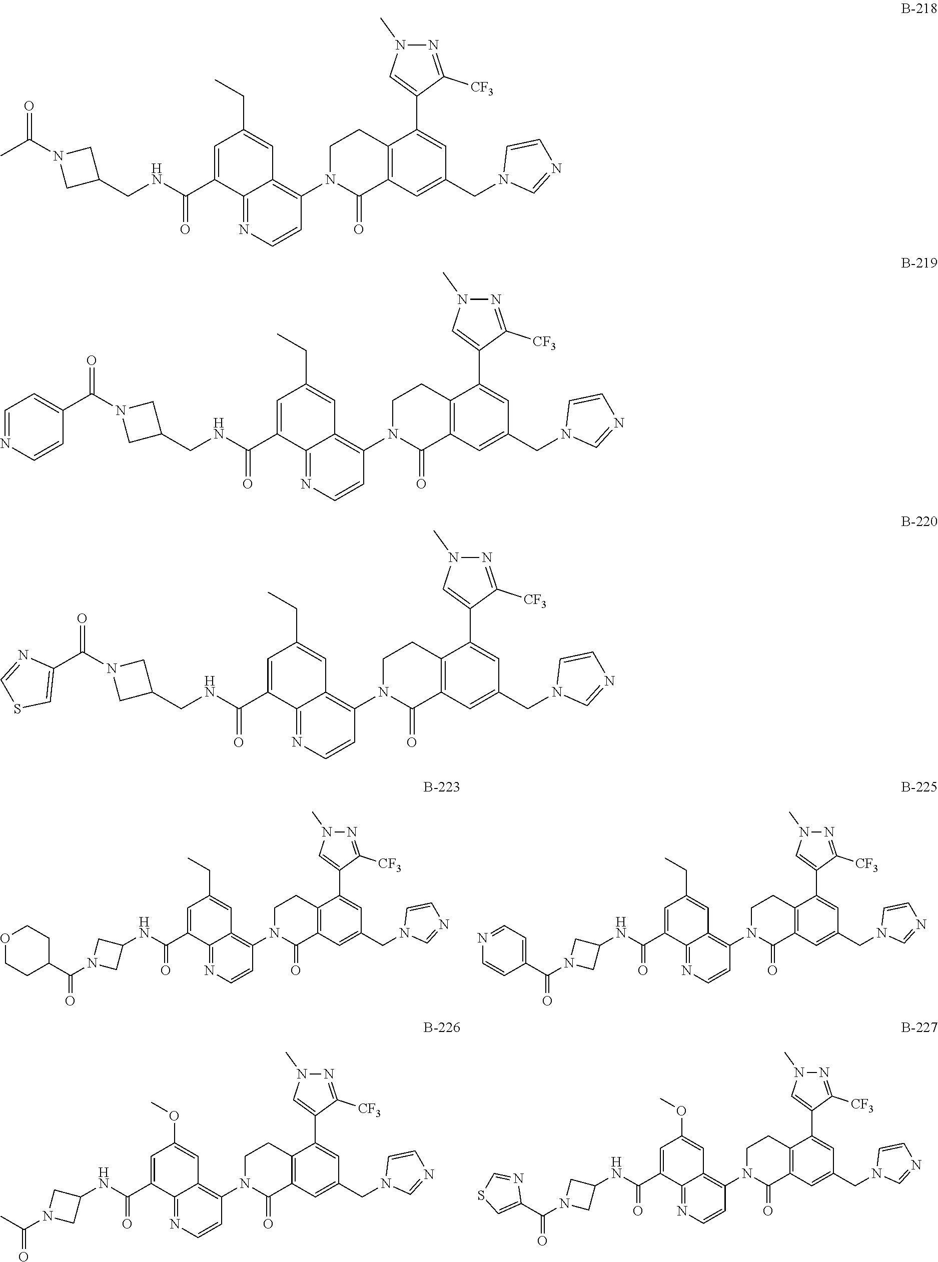

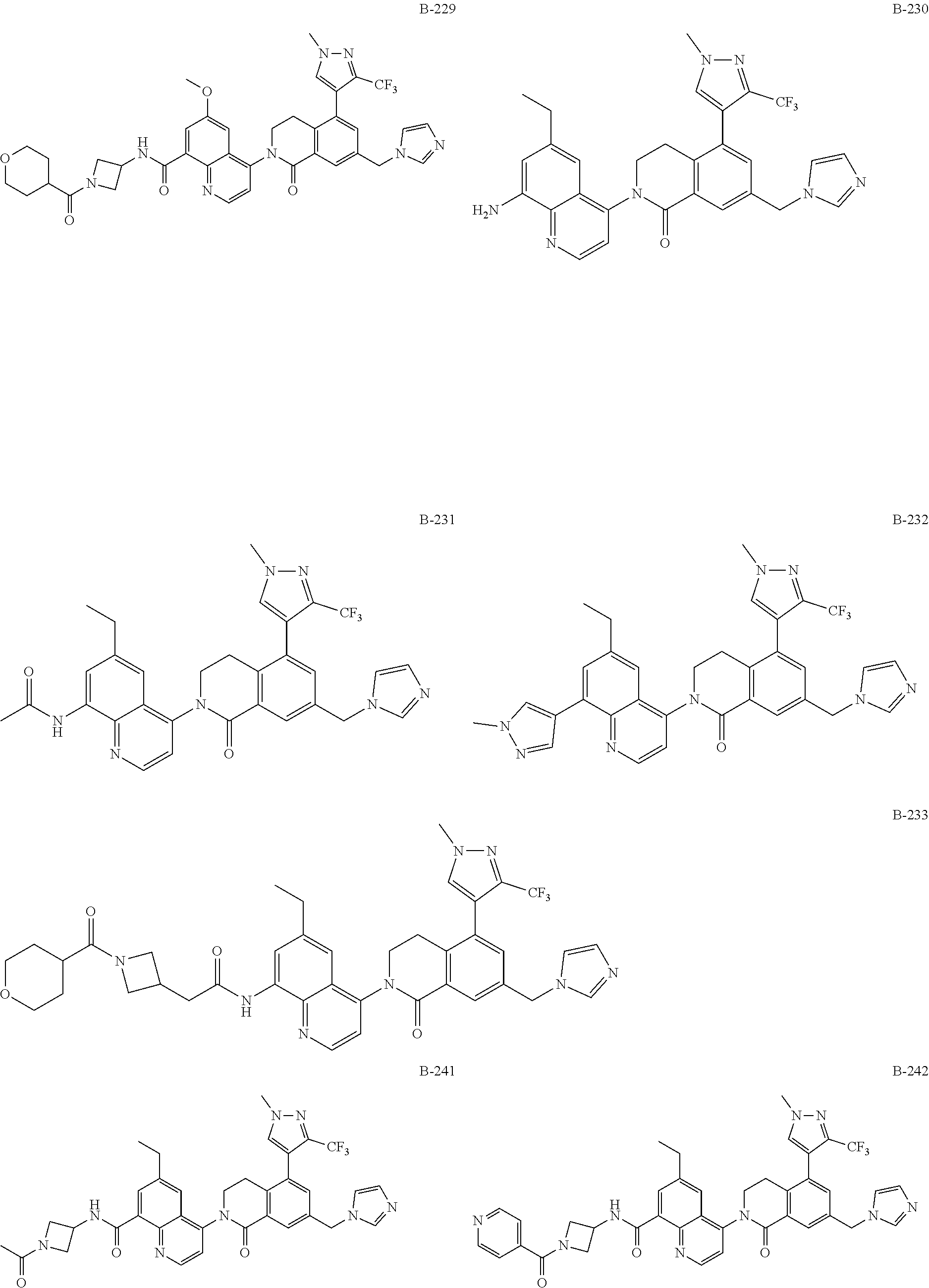

Kᵢ for additional WDR5 inhibitor compounds for Inhibition of WDR5 by TR-FRET assay.

| Compound | Ki (nM) |
|---|---|
| B-30 | <0.02 |
| B-31 | <0.02 |
| B-32 | 0.025 |
| B-33 | 0.022 |
| B-34 | 0.020 |
| B-35 | <0.02 |
| B-36 | <0.02 |
| B-37 | <0.02 |
| B-38 | <0.02 |
| B-40 | 0.062 |
| B-41 | 0.023 |
| B-42 | <0.02 |
| B-43 | 0.032 |
| B-44 | <0.02 |
| B-45 | 0.037 |
| B-46 | 0.038 |
| B-47 | 0.022 |
| B-48 | 0.028 |
| B-49 | <0.02 |
| B-91 | 0.023 |
| B-92 | 0.062 |
| B-93 | <0.02 |
| B-95 | <0.02 |
| B-96 | <0.02 |
| B-97 | 0.021 |
| B-100 | 0.026 |
| B-102 | 0.025 |
| B-105 | 0.022 |
| B-111 | 0.027 |
| B-132 | 0.030 |
| B-137 | 0.032 |
| B-140 | <0.02 |
| B-145 | 0.020 |
| B-146 | 0.025 |
| B-147 | 0.022 |
| B-148 | 0.028 |
| B-151 | <0.02 |
| B-154 | 0.022 |
| B-157 | <0.02 |
| B-158 | <0.02 |
| B-160 | <0.02 |
| B-161 | 0.026 |
| B-162 | 0.026 |
| B-163 | 0.024 |
| B-164 | 0.028 |
| B-165 | 0.042 |
| B-166 | 0.029 |
| B-168 | 0.028 |
| B-170 | 0.023 |
| B-171 | <0.02 |
| B-172 | <0.02 |
| B-173 | <0.02 |
| B-175 | <0.02 |
| B-176 | <0.02 |
| B-178 | <0.02 |
| B-180 | 0.023 |
| B-183 | <0.02 |
| B-185 | 0.028 |
| B-186 | <0.02 |
| B-189 | <0.02 |
| B-190 | <0.02 |
| B-192 | 0.021 |
| B-193 | <0.02 |
| B-194 | <0.02 |
| B-195 | <0.02 |
| B-196 | 0.366 |
| B-197 | 0.020 |
| B-198 | 0.023 |
| B-199 | 0.035 |
| B-200 | <0.02 |
| B-201 | 0.023 |
| B-204 | <0.02 |
| B-205 | <0.02 |
| B-209 | <0.02 |
| B-210 | <0.02 |
| B-212 | <0.02 |
| B-213 | <0.02 |
| B-215 | <0.02 |
| B-216 | <0.02 |
| B-218 | <0.02 |
| B-219 | <0.02 |
| B-220 | <0.02 |
| B-223 | <0.02 |
| B-225 | <0.02 |
| B-226 | <0.02 |
| B-227 | <0.02 |
| B-228 | <0.02 |
| B-229 | <0.02 |
| B-230 | <0.02 |
| B-231 | <0.02 |
| B-232 | <0.02 |
| B-233 | <0.02 |
| B-241 | <0.02 |
| B-242 | <0.02 |

### Cellular Viability of Human Tumor Cell Lines

Anti-proliferative activity using MLL-harboring cell lines. MV-4-11 and K562 cells are grown in IMDM media supplemented with 10% FBS and 1% penicillin/streptomycin, Molm-13 cells are cultured in RPMI-1640 media supplemented with 10% FBS and 1% penicillin/streptomycin. Viability assays are performed by dispensing 200 cells at 7200 cells/mL into each well of an opaque 384-well plate and adding compounds at the indicated concentrations in a final volume of 32 μL and a final concentration of DMSO of 0.3% for all samples. A certain range of compound concentrations is made through a series of 2-fold dilutions starting 30 μM at the highest, total 22 dilutions. After a set incubation period, 5 day protocol, the viability of cells in each well is assessed using the CellTiter-Glo assay (Promega), read on a 96 Microplane Luminometer (Cytation 3, BioTek). Serial dilutions of each cell type are performed in all assays to generate standard curves and the final densities of cells are determined within the dynamic range of the instrument. GI₅₀ values are calculated based on XLfit software (IDBS, Guildford, UK) with Sigmoidal Dose-Response Model. Each compound is tested in minimum of two replicates. Data are expressed as mean.

TABLE 3
GI₅₀ (in nM) for representative compounds on cellular
proliferation of MV4:11 human cancer cell lines

| Example | GI₅₀ (nM) |
|---|---|
| 2 | <20 |
| 5 | <20 |
| 6 | <20 |
| 7 | 1988 |
| 9 | 102 |
| 10 | <20 |
| 11 | <20 |
| 12 | 42 |
| 13 | <20 |
| 14 | <20 |
| 15 | <20 |
| 16 | <20 |
| 17 | 362 |
| 20 | 290 |
| 21 | <20 |
| 22 | <20 |
| 23 | 1280 |
| 24 | 25 |
| 25 | 310 |
| 26 | 76 |
| 27 | <20 |
| 28 | 64 |
| 29 | 164 |
| 30 | 47 |
| 31 | <20 |
| 32 | <20 |
| 33 | <20 |
| 34 | 75 |
| 35 | 47 |
| 36 | <20 |
| 37 | 96 |
| 39 | <20 |
| 43 | <20 |
| 44 | <20 |
| 45 | 21 |
| 46 | <20 |
| 47 | 296 |
| 48 | <20 |
| 49 | <20 |
| 50 | <20 |
| 51 | <20 |
| 52 | 80 |
| 53 | <20 |
| 54 | 81 |
| 55 | 47 |
| 56 | <20 |
| 57 | 24 |
| 58 | 195 |
| 59 | 90 |
| 60 | 159 |
| 61 | 128 |
| 62 | 29 |
| 63 | <20 |
| 64 | <20 |
| 65 | 43 |
| 66 | 38 |
| 67 | <20 |

GI₅₀ (in nM) for additional WDR5 inhibitor compounds on cellular proliferation of MV4; 11 human cancer cell lines.

| Compound | GI₅₀ (nM) |
|---|---|
| B-30 | <20 |
| B-31 | <20 |
| B-32 | <20 |
| B-33 | <20 |
| B-34 | <20 |
| B-35 | 76 |
| B-36 | 23 |
| B-37 | <20 |
| B-38 | 31 |
| B-40 | 291 |
| B-41 | 24 |
| B-42 | <20 |
| B-43 | 563 |
| B-44 | 353 |
| B-45 | 326 |
| B-46 | 444 |
| B-47 | 54 |
| B-48 | 119 |
| B-49 | 34 |
| B-91 | <20 |
| B-92 | 388 |
| B-93 | <20 |
| B-95 | 23 |
| B-96 | <20 |
| B-97 | 83 |
| B-100 | <20 |
| B-102 | <20 |
| B-105 | <20 |
| B-111 | <20 |
| B-132 | <20 |
| B-137 | <20 |
| B-140 | <20 |
| B-145 | 36 |
| B-146 | 26 |
| B-147 | 43 |
| B-148 | <20 |
| B-151 | 55 |
| B-154 | 33 |
| B-157 | <20 |
| B-158 | 31 |
| B-160 | <20 |
| B-161 | 59 |
| B-162 | 88 |
| B-163 | 56 |
| B-164 | 155 |
| B-165 | 129 |
| B-166 | <20 |
| B-168 | 46 |
| B-170 | 212 |
| B-171 | <20 |
| B-172 | <20 |
| B-173 | <20 |
| B-175 | <20 |
| B-176 | 33 |
| B-178 | <20 |
| B-180 | 68 |
| B-183 | <20 |
| B-185 | 55 |
| B-186 | <20 |
| B-189 | <20 |
| B-190 | <20 |
| B-192 | <20 |
| B-193 | <20 |
| B-194 | <20 |
| B-195 | <20 |
| B-196 | >10000 |
| B-197 | <20 |
| B-198 | <20 |
| B-199 | 131 |
| B-200 | 28 |
| B-201 | 23 |
| B-204 | 22 |
| B-205 | <20 |
| B-209 | <20 |
| B-210 | <20 |
| B-212 | 35 |
| B-213 | <20 |
| B-215 | <20 |
| B-216 | <20 |
| B-218 | 21 |
| B-219 | <20 |
| B-220 | <20 |
| B-223 | <20 |
| B-225 | <20 |
| B-226 | 43 |
| B-227 | <20 |
| B-228 | <20 |
| B-229 | 26 |
| B-230 | 54 |
| B-231 | <20 |
| B-232 | 38 |
| B-233 | 26 |
| B-241 | <20 |
| B-242 | 29 |

It is understood that the foregoing detailed description and accompanying examples are merely illustrative and are not to be taken as limitations upon the scope of the invention. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A compound of formula (I) 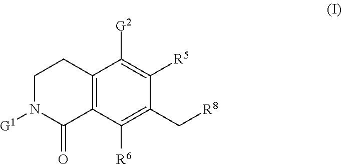
or a tautomer thereof or a pharmaceutically acceptable salt of the compound or tautomer, wherein:
G¹ is 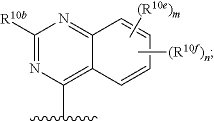
R^{10b} is C₁₋₃alkyl, a 4- to 8-membered monocyclic heterocyclyl, hydrogen, fluoro, chloro, C₁₋₃fluoroalkyl, C₃₋₆cycloalkyl, NH₂, —NHC₁₋₄alkyl, or —N(C₁₋₄alkyl)₂, wherein the heterocyclyl contains 1-2 heteroatoms independently selected from the group consisting of O, N, and S, and is optionally substituted with 1-4 substituents independently selected from the group consisting of halogen, cyano, oxo, C₁₋₄alkyl, C₁₋₄fluoroalkyl, OH, —OC₁₋₄alkyl, —OC₁₋₄fluoroalkyl, NH₂, —NHC₁₋₄alkyl, and —N(C₁₋₄alkyl)₂;
R^{10e} is —OC₁₋₄alkyl, halogen, cyano, C₁₋₄alkyl, C₁₋₄fluoroalkyl, OH, —OC₁₋₄fluoroalkyl, —OG^{1c}, —OC₁₋₃alkylene-G^{1c}, —O—C₂₋₃alkylene-Y, NH₂, —NHC₁₋₄alkyl, —N(C₁₋₄alkyl)₂, or a 4- to 8-membered monocyclic heterocyclyl containing 1-2 heteroatoms independently selected from the group consisting of N, O, and S, wherein the heterocyclyl is optionally substituted with 1-4 substituents independently selected from the group consisting of halogen, cyano, oxo, C₁₋₄alkyl, C₁₋₄fluoroalkyl, OH, —OC₁₋₄alkyl, —OC₁₋₄fluoroalkyl, NH₂, —NHC₁₋₄alkyl, and —N(C₁₋₄alkyl)₂;
R^{10f}, at each occurrence, is independently —OC₁₋₄alkyl, C₁₋₄alkyl, halogen, cyano, C₁₋₄fluoroalkyl, OH, —OC₁₋₄fluoroalkyl, —OC₃₋₄cycloalkyl, —OC₁₋₃alkylene-C₃₋₄cycloalkyl, —OPG, or —OSO₂CF₃;
m is 0 or 1;
n is 0, 1, or 2;
PG is a hydroxy protecting group;
Y is OH, —OC₁₋₄alkyl, —OC₁₋₄fluoroalkyl, —OC₃₋₄cycloalkyl, or —OC₁₋₃alkylene-C₃₋₄cycloalkyl;
G^{1c} is C₃₋₆cycloalkyl, a 4- to 7-membered monocyclic heterocyclyl containing 1-2 heteroatoms independently selected from O, N, and S, or a 5- to 6-membered heteroaryl containing 1-3 heteroatoms independently selected from O, N, and S, wherein G^{1c} is optionally substituted with 1-4 substituents independently selected from the group consisting of halogen, cyano, C₁₋₄alkyl, C₁₋₄fluoroalkyl, oxo, —OH, —OC₁₋₄alkyl, C₃₋₄cycloalkyl, and —C₁₋₃alkylene-C₃₋₄cycloalkyl;
G² is a 5- to 6-membered heteroaryl or phenyl, wherein G² is optionally substituted with 1-4 substituents independently selected from the group consisting of C₁₋₄alkyl, C₁₋₄fluoroalkyl, halogen, —OR^{4c}, —N(R⁴)₂, cyano, —C(O)OR^{4c}, —C(O)N(R^{4c})₂, —C(O)R^{4c}, —SO₂R^{4d}, —SO₂N(R^{4c})₂, —NR^{4c}C(O)R^{4c}, C₃₋₈cycloalkyl, and —C₁₋₃alkylene-C₃₋₈-cycloalkyl, wherein each C₃₋₈cycloalkyl is optionally substituted with 1-4 substituents independently selected from the group consisting of C₁₋₄alkyl and halogen;
R^{4c}, at each occurrence, is independently hydrogen, C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen, wherein alternatively two R^{4c}, together with a common nitrogen atom to which the R^{4c} attach form a 4- to 8-membered saturated or partially unsaturated heterocyclic ring, optionally substituted with 1-4 substituents independently selected from the group consisting of C₁₋₄alkyl, C₁₋₄fluoroalkyl, oxo, —OH, and —OC₁₋₄alkyl;
R^{4d}, at each occurrence, is independently C₁₋₄alkyl, C₁₋₄fluoroalkyl, C₃₋₈cycloalkyl, or —C₁₋₆alkylene-C₃₋₈cycloalkyl, wherein each C₃₋₈cycloalkyl is optionally substituted with 1-4 substituents independently selected from the group consisting of C₁₋₄alkyl and halogen;
R⁵ and R⁶ are each independently hydrogen, halogen, C₁₋₄alkyl, C₁₋₄fluoroalkyl, or —OC₁₋₄alkyl; and
R⁸ is an imidazolyl unsubstituted or substituted with 1-3 substituents independently selected from the group consisting of halogen, C₁₋₄alkyl, C₁₋₄fluoroalkyl, NO₂, NH₂, —NH(C₁₋₄alkyl), —N(C₁₋₄alkyl)₂, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of halogen, C₁₋₄alkyl, C₁₋₄fluoroalkyl, OH, and —OC₁₋₄alkyl;
provided the compound is not:
7-((1H-imidazol-1-yl)methyl)-2-(6-methoxyquinazolin-4-yl)-5-(1-methyl-3-(trifluoromethyl)-1H-pyrazol-4-yl)-3,4-dihydroisoquinolin-1(2H)-one.

2. The compound of claim 1, or a tautomer thereof or a pharmaceutically acceptable salt of the compound or tautomer, wherein:
R^{10b} is C₁₋₃alkyl, hydrogen, fluoro, chloro, or C₁₋₃fluoroalkyl;
R^{10c} is halogen, cyano, C₁₋₄alkyl, C₁₋₄fluoroalkyl, OH, —OC₁₋₄alkyl, —OC₁₋₄fluoroalkyl, —OG^{1c}, —OC₁₋₃alkylene-G^{1c}, or —O—C₂₋₃alkylene-Y; and
G² is a 5- to 6-membered heteroaryl, wherein G² is optionally substituted with 1-4 substituents independently selected from the group consisting of C₁₋₄alkyl, C₁₋₄fluoroalkyl, halogen, —OR^{4c}, —N(R^{4c})₂, cyano, —C(O)OR^{4c}, —C(O)N(R^{4c})₂, —C(O)R^{4c}, —SO₂R^{4d}, —SO₂N(R^{4c})₂, —NR^{4c}C(O)R^{4c}, C₃₋₈cycloalkyl, and —C₁₋₃alkylene-C₃₋₈cycloalkyl, wherein each C₃₋₈cycloalkyl is optionally substituted with 1-4 substituents independently selected from the group consisting of C₁₋₄alkyl and halogen.

3. The compound of claim 1 or 2, or a tautomer thereof or a pharmaceutically acceptable salt of the compound or tautomer, wherein
R⁸ is 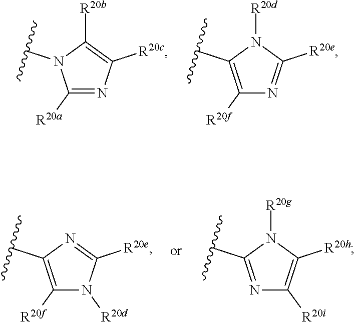
R^{20a} is hydrogen, C₁₋₄alkyl, NH₂, —NH(C₁₋₄alkyl), —N(C₁₋₄alkyl)₂, or C₃₋₈cycloalkyl; and
R^{20b}, R^{20c}, R^{20d}, R^{20e}, R^{20f}, R^{20g}, R^{20h}, and R²⁰ⁱ are each independently hydrogen, C₁₋₄alkyl, or C₃₋₈cycloalkyl.

4. The compound of claim 3, or a tautomer thereof or a pharmaceutically acceptable salt of the compound or tautomer, wherein R⁸ is

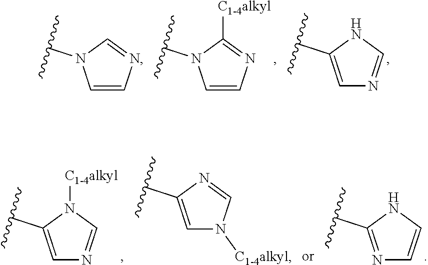

5. The compound of any of claims 1-4, or a tautomer thereof or a pharmaceutically acceptable salt of the compound or tautomer, wherein G¹ is

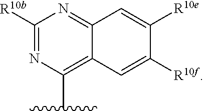

6. The compound of any of claims 1-4, or a tautomer thereof or a pharmaceutically acceptable salt of the compound or tautomer, wherein G¹ is

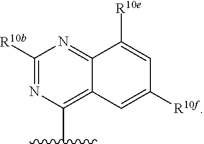

7. The compound of any of claims 1-6, or a tautomer thereof or a pharmaceutically acceptable salt of the compound or tautomer, wherein R^{10b} is C₁₋₃alkyl.

8. The compound of any of claims 1-6, or a tautomer thereof or a pharmaceutically acceptable salt of the compound or tautomer, wherein R^{10b} is the optionally substituted 4- to 8-membered monocyclic heterocyclyl.

9. The compound of any of claims 1-6 or 8, or a tautomer thereof or a pharmaceutically acceptable salt of the compound or tautomer, wherein the ring system of the optionally substituted 4- to 8-membered monocyclic heterocyclyl contains at least one ring nitrogen atom and is bonded to the parent molecular moiety at the at least one ring nitrogen atom.

10. The compound of claim 9, or a tautomer thereof or a pharmaceutically acceptable salt of the compound or tautomer, wherein R^{10b} is

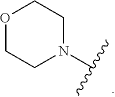 [SMILES: *N1CCOCC1]

11. The compound of any of claims 1-6 or 8, or a tautomer thereof or a pharmaceutically acceptable salt of the compound or tautomer, wherein the optionally substituted 4- to 8-membered monocyclic heterocyclyl is bonded to the parent molecular moiety at a ring carbon atom.

12. The compound of claim 11, or a tautomer thereof or a pharmaceutically acceptable salt of the compound or tautomer, wherein the ring system of the optionally substituted 4- to 8-membered monocyclic heterocyclyl contains one ring heteroatom that is oxygen.

13. The compound of claim 12, or a tautomer thereof or a pharmaceutically acceptable salt of the compound or tautomer, wherein R^{10b} is

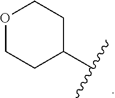 [SMILES: *C1CCOCC1]

14. The compound of any of claims 1-13, or a tautomer thereof or a pharmaceutically acceptable salt of the compound or tautomer, wherein
(a) R^{10e} is —OC₁₋₄alkyl, halogen, OH, —OC₁₋₃alkylene-G^{1c}, or —O—C₂₋₃alkylene-Y; or
(b) R^{10e} is NH₂, —NHC₁₋₄alkyl, —N(C₁₋₄alkyl)₂, or a 4- to 8-membered monocyclic heterocyclyl containing 1-2 heteroatoms independently selected from the group consisting of N, O, and S, wherein the heterocyclyl is optionally substituted with 1-4 substituents independently selected from the group consisting of halogen, cyano, oxo, C₁₋₄alkyl, C₁₋₄fluoroalkyl, OH, OC₁₋₄alkyl, OC₁₋₄fluoroalkyl, NH₂, —NHC₁₋₄alkyl, and —N(C₁₋₄alkyl)₂.

15. The compound of claim 14, or a tautomer thereof or a pharmaceutically acceptable salt of the compound or tautomer, wherein R^{10e} is —OC₁₋₄alkyl.

16. The compound of any of claims 1-15, or a tautomer thereof or a pharmaceutically acceptable salt of the compound or tautomer, wherein R^{10f}, at each occurrence, is independently —OC₁₋₄alkyl, C₁₋₄alkyl, OH, —OPG, or —OSO₂CF₃.

17. The compound of claim 16, or a tautomer thereof or a pharmaceutically acceptable salt of the compound or tautomer, wherein R^{10f} is C₁₋₄alkyl or —OC₁₋₄alkyl.

18. The compound of any of claims 1-5, or a tautomer thereof or a pharmaceutically acceptable salt of the compound or tautomer, wherein G¹ is

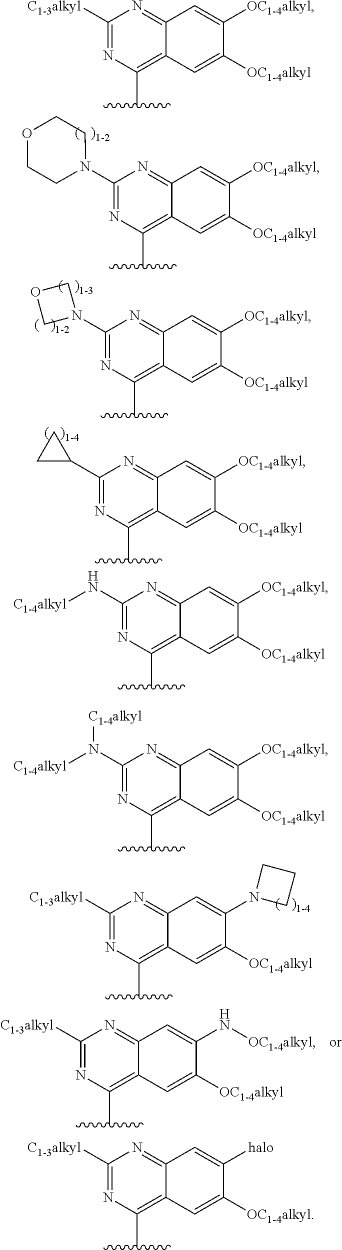

19. The compound of any of claims 1-5, or a tautomer thereof or a pharmaceutically acceptable salt of the compound or tautomer, wherein G¹ is

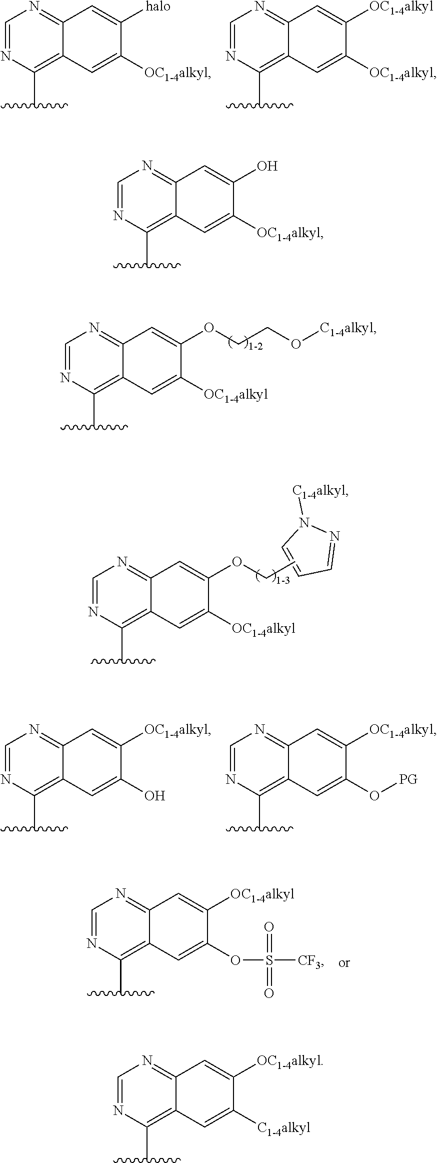

20. The compound of any of claims 1-4 or 6, or a tautomer thereof or a pharmaceutically acceptable salt of the compound or tautomer, wherein G¹ is

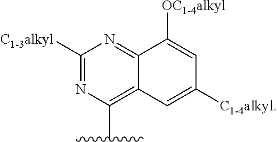

21. The compound of any of claims 1-4 or 6, or a tautomer thereof or a pharmaceutically acceptable salt of the compound or tautomer, wherein G¹ is

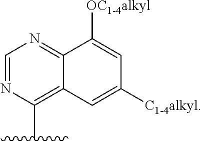

22. The compound of any of claims 1-21, or a tautomer thereof or a pharmaceutically acceptable salt of the compound or tautomer, wherein G² is the optionally substituted 5- to 6-membered heteroaryl.

23. The compound of any of claims 1-22, or a tautomer thereof or a pharmaceutically acceptable salt of the compound or tautomer, wherein the ring system of the optionally substituted 5- to 6-membered heteroaryl at G² is pyrazolyl.

24. The compound of any of claims 1-23, or a tautomer thereof or a pharmaceutically acceptable salt of the compound or tautomer, wherein G² is optionally substituted with 1-4 substituents independently selected from the group consisting of halogen, C₁₋₄alkyl, and C₁₋₄fluoroalkyl.

25. The compound of claim 24, or a tautomer thereof or a pharmaceutically acceptable salt of the compound or tautomer, wherein, G² is

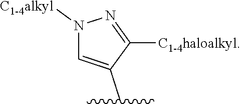

26. The compound of any of claims 1-25, or a tautomer thereof or a pharmaceutically acceptable salt of the compound or tautomer, wherein R⁵ is hydrogen.

27. The compound of any of claims 1-26, or a tautomer thereof or a pharmaceutically acceptable salt of the compound or tautomer, wherein R⁶ is hydrogen.

28. A compound selected from the group consisting of:
7-((1H-imidazol-1-yl)methyl)-2-(6,7-dimethoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(7-hydroxy-6-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxy-7-(2-methoxyethoxy)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxy-7-((1-methyl-1H-pyrazol-5-yl)methoxy)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-methoxyquinazolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(6-ethoxy-7-methoxyquinazolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(7-ethoxy-6-methoxyquinazolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(6,7-diethoxyquinazolin-4-yl)-5-(1-methyl-3-(trifluoromethyl)-1H-pyrazol-4-yl)-3,4-dihydroisoquinolin-(2H)-one;
7-((1H-imidazol-1-yl)methyl)-2-(7-fluoro-6-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-(benzyloxy)-7-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-hydroxy-7-methoxyquinazolin-4-yl)-5-(1-methyl-3-(trifluoromethyl)-1H-pyrazol-4-yl)-3,4-dihydroisoquinolin-1(2H)-one;
4-(7-((1H-imidazol-1-yl)methyl)-5-(1-methyl-3-(trifluoromethyl)-1H-pyrazol-4-yl)-1-oxo-3,4-dihydroisoquinolin-2(1H)-yl)-7-methoxyquinazolin-6-yl trifluoromethanesulfonate;
7-((1H-imidazol-1-yl)methyl)-2-(6-ethyl-7-methoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,8-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-ethyl-8-methoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(7-fluoro-6-methoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(7-(azetidin-1-yl)-6-methoxy-2-methylquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6-methoxy-2-methyl-7-(methylamino)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-morpholinoquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-(methylamino)quinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-methylquinazolin-4-yl)-5-(1-ethyl-3-methyl-1H-pyrazol-5-yl)-3,4-dihydroisoquinolin-1(2H)-one;
7-((1H-imidazol-1-yl)methyl)-2-(6,7-dimethoxy-2-methylquinazolin-4-yl)-5-(1,3-dimethyl-1H-pyrazol-5-yl)-3,4-dihydroisoquinolin-1(2H)-one;
7-((1H-imidazol-1-yl)methyl)-2-(6,7-dimethoxy-2-methylquinazolin-4-yl)-5-(1-methyl-3-(trifluoromethyl)-1H-pyrazol-5-yl)-3,4-dihydroisoquinolin-1(2H)-one;
7-((1H-imidazol-1-yl)methyl)-2-(2-(ethyl(methyl)amino)-6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(2-ethyl-6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(2-cyclopropyl-6,7-dimethoxyquinazolin-4-yl)-5-(1-methyl-3-(trifluoromethyl)-1H-pyrazol-4-yl)-3,4-dihydroisoquinolin-1(2H)-one;
7-((1H-imidazol-1-yl)methyl)-2-(6,7-dimethoxy-2-methylquinazolin-4-yl)-5-(1,3-dimethyl-1H-pyrazol-4-yl)-3,4-dihydroisoquinolin-1(2H)-one;
7-((1H-imidazol-1-yl)methyl)-2-(6,7-dimethoxy-2-methylquinazolin-4-yl)-5-(4-fluoro-2-methylphenyl)-3,4-dihydroisoquinolin-1(2H)-one;
7-((1H-imidazol-1-yl)methyl)-2-(6,7-dimethoxy-2-(tetrahydro-2H-pyran-4-yl)quinazolin-4-yl)-5-(1-methyl-3-(trifluoromethyl)-1H-pyrazol-4-yl)-3,4-dihydroisoquinolin-1(2H)-one;
or a pharmaceutically acceptable salt thereof.

29. A pharmaceutical composition comprising the compound of any of claims 1-28, or a tautomer thereof or a pharmaceutically acceptable salt of the compound or tautomer, and a pharmaceutically acceptable carrier.

30. The compound of any of claims 1-28, or a tautomer thereof or a pharmaceutically acceptable salt of the compound or tautomer, or the pharmaceutical composition of claim 29, for use in the treatment of cancer.

31. The compound of any of claims 1-28, or a tautomer thereof or a pharmaceutically acceptable salt of the compound or tautomer, or the pharmaceutical composition of claim 29, for use in the inhibition of cancer cell proliferation.

32. A method of treating cancer comprising administering to a subject in need thereof, a therapeutically effective amount of the compound of any of claims 1-28, or a tautomer thereof or a pharmaceutically acceptable salt of the compound or tautomer, or the pharmaceutical composition of claim 29.

33. A method of inhibiting cancer cell proliferation, comprising administering to a subject in need thereof, the compound of any of claims 1-28, or a tautomer thereof or a pharmaceutically acceptable salt of the compound or tautomer, or the pharmaceutical composition of claim 29, in an amount effective to inhibit the cancer cell proliferation.

34. Use of the compound of any of claims 1-28, or a tautomer thereof or a pharmaceutically acceptable salt of the compound or tautomer, or the pharmaceutical composition of claim 29, in the manufacture of a medicament for the treatment of cancer.

35. Use of the compound of any of claims 1-28, or a tautomer thereof or a pharmaceutically acceptable salt of the compound or tautomer, or the pharmaceutical composition of claim 29, in the manufacture of a medicament for the inhibition of cancer cell proliferation.
